SEQUENCE LISTING (231 sequences)

SEQ ID NO: 1 - DNA, 15 bp, Artificial Sequence
  Synthetic Polynucleotide
tggcggggaagtcag

SEQ ID NO: 2 - DNA, 17 bp, Artificial Sequence
  Synthetic Polynucleotide
cggaggagaaatcgggc

SEQ ID NO: 3 - DNA, 23 bp, Artificial Sequence
  Synthetic Polynucleotide
ttggtcatgatactgctgattgc

SEQ ID NO: 4 - DNA, 22 bp, Artificial Sequence
  Synthetic Polynucleotide
ccctccacgaagtctctattgc

SEQ ID NO: 5 - DNA, 20 bp, Artificial Sequenc
  Synthetic Polynucleotide
gacacgtggagctctatcca

SEQ ID NO: 6 - DNA, 20 bp, Artificial Sequence
  Synthetic Polynucleotide
ggaaggttggagacaatgct

SEQ ID NO: 7 - DNA, 21 bp, Artificial Sequence
  Synthetic Polynucleotide
tcccaaatctaaatctgaccg

SEQ ID NO: 8 - DNA, 21 bp, Artificial Sequence
  Synthetic Polynucleotide
ctctaaagccacctctttctc

SEQ ID NO: 9 - DNA, 19 bp, Artificial Sequence
  Synthetic Polynucleotide
agggaccctgctctgaaac

SEQ ID NO: 10 - DNA, 20 bp, Artificial Sequence
  Synthetic Polynucleotide
tccttgtccatctccaggat

SEQ ID NO: 11 - DNA, 18 bp, Artificial Sequence
  Synthetic Polynucleotide
ccgcaggtatgcacgcgt

SEQ ID NO: 12 - DNA, 58 bp, Artificial Sequence
  Synthetic Polynucleotide
ccgggcaagtatgttcactatgaaactcgagtttcatagtgaacatacttgctttttg

SEQ ID NO: 13 - DNA, 58 bp, Artificial Sequence
  Synthetic Polynucleotide
ccgggccaaggaacaatccattgttctcgagaacaatggattgttccttggctttttg

SEQ ID NO: 14 - DNA, 57 bp, Artificial Sequence
  Synthetic Polynucleotide
ccggccatgtacttacaagccgatactcgagtatcggcttgtaagtacatggttttt

SEQ ID NO: 15 - DNA, 57 bp, Artificial Sequence
  Synthetic Polynucleotide
ccgggccgtggacgagaaagaaatactcgagtatttctttctcgtccacggcttttt

SEQ ID NO: 16 - DNA, 58 bp, Artificial Sequence
  Synthetic Polynucleotide
ccgggaaaggctgttggcatcaatactcgagtattgatgccaacagcctttctttttg

SEQ ID NO: 17 - DNA, 58 bp, Artificial Sequence
  Synthetic Polynucleotide
ccggccacccagctatgcttcagatctcgagatctgaagcatagctgggtggtttttg

SEQ ID NO: 18 - DNA, 58 bp, Artificial Sequence
  Synthetic Polynucleotide
ccggcggttcacaacctcggtttagctcgagctaaaccgaggttgtgaaccgtttttg

SEQ ID NO: 19 - DNA, 58 bp, Artificial Sequence
  Synthetic Polynucleotide
ccggtcaccaaggagactgctatttctcgagaaatagcagtctccttggtgatttttg

SEQ ID NO: 20 - DNA, 58 bp, Artificial Sequence
  Synthetic Polynucleotide
ccggccctacctgtccagctattacctcgaggtaatagctggacaggtagggtttttg

SEQ ID NO: 21 - DNA, 58 bp, Artificial Sequence
  Synthetic Polynucleotide
ccggcccgaaagagtttgagtggaactcgagttccactcaaactctttcgggtttttg

SEQ ID NO: 22 - DNA, 58 bp, Artificial Sequence
  Synthetic Polynucleotide
ccgggctactctgaggacgatattcctcgaggaatatcgtcctcagagtagctttttg

SEQ ID NO: 23 - DNA, 58 bp, Artificial Sequence
  Synthetic Polynucleotide
ccggcggtccattaataactataacctcgaggttatagttattaatggaccgtttttg

SEQ ID NO: 24 - DNA, 58 bp, Artificial Sequence
  Synthetic Polynucleotide
ccggtaagtcaaagaagacgtattactcgagtaatacgtcttctttgacttatttttg

SEQ ID NO: 25 - DNA, 58 bp, Artificial Sequence
  Synthetic Polynucleotide
ccgggaagtctgttgtggactataactcgagttatagtccacaacagacttctttttg

SEQ ID NO: 26 - RNA, 5 nt, Artificial Sequence
  Synthetic Polynucleotide
gacug

SEQ ID NO: 27 - RNA, 7 nt, Artificial Sequence
  Synthetic Polynucleotide
aacggac

SEQ ID NO: 28 - RNA, 7 nt, Artificial Sequence
  Synthetic Polynucleotide
auugcgg

SEQ ID NO: 29 - RNA, 7 nt, Artificial Sequence
  Synthetic Polynucleotide
ucgggac

SEQ ID NO: 30 - RNA, 7 nt, Artificial Sequence
  Synthetic Polynucleotide
gaaccgg

SEQ ID NO: 31 - RNA, 6 nt, Artificial Sequence
  Synthetic Polynucleotide
gaaccu

SEQ ID NO: 32 - RNA, 5 nt, Artificial Sequence
  Synthetic Polynucleotide
uacgg

SEQ ID NO: 33 - RNA, 7 nt, Artificial Sequence
  Synthetic polynucleotide
ucgcaag

SEQ ID NO: 34 - RNA, 5 nt, Artificial Sequence
  Synthetic polynucleotide
ggacu

SEQ ID NO: 35 - RNA, 7 nt, Artificial Sequence
  Synthetic polynucleotide
agacuuc

SEQ ID NO: 36 - DNA, 25 bp, Artificial Sequence
  Synthetic polynucleotide
caccgtaggcacgggactatcacta

SEQ ID NO: 37 - DNA, 25 bp, Artificial Sequence
  Synthetic polynucleotide
caccgtcaggtgattaccgtagaga

SEQ ID NO: 38 - DNA, 25 bp, Artificial Sequence
  Synthetic polynucleotide
caccgaggtagcagggaccatcgca

SEQ ID NO: 39 - DNA, 25 bp, Artificial Sequence
  Synthetic polynucleotide
caccgctgaagtgcagcttgcgaca

SEQ ID NO: 40 - DNA, 25 bp, Artificial Sequence
  Synthetic polynucleotide
caccgacgccgtttctgccctgcga

SEQ ID NO: 41 - DNA, 25 bp, Artificial Sequence
  Synthetic polynucleotide
caccgcttgtcgcaagctgcacttc

SEQ ID NO: 42 - DNA, 25 bp, Artificial Sequence
  Synthetic polynucleotide
caccggttgaaaagtttcgctctcg

SEQ ID NO: 43 - DNA, 25 bp, Artificial Sequence
  Synthetic polynucleotide
caccggttggggacagtgccgcttc

SEQ ID NO: 44 - DNA, 25 bp, Artificial Sequence
  Synthetic polynucleotide
caccggcagaaacggcgtgcagaac

SEQ ID NO: 45 - DNA, 25 bp, Artificial Sequence
  Synthetic polynucleotide
aaactagtgatagtcccgtgcctac

SEQ ID NO: 46 - DNA, 25 bp, Artificial Sequence
  Synthetic polynucleotide
aaactctctacggtaatcacctgac

SEQ ID NO: 47 - DNA, 25 bp, Artificial Sequence
  Synthetic polynucleotide
aaactgcgatggtccctgctacctc

SEQ ID NO: 48 - DNA, 25 bp, Artificial Sequence
  Synthetic polynucleotide
aaactgtcgcaagctgcacttcagc

SEQ ID NO: 49 - DNA, 25 bp, Artificial Sequence
  Synthetic polynucleotide
aaactcgcagggcagaaacggcgtc

SEQ ID NO: 50 - DNA, 25 bp, Artificial Sequence
  Synthetic polynucleotide
aaacgaagtgcagcttgcgacaagc

SEQ ID NO: 51 - DNA, 25 bp, Artificial Sequence
  Synthetic polynucleotide
aaaccgagagcgaaacttttcaacc

SEQ ID NO: 52 - DNA, 25 bp, Artificial Sequence
  Synthetic polynucleotide
aaacgaagcggcactgtccccaacc

SEQ ID NO: 53 - DNA, 25 bp, Artificial Sequence
  Synthetic polynucleotide
aaacgttctgcacgccgtttctgcc

SEQ ID NO: 54 - DNA, 25 bp, Artificial Sequence
  Synthetic polynucleotide
caccggtccagtgtctacaaaatgt

SEQ ID NO: 55 - DNA, 25 bp, Artificial Sequence
  Synthetic polynucleotide
caccgcactgaactacttacatggg

SEQ ID NO: 56 - DNA, 25 bp, Artificial Sequence
  Synthetic polynucleotide
caccgatcaacttactactctccca

SEQ ID NO: 57 - DNA, 25 bp, Artificial Sequence
  Synthetic polynucleotide
caccggctggacctgggatgatgta

SEQ ID NO: 58 - DNA, 25 bp, Artificial Sequence
  Synthetic polynucleotide
caccgcgcatacctgcaggtttctc

SEQ ID NO: 59 - DNA, 25 bp, Artificial Sequence
  Synthetic polynucleotide
caccgacagacctcagaatttcatc

SEQ ID NO: 60 - DNA, 25 bp, Artificial Sequence
  Synthetic polynucleotide
caccgagattccagtacctttctta

SEQ ID NO: 61 - DNA, 25 bp, Artificial Sequence
  Synthetic polynucleotide
caccgcatctgctccaaactcaaaa

SEQ ID NO: 62 - DNA, 25 bp, Artificial Sequence
  Synthetic polynucleotide
caccgacatccctgatgaaattctg

SEQ ID NO: 63 - DNA, 25 bp, Artificial Sequence
  Synthetic polynucleotide
caccgctagttgggagctgagagtg

SEQ ID NO: 64 - DNA, 25 bp, Artificial Sequence
  Synthetic polynucleotide
aaacacattttgtagacactggacc

SEQ ID NO: 65 - DNA, 25 bp, Artificial Sequence
  Synthetic polynucleotide
aaaccccatgtaagtagttcagtgc

SEQ ID NO: 66 - DNA, 25 bp, Artificial Sequence
  Synthetic polynucleotide
aaactgggagagtagtaagttgatc

SEQ ID NO: 67 - DNA, 25 bp, Artificial Sequence
  Synthetic polynucleotide
aaactacatcatcccaggtccagcc

SEQ ID NO: 68 - DNA, 25 bp, Artificial Sequence
  Synthetic polynucleotide
aaacgagaaacctgcaggtatgcgc

SEQ ID NO: 69 - DNA, 25 bp, Artificial Sequence
  Synthetic polynucleotide
aaacgatgaaattctgaggtctgtc

SEQ ID NO: 70 - DNA, 25 bp, Artificial Sequence
  Synthetic polynucleotide
aaactaagaaaggtactggaatctc

SEQ ID NO: 71 - DNA, 25 bp, Artificial Sequence
  Synthetic polynucleotide
aaacttttgagtttggagcagatgc

SEQ ID NO: 72 - DNA, 25 bp, Artificial Sequence
  Synthetic polynucleotide
aaaccagaatttcatcagggatgtc

SEQ ID NO: 73 - DNA, 25 bp, Artificial Sequence
  Synthetic polynucleotide
aaaccactctcagctcccaactagc

SEQ ID NO: 74 - DNA, 25 bp, Artificial Sequence
  Synthetic polynucleotide
caccgccccgagcgcagcgactacg

SEQ ID NO: 75 - DNA, 25 bp, Artificial Sequence
  Synthetic polynucleotide
caccgagaaagacacggacacgatg

SEQ ID NO: 76 - DNA, 25 bp, Artificial Sequence
  Synthetic polynucleotide
caccgcctgtacaacgagcacacgg

SEQ ID NO: 77 - DNA, 25 bp, Artificial Sequence
  Synthetic polynucleotide
caccggtcacgctccccctgcgcac

SEQ ID NO: 78 - DNA, 25 bp, Artificial Sequence
  Synthetic polynucleotide
caccgggtggtgtcccgcgccgaga

SEQ ID NO: 79 - DNA, 25 bp, Artificial Sequence
  Synthetic polynucleotide
caccgatctcgtcgacgtcgcccgg

SEQ ID NO: 80 - DNA, 25 bp, Artificial Sequence
  Synthetic polynucleotide
caccgaagattagatgcaccgcgat

SEQ ID NO: 81 - DNA, 25 bp, Artificial Sequence
  Synthetic polynucleotide
caccgtatgcttcagatcgcctgtc

SEQ ID NO: 82 - DNA, 25 bp, Artificial Sequence
  Synthetic polynucleotide
caccgctcaggacattaaggaacgc

SEQ ID NO: 83 - DNA, 25 bp, Artificial Sequence
  Synthetic polynucleotide
caccggcaatcttccgaggactcat

SEQ ID NO: 84 - DNA, 25 bp, Artificial Sequence
  Synthetic polynucleotide
aaaccgtagtcgctgcgctcggggc

SEQ ID NO: 85 - DNA, 25 bp, Artificial Sequence
  Synthetic polynucleotide
aaaccatcgtgtccgtgtctttctc

SEQ ID NO: 86 - DNA, 25 bp, Artificial Sequence
  Synthetic polynucleotide
aaacccgtgtgctcgttgtacaggc

SEQ ID NO: 87 - DNA, 25 bp, Artificial Sequence
  Synthetic polynucleotide
aaacgtgcgcagggggagcgtgacc

SEQ ID NO: 88 - DNA, 25 bp, Artificial Sequence
  Synthetic polynucleotide
aaactctcggcgcgggacaccaccc

SEQ ID NO: 89 - DNA, 25 bp, Artificial Sequence
  Synthetic polynucleotide
aaacccgggcgacgtcgacgagatc

SEQ ID NO: 90 - DNA, 25 bp, Artificial Sequence
  Synthetic polynucleotide
aaacatcgcggtgcatctaatcttc

SEQ ID NO: 91 - DNA, 25 bp, Artificial Sequence
  Synthetic polynucleotide
aaacgacaggcgatctgaagcatac

SEQ ID NO: 92 - DNA, 25 bp, Artificial Sequence
  Synthetic polynucleotide
aaacgcgttccttaatgtcctgagc

SEQ ID NO: 93 - DNA, 25 bp, Artificial Sequence
  Synthetic polynucleotide
aaacatgagtcctcggaagattgcc

SEQ ID NO: 94 - DNA, 25 bp, Artificial Sequence
  Synthetic polynucleotide
caccgcggtgagtggaactaaaccg

SEQ ID NO: 95 - DNA, 25 bp, Artificial Sequence
  Synthetic polynucleotide
caccggcagtgtgagaaaggcctcg

SEQ ID NO: 96 - DNA, 25 bp, Artificial Sequence
  Synthetic polynucleotide
caccgaggagccctatttcggcatg

SEQ ID NO: 97 - DNA, 25 bp, Artificial Sequence
  Synthetic polynucleotide
caccgagtgtctcgcatcctcatcg

SEQ ID NO: 98 - DNA, 25 bp, Artificial Sequence
  Synthetic polynucleotide
caccggcttgtttcgggacgtggtg

SEQ ID NO: 99 - DNA, 25 bp, Artificial Sequence
  Synthetic polynucleotide
caccggtgtacagctatagctgcga

SEQ ID NO: 100 - DNA, 25 bp, Artificial Sequence
  Synthetic polynucleotide
caccggtttagttccactcaccgtg

SEQ ID NO: 101 - DNA, 25 bp, Artificial Sequence
  Synthetic polynucleotide
caccgtgaagagggattgttaatcc

SEQ ID NO: 102 - DNA, 25 bp, Artificial Sequence
  Synthetic polynucleotide
caccgagatgatgagttctatcagc

SEQ ID NO: 103 - DNA, 25 bp, Artificial Sequence
  Synthetic polynucleotide
caccgattaacaatccctcttcacc

SEQ ID NO: 104 - DNA, 25 bp, Artificial Sequence
  Synthetic polynucleotide
aaaccggtttagttccactcaccgc

SEQ ID NO: 105 - DNA, 25 bp, Artificial Sequence
  Synthetic polynucleotide
aaaccgaggcctttctcacactgcc

SEQ ID NO: 106 - DNA, 25 bp, Artificial Sequence
  Synthetic polynucleotide
aaaccatgccgaaatagggctcctc

SEQ ID NO: 107 - DNA, 25 bp, Artificial Sequence
  Synthetic polynucleotide
aaaccgatgaggatgcgagacactc

SEQ ID NO: 108 - DNA, 25 bp, Artificial Sequence
  Synthetic polynucleotide
aaaccaccacgtcccgaaacaagcc

SEQ ID NO: 109 - DNA, 25 bp, Artificial Sequence
  Synthetic polynucleotide
aaactcgcagctatagctgtacacc

SEQ ID NO: 110 - DNA, 25 bp, Artificial Sequence
  Synthetic polynucleotide
aaaccacggtgagtggaactaaacc

SEQ ID NO: 111 - DNA, 25 bp, Artificial Sequence
  Synthetic polynucleotide
aaacggattaacaatccctcttcac

SEQ ID NO: 112 - DNA, 25 bp, Artificial Sequence
  Synthetic polynucleotide
aaacgctgatagaactcatcatctc

SEQ ID NO: 113 - DNA, 25 bp, Artificial Sequence
  Synthetic polynucleotide
aaacggtgaagagggattgttaatc

SEQ ID NO: 114 - DNA, 25 bp, Artificial Sequence
  Synthetic polynucleotide
caccggcgaggtattcggctccgcg

SEQ ID NO: 115 - DNA, 25 bp, Artificial Sequence
  Synthetic polynucleotide
caccggctttcacggaggttcgacg

SEQ ID NO: 116 - DNA, 25 bp, Artificial Sequence
  Synthetic polynucleotide
caccgatgttgcagttcggctcgat

SEQ ID NO: 117 - DNA, 25 bp, Artificial Sequence
  Synthetic polynucleotide
caccgacgtgtaaggcgaacgcctt

SEQ ID NO: 118 - DNA, 25 bp, Artificial Sequence
  Synthetic polynucleotide
caccggactccgggtactaaatgtc

SEQ ID NO: 119 - DNA, 25 bp, Artificial Sequence
  Synthetic polynucleotide
caccgccgcgccgttagggaacgag

SEQ ID NO: 120 - DNA, 25 bp, Artificial Sequence
  Synthetic polynucleotide
caccgattgttcgaccgtctacggg

SEQ ID NO: 121 - DNA, 25 bp, Artificial Sequence
  Synthetic polynucleotide
caccgacccatcgggtgcgatatgg

SEQ ID NO: 122 - DNA, 25 bp, Artificial Sequence
  Synthetic polynucleotide
caccgcgggcgtcacctgctagtaa

SEQ ID NO: 123 - DNA, 25 bp, Artificial Sequence
  Synthetic polynucleotide
caccggcttctactcgcaacgtatt

SEQ ID NO: 124 - DNA, 25 bp, Artificial Sequence
  Synthetic polynucleotide
aaaccgcggagccgaatacctcgcc

SEQ ID NO: 125 - DNA, 25 bp, Artificial Sequence
  Synthetic polynucleotide
aaaccgtcgaacctccgtgaaagcc

SEQ ID NO: 126 - DNA, 25 bp, Artificial Sequence
  Synthetic polynucleotide
aaacatcgagccgaactgcaacatc

SEQ ID NO: 127 - DNA, 25 bp, Artificial Sequence
  Synthetic polynucleotide
aaacaaggcgttcgccttacacgtc

SEQ ID NO: 128 - DNA, 25 bp, Artificial Sequence
  Synthetic polynucleotide
aaacgacatttagtacccggagtcc

SEQ ID NO: 129 - DNA, 25 bp, Artificial Sequence
  Synthetic polynucleotide
aaacctcgttccctaacggcgcggc

SEQ ID NO: 130 - DNA, 25 bp, Artificial Sequence
  Synthetic polynucleotide
aaaccccgtagacggtcgaacaatc

SEQ ID NO: 131 - DNA, 25 bp, Artificial Sequence
  Synthetic polynucleotide
aaacccatatcgcacccgatgggtc

SEQ ID NO: 132 - DNA, 25 bp, Artificial Sequence
  Synthetic polynucleotide
aaacttactagcaggtgacgcccgc

SEQ ID NO: 133 - DNA, 25 bp, Artificial Sequence
  Synthetic polynucleotide
aaacaatacgttgcgagtagaagcc

SEQ ID NO: 134 - DNA, 20 bp, Artificial Sequence
  Synthetic polynucleotide
taggcacgggactatcacta

SEQ ID NO: 135 - DNA, 20 bp, Artificial Sequence
  Synthetic polynucleotide
tcaggtgattaccgtagaga

SEQ ID NO: 136 - DNA, 20 bp, Artificial Sequence
  Synthetic polynucleotide
aggtagcagggaccatcgca

SEQ ID NO: 137 - DNA, 20 bp, Artificial Sequence
  Synthetic polynucleotide
ctgaagtgcagcttgcgaca

SEQ ID NO: 138 - DNA, 20 bp, Artificial Sequence
  Synthetic polynucleotide
acgccgtttctgccctgcga

SEQ ID NO: 139 - DNA, 20 bp, Artificial Sequence
  Synthetic polynucleotide
cttgtcgcaagctgcacttc

SEQ ID NO: 140 - DNA, 20 bp, Artificial Sequence
  Synthetic polynucleotide
gttgaaaagtttcgctctcg

SEQ ID NO: 141 - DNA, 20 bp, Artificial Sequence
  Synthetic polynucleotide
gttggggacagtgccgcttc

SEQ ID NO: 142 - DNA, 20 bp, Artificial Sequence
  Synthetic polynucleotide
gcagaaacggcgtgcagaac

SEQ ID NO: 143 - DNA, 21 bp, Artificial Sequence
  Synthetic polynucleotide
tagtgatagtcccgtgcctac

SEQ ID NO: 144 - DNA, 21 bp, Artificial Sequence
  Synthetic polynucleotide
tctctacggtaatcacctgac

SEQ ID NO: 145 - DNA, 21 bp, Artificial Sequence
  Synthetic polynucleotide
tgcgatggtccctgctacctc

SEQ ID NO: 146 - DNA, 21 bp, Artificial Sequence
  Synthetic polynucleotide
tgtcgcaagctgcacttcagc

SEQ ID NO: 147 - DNA, 21 bp, Artificial Sequence
  Synthetic polynucleotide
tcgcagggcagaaacggcgtc

SEQ ID NO: 148 - DNA, 21 bp, Artificial Sequence
  Synthetic polynucleotide
gaagtgcagcttgcgacaagc

SEQ ID NO: 149 - DNA, 21 bp, Artificial Sequence
  Synthetic polynucleotide
cgagagcgaaacttttcaacc

SEQ ID NO: 150 - DNA, 21 bp, Artificial Sequence
  Synthetic polynucleotide
gaagcggcactgtccccaacc

SEQ ID NO: 151 - DNA, 21 bp, Artificial Sequence
  Synthetic polynucleotide
gttctgcacgccgtttctgcc

SEQ ID NO: 152 - DNA, 20 bp, Artificial Sequence
  Synthetic polynucleotide
gtccagtgtctacaaaatgt

SEQ ID NO: 153 - DNA, 20 bp, Artificial Sequence
  Synthetic polynucleotide
cactgaactacttacatggg

SEQ ID NO: 154 - DNA, 20 bp, Artificial Sequence
  Synthetic polynucleotide
atcaacttactactctccca

SEQ ID NO: 155 - DNA, 20 bp, Artificial Sequence
  Synthetic polynucleotide
gctggacctgggatgatgta

SEQ ID NO: 156 - DNA, 20 bp, Artificial Sequence
  Synthetic polynucleotide
cgcatacctgcaggtttctc

SEQ ID NO: 157 - DNA, 20 bp, Artificial Sequence
  Synthetic polynucleotide
acagacctcagaatttcatc

SEQ ID NO: 158 - DNA, 20 bp, Artificial Sequence
  Synthetic polynucleotide
agattccagtacctttctta

SEQ ID NO: 159 - DNA, 20 bp, Artificial Sequence
  Synthetic polynucleotide
catctgctccaaactcaaaa

SEQ ID NO: 160 - DNA, 20 bp, Artificial Sequence
  Synthetic polynucleotide
acatccctgatgaaattctg

SEQ ID NO: 161 - DNA, 20 bp, Artificial Sequence
  Synthetic polynucleotide
ctagttgggagctgagagtg

SEQ ID NO: 162 - DNA, 21 bp, Artificial Sequence
  Synthetic polynucleotide
acattttgtagacactggacc

SEQ ID NO: 163 - DNA, 21 bp, Artificial Sequence
  Synthetic polynucleotide
cccatgtaagtagttcagtgc

SEQ ID NO: 164 - DNA, 21 bp, Artificial Sequence
  Synthetic polynucleotide
tgggagagtagtaagttgatc

SEQ ID NO: 165 - DNA, 21 bp, Artificial Sequence
  Synthetic polynucleotide
tacatcatcccaggtccagcc

SEQ ID NO: 166 - DNA, 21 bp, Artificial Sequence
  Synthetic polynucleotide
gagaaacctgcaggtatgcgc

SEQ ID NO: 167 - DNA, 21 bp, Artificial Sequence
  Synthetic polynucleotide
gatgaaattctgaggtctgtc

SEQ ID NO: 168 - DNA, 21 bp, Artificial Sequence
  Synthetic polynucleotide
taagaaaggtactggaatctc

SEQ ID NO: 169 - DNA, 21 bp, Artificial Sequence
  Synthetic polynucleotide
ttttgagtttggagcagatgc

SEQ ID NO: 170 - DNA, 21 bp, Artificial Sequence
  Synthetic polynucleotide
cagaatttcatcagggatgtc

SEQ ID NO: 171 - DNA, 21 bp, Artificial Sequence
  Synthetic polynucleotide
cactctcagctcccaactagc

SEQ ID NO: 172 - DNA, 20 bp, Artificial Sequence
  Synthetic polynucleotide
ccccgagcgcagcgactacg

SEQ ID NO: 173 - DNA, 20 bp, Artificial Sequence
  Synthetic polynucleotide
agaaagacacggacacgatg

SEQ ID NO: 174 - DNA, 20 bp, Artificial Sequence
  Synthetic polynucleotide
cctgtacaacgagcacacgg

SEQ ID NO: 175 - DNA, 20 bp, Artificial Sequence
  Synthetic polynucleotide
gtcacgctccccctgcgcac

SEQ ID NO: 176 - DNA, 20 bp, Artificial Sequence
  Synthetic polynucleotide
ggtggtgtcccgcgccgaga

SEQ ID NO: 177 - DNA, 20 bp, Artificial Sequence
  Synthetic polynucleotide
atctcgtcgacgtcgcccgg

SEQ ID NO: 178 - DNA, 20 bp, Artificial Sequence
  Synthetic polynucleotide
aagattagatgcaccgcgat

SEQ ID NO: 179 - DNA, 20 bp, Artificial Sequence
  Synthetic polynucleotide
tatgcttcagatcgcctgtc

SEQ ID NO: 180 - DNA, 20 bp, Artificial Sequence
  Synthetic polynucleotide
ctcaggacattaaggaacgc

SEQ ID NO: 181 - DNA, 20 bp, Artificial Sequence
  Synthetic polynucleotide
gcaatcttccgaggactcat

SEQ ID NO: 182 - DNA, 21 bp, Artificial Sequence
  Synthetic polynucleotide
cgtagtcgctgcgctcggggc

SEQ ID NO: 183 - DNA, 21 bp, Artificial Sequence
  Synthetic polynucleotide
catcgtgtccgtgtctttctc

SEQ ID NO: 184 - DNA, 21 bp, Artificial Sequence
  Synthetic polynucleotide
ccgtgtgctcgttgtacaggc

SEQ ID NO: 185 - DNA, 21 bp, Artificial Sequence
  Synthetic polynucleotide
gtgcgcagggggagcgtgacc

SEQ ID NO: 186 - DNA, 21 bp, Artificial Sequence
  Synthetic polynucleotide
tctcggcgcgggacaccaccc

SEQ ID NO: 187 - DNA, 21 bp, Artificial Sequence
  Synthetic polynucleotide
ccgggcgacgtcgacgagatc

SEQ ID NO: 188 - DNA, 21 bp, Artificial Sequence
  Synthetic polynucleotide
atcgcggtgcatctaatcttc

SEQ ID NO: 189 - DNA, 21 bp, Artificial Sequence
  Synthetic polynucleotide
gacaggcgatctgaagcatac

SEQ ID NO: 190 - DNA, 21 bp, Artificial Sequence
  Synthetic polynucleotide
gcgttccttaatgtcctgagc

SEQ ID NO: 191 - DNA, 21 bp, Artificial Sequence
  Synthetic polynucleotide
atgagtcctcggaagattgcc

SEQ ID NO: 192 - DNA, 20 bp, Artificial Sequence
  Synthetic polynucleotide
cggtgagtggaactaaaccg

SEQ ID NO: 193 - DNA, 20 bp, Artificial Sequence
  Synthetic polynucleotide
gcagtgtgagaaaggcctcg

SEQ ID NO: 194 - DNA, 20 bp, Artificial Sequence
  Synthetic polynucleotide
aggagccctatttcggcatg

SEQ ID NO: 195 - DNA, 20 bp, Artificial Sequence
  Synthetic polynucleotide
agtgtctcgcatcctcatcg

SEQ ID NO: 196 - DNA, 20 bp, Artificial Sequence
  Synthetic polynucleotide
gcttgtttcgggacgtggtg

SEQ ID NO: 197 - DNA, 20 bp, Artificial Sequence
  Synthetic polynucleotide
gtgtacagctatagctgcga

SEQ ID NO: 198 - DNA, 20 bp, Artificial Sequence
  Synthetic polynucleotide
gtttagttccactcaccgtg

SEQ ID NO: 199 - DNA, 20 bp, Artificial Sequence
  Synthetic polynucleotide
tgaagagggattgttaatcc

SEQ ID NO: 200 - DNA, 20 bp, Artificial Sequence
  Synthetic polynucleotide
agatgatgagttctatcagc

SEQ ID NO: 201 - DNA, 20 bp, Artificial Sequence
  Synthetic polynucleotide
attaacaatccctcttcacc

SEQ ID NO: 202 - DNA, 21 bp, Artificial Sequence
  Synthetic polynucleotide
cggtttagttccactcaccgc

SEQ ID NO: 203 - DNA, 21 bp, Artificial Sequence
  Synthetic polynucleotide
cgaggcctttctcacactgcc

SEQ ID NO: 204 - DNA, 21 bp, Artificial Sequence
  Synthetic polynucleotide
catgccgaaatagggctcctc

SEQ ID NO: 205 - DNA, 21 bp, Artificial Sequence
  Synthetic polynucleotide
cgatgaggatgcgagacactc

SEQ ID NO: 206 - DNA, 21 bp, Artificial Sequence
  Synthetic polynucleotide
caccacgtcccgaaacaagcc

SEQ ID NO: 207 - DNA, 21 bp, Artificial Sequence
  Synthetic polynucleotide
tcgcagctatagctgtacacc

SEQ ID NO: 208 - DNA, 21 bp, Artificial Sequence
  Synthetic polynucleotide
cacggtgagtggaactaaacc

SEQ ID NO: 209 - DNA, 21 bp, Artificial Sequence
  Synthetic polynucleotide
ggattaacaatccctcttcac

SEQ ID NO: 210 - DNA, 21 bp, Artificial Sequence
  Synthetic polynucleotide
gctgatagaactcatcatctc

SEQ ID NO: 211 - DNA, 21 bp, Artificial Sequence
  Synthetic polynucleotide
ggtgaagagggattgttaatc

SEQ ID NO: 212 - DNA, 20 bp, Artificial Sequence
  Synthetic polynucleotide
gcgaggtattcggctccgcg

SEQ ID NO: 213 - DNA, 20 bp, Artificial Sequence
  Synthetic polynucleotide
gctttcacggaggttcgacg

SEQ ID NO: 214 - DNA, 20 bp, Artificial Sequence
  Synthetic polynucleotide
atgttgcagttcggctcgat

SEQ ID NO: 215 - DNA, 20 bp, Artificial Sequence
  Synthetic polynucleotide
acgtgtaaggcgaacgcctt

SEQ ID NO: 216 - DNA, 20 bp, Artificial Sequence
  Synthetic polynucleotide
gactccgggtactaaatgtc

SEQ ID NO: 217 - DNA, 20 bp, Artificial Sequence
  Synthetic polynucleotide
ccgcgccgttagggaacgag

SEQ ID NO: 218 - DNA, 20 bp, Artificial Sequence
  Synthetic polynucleotide
attgttcgaccgtctacggg

SEQ ID NO: 219 - DNA, 20 bp, Artificial Sequence
  Synthetic polynucleotide
acccatcgggtgcgatatgg

SEQ ID NO: 220 - DNA, 20 bp, Artificial Sequence
  Synthetic polynucleotide
cgggcgtcacctgctagtaa

SEQ ID NO: 221 - DNA, 20 bp, Artificial Sequence
  Synthetic polynucleotide
gcttctactcgcaacgtatt

SEQ ID NO: 222 - DNA, 21 bp, Artificial Sequence
  Synthetic polynucleotide
cgcggagccgaatacctcgcc

SEQ ID NO: 223 - DNA, 21 bp, Artificial Sequence
  Synthetic polynucleotide
cgtcgaacctccgtgaaagcc

SEQ ID NO: 224 - DNA, 21 bp, Artificial Sequence
  Synthetic polynucleotide
atcgagccgaactgcaacatc

SEQ ID NO: 225 - DNA, 21 bp, Artificial Sequence
  Synthetic polynucleotide
aaggcgttcgccttacacgtc

SEQ ID NO: 226 - DNA, 21 bp, Artificial Sequence
  Synthetic polynucleotide
gacatttagtacccggagtcc

SEQ ID NO: 227 - DNA, 21 bp, Artificial Sequence
  Synthetic polynucleotide
ctcgttccctaacggcgcggc

SEQ ID NO: 228 - DNA, 21 bp, Artificial Sequence
  Synthetic polynucleotide
cccgtagacggtcgaacaatc

SEQ ID NO: 229 - DNA, 21 bp, Artificial Sequence
  Synthetic polynucleotide
ccatatcgcacccgatgggtc

SEQ ID NO: 230 - DNA, 21 bp, Artificial Sequence
  Synthetic polynucleotide
ttactagcaggtgacgcccgc

SEQ ID NO: 231 - DNA, 21 bp, Artificial Sequence
  Synthetic polynucleotide
aatacgttgcgagtagaagcc

(19) United States
(12) Patent Application Publication
(10) Pub. No.: US 2019/0262371 A1
(43) Pub. Date: Aug. 29, 2019

(54) METHODS AND COMPOSITION FOR THE TREATMENT OF RNA VIRAL INFECTIONS

(71) Applicant: The Regents of the University of California, Oakland, CA (US)

(72) Inventor: Tariq M. Rana, San Diego, CA (US)

(21) Appl. No.: 16/343,723

(22) PCT Filed: Oct. 20, 2017
(86) PCT No.: PCT/US17/57699

§ 371 Date: Apr. 19, 2019

Related U.S. Application Data
(60) Provisional application No. 62/410,796, filed on Oct. 20, 2016.

(51) Int. Cl. A61K 31/7076; A61K 39/42; A61K 39/295; A61P 31/14; A61K 39/39; A61K 31/52

(52) U.S. Cl. CPC A61K 31/7076; A61K 39/42; A61K 31/52; A61P 31/14; A61K 39/39; A61K 39/295

(57) ABSTRACT

Disclosed herein, inter alia, are agents having antiviral activity and methods of use thereof.

### CROSS-REFERENCES TO RELATED APPLICATIONS

This application claims the benefit of U.S. Provisional Application No. 62/410,796, filed Oct. 20, 2016, which is incorporated herein by reference in its entirety and for all purposes.

### STATEMENT AS TO RIGHTS TO INVENTIONS MADE UNDER FEDERALLY SPONSORED RESEARCH AND DEVELOPMENT

This invention was made with government support under DA039562 awarded by the National Institute of Health. The government has certain rights in the invention.

### REFERENCE TO A “SEQUENCE LISTING,” A TABLE, OR A COMPUTER PROGRAM LISTING APPENDIX SUBMITTED AS AN ASCII FILE

The Sequence Listing written in file 048537-586001WO_SL_ST25.txt, created Oct. 20, 2017, 46,062 bytes, machine format IBM-PC, MS Windows operating system, is hereby incorporated by reference.

### BACKGROUND

Human infection with ZIKA virus (ZIKV), a mosquito-borne flavivirus, has spread rapidly since the 2015 outbreak in Brazil, and the World Health Organization declared ZIKV infection an International Public Health Emergency in 2016 (Fauci and Morens, 2016; Heymann et al., 2016; Petersen et al., 2016). ZIKV was discovered in 1947 (Driggers et al., 2016a) and, although it had previously caused only sporadic disease in Africa and Asia, more recent outbreaks occurred in Micronesia in 2007 and in French Polynesia in 2013 (Broutet et al., 2016). ZIKV infection has been identified as the etiological agent of severe neurological defects, including microcephaly during fetal development (Driggers et al., 2016b) and neuronal injury associated with Guillain-Barré syndrome in adults (Dejnirattisai et al., 2016). New modes of viral transmission, including maternal-fetal (Brasil et al., 2016) and sexual transmission (Hills et al., 2016), have been reported. ZIKV can infect human skin explants, peripheral blood mononuclear cells, human neuroprogenitor cells, and human cerebral organoids (Dang et al., 2016a; Hamel et al., 2015; Tang et al., 2016). In mouse models, ZIKV may be neurotropic (Cugola et al., 2016; Lazear et al., 2016; Li et al., 2016; Mlakar et al., 2016; Sarno et al., 2016).

ZIKV and other members of the Flaviviridae family, such as dengue (DENV), West Nile (WNV), yellow fever (YFV), and Japanese encephalopathy (JEV), are positive (+) single-stranded RNA viruses. The ZIKV genome encodes a single polyprotein precursor that is cleaved by viral and host proteases to produce three structural and seven nonstructural proteins. Although our understanding of the molecular mechanisms involved in ZIKV infection of human cells has increased dramatically in the past few years, key determinants of ZIKV pathogenicity, such as cell-type specificity, mode of entry, and host factors essential for replication, are still largely unknown. In particular, there is a large gap in our understanding of the genetic and epigenetic regulatory mechanisms governing the viral life cycle and viral interactions with host cells.

As is the case with proteins and DNA, chemical modification of RNA affects its metabolism, function, and localization. More than 100 diverse chemical modifications of RNA nucleotides have been identified, most of which affect ribosomal and transfer RNAs. Modifications of mRNAs and long noncoding RNAs include the 5′-cap structure, N6-methylation of adenosine (m6A), and methylation of C5 of cytosine (m5C) (Fu et al., 2014; Squires et al., 2012; Yi and Pan, 2011). m6A is the most prevalent internal modification of eukaryotic mRNA with unique distribution patterns (Dominissini et al., 2012; Meyer et al., 2012; Schwartz et al., 2014). While it is becoming increasingly clear that m6A plays an important regulatory role in physiological and pathological processes (Frayling et al., 2007; Jia et al., 2011; Zheng et al., 2013), little is known of the function of m6A in the mammalian immune system or its influence on host-pathogen interactions.

Adenosine methylation is catalyzed by a large RNA methyltransferase complex (MTase), composed of two catalytic subunits (METTL3 and METTL14), a splicing factor (WTAP), a protein (KIAA1429), and other subunits not yet identified (Bokar et al., 1997; Liu et al., 2014; Ping et al., 2014; Schwartz et al., 2014), while removal of methyl groups is catalyzed by two RNA demethylases, FTO and ALKBH5 (Jia et al., 2011; Zheng et al., 2013). m6A is most abundant in translation start sites, stop codons, and 3′-UTRs (Dominissini et al., 2012; Meyer et al., 2012; Schwartz et al., 2014), suggesting that it plays important roles in mRNA biology. Indeed, m6A has been shown to contribute to mRNA stability (Geula et al., 2015; Wang et al., 2014a; Xu et al., 2014); RNA structure, with subsequent effects on RNA-protein interactions (Liu et al., 2015); translation (Meyer et al., 2015; Wang et al., 2015); mRNA nuclear export (Zheng et al., 2013); exon splicing (Zhao et al., 2014) by promoting binding of splicing factor SRSF2 (Zhao et al., 2014); circadian gene expression upon METTL3 depletion (Fustin et al., 2013); and embryonic stem cell pluripotency upon modulation of either METTL3 (Batista et al., 2014; Geula et al., 2015) or METTL14 (Wang et al., 2014b) expression. The precise sites and abundance of m6A are highly regulated under normal conditions.

Although the m6A modification was identified in viral RNA several decades ago (e.g., Rous sarcoma virus, (Kane and Beemon, 1985) influenza virus (Krug et al., 1976) SV40 virus, (Finkel and Groner, 1983), its function and relevance to viral replication had remained unclear. Similarly, whether and how viral infections influence the dynamics of the host and viral RNA methylomes was relatively unknown. Three published reports describe m6A modification of HIV-1 RNA (Kennedy et al., 2016; Lichinchi et al., 2016; Tirumuru et al., 2016).

The epitranscriptomic landscape of flaviviruses, including ZIKV, remains largely unexplored. N-7 and 2′-O ribose methylations (2′-O-Me) in the cap structure by the viral NS5 protein are required for the efficient translation of viral proteins and for evasion from host antiviral responses. NS5 mutation and loss of N7-methylation are lethal for WNV (Kroschewski et al., 2008; Zhang et al., 2008), and defects in 2′-O-Me dramatically decrease WNV fitness due to enhanced restriction by the host factor IFIT (Daffis et al., 2010). 2′-O-Me of internal adenosines have been detected in DENV and WNV RNA, suggesting a further layer of regulation (Dong et al., 2012). Indeed, there are no available agents targeting ZIKA virus enzymatic functions.

Disclosed herein, inter alia, are solutions to these and other problems in the art.

### BRIEF SUMMARY

In an aspect is provided a method of treating or preventing a Zika viral infection in a subject in need thereof, the method including administering an effective amount of an adenosine N-6 methylation agonist or an adenosine N-6 demethylation antagonist.

In an aspect is provided a method of treating or preventing a Zika viral infection in a subject in need thereof, the method including administering an effective amount of an RNA 2′-O-methyl transferase inhibitor (e.g., an inhibitor of viral 2′-O-methyl transferase).

In an aspect is provided a vaccine formulation, wherein the vaccine formulation includes an immunogenic agent, an adenosine N-6 methylation agonist or an adenosine N-6 demethylation antagonist, and an adjuvant (e.g., a vaccine adjuvant).

In an aspect is provided a method of treating or preventing an RNA virus infection in a subject in need thereof, the method including administering an effective amount of a compound of the formula:

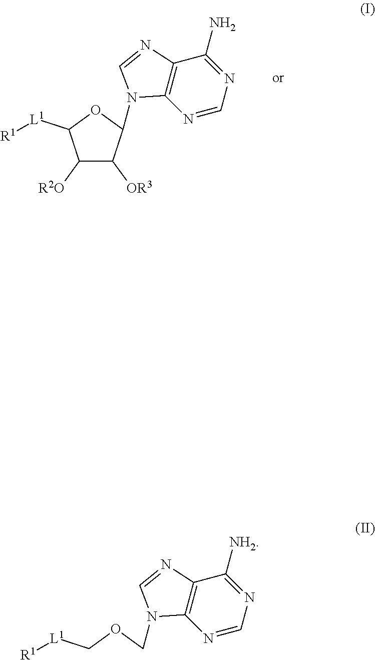

L¹ is a bond, —O—, —S—, —NH—, —C(O)NH—, —C(O)—, —S(O)₂NH—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene. R¹ is hydrogen, halogen, —CF₃, —CN, —OR^{1A}, —NHR^{1A}, —N₃, —SR^{1A}, —COOR^{1A}, —CONHR^{1A}, —NHC(O)R^{1A}, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl. R^{1A} is hydrogen, halogen, —CF₃, —CN, —OH, —NH₂, —N₃, —SH, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl. R² is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl. R³ is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

In an aspect is provided a compound having the formula:

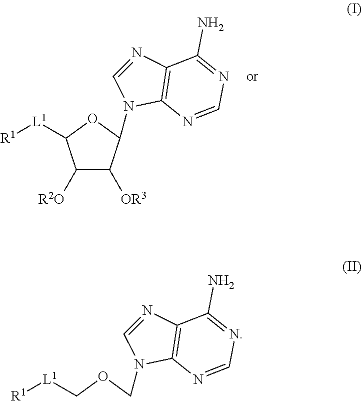

L¹ is a bond, —O—, —S—, —NH—, —C(O)NH—, —C(O)—, —S(O)₂NH—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene. R¹ is hydrogen, halogen, —CF₃, —CN, —OR^{1A}, —NHR^{1A}, —N₃, —SR^{1A}, —COOR^{1A}, —CONHR^{1A}, —NHC(O)R^{1A}, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl. R^{1A} is hydrogen, halogen, —CF₃, —CN, —OH, —NH₂, —N₃, —SH, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl. R² is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl. R³ is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

In another aspect, there is provided a pharmaceutical composition including a agent (e.g., a compound described herein, nucleic acid, antibody) and a pharmaceutically acceptable excipient.

In another aspect, there is provided a pharmaceutical composition comprising an RNA compound, the RNA compound encoding an RNA virus structural protein. In embodiments, the RNA compound is a virus. In embodiments, the RNA compound is a nucleic acid (e.g., a nucleic acid described herein).

### BRIEF DESCRIPTION OF THE DRAWINGS

FIGS. 1A-ID. ZIKV RNA Contains m⁶A and 2′-O-Me Modifications, and Methylation is Regulated by Host METTL3, METTL14, and ALKBH5. (FIG. 1A) LC-MS/MS quantification of m⁶A and 2′-O-Me modifications on all four bases of ZIKV genomic RNA (RNA, 50 ng/sample). Data are expressed as the ratio of modified to unmodified bases (m⁶A/A, Am/A, Gm/G, Um/U, and Cm/C). N=3. (FIG. 1B) m⁶A-seq of ZIKV RNA showing the distribution of m⁶A reads mapped to the ZIKV genome (red line). The baseline signal from input samples is shown as a black line, and the m⁶A peaks are shown as rectangles along the x-axis. A schematic diagram of the ZIKV genome is shown below to indicate the location of the m⁶A-enriched sequences. Data are representative of n=3 determinations. FIG. 1B is split at approximately 6000 nt, the arrow indicates it continues immediately below. (FIG. 1C) Modulation of ZIKV RNA methylation by METTL3/METTL14 and ALKBH5. 293T cells were transfected with a non-targeting control shRNA (NTC) or shRNAs targeting METTL3, METTL14, ALKBH5 or FTO (knockdown, KD). RNA was isolated by Me-RIP and quantified by qRT-PCR. N=3. (FIG. 1D) Localization of METTL3, METTL14, and ALKBH5 in the nucleus and cytoplasm of ZIKV-infected cells. Nuclear and cytoplasmic fractions of mock- or ZIKV-infected 293T cells were subjected to western blot analysis using antibodies against METTL3, METTL14, and ALKBH5 enzymes. Histone H3 and GAPDH were probed as controls for each fraction. Data are the mean±SEM of the indicated number of replicates. Student's t-test: *p<0.05, **p<0.005, ***p<0.0005.

FIGS. 2A-2G. m⁶A RNA Methylation Modulates the ZIKV Life Cycle. (FIG. 2A) Enhancement of ZIKV replication by METTL3/METTL14 silencing and reduction by ALKBH5/FTO silencing. 293T cells expressing a non-targeting control shRNA (NTC) or shRNAs targeting METTL3, METTL14, ALKBH5 or FTO shRNA (KD) were infected with ZIKV. Supernatants were harvested 6, 12 and 24 h later for quantification of ZIKV RNA by qRT-PCR. N=3. (FIG. 2B) Viral titers (PFU/ml) at 24 h post-infection. Cells were treated as described in (FIG. 2A). N=3. (FIG. 2C) Immunostaining of viral envelope protein in cells treated as described in (FIG. 2A). Scale bars, 100 m. (FIG. 2D) Enhancement of ZIKV RNA expression by YTHDF1-3 silencing. 293T cells were transduced with shRNAs targeting YTHDF1-3 or control shRNA. Supernatants were harvested 24 h later for quantification of ZIKV RNA by qRT-PCR. N=5. (FIG. 2E) Decrease in ZIKV RNA expression by overexpression of YTHDF 1, 2, and 3 proteins. 293T cells were transfected with FLAG-tagged YTHDF1-3 or control pcDNA vectors. Supernatants were harvested 24 h later for quantification of ZIKV RNA by qRT-PCR. N=5. (FIG. 2F) Binding of YTHDF1-3 proteins to ZIKV RNA. 293T cells transfected as described for (FIG. 2E) were immunoprecipitated with an anti-FLAG antibody and immunoblotted for FLAG proteins (top). “IN” (input) lanes contained 5% of the lysate. ZIKV RNA in the FLAG-immunoprecipitates was quantified by qRT-PCR and normalized to percentage of total intracellular ZIKV RNA (bottom). N=3. (FIG. 2G) Reduction and enhancement of YTHDF2-RNA binding by RNA methylation status. 293T cells were transfected with control or FLAG-YTHDF2 overexpression vector and co-transfected with the indicated shRNAs. Lysates were immunoprecipitated with an anti-FLAG antibody and immunoblotted for FLAG protein (top). Input lanes contained 5% of the lysate. ZIKV RNA in YTHDF2 immunoprecipitates was quantified by qRT-PCR and normalized to the level in cells expressing NTC shRNA (bottom). N=3 All data are the mean±SEM of the indicated number of replicates. Student's t-test *p<0.05, **p<0.005, ***p<0.0005.

FIGS. 3A-3D. ZIKV Infection Influences RNA Methylation of Host Cell Transcripts. (FIG. 3A) Metagene analysis of normalized m⁶A peak distribution along a reference mRNA. (FIG. 3B) Distribution of m⁶A peaks are as follows, in order from top to bottom in the legend: in the 5′-UTR (top most), coding sequence (CDS, second from the top), exon junction (third from the top), and 3′-UTR (bottom) of host cell RNA transcripts. 293T cells were mock- or ZIKV-infected, and m⁶A peaks in total cellular RNA were analyzed at 24 h after infection. Charts show the proportion of m⁶A peaks in the indicated regions in uninfected and ZIKV-infected cells (top) and the appearance of newly emerged m⁶A peaks or loss of existing m⁶A peaks after ZIKV infection (bottom). Representative of N=2 determinations. (FIG. 3C) GSEA analysis of reactome analysis of pathways associated with newly emerged m⁶A modifications (top, blue) and loss of existing m⁶A modifications (bottom, red) at 24 h after ZIKV infection of 293T cells. The top 10 enriched categories for each condition are shown. (FIG. 3D) Motif analysis to identify consensus sequences for m⁶A methylation in uninfected and ZIKV-infected 293T cells. The top 5 motifs for each are shown and are as follows, in order from top to bottom for the Uninfected unique peaks: GACUG (SEQ ID NO:26), AACGGAC (SEQ ID NO:27), AUUGCGG (SEQ ID NO:28), UCGGGAC (SEQ ID NO:29), and GAACCGG (SEQ ID NO:30). The top 5 motifs for each are shown and are as follows, in order from top to bottom for the ZIKV-infected unique peaks: GAACCU (SEQ ID NO:31), UACGG (SEQ ID NO:32), UCGCAAG (SEQ ID NO:33), GGACU (SEQ ID NO:34), and AGACUUC (SEQ ID NO:35).

FIGS. 4A-4C. (FIG. 4A) Correlation test between two biological replicates of vgRNA is shown. (FIG. 4B) Alignment and m⁶A motif identity/conservation is shown for the 12 m⁶A peaks identified in ZIKV RNA among five ZIKV strains (MR766, Paraiba, KX156774, KU501215 and FSS13025). The DRACH, MGACK, and UGAC consensus motifs are identified with a black box. (FIG. 4C) Nuclear and cytoplasmic localization of METTL3, METTL14 and ALKBH5 visualized by immunostaining of uninfected (Mock) and ZIKV-infected 293T cells at 24 h after infection. Cells were counterstained with DAPI. Scale bar, 100 m. Arrows indicate cells with evident cytoplasmic localization of the indicated protein.

FIGS. 5A-5G. (FIG. 5A) Silencing efficiency of shRNAs. METTL3, METTL14, and ALKBH5 expression in 293T cells expressing non-targeting shRNA (NTC) or the indicated gene-specific shRNAs, analyzed by qRT-PCR (left) or western blotting (right). N=3. (FIGS. 5B-5C Viral titers (particle production) (FIG. 5) and ZIKV RNA levels in supernatants (FIG. 5C) of 293T cells overexpressing METTL3, METTL14, or ALKBHS proteins. N=3. (FIG. 5D) Western blot analysis of METTL3, METTL14, and ALKBHS proteins in 293T cells expressing control pcDNA or the indicated overexpression vectors. GAPDH was probed as a loading control. All data are the mean±SEM of the indicated number of replicates. Student's t-test *p<0.05, **p<0.005, ***p<0.0005. (FIG. 5E) Silencing efficiency of YTHDF1, 2, and 3 expression in 293T cells expressing indicated gene-specific shRNAs or non-targeting shRNA control (NTC) analyzed by qRT-PCR. (FIGS. 5F-5G) Cell viability analyses by MTS assays in uninfected (FIG. 5F) and ZIKV infected (FIG. 5G) for specific shRNA knockdown (KD) or overexpression (OE) experiments as indicated.

FIGS. 6A-6G. (FIGS. 6A-6F) Paired correlation analysis of the three biological replicates of (FIG. 6A-6C) uninfected (control R1, R2, R3) and (FIG. 6D-FIG. 6F) ZIKV-infected samples (infect R1, R2, R3). Each paired results exhibit high correlation values, indicating good replicability. (FIG. 6G) Relative mRNA level of genes listed in Table 2 in mock or ZIKV-infected cells quantified by qRT-PCR.

FIG. 7. Microglial cell-line was plated 1×10⁶ cells per well in 24 well plate and cultured for 24 h. Cell were then treated in triplicate with series of methyltransferase inhibitors (10 M) for one hour followed by infection with ZIKV (MR766, MOI: 1) and changed the fresh medium after 2 hrs of ZIKV infection and re-added the drugs. After 24 h of the infection, we collected the supernatant and did one-step qRT-PCR. Fold inhibition of ZIKV infection was normalized from DMSO control.

FIG. 8. Microglial cell-line was plated 1×10⁶ cells per well in 24 well plate and cultured for 24 h. Cell were then treated in triplicate with series of methyltransferase inhibitors (10 μM) for one hour followed by infection with ZIKV (MR766, MOI: 1) and changed the fresh medium after 2 hrs of ZIKV infection and re-added the drugs. After 48 h of the infection, we collected the supernatant and did one-step qRT-PCR. Fold inhibition of ZIKV infection was normalized from DMSO control.

### DETAILED DESCRIPTION

### I. Definitions

The following definitions are provided to facilitate understanding of certain terms used frequently herein and are not meant to limit the scope of the present disclosure. The abbreviations used herein have their conventional meaning within the chemical and biological arts. The chemical structures and formulae set forth herein are constructed according to the standard rules of chemical valency known in the chemical arts.

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di- and multivalent radicals. The alkyl may include a designated number of carbons (e.g., C₁-C₁₀ means one to ten carbons). Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkyl moiety may be fully saturated. An alkenyl may include more than one double bond and/or one or more triple bonds in addition to the one or more double bonds. An alkynyl may include more than one triple bond and/or one or more double bonds in addition to the one or more triple bonds.

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene.

The term “heteroalkyl,” by itself or in combination with another term, means, unless otherwise stated, a stable straight or branched chain, or combinations thereof, including at least one carbon atom and at least one heteroatom (e.g., O, N, P, Si, and S), and wherein the nitrogen and sulfur atoms may optionally be oxidized, and the nitrogen heteroatom may optionally be quaternized. The heteroatom(s) (e.g., N, S, Si, or P) may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., O, N, S, Si, or P). A heteroalkyl moiety may include two optionally different heteroatoms (e.g., O, N, S, Si, or P). A heteroalkyl moiety may include three optionally different heteroatoms (e.g., O, N, S, Si, or P). A heteroalkyl moiety may include four optionally different heteroatoms (e.g., O, N, S, Si, or P). A heteroalkyl moiety may include five optionally different heteroatoms (e.g., O, N, S, Si, or P). A heteroalkyl moiety may include up to 8 optionally different heteroatoms (e.g., O, N, S, Si, or P). The term “heteroalkenyl,” by itself or in combination with another term, means, unless otherwise stated, a heteroalkyl including at least one double bond. A heteroalkenyl may optionally include more than one double bond and/or one or more triple bonds in additional to the one or more double bonds. The term “heteroalkynyl,” by itself or in combination with another term, means, unless otherwise stated, a heteroalkyl including at least one triple bond. A heteroalkynyl may optionally include more than one triple bond and/or one or more double bonds in additional to the one or more triple bonds.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R‘- and —R’C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzoxazoyl benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be —O— bonded to a ring heteroatom nitrogen.

Spirocyclic rings are two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings. Possible substituents for individual rings within spirocyclic rings are the possible substituents for the same ring when not part of spirocyclic rings (e.g. substituents for cycloalkyl or heterocycloalkyl rings). Spirocylic rings may be substituted or unsubstituted cycloalkyl, substituted or unsubstituted cycloalkylene, substituted or unsubstituted heterocycloalkyl or substituted or unsubstituted heterocycloalkylene and individual rings within a spirocyclic ring group may be any of the immediately previous list, including having all rings of one type (e.g. all rings being substituted heterocycloalkylene wherein each ring may be the same or different substituted heterocycloalkylene). When referring to a spirocyclic ring system, heterocyclic spirocyclic rings means a spirocyclic rings wherein at least one ring is a heterocyclic ring and wherein each ring may be a different ring. When referring to a spirocyclic ring system, substituted spirocyclic rings means that at least one ring is substituted and each substituent may optionally be different.

The symbol “ [symbol] ” denotes the point of attachment of a chemical moiety to the remainder of a molecule or chemical formula.

The term “oxo,” as used herein, means an oxygen that is double bonded to a carbon atom.

The term “alkylarylene” as an arylene moiety covalently bonded to an alkylene moiety (also referred to herein as an alkylene linker). In embodiments, the alkylarylene group has the formula:

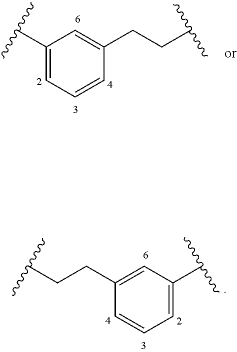 [SMILES: *CCc1cccc(*)c1 ; *CCc1cccc(*)c1]

An alkylarylene moiety may be substituted (e.g. with a substituent group) on the alkylene moiety or the arylene linker (e.g. at carbons 2, 3, 4, or 6) with halogen, oxo, —N₃, —CF₃, —CCl₃, —CBr₃, —CI₃, —CN, —CHO, —OH, —NH₂, —COOH, —CONH₂, —NO₂, —SH, —SO₂CH₃—SO₃H, —OSO₃H, —SO₂NH₂, —NHNH₂, —ONH₂, —NHC(O)NHNH₂, substituted or unsubstituted C₁-C₅ alkyl or substituted or unsubstituted 2 to 5 membered heteroalkyl). In embodiments, the alkylarylene is unsubstituted.

Each of the above terms (e.g., “alkyl,” “heteroalkyl,” “cycloalkyl,” “heterocycloalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, -halogen, —SiR′R″R″′, —OC(O)R′, —C(O)R′, —CO₂R′, —CONR′R″, —OC(O)NR′R″, —NR″C(O)R′, —NR′—C(O)NR″R″′, —NR″C(O)₂R′, —NR—C(NR′R″R″′)═NR″ ″, —NR—C(NR′R″)═NR″′, —S(O)R′, —S(O)₂R′, —S(O)₂NR′R″, —NRSO₂R′, —NR′NR″R″′, —ONR′R″, —NR′C(O)NR″NR″′R″″, —CN, —NO₂, —NR′SO₂R″, —NR′C(O)R″, —NR′C(O)—OR″, —NR′OR″, in a number ranging from zero to (2m′+1), where m′ is the total number of carbon atoms in such radical. R, R′, R″, R″′, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound described herein includes more than one R group, for example, each of the R groups is independently selected as are each R′, R″, R″′, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, -halogen, —SiR′R″R″′, —OC(O)R′, —C(O)R′, —CO₂R′, —CONR′R″, —OC(O)NR′R″, —NR″C(O)R′, —NR′— C(O)NR″R″′, —NR″C(O)₂R′, —NR—C(NR′R″R″′)═NR″″, —NR—C(NR′R″)═NR″′, —S(O)R′, —S(O)₂R′, —S(O)₂NR′R″, —NRSO₂R′, —NR′NR″R″′, —ONR′R″, —NR′C(O)NR″NR″′R″″, —CN, —NO₂, —R′, —N₃, —CH(Ph)₂, fluoro(C₁-C₄)alkoxy, and fluoro(C₁-C₄)alkyl, —NR′SO₂R″, —NR′C(O)R″, —NR′C(O)—OR″, —NR′OR″, in a number ranging from zero to the total number of open valences on the aromatic ring system; and where R′, R″, R″′, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound described herein includes more than one R group, for example, each of the R groups is independently selected as are each R′, R″, R″′, and R″″ groups when more than one of these groups is present.

Substituents for rings (e.g. cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more floating substituents (including, but not limited to, points of attachment to the remainder of the molecule), the floating substituents may be bonded to the heteroatoms. Where the ring heteroatoms are shown bound to one or more hydrogens (e.g. a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′— (C″R″R″′)_{d}—, where s and d are independently integers of from 0 to 3, and X′ is —O—, —NR′—, —S—, —S(O)—, —S(O)₂—, or —S(O)₂NR′—. The substituents R, R′, R″, and R″′ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include oxygen (O), nitrogen (N), sulfur (S), phosphorus (P), and silicon (Si).

A “substituent group,” as used herein, means a group selected from the following moieties:
(A) oxo,
halogen, —CCl₃, —CBr₃, —CF₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr3, —OCI₃, —OCHCl₂, —OCHBr₂, —OCHI₂, —OCHF₂, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C3-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
(B) alkyl, heteroalkyl, cycloalkyl, heterocycloalkyl, aryl, heteroaryl, substituted with at least one substituent selected from:
   (i) oxo,
   halogen, —CCl₃, —CBr₃, —CF₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₅ cycloalkyl, C3-C6 cycloalkyl, or C5-C6 cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C6-C10 aryl, C₁₀ aryl, or phenyl), or unsubstituted heteroaryl (e.g., 5 to 10 membered heteroaryl, 5 to 9 membered heteroaryl, or 5 to 6 membered heteroaryl), and
   (ii) alkyl, heteroalkyl, cycloalkyl, heterocycloalkyl, aryl, heteroaryl, substituted with at least one substituent selected from:
      (a) oxo,
      halogen, —CCl₃, —CBr₃, —CF₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCH Cl₂, —OCHBr₂, —OCHI₂, —OCHF₂, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
      (b) alkyl, heteroalkyl, cycloalkyl, heterocycloalkyl, aryl, heteroaryl, substituted with at least one substituent selected from: oxo,
   halogen, —CCl₃, —CBr₃, —CF₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C3-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C6-C10 aryl, C₁₀ aryl, or phenyl), or unsubstituted heteroaryl (e.g., 5 to 10 membered heteroaryl, 5 to 9 membered heteroaryl, or 5 to 6 membered heteroaryl).

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and each substituted or unsubstituted heteroaryl is a substituted or unsubstituted 5 to 9 membered heteroaryl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In some embodiments of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In some embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 9 membered heteroaryl. In some embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 9 membered heteroarylene. In some embodiments, the compound is a chemical species set forth in the Examples section, figures, or tables below.

In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is unsubstituted (e.g., is an unsubstituted alkyl, unsubstituted heteroalkyl, unsubstituted cycloalkyl, unsubstituted heterocycloalkyl, unsubstituted aryl, unsubstituted heteroaryl, unsubstituted alkylene, unsubstituted heteroalkylene, unsubstituted cycloalkylene, unsubstituted heterocycloalkylene, unsubstituted arylene, and/or unsubstituted heteroarylene, respectively). In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is substituted (e.g., is a substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene, respectively).

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, wherein if the substituted moiety is substituted with a plurality of substituent groups, each substituent group may optionally be different. In embodiments, if the substituted moiety is substituted with a plurality of substituent groups, each substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one size-limited substituent group, wherein if the substituted moiety is substituted with a plurality of size-limited substituent groups, each size-limited substituent group may optionally be different. In embodiments, if the substituted moiety is substituted with a plurality of size-limited substituent groups, each size-limited substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one lower substituent group, wherein if the substituted moiety is substituted with a plurality of lower substituent groups, each lower substituent group may optionally be different. In embodiments, if the substituted moiety is substituted with a plurality of lower substituent groups, each lower substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted moiety is substituted with a plurality of groups selected from substituent groups, size-limited substituent groups, and lower substituent groups; each substituent group, size-limited substituent group, and/or lower substituent group may optionally be different. In embodiments, if the substituted moiety is substituted with a plurality of groups selected from substituent groups, size-limited substituent groups, and lower substituent groups; each substituent group, size-limited substituent group, and/or lower substituent group is different.

Certain compounds of the present disclosure possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present disclosure. The compounds of the present disclosure do not include those that are known in art to be too unstable to synthesize and/or isolate. The present disclosure is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds of this invention may exist in tautomeric forms, all such tautomeric forms of the compounds being within the scope of the invention.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the invention.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this invention.

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present invention, whether radioactive or not, are encompassed within the scope of the present invention.

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

The symbol “ [symbol] ” denotes the point of attachment of a chemical moiety to the remainder of a molecule or chemical formula.

The terms “a” or “an,” as used in herein means one or more. In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls. Moreover, where a moiety is substituted with an R substituent, the group may be referred to as “R-substituted.” Where a moiety is R-substituted, the moiety is substituted with at least one R substituent and each R substituent is optionally different. Where a particular R group is present in the description of a chemical genus, a Roman alphabetic symbol may be used to distinguish each appearance of that particular R group. For example, where multiple R¹³ substituents are present, each R¹³ substituent may be distinguished as R^{13A}, R^{13B}, R^{13C}, R^{13D}, etc., wherein each of R^{13A}, R^{13B}, R^{13C}, R^{13D}, etc. is defined within the scope of the definition of R¹³ and optionally differently.

Descriptions of compounds of the present invention are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

“Analog” or “analogue” or “chemical analog” or chemical analogue” or “structural analog” or “structural analogue”, as may be used interchangeably herein and used in accordance with plain ordinary meaning within Chemistry and Biology, refer to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an “analog” or “analogue” or “chemical analog” or chemical analogue” is a compound that is similar or comparable in function and appearance but not in structure or origin to a reference compound.

The terms “functional analog” or “functional analogue” or “small molecule functional analog” or “small molecule functional analogue”, as may be used interchangeably herein and used in accordance with plain ordinary meaning, refer to chemical compounds that have a similar effect on a target (e.g., increasing the function or activity of a protein). Functional analogs are not necessarily structural analogs with a similar chemical structure. An example of pharmacological functional analogs are morphine, heroin, and fentanyl, which have the same mechanism of action, but fentanyl is structurally different from the other two. In embodiments, the small molecule functional analog or small molecule functional analogue is a chemical compound with a molecular weight less than about 900 Daltons.

Moreover, where a moiety is substituted with an R substituent, the group may be referred to as “R-substituted.” Where a moiety is R-substituted, the moiety is substituted with at least one R substituent and each R substituent is optionally different. Where a particular R group is present in the description of a chemical genus (such as Formula (I)), a Roman alphabetic symbol may be used to distinguish each appearance of that particular R group. For example, where multiple R¹³ substituents are present, each R¹³ substituent may be distinguished as R^{13A}, R^{13B}, R^{13C}, R^{13D}, etc., wherein each of R^{13A}, R^{13B}, R^{13C}, R^{13D}, etc. is defined within the scope of the definition of R¹³ and optionally differently.

Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

The term “pharmaceutically acceptable salts” is meant to include salts of the active compounds that are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

Thus, the compounds of the present disclosure may exist as salts, such as with pharmaceutically acceptable acids. The present disclosure includes such salts. Non-limiting examples of such salts include hydrochlorides, hydrobromides, phosphates, sulfates, methanesulfonates, nitrates, maleates, acetates, citrates, fumarates, proprionates, tartrates (e.g., (+)-tartrates, (−)-tartrates, or mixtures thereof including racemic mixtures), succinates, benzoates, and salts with amino acids such as glutamic acid, and quaternary ammonium salts (e.g. methyl iodide, ethyl iodide, and the like). These salts may be prepared by methods known to those skilled in the art.

The neutral forms of the compounds are preferably regenerated by contacting the salt with a base or acid and isolating the parent compound in the conventional manner. The parent form of the compound may differ from the various salt forms in certain physical properties, such as solubility in polar solvents.

In addition to salt forms, the present disclosure provides compounds, which are in a prodrug form. Prodrugs of the compounds described herein are those compounds that readily undergo chemical changes under physiological conditions to provide the compounds of the present disclosure. Prodrugs of the compounds described herein may be converted in vivo after administration. Additionally, prodrugs can be converted to the compounds of the present disclosure by chemical or biochemical methods in an ex vivo environment, such as, for example, when contacted with a suitable enzyme or chemical reagent.

Certain compounds disclosed herein can exist in unsolvated forms as well as solvated forms, including hydrated forms. In general, the solvated forms are equivalent to unsolvated forms and are encompassed within the scope disclosed herein. Certain compounds disclosed herein may exist in multiple crystalline or amorphous forms. In general, all physical forms are equivalent for the uses disclosed herein and are intended to be within the scope of the compounds and methods disclosed herein.

“Pharmaceutically acceptable excipient” and “pharmaceutically acceptable carrier” refer to a substance that aids the administration of an active agent to and absorption by a subject and can be included in the compositions of the present disclosure without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the disclosure. One of skill in the art will recognize that other pharmaceutical excipients are useful in the present disclosure.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

The terms “treating”, or “treatment” refer to any indicia of success in the treatment or amelioration of an injury, disease, infection (e.g., Zika viral infection or RNA virus infection), pathology or condition, including any objective or subjective parameter such as abatement; remission; diminishing of symptoms or making the injury, pathology or condition more tolerable to the patient; slowing in the rate of degeneration or decline; making the final point of degeneration less debilitating; or improving a patient's physical or mental well-being. The treatment or amelioration of symptoms can be based on objective or subjective parameters, including the results of a physical examination, neuropsychiatric exams, and/or a psychiatric evaluation. The term “treating” and conjugations thereof, include prevention of an injury, pathology, condition, or disease. In embodiments, treating is not preventing. In embodiments, preventing refers to the inability of an infectious agent (e.g., Zika virus or virus particles or RNA virus infection) to spread to a second subject.

An “effective amount” is an amount sufficient to accomplish a stated purpose (e.g., achieve the effect for which it is administered, treat a disease, treat an infection (e.g., Zika viral infection or RNA virus infection), reduce enzyme activity (e.g., methylation), increase enzyme activity (e.g., demethylation), reduce one or more symptoms of a disease, infection (e.g., fever, neurological, immunological, or developmental defects), or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

For any compound described herein, the therapeutically effective amount can be initially determined from cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

Dosages may be varied depending upon the requirements of the patient and the compound being employed. The dose administered to a patient, in the context of the methods disclosed herein should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached.

Dosage amounts and intervals can be adjusted individually to provide levels of the administered compound effective for the particular clinical indication being treated. This will provide a therapeutic regimen that is commensurate with the severity of the individual's disease state.

Utilizing the teachings provided herein, an effective prophylactic or therapeutic treatment regimen can be planned that does not cause substantial toxicity and yet is effective to treat the clinical symptoms demonstrated by the particular patient. This planning should involve the careful choice of active compound by considering factors such as compound potency, relative bioavailability, patient body weight, presence and severity of adverse side effects, preferred mode of administration and the toxicity profile of the selected agent.

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In embodiments, a control is the measurement of the activity of a protein in the absence of a compound as described herein (including embodiments and examples).

“Contacting” is used in accordance with its plain ordinary meaning and refers to the process of allowing at least two distinct species (e.g., chemical compounds including biomolecules or cells) to become sufficiently proximal to react, interact or physically touch. It should be appreciated; however, the resulting reaction product can be produced directly from a reaction between the added reagents or from an intermediate from one or more of the added reagents which can be produced in the reaction mixture.

The term “contacting” may include allowing two species to react, interact, or physically touch, wherein the two species may be a compound as described herein and a protein or enzyme. Contacting may include allowing a compound described herein to interact with a protein or enzyme that is involved in a signaling pathway.

As defined herein, the term “inhibition”, “inhibit”, “inhibiting” or the like refer, in the usual and customary sense, to decreasing the amount or activity of a target relative to the absence of the recited inhibitor.

As defined herein, the term “activation”, “activate”, “activating” and the like in reference to a protein refers to conversion of a protein into a biologically active derivative from an initial inactive or deactivated state. The terms reference activation, or activating, sensitizing, or up-regulating signal transduction or enzymatic activity or the amount of a protein decreased in a disease.

The terms “agonist,” or “agonize” or activator” or “upregulator”, and the like refer to a substance capable of detectably increasing the expression or activity of a given gene, protein (e.g., METTL3 or METTL14) or target molecule, relative to the activity or function of the protein in the absence of the agonist. The agonist can increase expression or activity 10%, 20%, 30%, 40%, 50%, 60%, 70%, 80%, 90% or more in comparison to a control in the absence of the agonist. In certain instances, expression or activity is 1.5-fold, 2-fold, 3-fold, 4-fold, 5-fold, 10-fold or higher than the expression or activity in the absence of the agonist.

As defined herein, the term “inhibition”, “inhibit”, “inhibiting” and the like in reference to a protein-inhibitor interaction means negatively affecting (e.g. decreasing) the activity or function of the protein (e.g., ALKBH5 or FTO) relative to the activity or function of the protein in the absence of the inhibitor. In embodiments inhibition means negatively affecting (e.g. decreasing) the concentration or levels of the protein relative to the concentration or level of the protein in the absence of the inhibitor. In embodiments inhibition refers to reduction of a disease or infection or symptoms of disease or infection. In embodiments, inhibition refers to a reduction in the activity of a particular protein target (e.g., ALKBH5 or FTO). Thus, inhibition includes, at least in part, partially or totally blocking stimulation, decreasing, preventing, or delaying activation, or inactivating, desensitizing, or down-regulating signal transduction or enzymatic activity or the amount of a protein. In embodiments, inhibition refers to a reduction of activity of a target protein resulting from a direct interaction (e.g. an inhibitor binds to the target protein). In embodiments, inhibition refers to a reduction of activity of a target protein from an indirect interaction (e.g. an inhibitor binds to a protein that activates the target protein, thereby preventing target protein activation).

The terms “inhibitor,” “repressor” or “antagonist” or “antagonize” or “downregulator” interchangeably refer to a substance capable of detectably decreasing the expression or activity of a given gene, protein (e.g., ALKBH5 or FTO) or target molecule. The antagonist can decrease expression or activity 10%, 20%, 30%, 40%, 50%, 60%, 70%, 80%, 90% or more in comparison to a control in the absence of the antagonist. In certain instances, expression or activity is 1.5-fold, 2-fold, 3-fold, 4-fold, 5-fold, 10-fold or lower than the expression or activity in the absence of the antagonist.

The term “modulate” is used in accordance with its plain ordinary meaning and refers to the act of changing or varying one or more properties. “Modulation” refers to the process of changing or varying one or more properties. For example, a modulator of a target protein changes by increasing or decreasing a property or function of the target molecule or the amount of the target molecule. A modulator of a disease decreases a symptom, cause, or characteristic of the targeted disease.

As used herein, the term “administering” means oral administration, administration as a suppository, topical contact, intravenous, parenteral, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc.

By “co-administer” it is meant that a compound described herein is administered at the same time, just prior to, or just after the administration of one or more additional therapies. The compounds described herein can be administered alone or can be co-administered to the patient. Co-administration is meant to include simultaneous or sequential administration of the compound individually or in combination (more than one compound or agent). Thus, the preparations can also be combined, when desired, with other active substances.

Co-administration includes administering one active agent within 0.5, 1, 2, 4, 6, 8, 10, 12, 16, 20, or 24 hours of a second active agent. Also contemplated herein, are embodiments, where co-administration includes administering one active agent within 0.5, 1, 2, 4, 6, 8, 10, 12, 16, 20, or 24 hours of a second active agent. Co-administration includes administering two active agents simultaneously, approximately simultaneously (e.g., within about 1, 5, 10, 15, 20, or 30 minutes of each other), or sequentially in any order. Co-administration can be accomplished by co-formulation, i.e., preparing a single pharmaceutical composition including both active agents. In other embodiments, the active agents can be formulated separately. The active and/or adjunctive agents may be linked or conjugated to one another. Co-administration is meant to include simultaneous or sequential administration of the compounds individually or in combination (more than one composition) and includes vaccine administration in a prime-boost method. Thus, the preparations can also be combined, when desired, with other active substances (e.g. to reduce metabolic degradation, increase immune response (e.g. adjuvant)). The compositions of the present invention can be delivered by transdermally, by a topical route, transcutaneously, formulated as applicator sticks, solutions, suspensions, emulsions, gels, creams, ointments, pastes, jellies, paints, powders, and aerosols.

The term “associated” or “associated with” in the context of a substance or substance activity or function associated with a disease or infection (e.g., Zika viral infection or RNA virus infection) means that the disease or infection is caused by (in whole or in part), a symptom of the disease or infection is caused by (in whole or in part) the substance or substance activity or function, or a side-effect of the compound (e.g., toxicity) is caused by (in whole or in part) the substance or substance activity or function.

“Patient,” “subject,” “patient in need thereof,” and “subject in need thereof” are herein used interchangeably and refer to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals.

A “therapeutic agent” as used herein refers to an agent (e.g., compound or composition) that when administered to a subject will have the intended prophylactic effect, e.g., preventing or delaying the onset (or reoccurrence) of an injury, disease, infection, pathology or condition, or reducing the likelihood of the onset (or reoccurrence) of an injury, disease, infection, pathology, or condition, or their symptoms or the intended therapeutic effect, e.g., treatment or amelioration of an injury, disease, pathology or condition, or their symptoms including any objective or subjective parameter of treatment such as abatement; remission; diminishing of symptoms or making the injury, pathology or condition more tolerable to the patient; slowing in the rate of degeneration or decline; making the final point of degeneration less debilitating; or improving a patient's physical or mental well-being.

As defined herein, a therapeutically effective amount of a compound provided herein (an effective dosage) can range from about 0.001 to 3000 mg/kg body weight. Skilled artisans will appreciate that a variety of factors can influence the dosage required to effectively treat a subject, including but not limited to the severity of the disease or condition being treated, history of previous treatments, general health and/or age of the subject, and the like. Accordingly, exact dosages for any particular subject will typically be determined empirically.

“Disease” or “condition” refer to a state of being or health status of a patient or subject capable of being treated with a compound, pharmaceutical composition, or method provided herein. In embodiments, the disease is an RNA virus infection, HIV viral infection, West Nile viral infection, Dengue viral infection, Japanese encephalitis or a Zika viral infection.

The term “signaling pathway” as used herein refers to a series of interactions between cellular and optionally extra-cellular components (e.g. proteins, nucleic acids, small molecules, ions, lipids) that conveys a change in one component to one or more other components, which in turn may convey a change to additional components, which is optionally propagated to other signaling pathway components. For example, binding of METTL14 with a compound as described herein may reduce the level of a product of the METTL14 catalyzed reaction or the level of a downstream derivative of the product or binding may reduce the interactions between METTL14 or a METTL14 reaction product and downstream effectors or signaling pathway components, resulting in changes in cell growth, proliferation, or survival.

The terms “polypeptide,” “peptide” and “protein” are used interchangeably herein to refer to a polymer of amino acid residues, wherein the polymer may In embodiments be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

As may be used herein, the terms “nucleic acid,” “nucleic acid molecule,” “nucleic acid oligomer,” “oligonucleotide,” “nucleic acid sequence,” “nucleic acid fragment” and “polynucleotide” are used interchangeably and are intended to include, but are not limited to, a polymeric form of nucleotides covalently linked together that may have various lengths, either deoxyribonucleotides or ribonucleotides, or analogs, derivatives or modifications thereof. Different polynucleotides may have different three-dimensional structures, and may perform various functions, known or unknown. Non-limiting examples of polynucleotides include a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, a nucleic acid probe, and a primer. Polynucleotides useful in the methods of the disclosure may comprise natural nucleic acid sequences and variants thereof, artificial nucleic acid sequences, or a combination of such sequences.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

“Conservatively modified variants” applies to both amino acid and nucleic acid sequences. With respect to particular nucleic acid sequences, “conservatively modified variants” refers to those nucleic acids that encode identical or essentially identical amino acid sequences. Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site http://www.ncbi.nlm.nih.gov/BLAST/or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence.

The term “antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Typically, the antigen-binding region of an antibody will be most critical in specificity and affinity of binding. In some embodiments, antibodies or fragments of antibodies may be derived from different organisms, including humans, mice, rats, hamsters, camels, etc. Antibodies of the invention may include antibodies that have been modified or mutated at one or more amino acid positions to improve or modulate a desired function of the antibody (e.g. glycosylation, expression, antigen recognition, effector functions, antigen binding, specificity, etc.).

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL) and variable heavy chain (VH) refer to these light and heavy chains respectively.

For preparation of suitable antibodies of the invention and for use according to the invention, e.g., recombinant, monoclonal, or polyclonal antibodies, many techniques known in the art can be used (see, e.g., Kohler & Milstein, Nature 256:495-497 (1975); Kozbor et al., Immunology Today 4: 72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); and Goding, Monoclonal Antibodies: Principles and Practice (2d ed. 1986)). The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Random combinations of the heavy and light chain gene products generate a large pool of antibodies with different antigenic specificity (see, e.g., Kuby, Immunology (3rd ed. 1997)). Techniques for the production of single chain antibodies or recombinant antibodies (U.S. Pat. Nos. 4,946,778, 4,816,567) can be adapted to produce antibodies to polypeptides of this invention. Also, transgenic mice, or other organisms such as other mammals, may be used to express humanized or human antibodies (see, e.g., U.S. Pat. Nos. 5,545,807; 5,545,806; 5,569,825; 5,625,126; 5,633,425; 5,661,016, Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); and Lonberg & Huszar, Intern. Rev. Immunol. 13:65-93 (1995)). Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)). Antibodies can also be made bispecific, i.e., able to recognize two different antigens (see, e.g., WO 93/08829, Traunecker et al., EMBO J. 10:3655-3659 (1991); and Suresh et al., Methods in Enzymology 121:210 (1986)). Antibodies can also be heteroconjugates, e.g., two covalently joined antibodies, or immunotoxins (see, e.g., U.S. Pat. No. 4,676,980, WO 91/00360; WO 92/200373; and EP 03089).

Methods for humanizing or primatizing non-human antibodies are well known in the art (e.g., U.S. Pat. Nos. 4,816,567; 5,530,101; 5,859,205; 5,585,089; 5,693,761; 5,693,762; 5,777,085; 6,180,370; 6,210,671; and 6,329,511; WO 87/02671; EP Patent Application 0173494; Jones et al. (1986) Nature 321:522; and Verhoyen et al. (1988) Science 239:1534). Humanized antibodies are further described in, e.g., Winter and Milstein (1991) Nature 349:293. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers (see, e.g., Morrison et al., PNAS USA, 81:6851-6855 (1984), Jones et al., Nature 321:522-525 (1986); Riechmann et al., Nature 332:323-327 (1988); Morrison and Oi, Adv. Immunol., 44:65-92 (1988), Verhoeyen et al., Science 239:1534-1536 (1988) and Presta, Curr. Op. Struct. Biol. 2:593-596 (1992), Padlan, Molec. Immun., 28:489-498 (1991); Padlan, Molec. Immun., 31(3):169-217 (1994)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies. For example, polynucleotides comprising a first sequence coding for humanized immunoglobulin framework regions and a second sequence set coding for the desired immunoglobulin complementarity determining regions can be produced synthetically or by combining appropriate cDNA and genomic DNA segments. Human constant region DNA sequences can be isolated in accordance with well known procedures from a variety of human cells.

The term “aptamer” as provided herein refers to oligonucleotides (e.g. short oligonucleotides or deoxyribonucleotides), that bind (e.g. with high affinity and specificity) to proteins, peptides, and small molecules. Aptamers may be RNA. Aptamers may have secondary or tertiary structure and, thus, may be able to fold into diverse and intricate molecular structures. Aptamers can be selected in vitro from very large libraries of randomized sequences by the process of systemic evolution of ligands by exponential enrichment (SELEX as described in Ellington A D, Szostak J W (1990). In vitro selection of RNA molecules that bind specific ligands. Nature 346:818-822; Tuerk C, Gold L (1990) Systematic evolution of ligands by exponential enrichment: RNA ligands to bacteriophage T4 DNA polymerase. Science 249:505-510) or by developing SOMAmers (slow off-rate modified aptamers) (Gold L et al. (2010) Aptamer-based multiplexed proteomic technology for biomarker discovery. PLoS ONE 5(12):e15004). Applying the SELEX and the SOMAmer technology includes for instance adding functional groups that mimic amino acid side chains to expand the aptamer's chemical diversity. As a result high affinity aptamers for a protein may be enriched and identified. Aptamers may exhibit many desirable properties for targeted drug delivery, such as ease of selection and synthesis, high binding affinity and specificity, low immunogenicity, and versatile synthetic accessibility. Anti-cancer agents (e.g. chemotherapy drugs, toxins, and siRNAs) may be successfully delivered to cancer cells in vitro using apatmers.

An “antisense nucleic acid” as referred to herein is a nucleic acid (e.g., DNA or RNA molecule) that is complementary to at least a portion of a specific target nucleic acid (e.g. an mRNA translatable into a protein) and is capable of reducing transcription of the target nucleic acid (e.g. mRNA from DNA) or reducing the translation of the target nucleic acid (e.g. mRNA) or altering transcript splicing (e.g. single stranded morpholino oligo). See, e.g., Weintraub, Scientific American, 262:40 (1990). Typically, synthetic antisense nucleic acids (e.g. oligonucleotides) are generally between 15 and 25 bases in length. Thus, antisense nucleic acids are capable of hybridizing to (e.g. selectively hybridizing to) a target nucleic acid (e.g. target mRNA). In embodiments, the antisense nucleic acid hybridizes to the target nucleic acid sequence (e.g. mRNA) under stringent hybridization conditions. In embodiments, the antisense nucleic acid hybridizes to the target nucleic acid (e.g. mRNA) under moderately stringent hybridization conditions. Antisense nucleic acids may comprise naturally occurring nucleotides or modified nucleotides such as, e.g., phosphorothioate, methylphosphonate, and -anomeric sugar-phosphate, backbonemodified nucleotides. Antisense nucleic acids include, for example, siRNA, mircoRNA and the like.

A “siRNA,” “small interfering RNA,” “small RNA,” or “RNAi” as provided herein, refers to a nucleic acid that forms a double stranded RNA, which double stranded RNA has the ability to reduce or inhibit expression of a gene or target gene when present in the same cell as the gene or target gene. The complementary portions of the nucleic acid that hybridize to form the double stranded molecule typically have substantial or complete identity. In one embodiment, a siRNA or RNAi is a nucleic acid that has substantial or complete identity to a target gene and forms a double stranded siRNA. In embodiments, the siRNA inhibits gene expression by interacting with a complementary cellular mRNA thereby interfering with the expression of the complementary mRNA. Typically, the nucleic acid is at least about 15-50 nucleotides in length (e.g., each complementary sequence of the double stranded siRNA is 15-50 nucleotides in length, and the double stranded siRNA is about 15-50 base pairs in length). In other embodiments, the length is 20-30 base nucleotides, preferably about 20-25 or about 24-29 nucleotides in length, e.g., 20, 21, 22, 23, 24, 25, 26, 27, 28, 29, or 30 nucleotides in length.

The term “CRISPR,” “Clustered regularly interspaced short palindrome repeats” or the like refer, in the usual and customary sense, to segments of DNA (e.g., prokaryotic DNA) containing short repetitions of base sequences. Each repetition is typically followed by short segments of spacer DNA, as known in the art, from previous exposures to an infectious agent, e.g., a bacteriophage virus or plasmid. The term “CRISPR/Cas system” or the like refers, in the usual and customary sense, to a prokaryotic immune system that confers resistance to foreign genetic elements such as those present within plasmids and phages, providing a form of acquired immunity. As known in the art, CRISPR associated proteins (Cas) use the CRISPR spacers to recognize and cut these exogenous genetic elements in a manner analogous to RNA interference in eukaryotic organisms. Accordingly, delivery of the Cas9 nuclease and appropriate guide RNAs (e.g., nucleic acid sequences described herein) into a cell can result in scission of the genome of the cell at a desired location, allowing existing genes to be removed and/or new genes or fragments thereof to be added. A CRISPR may be a nucleic acid described herein, or the RNA nucleic acid sequence corresponding to a DNA nucleic acid described herein, wherein all instances of thymine are replaced with uracil.

The term “guide RNA” or “gRNA” as provided herein refers, in the usual and customary sense, to a ribonucleotide sequence capable of binding a nucleoprotein, thereby forming ribonucleoprotein complex. In embodiments, the guide RNA includes one or more RNA molecules. In embodiments, the gRNA includes a nucleotide sequence complementary to a target site. The complementary nucleotide sequence may mediate binding of the ribonucleoprotein complex to the target site thereby providing the sequence specificity of the ribonucleoprotein complex. Thus, in embodiments, the guide RNA is complementary to a target nucleic acid. In embodiments, the guide RNA binds a target nucleic acid sequence. In embodiments, the guide RNA is complementary to a CRISPR nucleic acid sequence. In embodiments, the complement of the guide RNA has a sequence identity of about 50%, 55%, 60%, 65%, 70%, 75%, 80%, 85%, 90%, 95%, 96%, 97%, 98% or 99% to a target nucleic acid. In embodiments, a target nucleic acid sequence as provided herein is a nucleic acid sequence expressed by a cell. In embodiments, the target nucleic acid sequence is an exogenous nucleic acid sequence. In embodiments, the target nucleic acid sequence is an endogenous nucleic acid sequence. In embodiments, the target nucleic acid sequence forms part of a cellular gene. Thus, in embodiments, the guide RNA is complementary to a cellular gene or fragment thereof. In embodiments, the guide RNA binds a cellular gene sequence.

The term “ZIKA infection,” “Zika fever”, “Zika viral infection”, or the like refer, in the usual and customary sense, to a viral infection caused by the Zika virus (ZIKV), a member of the Flaviviridae family. The Zika virus is enveloped and icosahedral, having a nonsegmented, single-stranded, 10 kilobase positive-sense RNA genome. The term “Zika virus structural protein” or the like refer, in the usual and customary sense, to structural proteins encoded within the Zika virus genome. See e.g., Abbink, P., et al., Science 2016, 353:1129-1132, which is incorporated herein in its entirety for all purposes.

The terms “HIV infection” “HIV”, “HIV viral infection”, or the like refer, in the usual and customary sense, to a viral infection caused by the Human Immunodeficiency Virus (HIV).

The terms “Dengue infection” “Dengue Fever”, “Dengue viral infection”, “Dengue Disease”, or the like refer, in the usual and customary sense, to a viral infection caused by the Dengue Virus (DENV).

The terms “West Nile infection” or “West Nile viral infection”, or the like refer, in the usual and customary sense, to a viral infection caused by the West Nile virus (WNV).

The term “Japanese encephalitis” refers, in the usual and customary sense, to a viral infection caused by the mosquito-borne Japanese encephalitis virus (JEV).

The term “RNA compound” as used herein, refers to an agent (e.g., compound, nucleic acid, molecule, or virus) that encodes an RNA virus structural protein. In embodiments, the RNA compound is modified on at least one 2′ position with a 2′O-methyl and/or modified on at least one adenosine at the N6 adenine position with a methyl functionality. In embodiments, the RNA compound is a nucleic acid. In embodiments, the RNA compound is viral RNA. In embodiments, the RNA compound is a nucleic acid which includes a nucleotide which has 2′-O ribose methylation (2′-O-Me). In embodiments, the RNA compound is a nucleic acid which includes a nucleotide which has an adenosine which has been methylated on the N-6 position of adenosine. In embodiments, the RNA compound is a viral gene.

The terms “m6A” “m⁶A” or “adenosine N-6 methylation” “adenosine N-6 methylation event” or the like as used herein, refer to methylation at the N-6 position of adenosine. For example, the term may refer to the process of methylating adenosine (i.e. adding a —CH₃ moiety to the 6-position of adenosine), or:

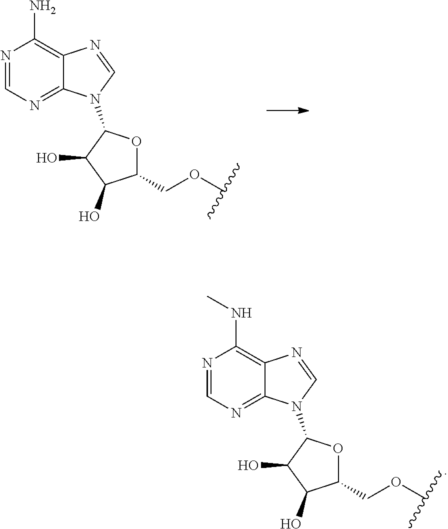 [SMILES: *OC[C@H]1O[C@@H](n2cnc3c(N)ncnc32)[C@H](O)[C@@H]1O ; *OC[C@H]1O[C@@H](n2cnc3c(NC)ncnc32)[C@H](O)[C@@H]1O]

The term “adenosine N-6 methylation agonist” as used herein refers to an agent (e.g., nucleic acid, antibody, or compound) capable of detectably increasing the quantity of adenosine N-6 methylation or activity of a given gene, protein or target molecule associated with adenosine N-6 methylation, relative to the absence of the adenosine N-6 methylation agonist. In embodiments, the adenosine N-6 methylation agonist increases the overall quantity of methylation at the N-6 position of adenosine. Non-limiting examples of adenosine N-6 methylation agonists include a RNA methyltransferase complex agonist, N6-adenosine-methyltransferase (METTL3) agonist, or Methyltransferase-Like Protein 14 (METTL14) agonist, adenosine N-6 methylation antisense nucleic acid agonist (e.g., N-6 methylation RNAi agonist), adenosine N-6 methylation aptamer agonist, and adenosine N-6 methylation antibody agonist.

The term “adenosine N-6 demethylation antagonist” as used herein refers to an agent (e.g., nucleic acid, antibody, or compound) capable of detectably decreasing the quantity of adenosine N-6 demethylation or activity of a given gene, protein or target molecule associated with adenosine N-6 demethylation, relative to the absence of the adenosine N-6 demethylation antagonist. In embodiments, the adenosine N-6 demethylation antagonist increases the overall quantity of methylation at the N-6 position of adenosine Non-limiting examples of adenosine N-6 demethylation antagonists include alkB homolog 5 RNA demethylase (ALKBH5) antagonist or an alpha-ketoglutarate-dependent dioxygenase (FTO) antagonist, N-6 demethylation antisense nucleic acid antagonist (e.g., adenosine N-6 demethylation RNAi antagonist), adenosine N-6 demethylation aptamer antagonist, adenosine N-6 demethylation antibody antagonist, and adenosine N-6 demethylation CRISPR antagonist.

The term “RNA 2′-O-methyl transferase inhibitor” as used herein refers to a compound (e.g., a compound described herein, including embodiments) capable of inhibiting (e.g., reducing the activity relative to the absence of the inhibitor) the activity or function of viral RNA 2′-O-methyl transferase (e.g., a Flavivirus RNA 2′-O-methyl transferase). Non-limiting examples of RNA 2′-O-methyl transferase inhibitors include RNA 2′-O-methyl transferase antisense inhibitor, RNA 2′-O-methyl transferase CRISPR inhibitor, RNA 2′-O-methyl aptamer inhibitor, or an RNA 2′-O-methyl transferase antibody inhibitor, nonstructural protein 5 (NS5) inhibitor, S-adenosylmethionine, a chemical analogue of S-adenosylmethionine, or a small molecule functional analogue of S-adenosylmethionine, or a compound described herein. Additional exemplary RNA 2-‘O’methyl transferase inhibitors include the compounds set forth in Example 3 (Table 4) disclosed herein. Additional RNA 2-‘O’methyl transferase inhibitors include those disclosed in: U.S. Pat. Nos. 7,465,544 and 7,670,777; Zhang & Zheng (ACS Chem. Biol. 2016, 11:583-597; Coutard et al. J. of Virology 91(5); e02202-16; and Wertheimer, A. M., et al., J. Biol. Chem., 1980, 255:5924-5930, which are incorporated herein in their entirety for all purposes. In embodiments, the RNA 2′-O-methyl transferase inhibitor is a compound described herein, including embodiments.

The terms “adenosine N-6 methylation CRISPR agonist” as used herein refers to a nucleic acid sequence (e.g., an nucleic acid sequence described herein, including Tables 5 and 6) which is capable of agonizing adenosine N-6 methylation, relative to the absence of the adenosine N-6 methylation CRISPR agonist. In embodiments, the adenosine N-6 methylation CRISPR agonist is a nucleic acid sequence (e.g., a nucleic acid sequence described herein, including embodiments) which increases methylation of a nucleotide (e.g., adenosine N-6 methylation) by silencing a gene responsible for encoding a protein in the demethylation of a nucleotide (e.g., adenosine N-6 demethylation). In embodiments, the adenosine N-6 methylation CRISPR agonist is a nucleic acid sequence (e.g., a nucleic acid sequence described herein, including embodiments) which increases adenosine N-6 methylation by a silencing a gene responsible for encoding a protein responsible for adenosine N-6 demethylation (e.g., ALKBH5 or FTO). In embodiments, the adenosine N-6 methylation CRISPR agonist is a nucleic acid described herein, or the RNA nucleic acid sequence corresponding to a DNA nucleic acid described herein, wherein all instances of thymine are replaced with uracil.

The term “adenosine N-6 demethylation CRISPR antagonist” as used herein refers to a nucleic acid sequence (e.g., an nucleic acid sequence described herein, including Tables 5 and 6) which is capable of antagonizing adenosine N-6 demethylation, relative to the absence of the adenosine N-6 demethylation CRISPR antagonist. In embodiments, the adenosine N-6 demethylation CRISPR antagonist is a nucleic acid sequence (e.g., a nucleic acid sequence described herein, including embodiments) which reduces adenosine N-6 demethylation by inactivating or silencing a gene responsible for encoding a protein (e.g., ALKBH5 or FTO) which is capable of demethylating adenosine. In embodiments, the adenosine N-6 demethylation CRISPR antagonist is a nucleic acid described herein, or the RNA nucleic acid sequence corresponding to a DNA nucleic acid described herein, wherein all instances of thymine are replaced with uracil.

The term “RNA 2′-O-methyl transferase CRISPR inhibitor” as used herein refers to a nucleic acid sequence (e.g., an nucleic acid sequence described herein, including Tables 5 and 6) which is capable of inhibiting RNA 2′-O-methyl transferase relative to the absence of the RNA 2′-O-methyl transferase CRISPR inhibitor. In embodiments, the RNA 2′-O-methyl transferase CRISPR inhibitor is a nucleic acid sequence (e.g., a nucleic acid sequence described herein, including embodiments) which reduces the methylation of a 2′-OH of one or more of nucleotide (e.g., adenosine, guanosine, uracil or cytosine nucleotide) in viral RNA. In embodiments, the RNA 2′-O-methyl transferase CRISPR inhibitor is a nucleic acid described herein, or the RNA nucleic acid sequence corresponding to a DNA nucleic acid described herein, wherein all instances of thymine are replaced with uracil.

The terms “RNA methyltransferase complex” and “MTase”, as used interchangeably herein, refer to a complex comprising two catalytic subunits (METTL3 and METTL14), a splicing factor (WTAP), a protein (KIAA1429), and other subunits not yet identified, as described in the following references: Bokar, J. A., Shambaugh, M. E., Polayes, D., Matera, A. G., and Rottman, F. M. (1997). Purification and cDNA cloning of the AdoMet-binding subunit of the human mRNA (N6-adenosine)-methyltransferase. RNA 3, 1233-1247; Liu, J., Yue, Y., Han, D., Wang, X., Fu, Y., Zhang, L., Jia, G., Yu, M., Lu, Z., Deng, X., et al. (2014). A METTL3-METTL14 complex mediates mammalian nuclear RNA N6-adenosine methylation. Nature chemical biology 10, 93-95; Ping, X. L., Sun, B.F., Wang, L., Xiao, W., Yang, X., Wang, W. J., Adhikari, S., Shi, Y., Lv, Y., Chen, Y. S., et al. (2014). Mammalian WTAP is a regulatory subunit of the RNA N6-methyladenosine methyltransferase. Cell research 24, 177-189; Schwartz, S., Mumbach, M. R., Jovanovic, M., Wang, T., Maciag, K., Bushkin, G. G., Mertins, P., Ter-Ovanesyan, D., Habib, N., Cacchiarelli, D., et al. (2014). Perturbation of m6A writers reveals two distinct classes of mRNA methylation at internal and 5′ sites. Cell Rep 8, 284-296, which are incorporated herein in their entirety for all purposes.

The term “METTL3” refers to the protein N6-adenosine-methyltransferase involved in the posttranscriptional methylation of internal adenosine residues in eukaryotic mRNAs and involved in mRNA biogenesis, decay, and translation control through N(6)-methyladenosine (m(6)A) modification. The term “METTL3” may refer to the nucleotide sequence or protein sequence of human METTL3 (e.g., Entrez 56339, Uniprot Q86U44, RefSeq NM_019852, or RefSeq NP_062826). The term “METTL3” includes both the wild-type form of the nucleotide sequences or proteins as well as any mutants thereof. In some embodiments, “METTL3” is wild-type METTL3. In some embodiments, “METTL3” is one or more mutant forms. In embodiments, an METTL3 is the human METTL3. In embodiments, the METTL3 has the nucleotide sequence corresponding to reference number GI:99077115. In embodiments, the METTL3 has the nucleotide sequence corresponding to RefSeq NM_019852.4. In embodiments, the METTL3 has the protein sequence corresponding to RefSeq NP_062826.2.

The terms “METTL14” and “methyltransferase-like 14”, as used interchangeably herein, refers to the protein methyltransferase like 14 involved in the posttranscriptional methylation of internal adenosine residues in eukaryotic mRNAs. Together with METTL3, these two proteins form a stable heterodimer core complex of METTL3-METTL14 that functions in cellular m(6)A deposition on mammalian nuclear RNAs. The term “METTL14” may refer to the nucleotide sequence or protein sequence of human METTL14 (e.g., Entrez 57721, Uniprot Q9HCE5, RefSeq NM_020961, or RefSeq NP_066012). The term “METTL14” includes both the wild-type form of the nucleotide sequences or proteins as well as any mutants thereof. In some embodiments, “METTL14” is wild-type METTL14. In some embodiments, “METTL14” is one or more mutant forms. In embodiments, an METTL14 is the human METTL14. In embodiments, the METTL14 has the nucleotide sequence corresponding to RefSeq NM_020961.3. In embodiments, the METTL14 has the protein sequence corresponding to RefSeq NP 066012.1.

The term “FTO”, as used herein, refers to fat mass and obesity-associated protein, also known as alpha-ketoglutarate-dependent dioxygenase. The term “FTO” may refer to the nucleotide sequence or protein sequence of human FTO (e.g., Entrez 79068, Uniprot Q9COB 1, RefSeq NM_001080432, or RefSeq NP_001073901). The term “FTO” includes both the wild-type form of the nucleotide sequences or proteins as well as any mutants thereof. In some embodiments, “FTO” is wild-type FTO. In some embodiments, “FTO” is one or more mutant forms. In embodiments, an FTO is the human FTO. In embodiments, the FTO has the nucleotide sequence corresponding to RefSeq NM_001080432.2. In embodiments, the FTO has the protein sequence corresponding to RefSeq NP_001073901.1.

The term “FTO antagonist” refers to a compound (e.g. compounds described herein) that antagonizes or inhibits the catalytic activity of FTO, relative to the absence of the FTO antagonist. Non-limiting examples include meclofenamic acid, N-oxalyglycine, cassic acid (i.e. Rhein), and 4-chloro-6-(6′-chloro-7′-hydroxy-2′,4′,4′-trimethyl-chroman-2′-yl)benzene-1,3-diol (CHTB). Additional inhibitors of FTO may be found in Qiao et al. Biochemistry. 55(10): 1514-1522 (2016); and McMurray et al., PLoS One. 2015 Apr. 1; 10(4):e0121829 (e.g., [(1-chloro-4-hydroxy-isoquinoline-3-carbonyl)-amino]-acetic acid); which are incorporated herein in its entirety for all purposes.

The term “ALKBH5”, refers to the protein Alkb homolog 5, ma demethylase encoded by the ALKBH5 gene or the ALKBH5 gene itself, which belongs to the AlkB family of dioxygenases. The term “ALKBH5” may refer to the nucleotide sequence or protein sequence of human ALKBH5 (e.g., Entrez 54890, Uniprot Q6P6C2, RefSeq NM_017758, or RefSeq NP_060228). The term “ALKBH5” includes both the wild-type form of the nucleotide sequences or proteins as well as any mutants thereof. In some embodiments, “ALKBH5” is wild-type ALKBH5. In some embodiments, “ALKBH5” is one or more mutant forms. In embodiments, an ALKBH5 is the human ALKBH5. In embodiments, the ALKBH5 has the nucleotide sequence corresponding to RefSeq NM_017758.3. In embodiments, the ALKBH5 has the protein sequence corresponding to RefSeq NP_060228.3.

The term “ALKBH5 antagonist” refers to a compound (e.g. compounds described herein) that antagonizes or inhibits the catalytic activity of ALKBH5 relative to the absence of the ALKBH5 antagonist. Non-limiting examples include meclofenamic acid, cassic acid (i.e. Rhein), citrate, pyridine-2,4-dicarboxylate (PDCA), N-oxalylglycine, and succinate.

The term “nonstructural protein 5(NS5) inhibitor”, as used herein, refers to a compound, an aptamer, an antibody, or a CRISPR (e.g., a nucleic acid sequence described herein) as disclosed herein, that reduces the activity of nonstructural protein 5 (NS5) when compared to a control, such as absence of the compound, an aptamer, an antibody, or a CRISPR, or a compound, an aptamer, an antibody, or a CRISPR with known inactivity. In embodiments, NS5 is human NS5 (e.g., Uniprot accession number B6VDJ7). In embodiments, an NS5 inhibitor is an inhibitor disclosed in Lira S P, Noble C G, Seh C C, Soh T S, El Sahili A, et al. (2016) Potent Allosteric Dengue Virus NS5 Polyrmerase Inhibitors: Mechanism of Action and Resistance Profiling, PLOS Pathogens 12(8): e1005737; and P. Niyomrattanakit et al. J. Virol. June 2010, vol. 84 no. 11, 5678-5686 (e.g., NITD-1, NITD-2, or NITD-29); which are incorporated herein in their entirety for all purposes.

The term “immunogenic agent” as used herein refers, in the usual and customary sense, to a particular substance (e.g., nucleic acid or compound described herein), such as an antigen or epitope, to provoke an immune response in the body of a human or animal, such as the ability to induce a humoral and/or cell-mediated immune responses. In embodiments, the immunogenic agent is an RNA virus. In embodiments, the immunogenic agent is a double stranded RNA (dsRNA). In embodiments, the immunogenic agent is a double stranded RNA (dsRNA) which mimic a viral infection (e.g., a Zika viral infection)

The terms “virus” or “virus particle” are used according to their plain ordinary meaning within Virology and refers to a virion including the viral genome (e.g. DNA, RNA, single strand, double strand), viral capsid and associated proteins, and in the case of enveloped viruses (e.g. Zika virus), an envelope including lipids and optionally components of host cell membranes, and/or viral proteins.

The term “viral structural protein” as used herein, refers to a viral protein that is a structural component of a virus (e.g., a virus which is capable of encoding a protein). In embodiments, the virus structural protein is an RNA virus structural protein. In embodiments, the RNA virus structural protein is a Zika virus structural protein. In embodiments, the RNA virus structural protein is a viral premembrane protein (prM), viral envelope protein (Env), a capsid protein (C) or a membrane protein (M).

The term “plaque forming units” is used according to its plain ordinary meaning in Virology and refers to a unit of measurement based on the number of plaques per unit volume of a sample. In some embodiments the units are based on the number of plaques that could form when infecting a monolayer of susceptible cells. Plaque forming unit equivalents are units of measure of inactivated virus. In some embodiments, plaque forming unit equivalents are derived from plaque forming units for a sample prior to inactivation. In embodiments, plaque forming units are abbreviated “Pfu.”

The term “RNA virus” as used herein refers, in the usual and customary sense, to a a virus that has RNA (ribonucleic acid) as its genetic material. In embodiments, the RNA is single-stranded RNA (e.g., ssRNA). In embodiments, the RNA is positive (+) single-stranded RNA (e.g., Bymoviruses, comoviruses, nepoviruses, nodaviruses, picornaviruses, potyviruses, sobemoviruses, luteoviruses (e.g., beet western yellows virus, barley yellow dwarf virus, potato leafroll virus), Carmoviruses, dianthoviruses, flaviviruses, pestiviruses, statoviruses, tombusviruses, single-stranded RNA bacteriophages, hepatitis C virus, Alphaviruses, carlaviruses, furoviruses, hordeiviruses, potexviruses, rubiviruses, tobraviruses, tricornaviruses, tymoviruses, apple chlorotic leaf spot virus, or hepatitis E virus). In embodiments, the RNA is double-stranded RNA (e.g., dsRNA). In embodiments, the RNA virus is a Picornavirata virus. In embodiments, the RNA virus is a Flavivirata virus. In embodiments, the RNA virus is a Rubivirata virus. In embodiments, the RNA virus is a Zika virus.

The terms “viral infection” or “viral disease” or “viral infectious disease” or “virus infection” as used interchangeably herein refers, in the usual and customary sense, to the presence of a virus (e.g., RNA virus) within a subject. In embodiments, a viral infection refers to the presence of a virus (e.g., RNA virus) within a subject that is capable of replicating and/or generating virus particles. In embodiments, the viral infection refers to the presence of a virus (e.g., RNA virus) within a subject that is capable of infecting a second subject. A viral infection can be present in any body issue and the subject may present symptoms such as fever, red eyes, joint pain, headache, and a maculopapular rash, or the subject may be asymptomatic. Diagnosis of a viral infection may be determined by testing bodily fluids (e.g., blood, urine, or saliva) for the presence of the virus's RNA or for antibodies. In embodiments, the virus may be present within a subject but may be latent.

The terms “multiplicity of infection” or “MOI” are used according to its plain ordinary meaning in Virology and refers to the ratio of components (e.g., Zika virus) to the target (e.g., cell) in a given area. In embodiments, the area is assumed to be homogeneous.

The term “adjuvant” is used in accordance with its plain ordinary meaning within Immunology and refers to a substance that is commonly used as a component of a vaccine. Adjuvants may increase an antigen specific immune response in a subject when administered to the subject with one or more specific antigens as part of a vaccine. In some embodiments, an adjuvant accelerates an immune response to an antigen. In some embodiments, an adjuvant prolongs an immune response to an antigen. In some embodiments, an adjuvant enhances an immune response to an antigen. Typically, adjuvants do not provide immunity alone. Non-limiting examples of adjuvants include aluminum-based mineral salt adjuvant (e.g., aluminum hydroxide adjuvant or aluminum phosphate adjuvant), calcium phosphate hydroxide, paraffin oil, squalene, or lipopolysaccharides. The term “lipopolysaccharides” is used according to its plain meaning in Biology, Biochemistry, and Immunology and refer to molecules comprising one or more lipids and one or more polysaccharides covalently bonded together.

The term “aluminum-based mineral salt adjuvant” refers to an adjuvant including aluminum. In some embodiments, an aluminum-based mineral salt adjuvant includes aluminum hydroxide. In some embodiments, an aluminum-based mineral salt adjuvant is aluminum hydroxide. In some embodiments, an aluminum-based mineral salt adjuvant includes aluminum phosphate. In some embodiments, an aluminum-based mineral salt adjuvant is aluminum phosphate. In some embodiments, an aluminum-based mineral salt adjuvant includes potassium aluminum sulfate. In some embodiments, an aluminum-based mineral salt adjuvant is potassium aluminum sulfate. In some embodiments, an aluminum-based mineral salt adjuvant is aluminum hydroxide adjuvant. In some embodiments, an aluminum-based mineral salt adjuvant is aluminum phosphate adjuvant. In some embodiments, an aluminum-based mineral salt adjuvant is potassium aluminum sulfate adjuvant. In some embodiments, an aluminum-based mineral salt adjuvant is Alum. In some embodiments, an aluminum-based mineral salt adjuvant is CAS no. 21645-51-2. In some embodiments, an aluminum-based mineral salt adjuvant is aluminum hydroxide gel. In some embodiments, an aluminum-based mineral salt adjuvant is aluminum hydroxide gel in the form of a white gelatinous precipitate. In some embodiments, an aluminum-based mineral salt adjuvant is CAS no. 7784-30-7. In some embodiments, an aluminum-based mineral salt adjuvant is aluminum phosphate gel. In some embodiments, an aluminum-based mineral salt adjuvant is aluminum phosphate gel in the form of a white gelatinous precipitate. In some embodiments, an aluminum-based mineral salt adjuvant is an aluminum containing adjuvant approved by the FDA for administration to humans. In some embodiments, an aluminum-based mineral salt adjuvant is an aluminum hydroxide adjuvant approved for administration to humans by the FDA. In some embodiments, an aluminum-based mineral salt adjuvant is an aluminum phosphate adjuvant approved for administration to humans by the FDA.

The term “aluminum hydroxide adjuvant” as used herein refers to the aluminum hydroxide adjuvant that includes aluminum hydroxide and is currently used in licensed human vaccines. In some embodiments, “aluminum hydroxide adjuvant” as used herein refers to the aluminum hydroxide adjuvant that is currently used in licensed human vaccines and is used in accordance with the use of that term in Hem S. L., Vaccine 23(2007) 4985-4986. In some embodiments, an aluminum hydroxide adjuvant includes CAS no. 21645-51-2. In some embodiments, an aluminum hydroxide adjuvant is aluminum hydroxide gel. In some embodiments, an aluminum hydroxide adjuvant is aluminum hydroxide gel in the form of a white gelatinous precipitate. In some embodiments, an aluminum hydroxide adjuvant includes aluminum hydroxide and does not include magnesium hydroxide. In some embodiments, an aluminum hydroxide adjuvant is Alhydrogel™. In some embodiments of an aluminum hydroxide adjuvant described above, the description is of the aluminum hydroxide adjuvant prior to inclusion in a vaccine.

The term “aluminum phosphate adjuvant” as used herein refers to the aluminum phosphate adjuvant that includes aluminum phosphate and is currently used in licensed human vaccines. In some embodiments, “aluminum phosphate adjuvant” as used herein refers to the aluminum phosphate adjuvant that is currently used in licensed human vaccines and is used in accordance with the use of that term in Hem S. L., Vaccine 23(2007) 4985-4986. In some embodiments, an aluminum phosphate adjuvant includes CAS no. 7784-30-7. In some embodiments, an aluminum phosphate adjuvant is aluminum phosphate gel. In some embodiments, an aluminum phosphate adjuvant is aluminum phosphate gel in the form of a white gelatinous precipitate. In some embodiments, an aluminum phosphate adjuvant is Adju-phos™ In some embodiments, an aluminum phosphate adjuvant is Adjuphos™. In some embodiments, an aluminum phosphate adjuvant includes amorphous aluminum hydroxyphosphate. In some embodiments of an aluminum phosphate adjuvant described above, the description is of the aluminum phosphate adjuvant prior to inclusion in a vaccine.

The term “vaccine” is used according to its plain ordinary meaning within medicine and Immunology and refers to a composition including an antigenic component for administration to a subject (e.g., human), which elicits an immune response to the antigenic component. In some embodiments a vaccine is a therapeutic. In some embodiments, a vaccine is prophylactic. In some embodiments a vaccine includes one or more adjuvants. Vaccines can be prophylactic (e.g. preventing or ameliorating the effects of a future infection by any natural or pathogen, or of an anticipated occurrence of cancer in a predisposed subject) or therapeutic (e.g., treating cancer in a subject who has been diagnosed with the cancer). The administration of vaccines is referred to vaccination. A vaccine typically contains an agent that resembles a disease-causing microorganism (e.g., RNA virus, viral structural protein, or virus particle) and is often made from weakened or killed forms of the virus (e.g., RNA virus or Zika virus), its toxins or one of its surface proteins. The agent stimulates the body's immune system to recognize the agent as a threat, destroy it, and recognize and destroy any of these microorganisms that it later encounters.

The term “vaccine formulation” as used herein refers, in the usual and customary sense, to a vaccine including an immunogenic agent (e.g., a compound as disclosed herein) and optionally one or more pharmaceutically acceptable excipients and vaccine adjuvants.

The terms “antigen” and “epitope” interchangeably refer to the portion of a molecule (e.g., a polypeptide) which is specifically recognized by a component of the immune system, e.g., an antibody, a T cell receptor, or other immune receptor such as a receptor on natural killer (NK) cells. As used herein, the term “antigen” encompasses antigenic epitopes and antigenic fragments thereof.

The term “immune response” used herein encompasses, but is not limited to, an “adaptive immune response”, also known as an “acquired immune response” in which adaptive immunity elicits immunological memory after an initial response to a specific pathogen or a specific type of cells that is targeted by the immune response, and leads to an enhanced response to that target on subsequent encounters. The induction of immunological memory can provide the basis of vaccination. The response can be mounted by the innate immune system or by the adaptive immune system, as well known in the art.

The terms “modulating immune response” and the like refer to a change in the immune response of a subject as a consequence of administration of an agent, e.g., a compound as disclosed herein, including embodiments thereof. Accordingly, an immune response can be activated or deactivated as a consequence of administration of an agent, e.g., a compound as disclosed herein, including embodiments thereof.

The term “viral shedding” is used according to its plain ordinary meaning in Medicine and Virology and refers to the production and release of virus from an infected cell. In some embodiments, the virus is released from a cell of a subject. In some embodiments virus is released into the environment from an infected subject. In some embodiments the virus is released from a cell within a subject. In some embodiments, the methods of treatment described herein refer to a reduction in viral shedding from a subject.

### II. Composition and Formulations

In an aspect is provided a compound having the formula:

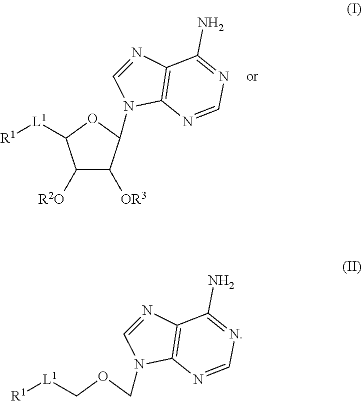

L¹ is a bond, —O—, —S—, —NH—, —C(O)NH—, —C(O)—, —NHC(O)—, —S(O)₂NH—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene. R¹ is hydrogen, oxo, halogen, —CCl₃, —CBr₃, —CF₃, —CI₃, CHCl₂, —CHBr₂, —CHF₂, —CHI₂, —CH₂C1, —CH₂Br, —CH₂F, —CH₂I, —CN, —OR^{1A} (e.g., —OH), —NHR^{1A}(e.g., —NH₂), —COOR^{1A} (e.g., —COOH)—CONR^{1A} (e.g., —CONH₂), —NHC(O)R^{1A} (e.g., —NHC(O)H), —NO₂, —SR^{1A} (e.g., —SH), —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂C1, —OC H₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl. R^{1A} is hydrogen, halogen, —CF₃, —CN, —OH, —NH₂, —N₃, —SH, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl. R² is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl. R³ is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

In embodiments, the compound has the formula:

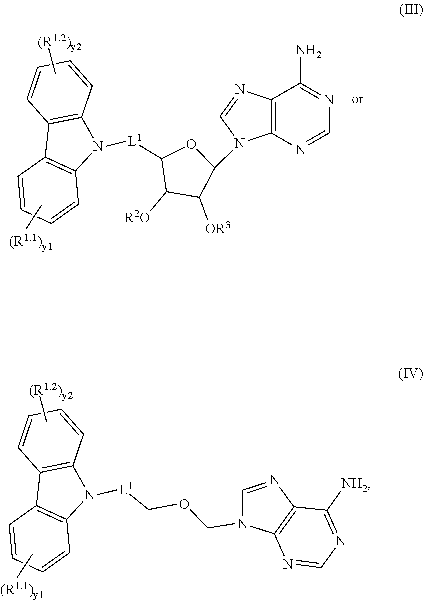

wherein R², R³, and L¹ are as described herein, including embodiments. The symbols y1 and y2 are independently an integer from 0 to 4. R^{1.1} and R^{1.2} independently are halogen, —CF₃, —CBr₃, —CCl₃, —CI₃, —CHF₂, —CHBr₂, —CHCl₂, —CHI₂, —CH₂F, —CH₂Br, —CH₂C1, —CH₂ I, —OCF₃, —OCH₃, —OCBr₃, —OCCl₃, —OCI₃, —OCHF₂, —OCHBr₂, —OCHCl₂, —OCHI₂, —OCH₂F, —OC H₂Br, —OCH₂C1, —OCH₂I, —CN, —OH, —NH₂, —COOH, —CONH₂, —NO₂, —SH, —SO₃H, —SO₄H, —SO₂N H₂, —NHNH₂, —ONH₂, —NHC(O)NHNH₂, —NHC(O)NH₂, —NHSO₂H, —NHC(O)H, —NHC(O)OH, —NHOH, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, the compound has the formula:

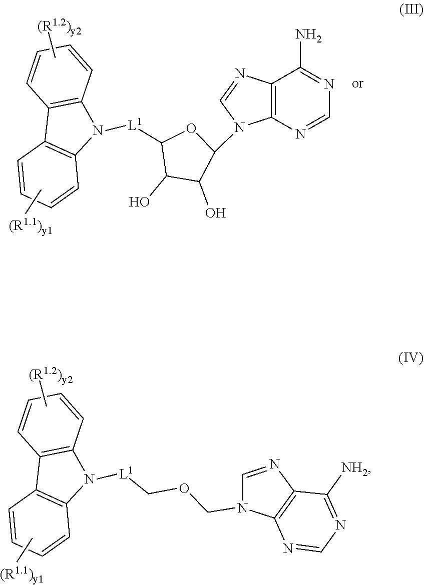

wherein R^{1.2}, R^{1.1}, y2, y1, and L¹ are as described herein, including embodiments.

In embodiments, the compound has the formula:

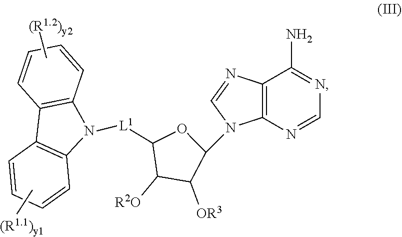

wherein R^{1.2}, y2, R^{1.1}, y1, R², R³, and L¹ are as described herein, including embodiments.

In embodiments, the compound has the formula:

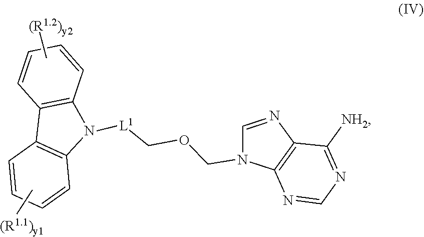

wherein R^{1.2}, R^{1.1}, y1, y2, and L¹ are as described herein, including embodiments.

In embodiments, the compound has the formula:

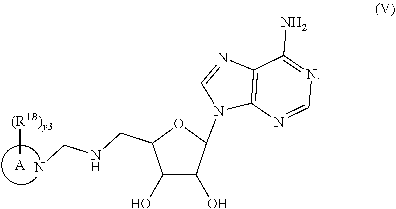

R^{1B} is independently oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHCONHNH₂, —NHCONH₂, —NHSO₂H, —NHCOH, —NHCOOH, —NHOH, —OCCl₃, —OCF₃, —OCBr₃, —OCI₃, —OCHCl₂, —OCHBr₂, —OCHI₂, —OCHF₂, —O CH₂Cl, —OCH₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. The symbol y3 is an integer from 0 to 8. Ring A is a heterocycloalkyl or heteroaryl. In embodiments, Ring A is a 3 to 14 membered heterocycloalkyl. In embodiments, Ring A is a 3 to 13 membered heterocycloalkyl. In embodiments, Ring A is a 3 to 12 membered heterocycloalkyl. In embodiments, Ring A is a 3 to 10 membered heterocycloalkyl. In embodiments, Ring A is a 3 to 8 membered heterocycloalkyl. In embodiments, Ring A is a 3 to 6 membered heterocycloalkyl. In embodiments, Ring A is a 5 to 14 membered heteroaryl. In embodiments, Ring A is a 5 to 13 membered heteroaryl. In embodiments, Ring A is a 5 to 12 membered heteroaryl. In embodiments, Ring A is a 5 to 10 membered heteroaryl. In embodiments, Ring A is a 5 to 8 membered heteroaryl. In embodiments, Ring A is a 5 to 6 membered heteroaryl. In embodiments, Ring A is a 12 membered heteroaryl. In embodiments, Ring A is a 13 membered heteroaryl. In embodiments, Ring A is an 11 membered heteroaryl. In embodiments, Ring A is a 6 membered heteroaryl. In embodiments, R^{1B} is independently halogen, —CF₃, —CN, —OH, —NH₂, —N₃, —NO₂, —SH, —COOH, —NHCOOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, Ring A is purinyl, imidazopyridinyl, imidazopyridinyl, benzoimidazolyl, benzotriazolyl, indazolyl, dihydropurinonyl, pyrazolopyridinyl, dihydropurinedionyl, furanyl, dihydrofuroquiozalinyl, pyrimidoindolyl, carbazolyl, indenyl, dihydrocyclopenta-isoindolyl, pyridinyl, pyrimidinyl, pyridazinyl, triazinyl, quinolinyl, quinazolinyl, quinoxalinyl, pyrrolyl, imdazolyl, oxazolyl, isoxazolyl, thiazolyl, isothizolyl, triazolyl, oxadiazolyl, thiadiazolyl, indolyl, or acridinyl. In embodiments, Ring A is optionally independently substituted with one or more (e.g., 1 to 8) R^{1B} substitutents.

In embodiments, L¹ is —(CH₂)_{z1}—NH—(CH₂)_{z2}—, —(CH₂)_{z1}—S—(CH₂)_{z2}—, —(CH₂)_{z1}—O—(CH₂)_{z2}—, —(CH₂)_{z1}—C(O)NH—(CH₂)_{z2}—, —(CH₂)_{z1}—C(O)O—(CH₂)_{z2}—, or —(CH₂)_{z1}—S(O)₂NH(CH₂)_{z2}, wherein z1 and z2 are independently integers from 0 to 10. In embodiments, L¹ is —(CH₂)_{z1}—NH—(CH₂)_{z2}—. In embodiments, L¹ is —(CH₂)_{z1}—S—(CH₂)_{z2}—. In embodiments, L¹ is —(CH₂)_{z1}—O—(CH₂)_{z2}—. In embodiments, L¹ is —(CH₂)_{z1}—C(O)NH—(CH₂)_{z2}—. In embodiments, L¹ is —(CH₂)_{z1}—C(O)O—(CH₂)_{z2}—. In embodiments, L¹ is —(CH₂)_{z1}—S(O)₂NH(CH₂)_{z2}. In embodiments, L¹ is a bond.

In embodiments, L¹ has the formula:

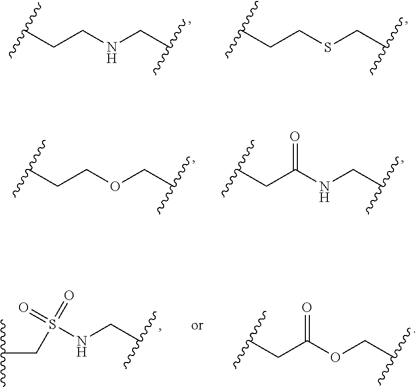 [SMILES: *CCNC* ; *CCSC* ; *CCOC* ; *CNC(=O)C* ; *CNS(=O)(=O)C* ; *COC(=O)C*]

In embodiments, L¹ has the formula:

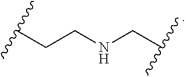 [SMILES: *CCNC*]

In embodiments, L¹ has the formula:

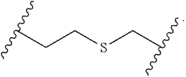 [SMILES: *CCSC*]

In embodiments, L¹ has the formula:

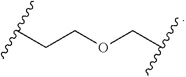 [SMILES: *CCOC*]

In embodiments, L¹ has the formula:

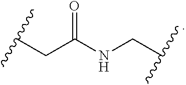 [SMILES: *CNC(=O)C*]

In embodiments, L¹ has the formula:

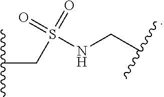 [SMILES: *CNS(=O)(=O)C*]

In embodiments, L¹ has the formula:

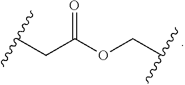 [SMILES: *COC(=O)C*]

In embodiments, L¹-R¹ has the formula:

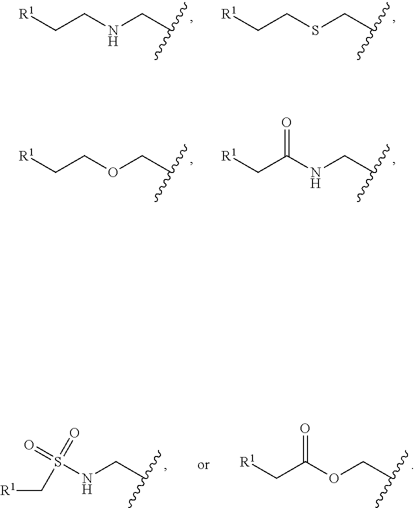

In embodiments, L¹-R¹ has the formula:

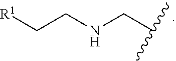

In embodiments, L¹-R¹ has the formula:

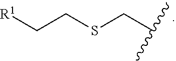

In embodiments, L¹-R¹ has the formula:

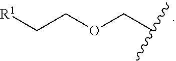

In embodiments, L¹-R¹ has the formula:

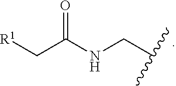

In embodiments, L¹-R¹ has the formula:

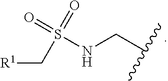

In embodiments, L¹-R¹ has the formula:

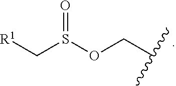

In embodiments, L¹ is a bond, —O—, —S—, —NH—, —C(O)NH—, —C(O)—, —S(O)₂NH—, R^{1D}-substituted or unsubstituted alkylene (e.g., C₁-C₈ alkylene, C₁-C₆ alkylene, or C₁-C₄ alkylene), R^{1D}-substituted or unsubstituted heteroalkylene (e.g., 2 to 8 membered heteroalkylene, 2 to 6 membered heteroalkylene, or 2 to 4 membered heteroalkylene), R^{1D}-substituted or unsubstituted cycloalkylene (e.g., C₃-C₈ cycloalkylene, C₃-C₆ cycloalkylene, or C₅-C₆ cycloalkylene), R^{1D}-substituted or unsubstituted heterocycloalkylene (e.g., 3 to 8 membered heterocycloalkylene, 3 to 6 membered heterocycloalkylene, or 5 to 6 membered heterocycloalkylene), R^{1D}-substituted or unsubstituted arylene (e.g., C₆-C₁₀ arylene, C₁₀ arylene, or phenylene), or R^{1D}-substituted or unsubstituted heteroarylene (e.g., 5 to 10 membered heteroarylene, 5 to 9 membered heteroarylene, or 5 to 6 membered heteroarylene). In embodiments, L¹ is a bond, —O—, —NH—, —C(O)NH—, —C(O)—, —S(O)₂NH—, R^{1D}-substituted or unsubstituted alkylene (e.g., C₁-C₈ alkylene, C₁-C₆ alkylene, or C₁-C₄ alkylene), R^{1D}-substituted or unsubstituted heteroalkylene (e.g., 2 to 8 membered heteroalkylene, 2 to 6 membered heteroalkylene, or 2 to 4 membered heteroalkylene), R^{1D}-substituted or unsubstituted cycloalkylene (e.g., C₃-C₈ cycloalkylene, C₃-C₆ cycloalkylene, or C₅-C₆ cycloalkylene), R^{1D}-substituted or unsubstituted heterocycloalkylene (e.g., 3 to 8 membered heterocycloalkylene, 3 to 6 membered heterocycloalkylene, or 5 to 6 membered heterocycloalkylene), R^{1D}-substituted or unsubstituted arylene (e.g., C₆-C₁₀ arylene, C₁₀ arylene, or phenylene), or R^{1D}-substituted or unsubstituted heteroarylene (e.g., 5 to 10 membered heteroarylene, 5 to 9 membered heteroarylene, or 5 to 6 membered heteroarylene).

R^{1D} is halogen, —CF₃, —CN, —OH, —NH₂, —N₃, —SH, —COOH, —NHCOOH, —CONH₂, R^{1E}-substituted or unsubstituted alkyl (e.g., C₁-C₈ alkyl, C₁-C₆ alkyl, or C₁-C₄ alkyl), R^{1E}-substituted or unsubstituted heteroalkyl (e.g., 2 to 8 membered heteroalkyl, 2 to 6 membered heteroalkyl, or 2 to 4 membered heteroalkyl), R^{1E}-substituted or unsubstituted cycloalkyl (e.g., C₃-C₈ cycloalkyl, C₃-C₆ cycloalkyl, or C₅-C₆ cycloalkyl), R^{1E}-substituted or unsubstituted heterocycloalkyl (e.g., 3 to 8 membered heterocycloalkyl, 3 to 6 membered heterocycloalkyl, or 5 to 6 membered heterocycloalkyl), R^{1E}-substituted or unsubstituted aryl (e.g., C₆-C₁₀ aryl, C₁₀ aryl, or phenyl), or R^{1E}-substituted or unsubstituted heteroaryl (e.g., 5 to 10 membered heteroaryl, 5 to 9 membered heteroaryl, or 5 to 6 membered heteroaryl).

R^{1E} is halogen, —CF₃, —CN, —OH, —NH₂, —N₃, —SH, —COOH, —NHCOOH, —CONH₂, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

In embodiments, L¹ is R^{1D}-substituted or unsubstituted alkylene (e.g., C₁-C₈ alkylene, C₁-C₆ alkylene, or C₁-C₄ alkylene). In embodiments, L¹ is R^{1D}-substituted alkylene (e.g., C₁-C₈ alkylene, C₁-C₆ alkylene, or C₁-C₄ alkylene). In embodiments, L¹ is an unsubstituted alkylene (e.g., C₁-C₈ alkylene, C₁-C₆ alkylene, or C₁-C₄ alkylene).

In embodiments, L¹ is R^{1D}-substituted or unsubstituted heteroalkylene (e.g., 2 to 8 membered heteroalkylene, 2 to 6 membered heteroalkylene, or 2 to 4 membered heteroalkylene). In embodiments, L¹ is R^{1D}-substituted heteroalkylene (e.g., 2 to 8 membered heteroalkylene, 2 to 6 membered heteroalkylene, or 2 to 4 membered heteroalkylene). In embodiments, L¹ is an unsubstituted heteroalkylene (e.g., 2 to 8 membered heteroalkylene, 2 to 6 membered heteroalkylene, or 2 to 4 membered heteroalkylene). In embodiments, L¹ is R^{1D}-substituted or unsubstituted 2 to 6 membered heteroalkylene. In embodiments, L¹ is R^{1D}-substituted 2 to 6 membered heteroalkylene. In embodiments, L¹ is an unsubstituted 2 to 6 membered heteroalkylene. In embodiments, L¹ is R^{1D}-substituted or unsubstituted 4 membered heteroalkylene. In embodiments, L¹ is R^{1D}-substituted 4 membered heteroalkylene. In embodiments, L¹ is an unsubstituted 4 membered heteroalkylene.

In embodiments, L¹ is R^{1D}-substituted or unsubstituted cycloalkylene (e.g., C₃-C₈ cycloalkylene, C₃-C₆ cycloalkylene, or C₅-C₆ cycloalkylene). In embodiments, L¹ is R^{1D}-substituted cycloalkylene (e.g., C₃-C₈ cycloalkylene, C₃-C₆ cycloalkylene, or C₅-C₆ cycloalkylene). In embodiments, L¹ is an unsubstituted cycloalkylene (e.g., C₃-C₈ cycloalkylene, C₃-C₆ cycloalkylene, or C₅-C₆ cycloalkylene).

In embodiments, L¹ is R^{1D}-substituted or unsubstituted heterocycloalkylene (e.g., 3 to 8 membered heterocycloalkylene, 3 to 6 membered heterocycloalkylene, or 5 to 6 membered heterocycloalkylene). In embodiments, L¹ is R^{1D}-substituted heterocycloalkylene (e.g., 3 to 8 membered heterocycloalkylene, 3 to 6 membered heterocycloalkylene, or 5 to 6 membered heterocycloalkylene). In embodiments, L¹ is an unsubstituted heterocycloalkylene (e.g., 3 to 8 membered heterocycloalkylene, 3 to 6 membered heterocycloalkylene, or 5 to 6 membered heterocycloalkylene).

In embodiments, L¹ is R^{1D}-substituted or unsubstituted arylene (e.g., C₆-C₁₀ arylene, C₁₀ arylene, or phenylene). In embodiments, L¹ is R^{1D}-substituted arylene (e.g., C₆-C₁₀ arylene, C₁₀ arylene, or phenylene). In embodiments, L¹ is an unsubstituted arylene (e.g., C₆-C₁₀ arylene, C₁₀ arylene, or phenylene).

In embodiments, L¹ is R^{1D}-substituted or unsubstituted heteroarylene (e.g., 5 to 10 membered heteroarylene, 5 to 1D membered heteroarylene, or 5 to 6 membered heteroarylene). In embodiments, L¹ is R^{1D}-substituted heteroarylene (e.g., 5 to 10 membered heteroarylene, 5 to 1D membered heteroarylene, or 5 to 6 membered heteroarylene). In embodiments, L¹ is an unsubstituted heteroarylene (e.g., 5 to 10 membered heteroarylene, 5 to 1D membered heteroarylene, or 5 to 6 membered heteroarylene)

In embodiments, R¹ is halogen, —CF₃, —CN, —OR^{1A}, —NHR^{1A}, —N₃, —SR^{1A}, —COOR^{1A}, —CONHR^{1A}, —NHC(O)R^{1A}, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl. In embodiments, R¹ is hydrogen. In embodiments, R¹ is halogen, —CF₃, —CN, —OH, —NH₂, —N₃, —SH, —COOH, —CONH₂, or —NHC(O)H.

In embodiments, R¹ is independently halogen. In embodiments, R¹ is independently —CF₃. In embodiments, R¹ is independently —CN. In embodiments, R¹ is independently —OR^{1A}. In embodiments, R¹ is independently —NHR^{1A}. In embodiments, R¹ is independently —N₃. In embodiments, R¹ is independently —SR^{1A}. In embodiments, R¹ is independently —COOR^{1A}. In embodiments, R¹ is independently —CONHR^{1A}. In embodiments, R¹ is independently or —NHC(O)R^{1A}. In embodiments. In embodiments, R¹ is independently R^{1A} is independently hydrogen. In embodiments, R¹ is independently halogen. In embodiments, R¹ is independently —CF₃. In embodiments, R¹ is independently —CN. In embodiments, R¹ is independently —OH. In embodiments, R¹ is independently —NH₂. In embodiments, R¹ is independently —N₃. In embodiments, R¹ is independently —SH. In embodiments, R¹ is independently —COOH. In embodiments, R¹ is independently —NHCOOH. In embodiments, R¹ is independently —CONH₂.

In embodiments, R¹ is independently R^{1B}-substituted or unsubstituted alkyl (e.g., C₁-C₈ alkyl, C₁-C₆ alkyl, or C₁-C₄ alkyl), R^{1B}-substituted or unsubstituted heteroalkyl (e.g., 2 to 8 membered heteroalkyl, 2 to 6 membered heteroalkyl, or 2 to 4 membered heteroalkyl), R^{1B}-substituted or unsubstituted cycloalkyl (e.g., C₃-C₈ cycloalkyl, C₃-C₆ cycloalkyl, or C₅-C₆ cycloalkyl), R^{1B}-substituted or unsubstituted heterocycloalkyl (e.g., 3 to 14 membered heterocycloalkyl, 3 to 12 membered heterocycloalkyl, 3 to 8 membered heterocycloalkyl, 3 to 6 membered heterocycloalkyl, or 5 to 6 membered heterocycloalkyl), R^{1B}-substituted or unsubstituted aryl (e.g., C₆-C₁₀ aryl, C₁₀ aryl, or phenyl), or R^{1B}-substituted or unsubstituted heteroaryl (e.g., 5 to 14 membered heteroaryl, 5 to 13 membered heteroaryl, 5 to 12 membered heteroaryl, 5 to 10 membered heteroaryl, 5 to 9 membered heteroaryl, or 5 to 6 membered heteroaryl).

In embodiments, R¹ is independently R^{1B}-substituted or unsubstituted alkyl (e.g., C₁-C₈ alkyl, C₁-C₆ alkyl, or C₁-C₄ alkyl). In embodiments, R¹ is independently R^{1B}-substituted alkyl (e.g., C₁-C₈ alkyl, C₁-C₆ alkyl, or C₁-C₄ alkyl). In embodiments, R¹ is independently an unsubstituted alkyl (e.g., C₁-C₈ alkyl, C₁-C₆ alkyl, or C₁-C₄ alkyl).

In embodiments, R¹ is independently R^{1B}-substituted or unsubstituted heteroalkyl (e.g., 2 to 8 membered heteroalkyl, 2 to 6 membered heteroalkyl, or 2 to 4 membered heteroalkyl). In embodiments, R¹ is independently R^{1B}-substituted heteroalkyl (e.g., 2 to 8 membered heteroalkyl, 2 to 6 membered heteroalkyl, or 2 to 4 membered heteroalkyl). In embodiments, R¹ is independently an unsubstituted heteroalkyl (e.g., 2 to 8 membered heteroalkyl, 2 to 6 membered heteroalkyl, or 2 to 4 membered heteroalkyl).

In embodiments, R¹ is independently R^{1B}-substituted or unsubstituted cycloalkyl (e.g., C₃-C₈ cycloalkyl, C₃-C₆ cycloalkyl, or C₅-C₆ cycloalkyl). In embodiments, R¹ is independently R^{1B}-substituted cycloalkyl (e.g., C₃-C₈ cycloalkyl, C₃-C₆ cycloalkyl, or C₅-C₆ cycloalkyl). In embodiments, R¹ is independently an unsubstituted cycloalkyl (e.g., C₃-C₅ cycloalkyl, C₃-C₆ cycloalkyl, or C₅-C₆ cycloalkyl).

In embodiments, R¹ is independently R^{1B}-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R¹ is independently R^{1B}-substituted heterocycloalkyl (e.g., 3 to 8 membered heterocycloalkyl, 3 to 6 membered heterocycloalkyl, or 5 to 6 membered heterocycloalkyl). In embodiments, R¹ is independently an unsubstituted heterocycloalkyl (e.g., 3 to 8 membered heterocycloalkyl, 3 to 6 membered heterocycloalkyl, or 5 to 6 membered heterocycloalkyl).

In embodiments, R¹ is independently R^{1B}-substituted or unsubstituted aryl (e.g., C₆-C₁₄ aryl, C₆-C₁₀ aryl, C₁₀ aryl, or phenyl). In embodiments, R¹ is independently R^{1B}-substituted aryl (e.g., C₆-C₁₄ aryl, C₆-C₁₀ aryl, C₁₀ aryl, or phenyl). In embodiments, R¹ is independently an unsubstituted aryl (e.g., C₆-C₁₄ aryl, C₆-C₁₀ aryl, C₁₀ aryl, or phenyl).

In embodiments, R¹ is independently R^{1B}-substituted or unsubstituted heteroaryl (e.g., 5 to 12 membered heteroaryl, 5 to 10 membered heteroaryl, 5 to 9 membered heteroaryl, or 5 to 6 membered heteroaryl). In embodiments, R¹ is independently R^{1B}-substituted heteroaryl (e.g., 5 to 14 membered heteroaryl, 5 to 13 membered heteroaryl, 5 to 12 membered heteroaryl, 5 to 10 membered heteroaryl, 5 to 9 membered heteroaryl, or 5 to 6 membered heteroaryl). In embodiments, R¹ is independently an unsubstituted heteroaryl (e.g., e.g., 5 to 14 membered heteroaryl, 5 to 13 membered heteroaryl, 5 to 12 membered heteroaryl, 5 to 10 membered heteroaryl, 5 to 9 membered heteroaryl, or 5 to 6 membered heteroaryl). In embodiments, R¹ is independently a R^{1B}-substituted 5 to 12 membered heteroaryl. In embodiments, R¹ is independently a R^{1B}-substituted 6 to 14 membered heteroaryl. In embodiments, R¹ is independently a R^{1B}-substituted 6 to 13 membered heteroaryl. In embodiments, R¹ is independently a R^{1B}-substituted 6 to 12 membered heteroaryl. In embodiments, R¹ is independently a R^{1B}-substituted 12 membered heteroaryl. In embodiments, R¹ is independently an unsubstituted 5 to 12 membered heteroaryl. In embodiments, R¹ is independently an unsubstituted 6 to 12 membered heteroaryl. In embodiments, R¹ is independently an unsubstituted 12 membered heteroaryl.

In embodiments, R¹ is independently substituted or unsubstituted cycloalkyl (e.g., C₃-C₈ cycloalkyl, C₃-C₆ cycloalkyl, or C₅-C₆ cycloalkyl), substituted or unsubstituted heterocycloalkyl (e.g., 3 to 8 membered heterocycloalkyl, 3 to 6 membered heterocycloalkyl, or 5 to 6 membered heterocycloalkyl), substituted or unsubstituted aryl (e.g., C₆-C₁₀ aryl, C₁₀ aryl, or phenyl), or substituted or unsubstituted heteroaryl (e.g., 5 to 10 membered heteroaryl, 5 to 9 membered heteroaryl, or 5 to 6 membered heteroaryl). In embodiments, R¹ is independently substituted or unsubstituted heterocycloalkyl (e.g., 3 to 8 membered heterocycloalkyl, 3 to 6 membered heterocycloalkyl, or 5 to 6 membered heterocycloalkyl), substituted or unsubstituted aryl (e.g., C₆-C₁₀ aryl, C₁₀ aryl, or phenyl), or substituted or unsubstituted heteroaryl (e.g., 5 to 12 membered heteroaryl, 5 to 10 membered heteroaryl, 5 to 9 membered heteroaryl, or 5 to 6 membered heteroaryl).

In embodiments, R¹ is independently substituted or unsubstituted purinyl, substituted or unsubstituted imidazopyridinyl, substituted or unsubstituted imidazopyridinyl, substituted or unsubstituted benzoimidazolyl, substituted or unsubstituted benzotriazolyl, substituted or unsubstituted indazolyl, substituted or unsubstituted dihydropurinonyl, substituted or unsubstituted pyrazolopyridinyl, substituted or unsubstituted dihydropurinedionyl, substituted or unsubstituted furanyl, substituted or unsubstituted dihydrofuroquiozalinyl, substituted or unsubstituted pyrimidoindolyl, substituted or unsubstituted carbazolyl, substituted or unsubstituted indenyl, substituted or unsubstituted dihydrocyclopenta-isoindolyl, substituted or unsubstituted pyridinyl, substituted or unsubstituted pyrimidinyl, substituted or unsubstituted pyridazinyl, substituted or unsubstituted triazinyl, substituted or unsubstituted quinolinyl, substituted or unsubstituted quinazolinyl, substituted or unsubstituted quinoxalinyl, substituted or unsubstituted pyrrolyl, substituted or unsubstituted imidazolyl, substituted or unsubstituted oxazolyl, substituted or unsubstituted isoxazolyl, substituted or unsubstituted thiazolyl, substituted or unsubstituted isothizolyl, substituted or unsubstituted triazolyl, substituted or unsubstituted oxadiazolyl, substituted or unsubstituted thiadiazolyl, substituted or unsubstituted indolyl, substituted or unsubstituted pyrazolopyridinyl, substituted or unsubstituted benzofuranyl, substituted or unsubstituted benzothiophenyl, substituted or unsubstituted acridinyl, or substituted or unsubstituted phenyl.

In embodiments, R¹ is independently substituted or unsubstituted purinyl. In embodiments, R¹ is independently substituted or unsubstituted imidazopyridinyl. In embodiments, R¹ is independently substituted or unsubstituted imidazopyridinyl. In embodiments, R¹ is independently substituted or unsubstituted benzoimidazolyl. In embodiments, R¹ is independently substituted or unsubstituted benzotriazolyl. In embodiments, R¹ is independently substituted or unsubstituted indazolyl. In embodiments, R¹ is independently substituted or unsubstituted dihydropurinonyl. In embodiments, R¹ is independently substituted or unsubstituted pyrazolopyridinyl. In embodiments, R¹ is independently substituted or unsubstituted dihydropurinedionyl. In embodiments, R¹ is independently substituted or unsubstituted furanyl. In embodiments, R¹ is independently substituted or unsubstituted dihydrofuroquiozalinyl. In embodiments, R¹ is independently substituted or unsubstituted pyrimidoindolyl. In embodiments, R¹ is independently substituted or unsubstituted carbazolyl. In embodiments, R¹ is independently substituted or unsubstituted indenyl. In embodiments, R¹ is independently substituted or unsubstituted dihydrocyclopenta-isoindolyl. In embodiments, R¹ is independently substituted or unsubstituted pyridinyl. In embodiments, R¹ is independently substituted or unsubstituted pyrimidinyl. In embodiments, R¹ is independently substituted or unsubstituted pyridazinyl. In embodiments, R¹ is independently substituted or unsubstituted triazinyl. In embodiments, R¹ is independently substituted or unsubstituted quinolinyl. In embodiments, R¹ is independently substituted or unsubstituted quinazolinyl. In embodiments, R¹ is independently substituted or unsubstituted quinoxalinyl. In embodiments, R¹ is independently substituted or unsubstituted pyrrolyl. In embodiments, R¹ is independently substituted or unsubstituted imidazolyl. In embodiments, R¹ is independently substituted or unsubstituted oxazolyl. In embodiments, R¹ is independently substituted or unsubstituted isoxazolyl. In embodiments, R¹ is independently substituted or unsubstituted thiazolyl. In embodiments, R¹ is independently substituted or unsubstituted isothizolyl. In embodiments, R¹ is independently substituted or unsubstituted triazolyl. In embodiments, R¹ is independently substituted or unsubstituted oxadiazolyl. In embodiments, R¹ is independently substituted or unsubstituted thiadiazolyl. In embodiments, R¹ is independently substituted or unsubstituted indolyl. In embodiments, R¹ is independently substituted or unsubstituted pyrazolopyridinyl. In embodiments, R¹ is independently substituted or unsubstituted benzofuranyl. In embodiments, R¹ is independently substituted or unsubstituted benzothiophenyl. In embodiments, R¹ is independently substituted or unsubstituted acridinyl. In embodiments, R¹ is independently substituted or unsubstituted phenyl.

In embodiments, R¹ is independently substituted purinyl. In embodiments, R¹ is independently substituted imidazopyridinyl. In embodiments, R¹ is independently substituted imidazopyridinyl. In embodiments, R¹ is independently substituted benzoimidazolyl. In embodiments, R¹ is independently substituted benzotriazolyl. In embodiments, R¹ is independently substituted indazolyl. In embodiments, R¹ is independently substituted dihydropurinonyl. In embodiments, R¹ is independently substituted pyrazolopyridinyl. In embodiments, R¹ is independently substituted dihydropurinedionyl. In embodiments, R¹ is independently substituted furanyl. In embodiments, R¹ is independently substituted dihydrofuroquiozalinyl. In embodiments, R¹ is independently substituted pyrimidoindolyl. In embodiments, R¹ is independently substituted carbazolyl. In embodiments, R¹ is independently substituted indenyl. In embodiments, R¹ is independently substituted dihydrocyclopenta-isoindolyl. In embodiments, R¹ is independently substituted pyridinyl. In embodiments, R¹ is independently substituted pyrimidinyl. In embodiments, R¹ is independently substituted pyridazinyl. In embodiments, R¹ is independently substituted triazinyl. In embodiments, R¹ is independently substituted quinolinyl. In embodiments, R¹ is independently substituted quinazolinyl. In embodiments, R¹ is independently substituted quinoxalinyl. In embodiments, R¹ is independently substituted pyrrolyl. In embodiments, R¹ is independently substituted imidazolyl. In embodiments, R¹ is independently substituted oxazolyl. In embodiments, R¹ is independently substituted isoxazolyl. In embodiments, R¹ is independently substituted thiazolyl. In embodiments, R¹ is independently substituted isothizolyl. In embodiments, R¹ is independently substituted triazolyl. In embodiments, R¹ is independently substituted oxadiazolyl. In embodiments, R¹ is independently substituted thiadiazolyl. In embodiments, R¹ is independently substituted indolyl. In embodiments, R¹ is independently substituted pyrazolopyridinyl. In embodiments, R¹ is independently substituted benzofuranyl. In embodiments, R¹ is independently substituted benzothiophenyl. In embodiments, R¹ is independently substituted acridinyl. In embodiments, R¹ is independently substituted phenyl.

In embodiments, R¹ is independently an unsubstituted purinyl. In embodiments, R¹ is independently an unsubstituted imidazopyridinyl. In embodiments, R¹ is independently an unsubstituted imidazopyridinyl. In embodiments, R¹ is independently an unsubstituted benzoimidazolyl. In embodiments, R¹ is independently an unsubstituted benzotriazolyl. In embodiments, R¹ is independently an unsubstituted indazolyl. In embodiments, R¹ is independently an unsubstituted dihydropurinonyl. In embodiments, R¹ is independently an unsubstituted pyrazolopyridinyl. In embodiments, R¹ is independently an unsubstituted dihydropurinedionyl. In embodiments, R¹ is independently an unsubstituted furanyl. In embodiments, R¹ is independently an unsubstituted dihydrofuroquiozalinyl. In embodiments, R¹ is independently an unsubstituted pyrimidoindolyl. In embodiments, R¹ is independently an unsubstituted carbazolyl. In embodiments, R¹ is independently an unsubstituted indenyl. In embodiments, R¹ is independently an unsubstituted dihydrocyclopenta-isoindolyl. In embodiments, R¹ is independently an unsubstituted pyridinyl. In embodiments, R¹ is independently an unsubstituted pyrimidinyl. In embodiments, R¹ is independently an unsubstituted pyridazinyl. In embodiments, R¹ is independently an unsubstituted triazinyl. In embodiments, R¹ is independently an unsubstituted quinolinyl. In embodiments, R¹ is independently an unsubstituted quinazolinyl. In embodiments, R¹ is independently an unsubstituted quinoxalinyl. In embodiments, R¹ is independently an unsubstituted pyrrolyl. In embodiments, R¹ is independently an unsubstituted imidazolyl. In embodiments, R¹ is independently an unsubstituted oxazolyl. In embodiments, R¹ is independently an unsubstituted isoxazolyl. In embodiments, R¹ is independently an unsubstituted thiazolyl. In embodiments, R¹ is independently an unsubstituted isothizolyl. In embodiments, R¹ is independently an unsubstituted triazolyl. In embodiments, R¹ is independently an unsubstituted oxadiazolyl. In embodiments, R¹ is independently an unsubstituted thiadiazolyl. In embodiments, R¹ is independently an unsubstituted indolyl. In embodiments, R¹ is independently an unsubstituted pyrazolopyridinyl. In embodiments, R¹ is independently an unsubstituted benzofuranyl. In embodiments, R¹ is independently an unsubstituted benzothiophenyl. In embodiments, R¹ is independently an unsubstituted acridinyl. In embodiments, R¹ is independently an unsubstituted phenyl.

In embodiments, R¹ is independently halogen, —CF₃, —CN, —OR^{1A}, —NHR^{1A}, —N₃, —SR^{1A}, —COOR^{1A}, —CONHR^{1A}, or —NHC(O)R^{1A}. In embodiments, R^{1A} is independently hydrogen, halogen, —CF₃, —CN, —OH, —NH₂, —N₃, —SH, —COOH, —NHCOOH, —CONH₂, R^{1B}-substituted or unsubstituted alkyl (e.g., C₁-C₈ alkyl, C₁-C₆ alkyl, or C₁-C₄ alkyl), R^{1B}-substituted or unsubstituted heteroalkyl (e.g., 2 to 8 membered heteroalkyl, 2 to 6 membered heteroalkyl, or 2 to 4 membered heteroalkyl), R^{1B}-substituted or unsubstituted cycloalkyl (e.g., C₃-C₈ cycloalkyl, C₃-C₆ cycloalkyl, or C₅-C₆ cycloalkyl), R^{1B}-substituted or unsubstituted heterocycloalkyl (e.g., 3 to 8 membered heterocycloalkyl, 3 to 6 membered heterocycloalkyl, or 5 to 6 membered heterocycloalkyl), R^{1B}-substituted or unsubstituted aryl (e.g., C₆-C₁₀ aryl, C₁₀ aryl, or phenyl), or R^{1B}-substituted or unsubstituted heteroaryl (e.g., 5 to 10 membered heteroaryl, 5 to 9 membered heteroaryl, or 5 to 6 membered heteroaryl).

In embodiments, R^{1B} is independently halogen, —CF₃, —CN, —OH, —NH₂, —N₃, —SH, —COOH, —NHCOOH, —CONH₂, R^{1C}-substituted or unsubstituted alkyl (e.g., C₁-C₈ alkyl, C₁-C₆ alkyl, or C₁-C₄ alkyl), R^{1C}-substituted or unsubstituted heteroalkyl (e.g., 2 to 8 membered heteroalkyl, 2 to 6 membered heteroalkyl, or 2 to 4 membered heteroalkyl), R^{1C}-substituted or unsubstituted cycloalkyl (e.g., C₃-C₈ cycloalkyl, C₃-C₆ cycloalkyl, or C₅-C₆ cycloalkyl), R^{1C} substituted or unsubstituted heterocycloalkyl (e.g., 3 to 8 membered heterocycloalkyl, 3 to 6 membered heterocycloalkyl, or 5 to 6 membered heterocycloalkyl), R^{1C}-substituted or unsubstituted aryl (e.g., C₆-C₁₀ aryl, C₁₀ aryl, or phenyl), or R^{1C}-substituted or unsubstituted heteroaryl (e.g., 5 to 10 membered heteroaryl, 5 to 9 membered heteroaryl, or 5 to 6 membered heteroaryl).

In embodiments, R^{1B} is independently halogen. In embodiments, R^{1B} is independently —CF₃. In embodiments, R^{1B} is independently —CN. In embodiments, R^{1B} is independently —OH. In embodiments, R^{1B} is independently —NH₂. In embodiments, R^{1B} is independently —N₃. In embodiments, R^{1B} is independently —SH. In embodiments, R^{1B} is independently —COOH. In embodiments, R^{1B} is independently —NHCOOH. In embodiments, R^{1B} is independently —CONH₂. In embodiments, R^{1B} is independently —NO₂.

In embodiments, R^{1B} is R^{1C}-substituted or unsubstituted alkyl (e.g., C₁-C₈ alkyl, C₁-C₆ alkyl, or C₁-C₄ alkyl). In embodiments, R^{1B} is R^{1C}-substituted alkyl (e.g., C₁-C₈ alkyl, C₁-C₆ alkyl, or C₁-C₄ alkyl). In embodiments, R^{1B} is an unsubstituted alkyl (e.g., C₁-C₈ alkyl, C₁-C₆ alkyl, or C₁-C₄ alkyl). In embodiments, R^{1B} is R^{1C}-substituted or unsubstituted heteroalkyl (e.g., 2 to 8 membered heteroalkyl, 2 to 6 membered heteroalkyl, or 2 to 4 membered heteroalkyl). In embodiments, R^{1B} is R^{1C}-substituted heteroalkyl (e.g., 2 to 8 membered heteroalkyl, 2 to 6 membered heteroalkyl, or 2 to 4 membered heteroalkyl). In embodiments, R^{1B} is an unsubstituted heteroalkyl (e.g., 2 to 8 membered heteroalkyl, 2 to 6 membered heteroalkyl, or 2 to 4 membered heteroalkyl). In embodiments, R^{1B} is R^{1C}-substituted or unsubstituted cycloalkyl (e.g., C₃-C₈ cycloalkyl, C₃-C₆ cycloalkyl, or C₅-C₆ cycloalkyl). In embodiments, R^{1B} is R^{1C}-substituted cycloalkyl (e.g., C₃-C₈ cycloalkyl, C₃-C₆ cycloalkyl, or C₅-C₆ cycloalkyl). In embodiments, R^{1B} is an unsubstituted cycloalkyl (e.g., C₃-C₈ cycloalkyl, C₃-C₆ cycloalkyl, or C₅-C₆ cycloalkyl). In embodiments, R^{1B} is R^{1C}-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R^{1B} is R^{1C}-substituted heterocycloalkyl (e.g., 3 to 8 membered heterocycloalkyl, 3 to 6 membered heterocycloalkyl, or 5 to 6 membered heterocycloalkyl). In embodiments, R^{1B} is an unsubstituted heterocycloalkyl (e.g., 3 to 8 membered heterocycloalkyl, 3 to 6 membered heterocycloalkyl, or 5 to 6 membered heterocycloalkyl). In embodiments, R^{1B} is R^{1C}-substituted or unsubstituted aryl (e.g., C₆-C₁₀ aryl, C₁₀ aryl, or phenyl). In embodiments, R^{1B} is R^{1C}-substituted aryl (e.g., C₆-C₁₀ aryl, C₁₀ aryl, or phenyl). In embodiments, R^{1B} is an unsubstituted aryl (e.g., C₆-C₁₀ aryl, C₁₀ aryl, or phenyl). In embodiments, R^{1B} is R^{1C}-substituted or unsubstituted heteroaryl (e.g., 5 to 10 membered heteroaryl, 5 to 9 membered heteroaryl, or 5 to 6 membered heteroaryl). In embodiments, R^{1B} is R^{1C}-substituted heteroaryl (e.g., 5 to 10 membered heteroaryl, 5 to 9 membered heteroaryl, or 5 to 6 membered heteroaryl). In embodiments, R^{1B} is an unsubstituted heteroaryl (e.g., 5 to 10 membered heteroaryl, 5 to 9 membered heteroaryl, or 5 to 6 membered heteroaryl).

R^{1C} is independently halogen, —CF₃, —CN, —OH, —NH₂, —N₃, —SH, —COOH, —NHCOOH, —CONH₂, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

In embodiments, R^{1.1} is independently

halogen, —CF₃, —CBr₃, —CCl₃, —CI₃, —CHF₂, —CHBr₂, —CHCl₂, —CHI₂, —CH₂F, —CH₂Br, —CH₂Cl, —CH₂ I, —OCF₃, —OCH₃, —OCBr₃, —OCCl₃, —OCI₃, —OCHF₂, —OCHBr₂, —OCHCl₂, —OCHI₂, —OCH₂F, —OC H2Br, —OCH₂Cl, —OCH₂I, —CN, —OH, —NH₂, —COOH, —CONH₂, —NO₂, —SH, —SO₃H, —SO₄H, —SO₂N H₂, —NHNH₂, —ONH₂, —NHC(O)NHNH₂, —NHC(O)NH₂, —NHSO₂H, —NHC(O)H, —NHC(O)OH, —NHOH, substituted or unsubstituted alkyl (e.g., C₁-C₈ alkyl, C₁-C₆ alkyl, or C₁-C₄ alkyl), substituted or unsubstituted heteroalkyl (e.g., 2 to 8 membered heteroalkyl, 2 to 6 membered heteroalkyl, or 2 to 4 membered heteroalkyl), substituted or unsubstituted cycloalkyl (e.g., C₃-C₈ cycloalkyl, C₃-C₆ cycloalkyl, or C₅-C₆ cycloalkyl), substituted or unsubstituted heterocycloalkyl (e.g., 3 to 8 membered heterocycloalkyl, 3 to 6 membered heterocycloalkyl, or 5 to 6 membered heterocycloalkyl), substituted or unsubstituted aryl (e.g., C₆-C₁₀ aryl, C₁₀ aryl, or phenyl), or substituted or unsubstituted heteroaryl (e.g., 5 to 10 membered heteroaryl, 5 to 9 membered heteroaryl, or 5 to 6 membered heteroaryl).

In embodiments, R^{1.1} is independently R^{1C}-substituted or unsubstituted alkyl (e.g., C₁-C₈ alkyl, C₁-C₆ alkyl, or C₁-C₄ alkyl). In embodiments, R^{1.1} is independently R^{1C}-substituted alkyl (e.g., C₁-C₈ alkyl, C₁-C₆ alkyl, or C₁-C₄ alkyl). In embodiments, R^{1.1} is independently an unsubstituted alkyl (e.g., C₁-C₈ alkyl, C₁-C₆ alkyl, or C₁-C₄ alkyl). In embodiments, R^{1.1} is independently R^{1C}-substituted C₁-C₈ alkyl. In embodiments, R^{1.1} is independently R^{1C}-substituted C₁ alkyl. In embodiments, R^{1.1} is independently R^{1C}-substituted C₂ alkyl. In embodiments, R^{1.1} is independently R^{1C}-substituted C₃ alkyl. In embodiments, R^{1′} is independently R^{1C}-substituted C₄ alkyl. In embodiments, R^{1.1} is independently R^{1C}-substituted C₅ alkyl. In embodiments, R^{1.1} is independently R^{1C}-substituted C₆ alkyl. In embodiments, R^{1.1} is independently R^{1C}-substituted C₇ alkyl. In embodiments, R^{1.1} is independently an unsubstituted C₈ alkyl. In embodiments, R^{1.1} is independently an unsubstituted C₁-C₈ alkyl. In embodiments, R^{1.1} is independently an unsubstituted C₁ alkyl. In embodiments, R^{1.1} is independently an unsubstituted C₂ alkyl. In embodiments, R^{1.1} is independently an unsubstituted C₃ alkyl. In embodiments, R^{1.1} is independently an unsubstituted C₄ alkyl. In embodiments, R^{1.1} is independently an unsubstituted C₅ alkyl. In embodiments, R^{1.1} is independently an unsubstituted C₆ alkyl. In embodiments, R^{1.1} is independently an unsubstituted C₇ alkyl. In embodiments, R^{1.1} is independently an unsubstituted C₈ alkyl.

In embodiments, R^{1.1} is independently R^{1C}-substituted or unsubstituted heteroalkyl (e.g., 2 to 8 membered heteroalkyl, 2 to 6 membered heteroalkyl, or 2 to 4 membered heteroalkyl). In embodiments, R^{1.1} is independently R^{1C}-substituted heteroalkyl (e.g., 2 to 8 membered heteroalkyl, 2 to 6 membered heteroalkyl, or 2 to 4 membered heteroalkyl). In embodiments, R^{1.1} is independently an unsubstituted heteroalkyl (e.g., 2 to 8 membered heteroalkyl, 2 to 6 membered heteroalkyl, or 2 to 4 membered heteroalkyl). In embodiments, R^{1.1} is independently R^{1C}-substituted 2 to 6 membered heteroalkyl. In embodiments, R^{1.1} is independently R^{1C}-substituted 2 membered heteroalkyl. In embodiments, R^{1.1} is independently R^{1C}-substituted 3 membered heteroalkyl. In embodiments, R^{1.1} is independently R^{1C}-substituted 4 membered heteroalkyl. In embodiments, R^{1.1} is independently R^{1C}-substituted 5 membered heteroalkyl. In embodiments, R^{1.1} is independently R^{1C}-substituted 6 membered heteroalkyl. In embodiments, R^{1.1} is independently an unsubstituted 2 to 6 membered heteroalkyl. In embodiments, R^{1.1} is independently an unsubstituted 2 membered heteroalkyl. In embodiments, R^{1.1} is independently an unsubstituted 3 membered heteroalkyl. In embodiments, R^{1.1} is independently an unsubstituted 4 membered heteroalkyl. In embodiments, R^{1.1} is independently an unsubstituted 5 membered heteroalkyl. In embodiments, R^{1.1} is independently an unsubstituted 6 membered heteroalkyl.

In embodiments, R^{1.1} is independently R^{1C}-substituted or unsubstituted cycloalkyl (e.g., C₃-C₈ cycloalkyl, C₃-C₆ cycloalkyl, or C₅-C₆ cycloalkyl). In embodiments, R^{1.1} is independently R^{1C}-substituted cycloalkyl (e.g., C₃-C₈ cycloalkyl, C₃-C₆ cycloalkyl, or C₅-C₆ cycloalkyl). In embodiments, R^{1.1} is independently an unsubstituted cycloalkyl (e.g., C₃-C₅ cycloalkyl, C₃-C₆ cycloalkyl, or C₅-C₆ cycloalkyl). In embodiments, R^{1.1} is independently R^{1C}-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R^{1.1} is independently R^{1C}-substituted heterocycloalkyl (e.g., 3 to 8 membered heterocycloalkyl, 3 to 6 membered heterocycloalkyl, or 5 to 6 membered heterocycloalkyl). In embodiments, R^{1.1} is independently an unsubstituted heterocycloalkyl (e.g., 3 to 8 membered heterocycloalkyl, 3 to 6 membered heterocycloalkyl, or 5 to 6 membered heterocycloalkyl). In embodiments, R^{1.1} is independently R^{1C}-substituted or unsubstituted aryl (e.g., C₆-C₁₀ aryl, C₁₀ aryl, or phenyl). In embodiments, R^{1.1} is independently R^{1C}-substituted aryl (e.g., C₆-C₁₀ aryl, C₁₀ aryl, or phenyl). In embodiments, R^{1.1} is independently an unsubstituted aryl (e.g., C₆-C₁₀ aryl, C₁₀ aryl, or phenyl). In embodiments, R^{1.1} is independently R^{1C}-substituted or unsubstituted heteroaryl (e.g., 5 to 10 membered heteroaryl, 5 to 9 membered heteroaryl, or 5 to 6 membered heteroaryl). In embodiments, R^{1.1} is independently R^{1C}-substituted heteroaryl (e.g., 5 to 10 membered heteroaryl, 5 to 9 membered heteroaryl, or 5 to 6 membered heteroaryl). In embodiments, R^{1.1} is independently an unsubstituted heteroaryl (e.g., 5 to 10 membered heteroaryl, 5 to 9 membered heteroaryl, or 5 to 6 membered heteroaryl).

In embodiments, R^{1.1} is independently

halogen, —CF₃, —CBr₃, —CCl₃, —CI₃, —CHF₂, —CHBr₂, —CHCl₂, —CHI₂, —CH₂F, —CH₂Br, —CH₂Cl, —CH₂ I, —OCF₃, —OCH₃, —OCBr₃, —OCCl₃, —OCI₃, —OCHF₂, —OCHBr₂, —OCHCl₂, —OCHI₂, —OCH₂F, —OC H₂Br, —OCH₂Cl, —OCH₂I, —CN, —OH, —NH₂, —COOH, —CONH₂, —NO₂, —SH, —SO₃H, —SO₄H, —SO₂N H₂, —NHNH₂, —ONH₂, —NHC(O)NHNH₂, —NHC(O)NH₂, —NHSO₂H, —NHC(O)H, —NHC(O)OH, or —NHOH. In embodiments, R^{1.1} is independently halogen. In embodiments, R^{1.1} is independently —CF₃. In embodiments, R^{1.1} is independently —CBr₃. In embodiments, R^{1.1} is independently —CCl₃. In embodiments, R^{1.1} is independently —CI₃. In embodiments, R^{1.1} is independently —CHF₂. In embodiments, R^{1.1} is independently —CHBr2. In embodiments, R^{1.1} is independently —CHCl₂. In embodiments, R^{1.1} is independently —CHI₂. In embodiments, R^{1.1} is independently —CH₂F. In embodiments, R^{1.1} is independently —CH2Br. In embodiments, R^{1.1} is independently —CH₂Cl. In embodiments, R^{1.1} is independently —CH₂I. In embodiments, R^{1.1} is independently —OCF₃. In embodiments, R^{1.1} is independently —OCH₃. In embodiments, R^{1.1} is independently —OCBr3. In embodiments, R^{1.1} is independently —OCCl₃. In embodiments, R^{1.1} is independently —OCI₃. In embodiments, R^{1.1} is independently —OCHF₂. In embodiments, R^{1.1} is independently —OCHBr₂. In embodiments, R^{1.1} is independently —OCHCl₂. In embodiments, R^{1.1} is independently —OCHI₂. In embodiments, R^{1.1} is independently —OCH₂F. In embodiments, R^{1.1} is independently —OCH₂Br. In embodiments, R^{1.1} is independently —OCH₂Cl. In embodiments, R^{1.1} is independently —OCH₂I. In embodiments, R^{1.1} is independently —CN. In embodiments, R^{1.1} is independently —OH. In embodiments, R^{1.1} is independently —NH₂. In embodiments, R^{1.1} is independently —COOH. In embodiments, R^{1.1} is independently —CONH₂. In embodiments, R^{1.1} is independently —NO₂. In embodiments, R^{1.1} is independently —SH. In embodiments, R^{1.1} is independently —SO₃H. In embodiments, R^{1.1} is independently —SO₄H. In embodiments, R^{1.1} is independently —SO₂NH₂. In embodiments, R^{1.1} is independently —NHNH₂. In embodiments, R^{1.1} is independently —ONH₂. In embodiments, R^{1.1} is independently —NHC(O)NHNH₂. In embodiments, R^{1.1} is independently —NHC(O)NH₂. In embodiments, R^{1.1} is independently —NHSO₂H. In embodiments, R^{1.1} is independently —NHC(O)H. In embodiments, R^{1.1} is independently —NHC(O)OH. In embodiments, R^{1.1} is independently —NHOH. In embodiments, R^{1.1} is independently unsubstituted t-butyl. In embodiments, R^{1.1} is independently unsubstituted isopropyl.

In embodiments, R^{1.2} is independently

halogen, —CF₃, —CBr₃, —CCl₃, —CI₃, —CHF₂, —CHBr₂, —CHCl₂, —CHI₂, —CH₂F, —CH₂Br, —CH₂Cl, —CH₂ I, —OCF₃, —OCH₃, —OCBr₃, —OCCl₃, —OCI₃, —OCHF₂, —OCHBr₂, —OCHCl₂, —OCHI₂, —OCH₂F, —OC H₂Br, —OCH₂Cl, —OCH₂I, —CN, —OH, —NH₂, —COOH, —CONH₂, —NO₂, —SH, —SO₃H, —SO₄H, —SO₂N H₂, —NHNH₂, —ONH₂, —NHC(O)NHNH₂, —NHC(O)NH₂, —NHSO₂H, —NHC(O)H, —NHC(O)OH, —NHOH, substituted or unsubstituted alkyl (e.g., C₁-C₈ alkyl, C₁-C₆ alkyl, or C₁-C₄ alkyl), substituted or unsubstituted heteroalkyl (e.g., 2 to 8 membered heteroalkyl, 2 to 6 membered heteroalkyl, or 2 to 4 membered heteroalkyl), substituted or unsubstituted cycloalkyl (e.g., C₃-C₈ cycloalkyl, C₃-C₆ cycloalkyl, or C₅-C₆ cycloalkyl), substituted or unsubstituted heterocycloalkyl (e.g., 3 to 8 membered heterocycloalkyl, 3 to 6 membered heterocycloalkyl, or 5 to 6 membered heterocycloalkyl), substituted or unsubstituted aryl (e.g., C₆-C₁₀ aryl, C₁₀ aryl, or phenyl), or substituted or unsubstituted heteroaryl (e.g., 5 to 10 membered heteroaryl, 5 to 9 membered heteroaryl, or 5 to 6 membered heteroaryl).

In embodiments, R^{1.2} is independently R^{1C}-substituted or unsubstituted alkyl (e.g., C₁-C₈ alkyl, C₁-C₆ alkyl, or C₁-C₄ alkyl). In embodiments, R^{1.2} is independently R^{1C}-substituted alkyl (e.g., C₁-C₈ alkyl, C₁-C₆ alkyl, or C₁-C₄ alkyl). In embodiments, R^{1.2} is independently an unsubstituted alkyl (e.g., C₁-C₈ alkyl, C₁-C₆ alkyl, or C₁-C₄ alkyl). In embodiments, R^{1.2} is independently R^{1C}-substituted C₁-C₈ alkyl. In embodiments, R^{1.2} is independently R^{1C}-substituted C₁ alkyl. In embodiments, R^{1.2} is independently R^{1C}-substituted C₂ alkyl. In embodiments, R^{1.2} is independently R^{1C}-substituted C₃ alkyl. In embodiments, R^{1.2} is independently R^{1C}-substituted C₄ alkyl. In embodiments, R^{1.2} is independently R^{1C}-substituted C₅ alkyl. In embodiments, R^{1.2} is independently R^{1C}-substituted C₆ alkyl. In embodiments, R^{1.2} is independently R^{1C}-substituted C₇ alkyl. In embodiments, R^{1.2} is independently an unsubstituted C₈ alkyl. In embodiments, R^{1.2} is independently an unsubstituted C₁-C₈ alkyl. In embodiments, R^{1.2} is independently an unsubstituted C₁ alkyl. In embodiments, R^{1.2} is independently an unsubstituted C₂ alkyl. In embodiments, R^{1.2} is independently an unsubstituted C₃ alkyl. In embodiments, R^{1.2} is independently an unsubstituted C₄ alkyl. In embodiments, R^{1.2} is independently an unsubstituted C₅ alkyl. In embodiments, R^{1.2} is independently an unsubstituted C₆ alkyl. In embodiments, R^{1.2} is independently an unsubstituted C₇ alkyl. In embodiments, R^{1.2} is independently an unsubstituted C₈ alkyl.

In embodiments, R^{1.2} is independently R^{1C}-substituted or unsubstituted heteroalkyl (e.g., 2 to 8 membered heteroalkyl, 2 to 6 membered heteroalkyl, or 2 to 4 membered heteroalkyl). In embodiments, R^{1.2} is independently R^{1C}-substituted heteroalkyl (e.g., 2 to 8 membered heteroalkyl, 2 to 6 membered heteroalkyl, or 2 to 4 membered heteroalkyl). In embodiments, R^{1.2} is independently an unsubstituted heteroalkyl (e.g., 2 to 8 membered heteroalkyl, 2 to 6 membered heteroalkyl, or 2 to 4 membered heteroalkyl). In embodiments, R^{1.2} is independently R^{1C}-substituted 2 to 6 membered heteroalkyl. In embodiments, R^{1.2} is independently R^{1C} substituted 2 membered heteroalkyl. In embodiments, R^{1.2} is independently R^{1C}-substituted 3 membered heteroalkyl. In embodiments, R^{1.2} is independently R^{1C}-substituted 4 membered heteroalkyl. In embodiments, R^{1.2} is independently R^{1C}-substituted 5 membered heteroalkyl. In embodiments, R^{1.2} is independently R^{1C}-substituted 6 membered heteroalkyl. In embodiments, R^{1.2} is independently an unsubstituted 2 to 6 membered heteroalkyl. In embodiments, R^{1.2} is independently an unsubstituted 2 membered heteroalkyl. In embodiments, R^{1.2} is independently an unsubstituted 3 membered heteroalkyl. In embodiments, R^{1.2} is independently an unsubstituted 4 membered heteroalkyl. In embodiments, R^{1.2} is independently an unsubstituted 5 membered heteroalkyl. In embodiments, R^{1.2} is independently an unsubstituted 6 membered heteroalkyl.

In embodiments, R^{1.2} is independently R^{1C}-substituted or unsubstituted cycloalkyl (e.g., C₃-C₈ cycloalkyl, C₃-C₆ cycloalkyl, or C₅-C₆ cycloalkyl). In embodiments, R^{1.2} is independently R^{1C}-substituted cycloalkyl (e.g., C₃-C₈ cycloalkyl, C₃-C₆ cycloalkyl, or C₅-C₆ cycloalkyl). In embodiments, R^{1.2} is independently an unsubstituted cycloalkyl (e.g., C₃-C₈ cycloalkyl, C₃-C₆ cycloalkyl, or C₅-C₆ cycloalkyl).

In embodiments, R^{1.2} is independently R^{1C}-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R^{1.2} is independently R^{1C}-substituted heterocycloalkyl (e.g., 3 to 8 membered heterocycloalkyl, 3 to 6 membered heterocycloalkyl, or 5 to 6 membered heterocycloalkyl). In embodiments, R^{1.2} is independently an unsubstituted heterocycloalkyl (e.g., 3 to 8 membered heterocycloalkyl, 3 to 6 membered heterocycloalkyl, or 5 to 6 membered heterocycloalkyl). In embodiments, R^{1.2} is independently R^{1C}-substituted or unsubstituted aryl (e.g., C₆-C₁₀ aryl, C₁₀ aryl, or phenyl). In embodiments, R^{1.2} is independently R^{1C}-substituted aryl (e.g., C₆-C₁₀ aryl, C₁₀ aryl, or phenyl). In embodiments, R^{1.2} is independently an unsubstituted aryl (e.g., C₆-C₁₀ aryl, C₁₀ aryl, or phenyl). In embodiments, R^{1.2} is independently R^{1C}-substituted or unsubstituted heteroaryl (e.g., 5 to 10 membered heteroaryl, 5 to 9 membered heteroaryl, or 5 to 6 membered heteroaryl). In embodiments, R^{1.2} is independently R^{1C} substituted heteroaryl (e.g., 5 to 10 membered heteroaryl, 5 to 9 membered heteroaryl, or 5 to 6 membered heteroaryl). In embodiments, R^{1.2} is independently an unsubstituted heteroaryl (e.g., 5 to 10 membered heteroaryl, 5 to 9 membered heteroaryl, or 5 to 6 membered heteroaryl).

In embodiments, R^{1.2} is independently halogen, —CF₃, —CBr₃, —CCl₃, —CI₃, —CHF₂, —CHBr₂, —CHCl₂, —CHI₂, —CH₂F, —CH₂Br, —CH₂Cl, —CH₂ I, —OCF₃, —OCH₃, —OCBr3, —OCCl₃, —OCI₃, —OCHF₂, —OCHBr₂, —OCHCl₂, —OCHI₂, —OCH₂F, —OC H₂Br, —OCH₂Cl, —OCH₂I, —CN, —OH, —NH₂, —COOH, —CONH₂, —NO₂, —SH, —SO₃H, —SO₄H, —SO₂N H₂, —NHNH₂, —ONH₂, —NHC(O)NHNH₂, —NHC(O)NH₂, —NHSO₂H, —NHC(O)H, —NHC(O)OH, or —NHOH. In embodiments, R^{1.2} is independently halogen. In embodiments, R^{1.2} is independently —CF₃. In embodiments, R^{1.2} is independently —CBr₃. In embodiments, R^{1.2} is independently —CCl₃. In embodiments, R^{1.2} is independently —CI₃. In embodiments, R^{1.2} is independently —CHF₂. In embodiments, R^{1.2} is independently —CHBr2. In embodiments, R^{1.2} is independently —CHCl₂. In embodiments, R^{1.2} is independently —CHI₂. In embodiments, R^{1.2} is independently —CH₂F. In embodiments, R^{1.2} is independently —CH₂Br. In embodiments, R^{1.2} is independently —CH₂Cl. In embodiments, R^{1.2} is independently —CH₂I. In embodiments, R^{1.2} is independently —OCF₃. In embodiments, R^{1.2} is independently —OCH₃. In embodiments, R^{1.2} is independently —OCBr₃. In embodiments, R^{1.2} is independently —OCCl₃. In embodiments, R^{1.2} is independently —OCI₃. In embodiments, R^{1.2} is independently —OCHF₂. In embodiments, R^{1.2} is independently —OCHBr₂. In embodiments, R^{1.2} is independently —OCHCl₂. In embodiments, R^{1.2} is independently —OCHI₂. In embodiments, R^{1.2} is independently —OCH₂F. In embodiments, R^{1.2} is independently —OCH₂Br. In embodiments, R^{1.2} is independently —OCH₂Cl. In embodiments, R^{1.2} is independently —OCH₂I. In embodiments, R^{1.2} is independently —CN. In embodiments, R^{1.2} is independently —OH. In embodiments, R^{1.2} is independently —NH₂. In embodiments, R^{1.2} is independently —COOH. In embodiments, R^{1.2} is independently —CONH₂. In embodiments, R^{1.2} is independently —NO₂. In embodiments, R^{1.2} is independently —SH. In embodiments, R^{1.2} is independently —SO₃H. In embodiments, R^{1.2} is independently —SO₄H. In embodiments, R^{1.2} is independently —SO₂NH₂. In embodiments, R^{1.2} is independently —NHNH₂. In embodiments, R^{1.2} is independently —ONH₂. In embodiments, R^{1.2} is independently —NHC(O)NHNH₂. In embodiments, R^{1.2} is independently —NHC(O)NH₂. In embodiments, R^{1.2} is independently —NHSO₂H. In embodiments, R^{1.2} is independently —NHC(O)H. In embodiments, R^{1.2} is independently —NHC(O)OH. In embodiments, R^{1.2} is independently —NHOH. In embodiments, R^{1.2} is independently unsubstituted t-butyl. In embodiments, R^{1.2} is independently unsubstituted isopropyl.

In embodiments, R^{1.1} is —CH₂F and R^{1.2} is an unsubstituted C₁-C₆ alkyl. In embodiments, R^{1.1} is —CH₂F and R^{1.2} is an unsubstituted C₁-C₄ alkyl. In embodiments, R^{1.1} is —CH₂F and R^{1.2} is an unsubstituted C₄ alkyl. In embodiments, R^{1.1} is —CH₃ and R^{1.2} is an unsubstituted C₁-C₆ alkyl. In embodiments, R^{1.1} is —CH₃ and R^{1.2} is an unsubstituted C₁-C₄ alkyl. In embodiments, R^{1.1} is —CH₃ and R^{1.2} is an unsubstituted C₄ alkyl. In embodiments, R^{1.2} is —CH₂F and R^{1.1} is an unsubstituted C₁-C₆ alkyl. In embodiments, R^{1.2} is —CH₂F and R^{1.1} is an unsubstituted C₁-C₄ alkyl. In embodiments, R^{1.2} is —CH₂F and R^{1.1} is an unsubstituted C₄ alkyl. In embodiments, R^{1.2} is —CH₃ and R^{1.1} is an unsubstituted C₁-C₆ alkyl. In embodiments, R^{1.2} is —CH₃ and R^{1.1} is an unsubstituted C₁-C₄ alkyl. In embodiments, R^{1.2} is —CH₃ and R^{1.1} is an unsubstituted C₄ alkyl. In embodiments, R^{1.1} is —OCH₃ and R^{1.2} is an unsubstituted C₁-C₆ alkyl. In embodiments, R^{1.1} is —OCH₃ and R^{1.2} is an unsubstituted C₁-C₄ alkyl. In embodiments, R^{1.1} is —OCH₃ and R^{1.2} is an unsubstituted C₄ alkyl. In embodiments, R^{1.2} is —OCH₃ and R^{1.1} is an unsubstituted C₁-C₆ alkyl. In embodiments, R^{1.2} is —OCH₃ and R^{1.1} is an unsubstituted C₁-C₄ alkyl. In embodiments, R^{1.2} is —OCH₃ and R^{1.1} is an unsubstituted C₄ alkyl. In embodiments, R^{1.1} and R^{1.2} are each unsubstituted C₁-C₆ alkyl. In embodiments, R^{1.1} and R^{1.2} are each unsubstituted C₁-C₄ alkyl. In embodiments, R^{1.1} is —CF₃ and R^{1.2} is an unsubstituted C₁-C₆ alkyl. In embodiments, R^{1.1} is —CF₃ and R^{1.2} is an unsubstituted C₁-C₄ alkyl. In embodiments, R^{1.1} is —CF₃ and R^{1.2} is an unsubstituted C₄ alkyl.

In embodiments, R^{1.1} is —CH₂F and R^{1.2} is —C(CH₃)₃. In embodiments, R^{1.1} is —CH₃ and R^{1.2} is —C(CH₃)₃. In embodiments, R^{1.1} is —CH₃ and R^{1.2} is —CF₃. In embodiments, R^{1.1} and R^{1.2} are each —CH₃. In embodiments, R^{1.1} is —CH₃ and R^{1.2} is —CH₂F. In embodiments, R^{1.1} is —CH₃ and R^{1.2} is —OCH₃. In embodiments, R^{1.1} is —CF₃ and R^{1.2} is —OCH₃. In embodiments, R^{1.1} is —CF₃ and R^{1.2} is —CH₃. In embodiments, R^{1.1} is —OCH₃ and R^{1.2} is —CH₃. In embodiments, R^{1.1} is —OCH₃ and R^{1.2} is —CH₃.

In embodiments, L¹-R¹ has the formula:

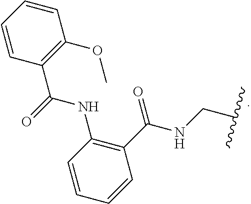 [SMILES: *CNC(=O)c1ccccc1NC(=O)c1ccccc1OC]

In embodiments, L¹-R¹ has the formula:

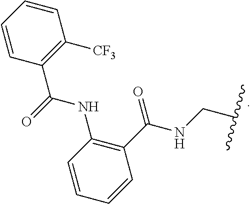 [SMILES: *CNC(=O)c1ccccc1NC(=O)c1ccccc1C(F)(F)F]

In embodiments, L¹-R¹ has the formula:

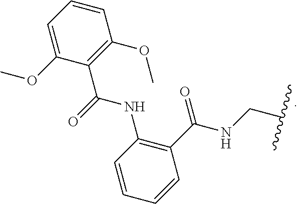 [SMILES: *CNC(=O)c1ccccc1NC(=O)c1c(OC)cccc1OC]

In embodiments, L¹-R¹ has the formula:

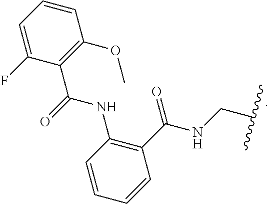 [SMILES: *CNC(=O)c1ccccc1NC(=O)c1c(F)cccc1OC]

In embodiments, L¹-R¹ has the formula:

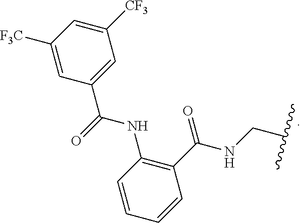 [SMILES: *CNC(=O)c1ccccc1NC(=O)c1cc(C(F)(F)F)cc(C(F)(F)F)c1]

In embodiments, L¹-R¹ has the formula:

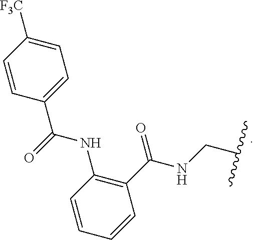 [SMILES: *CNC(=O)c1ccccc1NC(=O)c1ccc(C(F)(F)F)cc1]

In embodiments, L¹-R¹ has the formula:

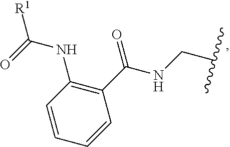

wherein R¹ is as described herein.

In embodiments, R¹ is

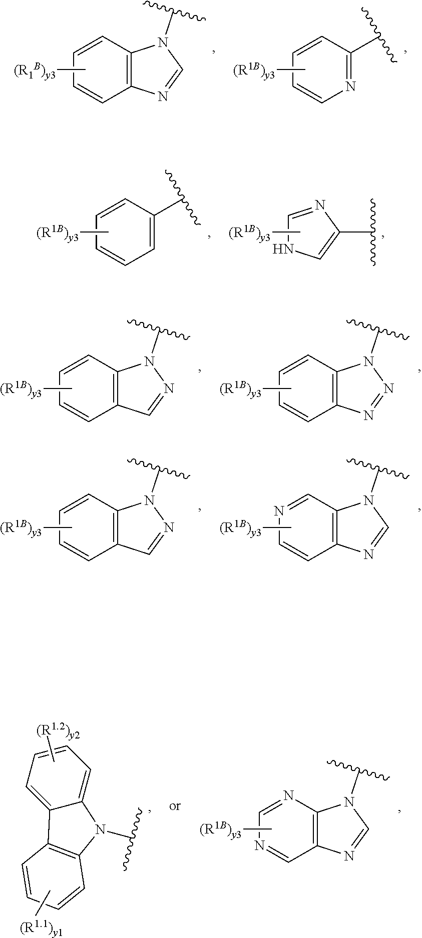

wherein R^{1B} and y3 are as described herein. It is understood that when R¹ is a fused ring, e.g., indazole, the location of a floating substituent does not restrict the location to one cyclic group. For example when R¹ is optionally R^{1B} substituted, it is understood

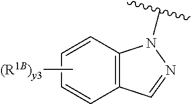

may alternatively be written as

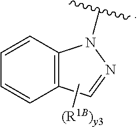

In embodiments, R¹ is

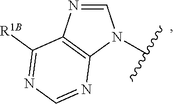

wherein R^{1B} is as described herein, including embodiments. In embodiments, R¹ is

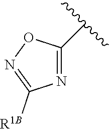

wherein R^{1B} is as described herein, including embodiments.

In embodiments, R¹ is

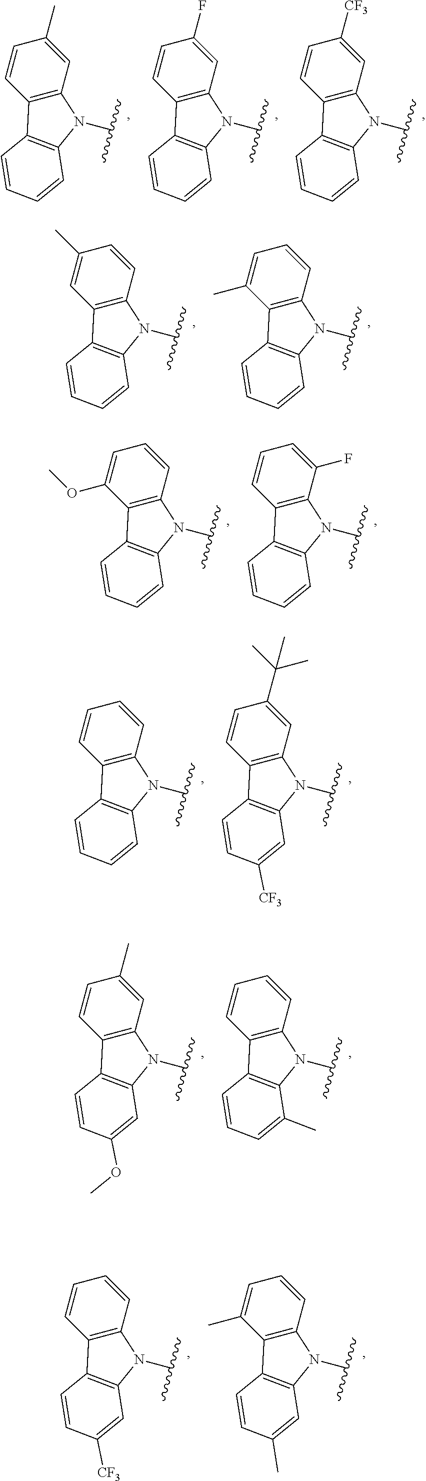

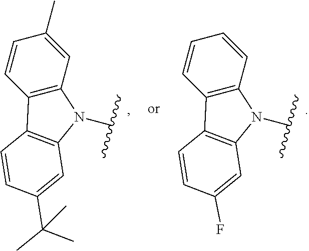 [SMILES: *n1c2cc(C)ccc2c2ccc(C(C)(C)C)cc21 ; "," ; "or" ; *n1c2ccccc2c2ccc(F)cc21 ; "."]

In embodiments, R¹ is

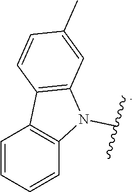 [SMILES: *n1c2ccccc2c2ccc(C)cc21]

In embodiments, R¹ is

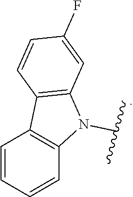 [SMILES: *n1c2ccccc2c2ccc(F)cc21]

In embodiments, R¹ is

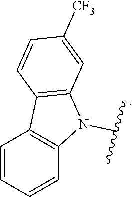 [SMILES: *n1c2ccccc2c2ccc(C(F)(F)F)cc21]

In embodiments, R¹ is

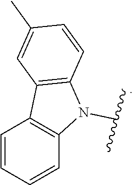 [SMILES: *n1c2ccccc2c2cc(C)ccc21]

In embodiments, R¹ is

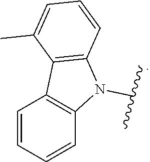 [SMILES: *n1c2ccccc2c2c(C)cccc21]

In embodiments, R¹ is

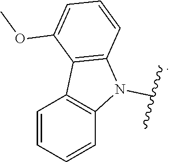 [SMILES: *n1c2ccccc2c2c(OC)cccc21]

In embodiments, R¹ is

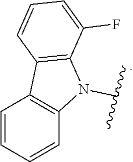 [SMILES: *n1c2ccccc2c2cccc(F)c21]

In embodiments, R¹ is

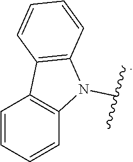 [SMILES: *n1c2ccccc2c2ccccc21]

In embodiments, R¹ is

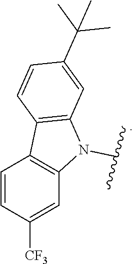 [SMILES: *n1c2cc(C(C)(C)C)ccc2c2ccc(C(F)(F)F)cc21]

In embodiments, R¹ is

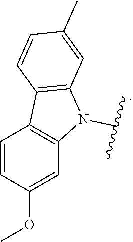 [SMILES: *n1c2cc(C)ccc2c2ccc(OC)cc21]

In embodiments, R¹ is

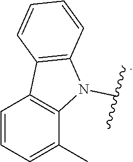 [SMILES: *n1c2ccccc2c2cccc(C)c21]

In embodiments, R¹ is

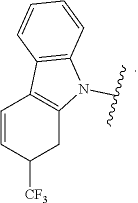 [SMILES: *n1c2c(c3ccccc31)C=CC(C(F)(F)F)C2]

In embodiments, R¹ is

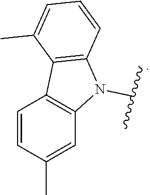 [SMILES: *n1c2cc(C)ccc2c2c(C)cccc21]

In embodiments, R¹ is

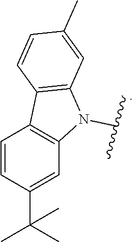 [SMILES: *n1c2cc(C)ccc2c2ccc(C(C)(C)C)cc21]

In embodiments, R¹ is

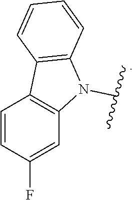 [SMILES: *n1c2ccccc2c2ccc(F)cc21]

In embodiments, R¹ is

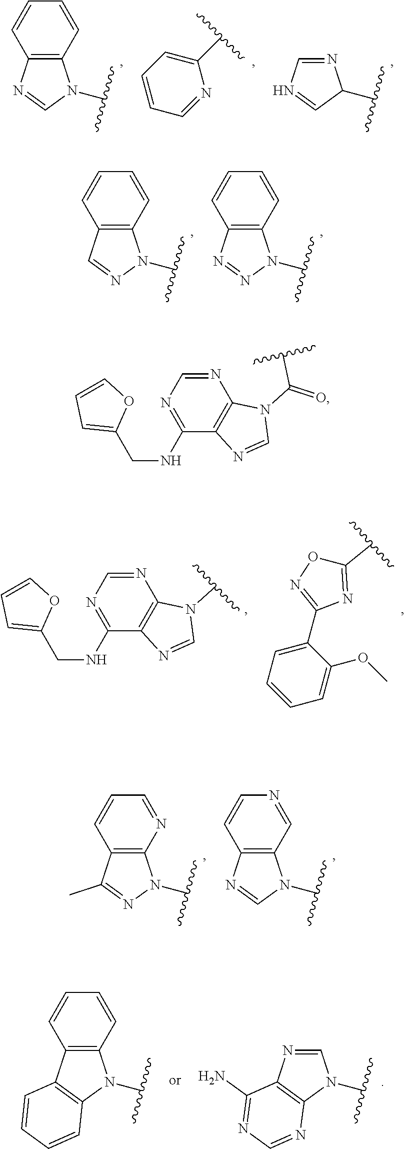

In embodiments, R¹ is

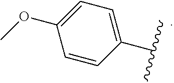 [SMILES: *c1ccc(OC)cc1]

In embodiments, R¹ is

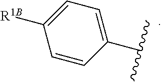

In embodiments, R¹ is

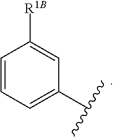

In embodiments, R¹ is

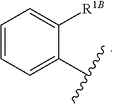

In embodiments, R¹ is

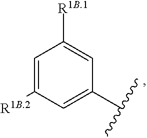

wherein R^{1B.1} and R^{1B.2} are each R^{1B} at a fixed position on the attached ring. R^{1B.1} and R^{1B.2} may be hydrogen or any R^{1B} substituent described herein, including in any aspect, embodiment, example, figure, or claim.

In embodiments, R¹ is

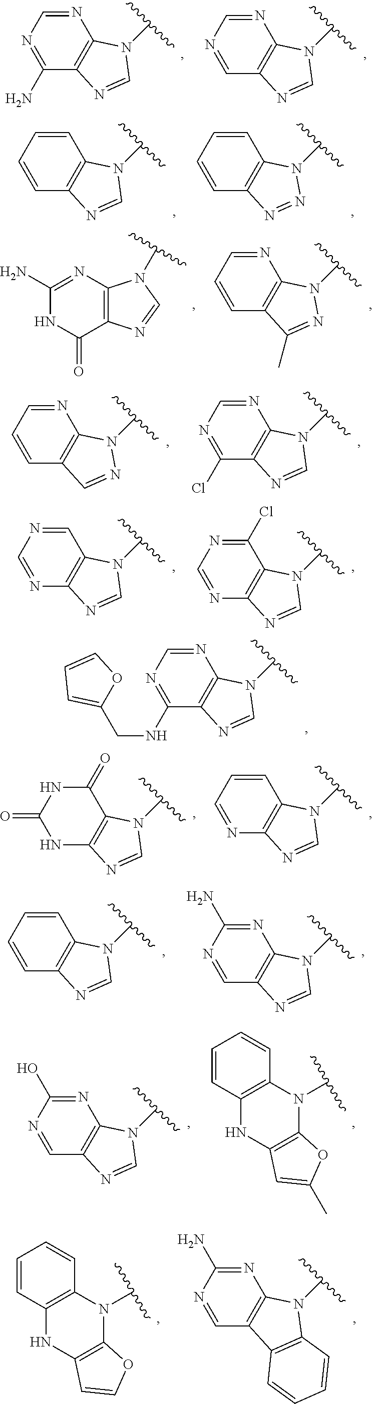

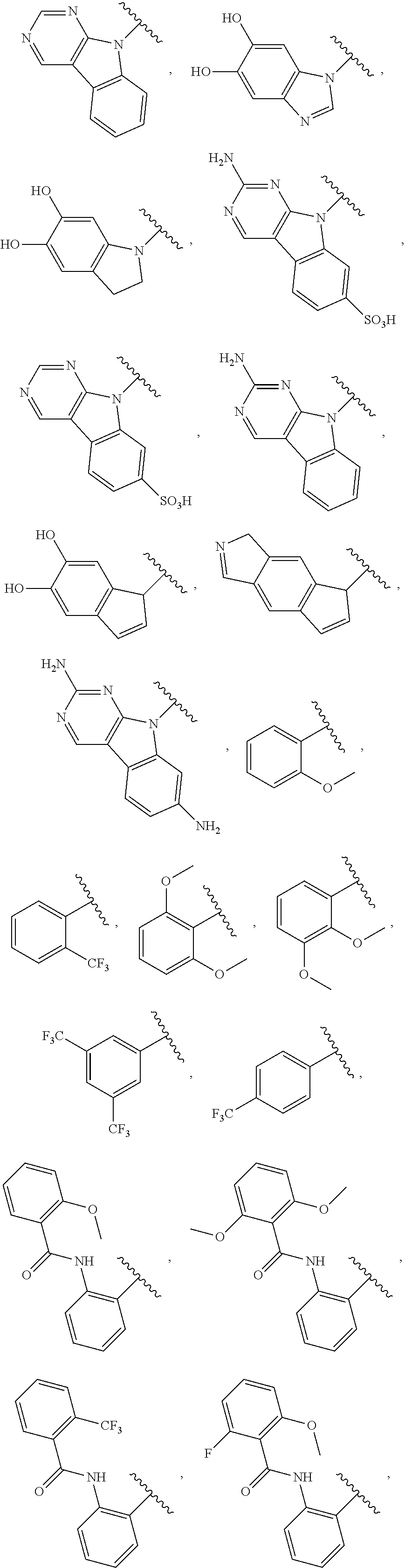

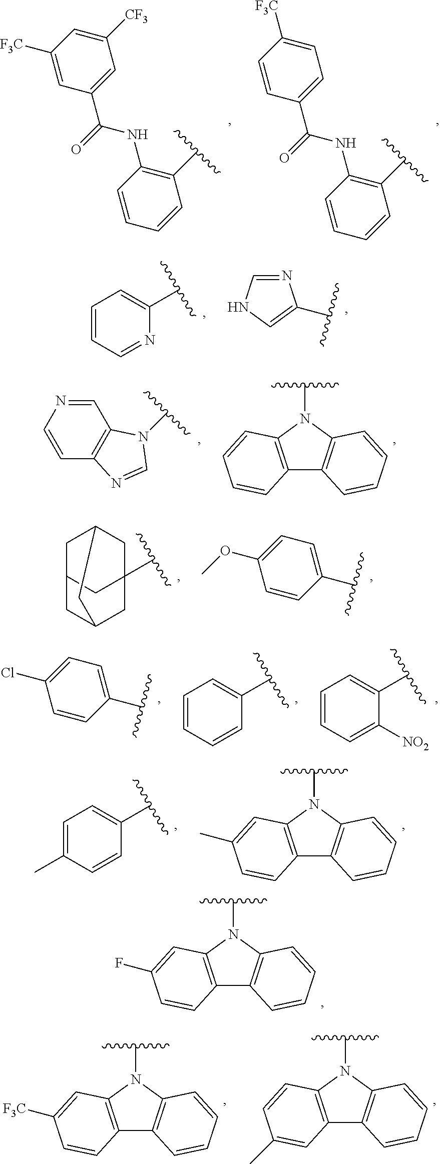

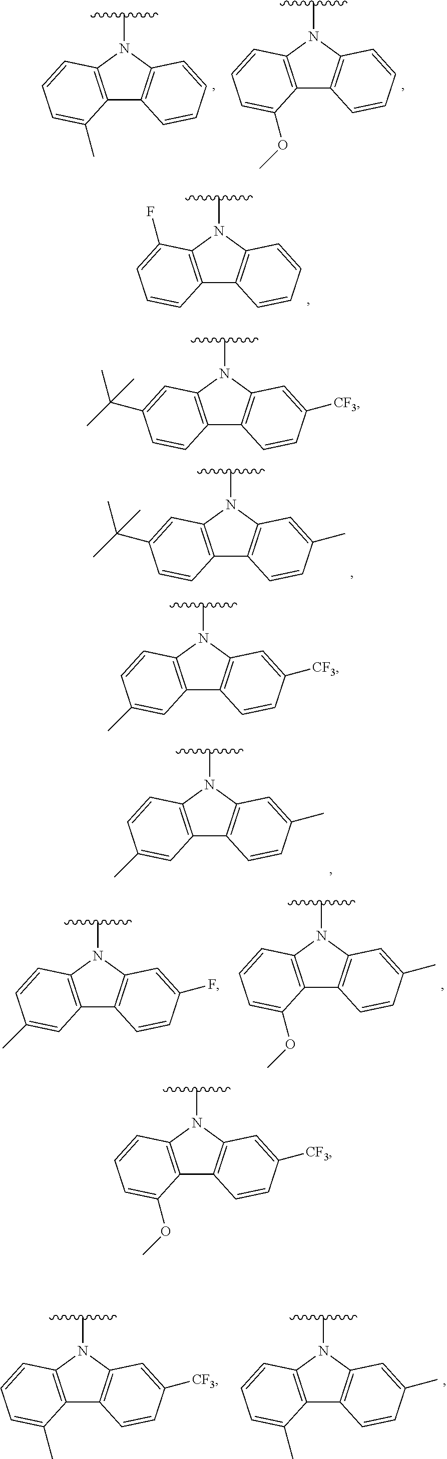

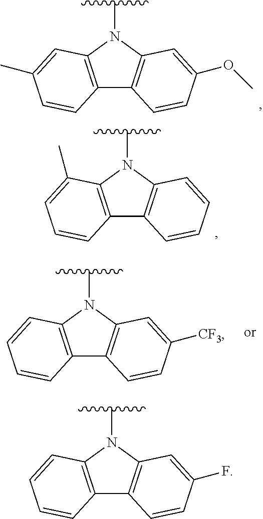

In embodiments, R¹ is

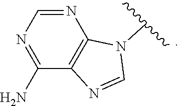 [SMILES: *n1cnc2c(N)ncnc21]

In embodiments, R¹ is

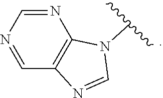 [SMILES: *n1cnc2cncnc21]

In embodiments, R¹ is

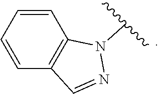 [SMILES: *n1ncc2ccccc21]

In embodiments, R¹ is

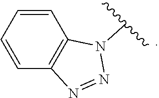 [SMILES: *n1nnc2ccccc21]

In embodiments, R¹ is

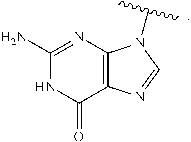 [SMILES: *n1cnc2c(=O)[nH]c(N)nc21]

In embodiments, R¹ is

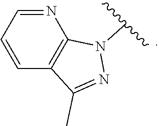 [SMILES: *n1nc(C)c2cccnc21]

In embodiments, R¹ is

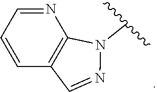 [SMILES: *n1ncc2cccnc21]

In embodiments, R¹ is

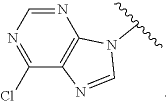 [SMILES: *n1cnc2c(Cl)ncnc21]

In embodiments, R¹ is

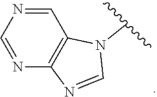 [SMILES: *n1cnc2ncncc21]

In embodiments, R¹ is

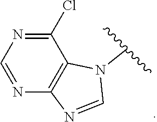 [SMILES: *n1cnc2ncnc(Cl)c21]

In embodiments, R¹ is

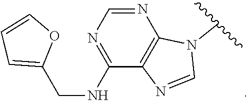 [SMILES: *n1cnc2c(NCc3ccco3)ncnc21]

In embodiments, R¹ is

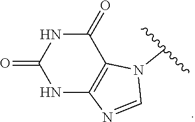 [SMILES: *n1cnc2[nH]c(=O)[nH]c(=O)c21]

In embodiments, R¹ is

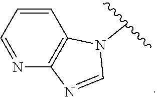 [SMILES: *n1cnc2ncccc21]

In embodiments, R¹ is

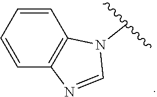 [SMILES: *n1cnc2ccccc21]

In embodiments, R¹ is

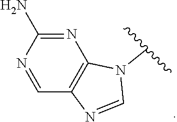 [SMILES: *n1cnc2cnc(N)nc21]

In embodiments, R¹ is

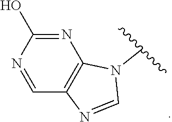 [SMILES: *n1cnc2cnc(O)nc21]

In embodiments, R¹ is

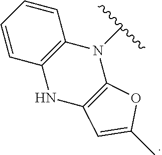 [SMILES: *N1c2ccccc2Nc2cc(C)oc21]

In embodiments, R¹ is

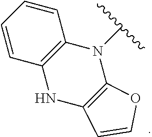 [SMILES: *N1c2ccccc2Nc2ccoc21]

In embodiments, R¹ is

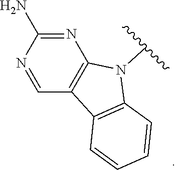 [SMILES: *n1c2ccccc2c2cnc(N)nc21]

In embodiments, R¹ is

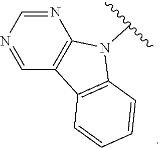 [SMILES: *n1c2ccccc2c2cncnc21]

In embodiments, R¹ is

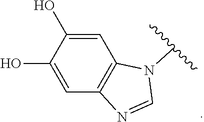 [SMILES: *n1cnc2cc(O)c(O)cc21]

In embodiments, R¹ is

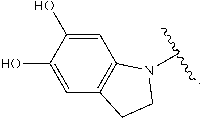 [SMILES: *N1CCc2cc(O)c(O)cc21]

In embodiments, R¹ is

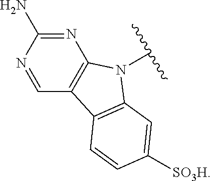 [SMILES: *n1c2cc(S(=O)(=O)O)ccc2c2cnc(N)nc21]

In embodiments, R¹ is

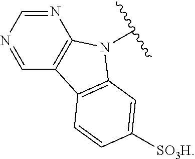 [SMILES: *n1c2cc(S(=O)(=O)O)ccc2c2cncnc21]

In embodiments, R¹ is

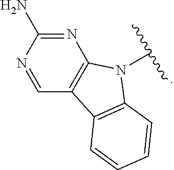 [SMILES: *n1c2ccccc2c2cnc(N)nc21]

In embodiments, R¹ is

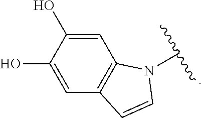 [SMILES: *n1ccc2cc(O)c(O)cc21]

In embodiments, R¹ is

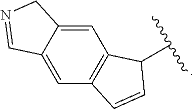 [SMILES: *C1C=Cc2cc3c(cc21)CN=C3]

In embodiments, R¹ is

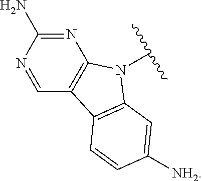 [SMILES: *n1c2cc(N)ccc2c2cnc(N)nc21]

In embodiments, R¹ is

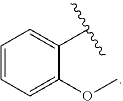 [SMILES: *c1ccccc1OC]

In embodiments, R¹ is.

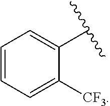 [SMILES: *c1ccccc1C(F)(F)F]

In embodiments, R¹ is

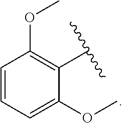 [SMILES: *c1c(OC)cccc1OC]

In embodiments, R¹ is

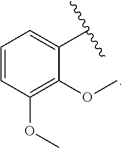 [SMILES: *c1cccc(OC)c1OC]

In embodiments, R¹ is

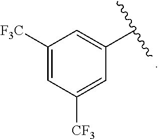 [SMILES: *c1cc(C(F)(F)F)cc(C(F)(F)F)c1]

In embodiments, R¹ is

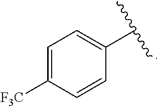 [SMILES: *c1ccc(C(F)(F)F)cc1]

In embodiments, R¹ is

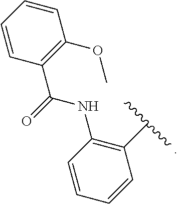 [SMILES: *c1ccccc1NC(=O)c1ccccc1OC]

In embodiments, R¹ is

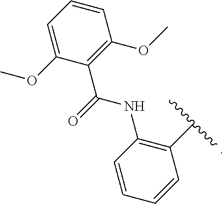 [SMILES: *c1ccccc1NC(=O)c1c(OC)cccc1OC]

In embodiments, R¹ is

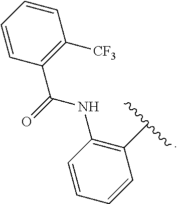 [SMILES: *c1ccccc1NC(=O)c1ccccc1C(F)(F)F]

In embodiments, R¹ is

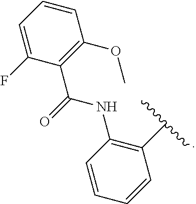 [SMILES: *c1ccccc1NC(=O)c1c(F)cccc1OC]

In embodiments, R¹ is

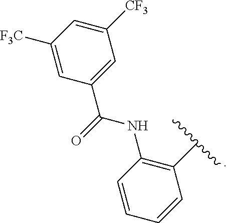 [SMILES: *c1ccccc1NC(=O)c1cc(C(F)(F)F)cc(C(F)(F)F)c1]

In embodiments, R¹ is

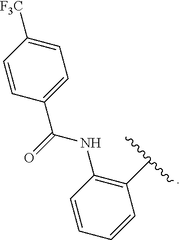 [SMILES: *c1ccccc1NC(=O)c1ccc(C(F)(F)F)cc1]

In embodiments, R¹ is

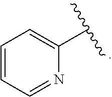 [SMILES: *c1ccccn1]

In embodiments, R¹ is

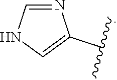 [SMILES: *c1c[nH]cn1]

In embodiments, R¹ is

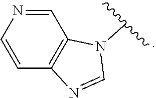 [SMILES: *n1cnc2ccncc21]

In embodiments, R¹ is

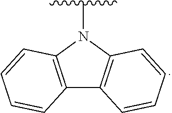 [SMILES: *n1c2ccccc2c2ccccc21]

In embodiments, R¹ is

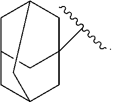 [SMILES: *C12CC3CC(CC(C3)C1)C2]

In embodiments, R¹ is

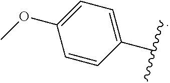 [SMILES: *c1ccc(OC)cc1]

In embodiments, R¹ is

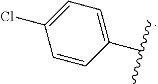 [SMILES: *c1ccc(Cl)cc1]

In embodiments, R¹ is

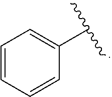 [SMILES: *c1ccccc1]

In embodiments, R¹ is

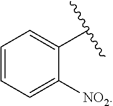 [SMILES: *c1ccccc1[N+](=O)[O-]]

In embodiments, R¹ is

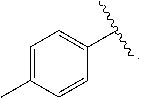 [SMILES: *c1ccc(C)cc1]

In embodiments, R¹ is

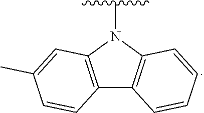 [SMILES: *n1c2ccccc2c2ccc(C)cc21]

In embodiments, R¹ is

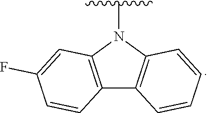 [SMILES: *n1c2ccccc2c2ccc(F)cc21]

In embodiments, R¹ is

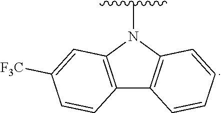 [SMILES: *n1c2ccccc2c2ccc(C(F)(F)F)cc21]

In embodiments, R¹ is

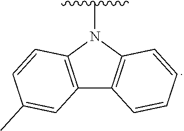 [SMILES: *n1c2ccccc2c2cc(C)ccc21]

In embodiments, R¹ is

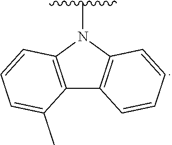 [SMILES: *n1c2ccccc2c2c(C)cccc21]

In embodiments, R¹ is

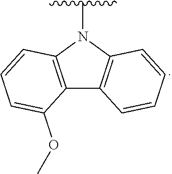 [SMILES: *n1c2ccccc2c2c(OC)cccc21]

In embodiments, R¹ is

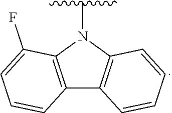 [SMILES: *n1c2ccccc2c2cccc(F)c21]

In embodiments, R¹ is

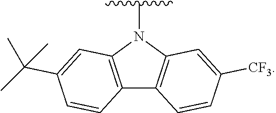 [SMILES: *n1c2cc(C(C)(C)C)ccc2c2ccc(C(F)(F)F)cc21]

In embodiments, R¹ is

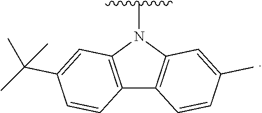 [SMILES: *n1c2cc(C)ccc2c2ccc(C(C)(C)C)cc21]

In embodiments, R¹ is

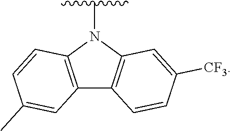 [SMILES: *n1c2ccc(C)cc2c2ccc(C(F)(F)F)cc21]

In embodiments, R¹ is

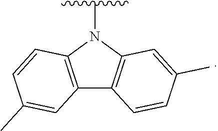

In embodiments, R¹ is

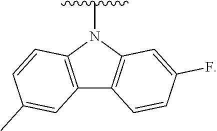 [SMILES: *n1c2ccc(C)cc2c2ccc(F)cc21]

In embodiments, R¹ is

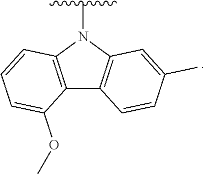 [SMILES: *n1c2cc(C)ccc2c2c(OC)cccc21]

In embodiments, R¹ is

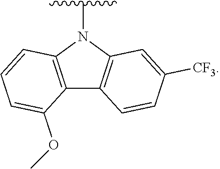 [SMILES: *n1c2cc(C(F)(F)F)ccc2c2c(OC)cccc21]

In embodiments, R¹ is

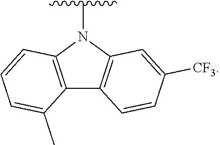 [SMILES: *n1c2cc(C(F)(F)F)ccc2c2c(C)cccc21]

In embodiments, R¹ is

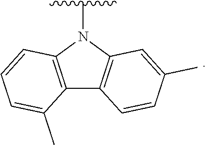 [SMILES: *n1c2cc(C)ccc2c2c(C)cccc21]

In embodiments, R¹ is

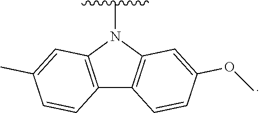

In embodiments, R¹ is

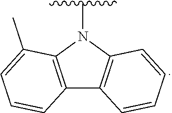 [SMILES: *n1c2ccccc2c2cccc(C)c21]

In embodiments, R¹ is

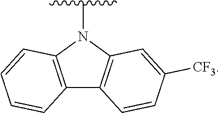 [SMILES: *n1c2ccccc2c2ccc(C(F)(F)F)cc21]

In embodiments, R¹ is or

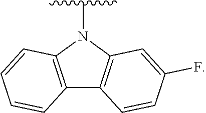 [SMILES: *n1c2ccccc2c2ccc(F)cc21]

In embodiments, R² and R³ are hydrogen. In embodiments, R² is hydrogen. In embodiments, R³ is hydrogen. In embodiments, R² and R³ are —CH₃. In embodiments, R² and R³ are unsubstituted C₁-C₆ alkyl. In embodiments, R² is unsubstituted C₁-C₆ alkyl. In embodiments, R³ is unsubstituted C₁-C₆ alkyl. In embodiments, R² is —CH₃. In embodiments, R³ is —CH₃.

In embodiments R² is substituted (e.g., substituted with a substituent group, a size-limited substituent group, or lower substituent group) or unsubstituted alkyl, substituted (e.g., substituted with a substituent group, a size-limited substituent group, or lower substituent group) or unsubstituted heteroalkyl, substituted (e.g., substituted with a substituent group, a size-limited substituent group, or lower substituent group) or unsubstituted cycloalkyl, substituted (e.g., substituted with a substituent group, a size-limited substituent group, or lower substituent group) or unsubstituted heterocycloalkyl, substituted (e.g., substituted with a substituent group, a size-limited substituent group, or lower substituent group) or unsubstituted aryl, or substituted (e.g., substituted with a substituent group, a size-limited substituent group, or lower substituent group) or unsubstituted heteroaryl. In embodiments, R² is unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

In embodiments R³ is substituted (e.g., substituted with a substituent group, a size-limited substituent group, or lower substituent group) or unsubstituted alkyl, substituted (e.g., substituted with a substituent group, a size-limited substituent group, or lower substituent group) or unsubstituted heteroalkyl, substituted (e.g., substituted with a substituent group, a size-limited substituent group, or lower substituent group) or unsubstituted cycloalkyl, substituted (e.g., substituted with a substituent group, a size-limited substituent group, or lower substituent group) or unsubstituted heterocycloalkyl, substituted (e.g., substituted with a substituent group, a size-limited substituent group, or lower substituent group) or unsubstituted aryl, or substituted (e.g., substituted with a substituent group, a size-limited substituent group, or lower substituent group) or unsubstituted heteroaryl. In embodiments, R³ is unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₅ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

In embodiments, the compound has the formula:

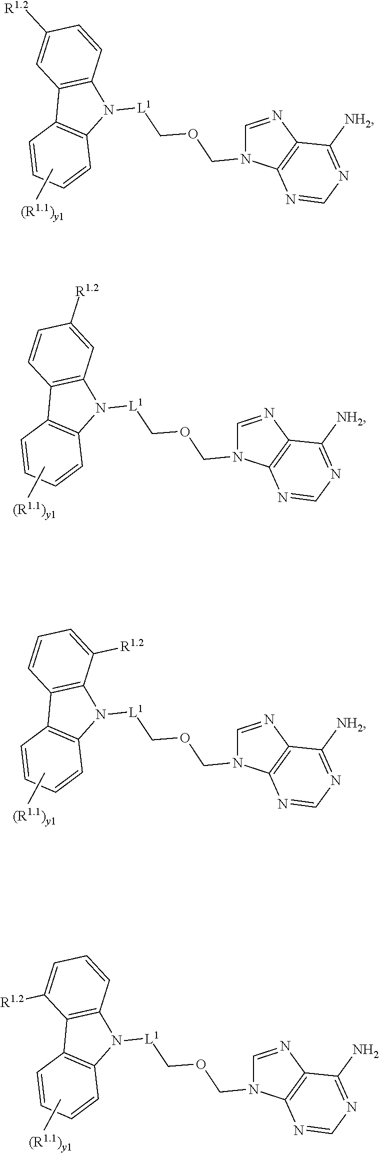

R^{1.2}, and L¹ are as described herein, including embodiments.

In embodiments, the compound has the formula:

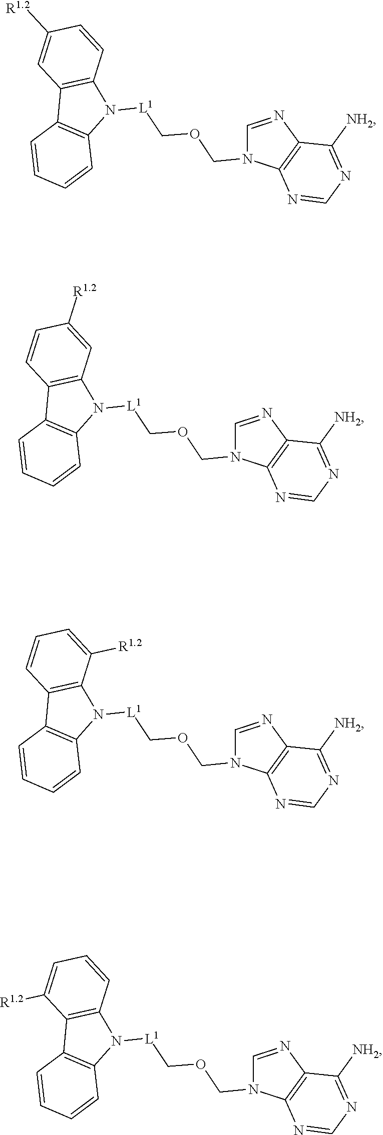

wherein R^{1.2} and L¹ are as described herein, including embodiments.

In embodiments, the compound has the formula:

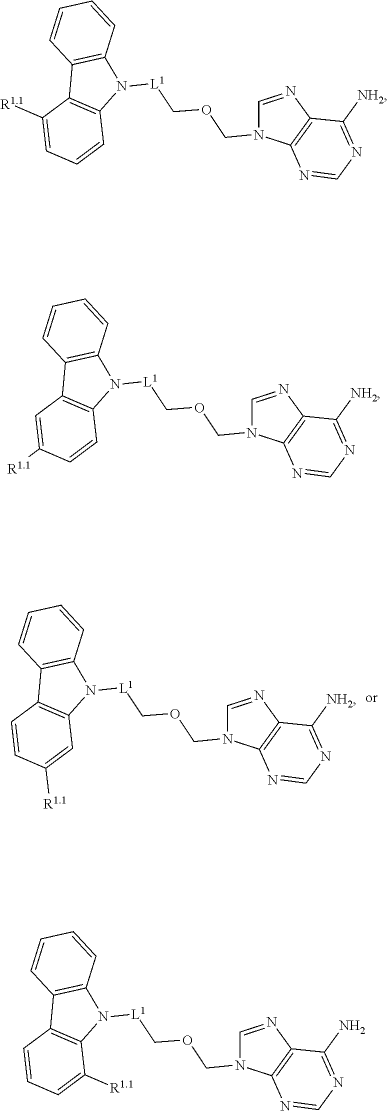

wherein R^{1.1} and L¹ are as described herein, including embodiments.

In embodiments, the compound has the formula:

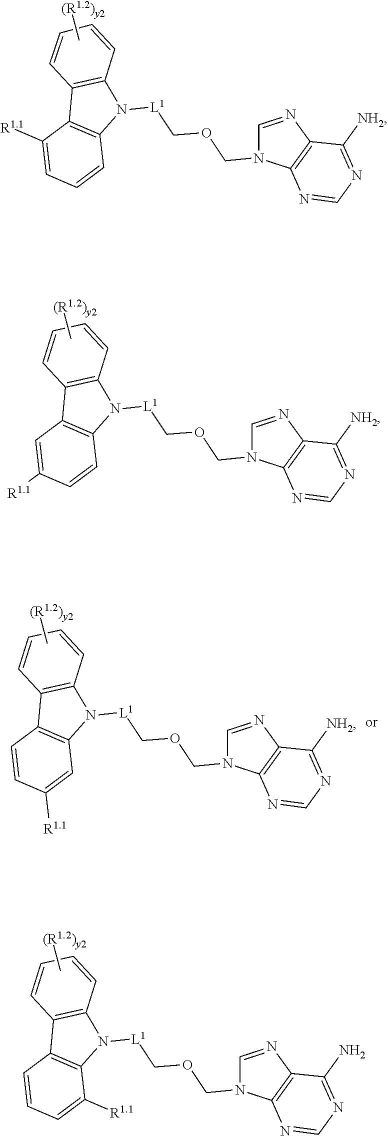

wherein R^{1.1}, y2, R^{1.2} and L¹ are as described herein, including embodiments.

In embodiments, the compound has the formula:

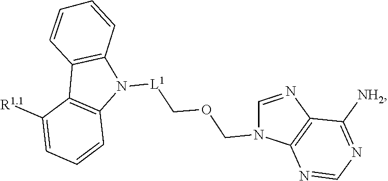

wherein L¹ and R^{1.1} are as described herein, including embodiments.

In embodiments, the compound has the formula:

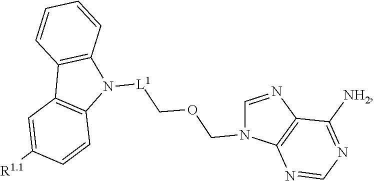

wherein L¹ and R^{1.1} are as described herein, including embodiments.

In embodiments, the compound has the formula:

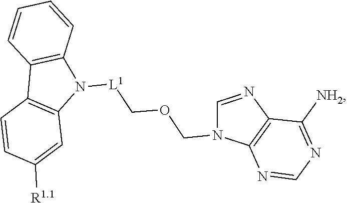

wherein L¹ and R^{1.1} are as described herein, including embodiments.

In embodiments, the compound has the formula:

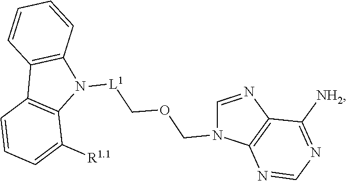

wherein L¹ and R^{1.1} are as described herein, including embodiments.

In embodiments, the compound has the formula:

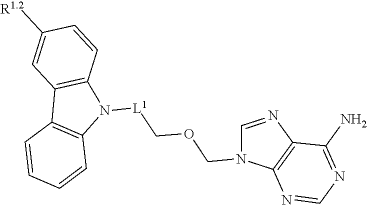

wherein L¹ and R^{1.2} are as described herein, including embodiments.

In embodiments, the compound has the formula:

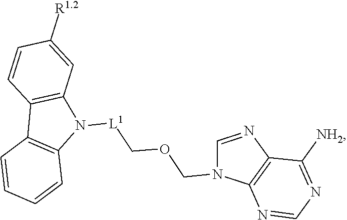

wherein L¹ and R^{1.2} are as described herein, including embodiments.

In embodiments, the compound has the formula:

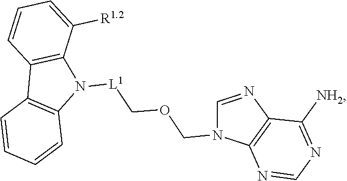

wherein L¹ and R^{1.2} are as described herein, including embodiments.

In embodiments, the compound has the formula:

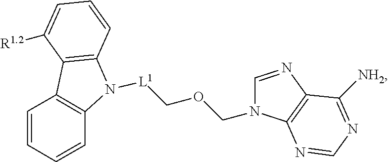

wherein L¹ and R^{1.2} are as described herein, including embodiments.

In embodiments, the compound has the formula:

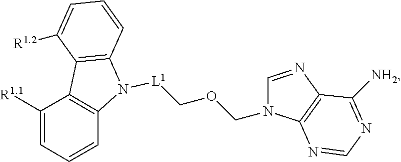

wherein L¹, R^{1.1}, and R^{1.2} are as described herein, including embodiments.

In embodiments, the compound has the formula:

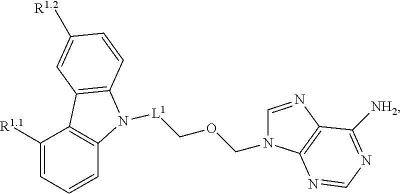

wherein L¹, R^{1.1}, and R^{1.2} are as described herein, including embodiments.

In embodiments, the compound has the formula:

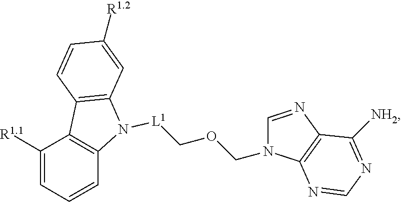

wherein L¹, R^{1.1}, and R^{1.2} are as described herein, including embodiments.

In embodiments, the compound has the formula:

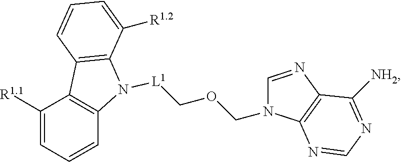

wherein L¹, R^{1.1}, and R^{1.2} are as described herein, including embodiments.

In embodiments, the compound has the formula:

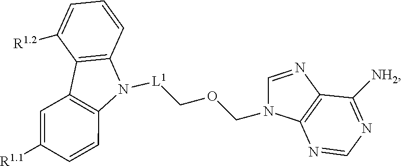

wherein L¹, R^{1.1}, and R^{1.2} are as described herein, including embodiments.

In embodiments, the compound has the formula:

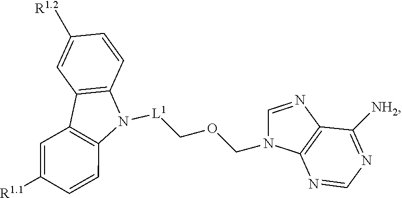

wherein L¹, R^{1.1}, and R^{1.2} are as described herein, including embodiments.

In embodiments, the compound has the formula:

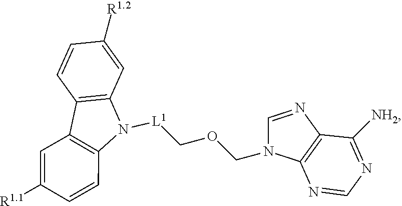

wherein L¹, R^{1.1}, and R^{1.2} are as described herein, including embodiments.

In embodiments, the compound has the formula:

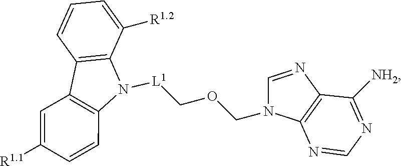

wherein L¹, R^{1.1}, and R^{1.2} are as described herein, including embodiments.

In embodiments, the compound has the formula:

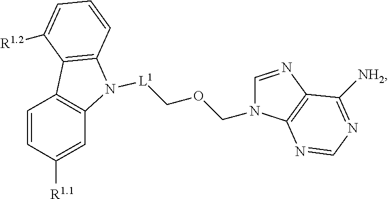

wherein L¹, R^{1.1}, and R^{1.2} are as described herein, including embodiments.

In embodiments, the compound has the formula:

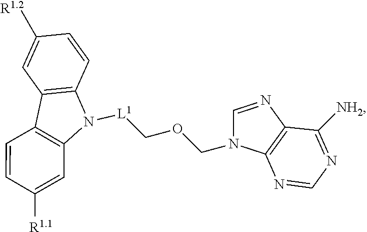

wherein L¹, R^{1.1}, and R^{1.2} are as described herein, including embodiments.

In embodiments, the compound has the formula:

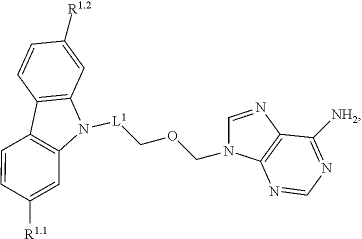

wherein L¹, R^{1.1}, and R^{1.2} are as described herein, including embodiments.

In embodiments, the compound has the formula:

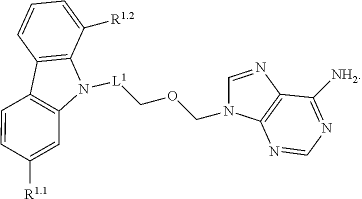

In embodiments, the compound has the formula:

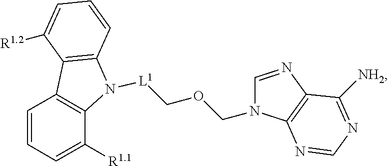

wherein L¹, R^{1.1}, and R^{1.2} are as described herein, including embodiments.

In embodiments, the compound has the formula:

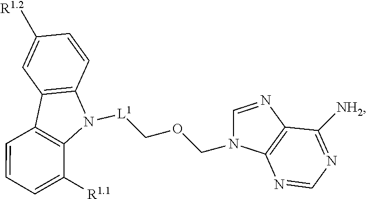

wherein L¹, R^{1.1}, and R^{1.2} are as described herein, including embodiments.

In embodiments, the compound has the formula:

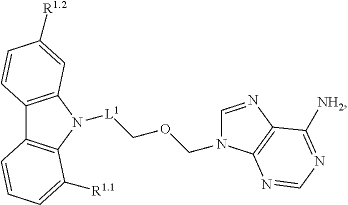

wherein L¹, R^{1.1}, and R^{1.2} are as described herein, including embodiments.

In embodiments, the compound has the formula:

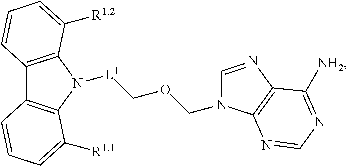

wherein L¹, R^{1.1}, and R^{1.2} are as described herein, including embodiments.

In embodiments, the compound has the formula:

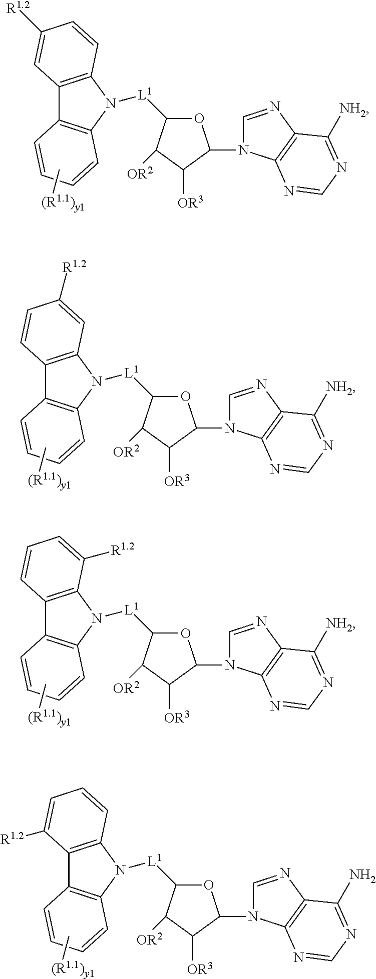

wherein R², R³, R^{1.1}, y1, R^{1.2}, and L¹ are as described herein, including embodiments.

In embodiments, the compound has the formula:

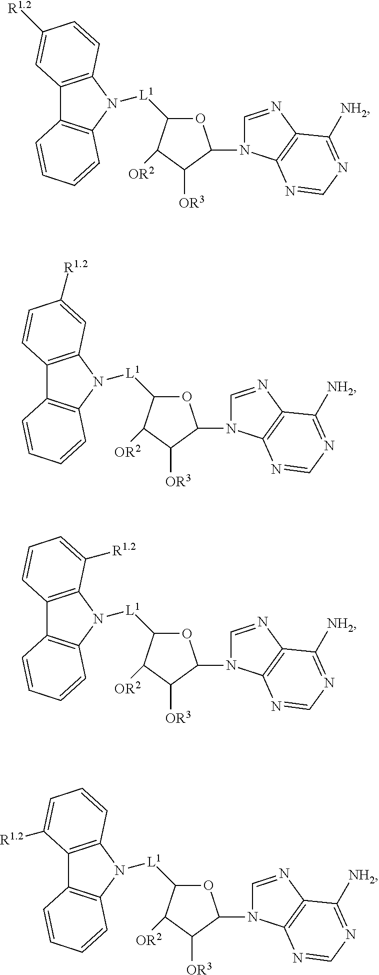

wherein R², R³, R^{1.2}, and L¹ are as described herein, including embodiments.

In embodiments, the compound has the formula:

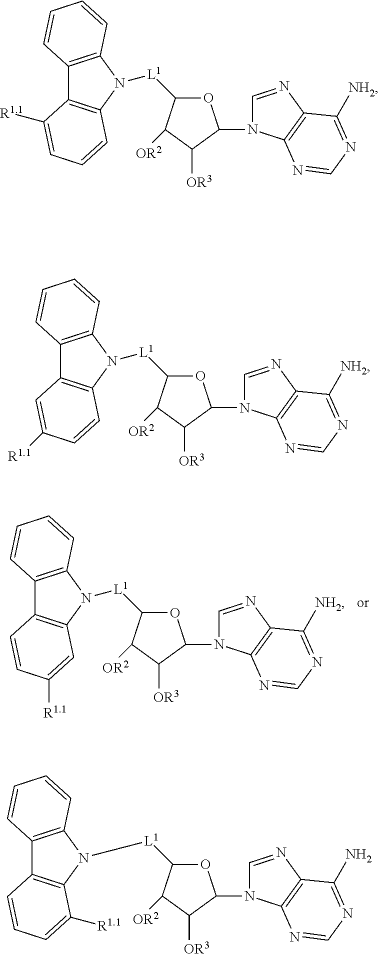

wherein R², R³, R^{1.1} and L¹ are as described herein, including embodiments.

In embodiments, the compound has the formula:

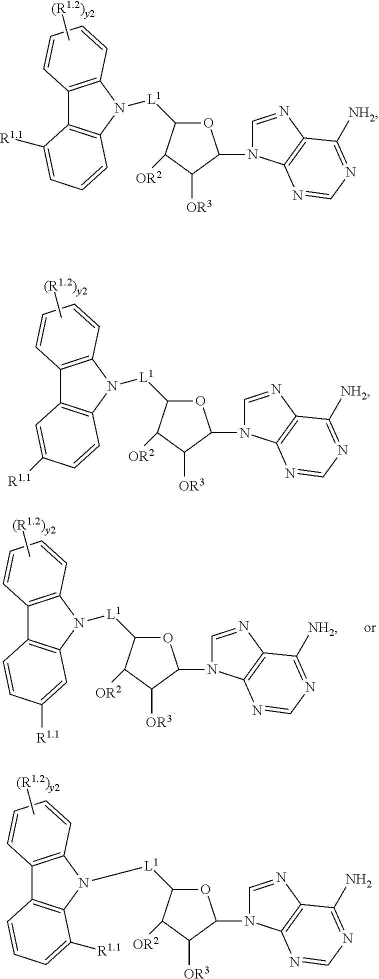

wherein R², R³, R^{1.1}, y2, R^{1.2}, and L¹ are as described herein, including embodiments.

In embodiments, the compound has the formula:

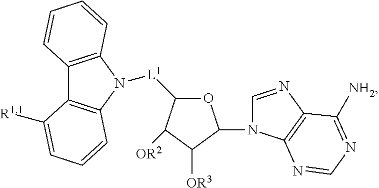

wherein R², R³, L¹, and R^{1.1} are as described herein, including embodiments.

In embodiments, the compound has the formula:

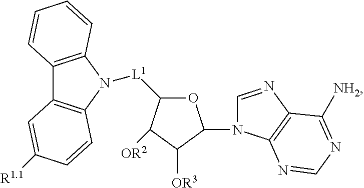

wherein R², R³, L¹, and R^{1.1} are as described herein, including embodiments.

In embodiments, the compound has the formula:

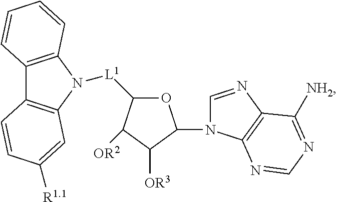

wherein R², R³, L¹, and R^{1.1} are as described herein, including embodiments.

In embodiments, the compound has the formula:

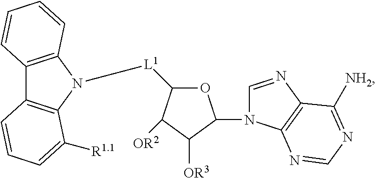

wherein R², R³, L¹, and R^{1.1} are as described herein, including embodiments.

In embodiments, the compound has the formula:

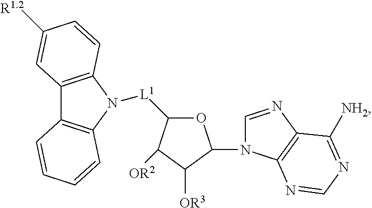

wherein R², R³, L¹, and R^{1.2} are as described herein, including embodiments.

In embodiments, the compound has the formula:

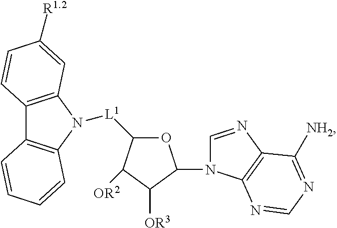

wherein R², R³, L¹, and R^{1.2} are as described herein, including embodiments.

In embodiments, the compound has the formula:

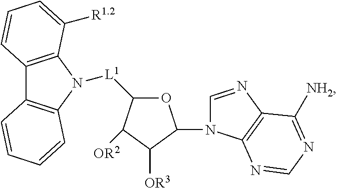

wherein R², R³, L¹, and R^{1.2} are as described herein, including embodiments.

In embodiments, the compound has the formula:

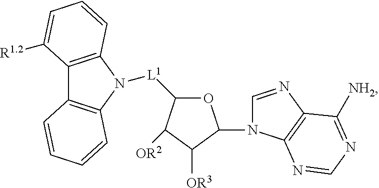

wherein R², R³, L¹, and R^{1.2} are as described herein, including embodiments.

In embodiments, the compound has the formula:

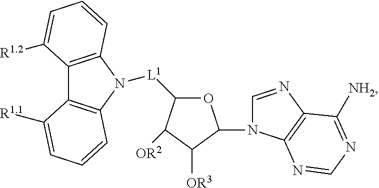

wherein R², R³, L¹, R^{1.1}, and R^{1.2} are as described herein, including embodiments.

In embodiments, the compound has the formula:

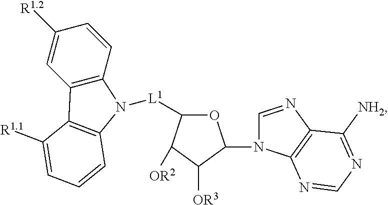

wherein R², R³, L¹, R^{1.1}, and R^{1.2} are as described herein, including embodiments.

In embodiments, the compound has the formula:

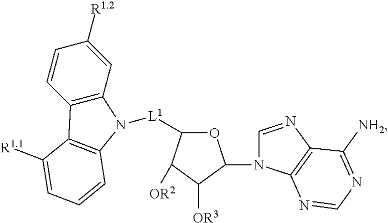

wherein R², R³, L¹, R^{1.1}, and R^{1.2} are as described herein, including embodiments.

In embodiments, the compound has the formula:

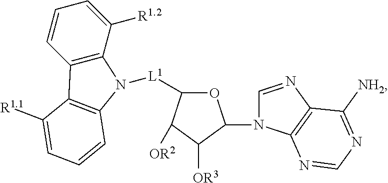

wherein R², R³, L¹, R^{1.1}, and R^{1.2} are as described herein, including embodiments.

In embodiments, the compound has the formula:

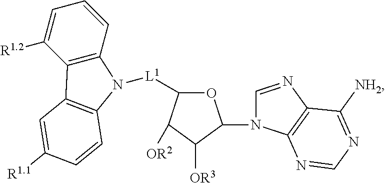

wherein R², R³, L¹, R^{1.1}, and R^{1.2} are as described herein, including embodiments.

In embodiments, the compound has the formula:

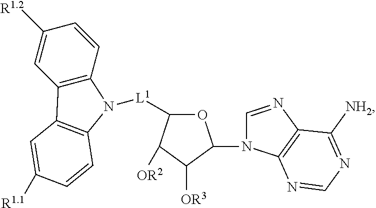

wherein R², R³, L¹, R^{1.1}, and R^{1.2} are as described herein, including embodiments.

In embodiments, the compound has the formula:

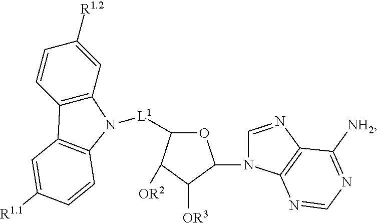

wherein R², R³, L¹, R^{1.1}, and R^{1.2} are as described herein, including embodiments.

In embodiments, the compound has the formula:

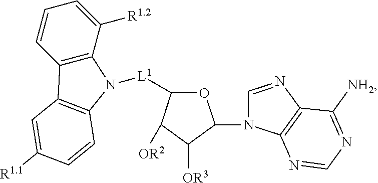

wherein R², R³, L¹, R^{1.1}, and R^{1.2} are as described herein, including embodiments.

In embodiments, the compound has the formula:

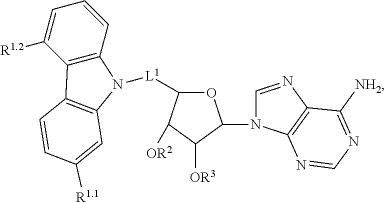

wherein R², R³, L¹, R^{1.1}, and R^{1.2} are as described herein, including embodiments.

In embodiments, the compound has the formula:

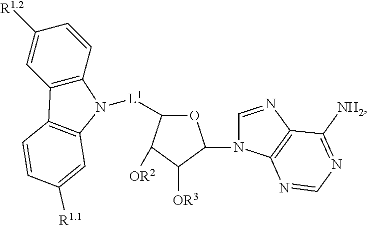

wherein R², R³, L¹, R^{1.1}, and R^{1.2} are as described herein, including embodiments.

In embodiments, the compound has the formula:

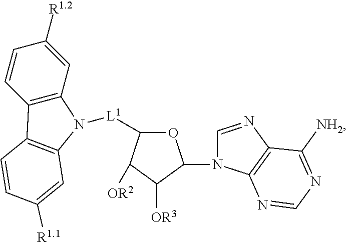

wherein R², R³, L¹, R^{1.1}, and R^{1.2} are as described herein, including embodiments.

In embodiments, the compound has the formula:

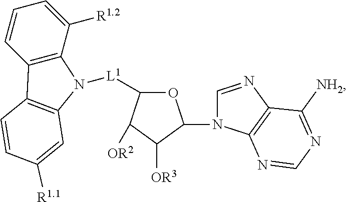

wherein R², R³, L¹, R^{1.1}, and R^{1.2} are as described herein, including embodiments.

In embodiments, the compound has the formula:

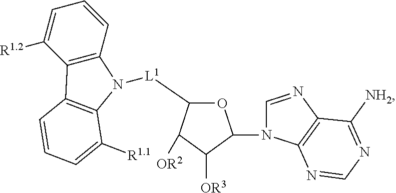

wherein R², R³, L¹, R^{1.1}, and R^{1.2} are as described herein, including embodiments.

In embodiments, the compound has the formula:

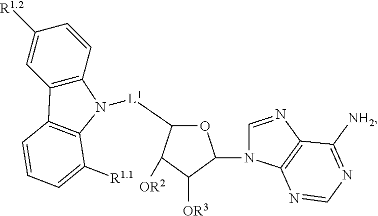

wherein R², R³, L¹, R^{1.1}, and R^{1.2} are as described herein, including embodiments.

In embodiments, the compound has the formula:

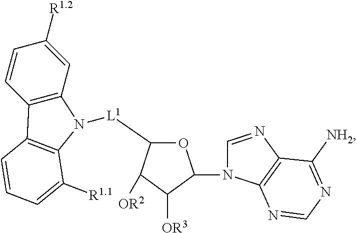

wherein R², R³, L¹, R^{1.1}, and R^{1.2} are as described herein, including embodiments.

In embodiments, the compound has the formula:

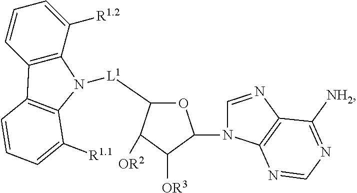

wherein R², R³, L¹, R^{1.1}, and R^{1.2} are as described herein, including embodiments.

In embodiments, the compound has the formula:

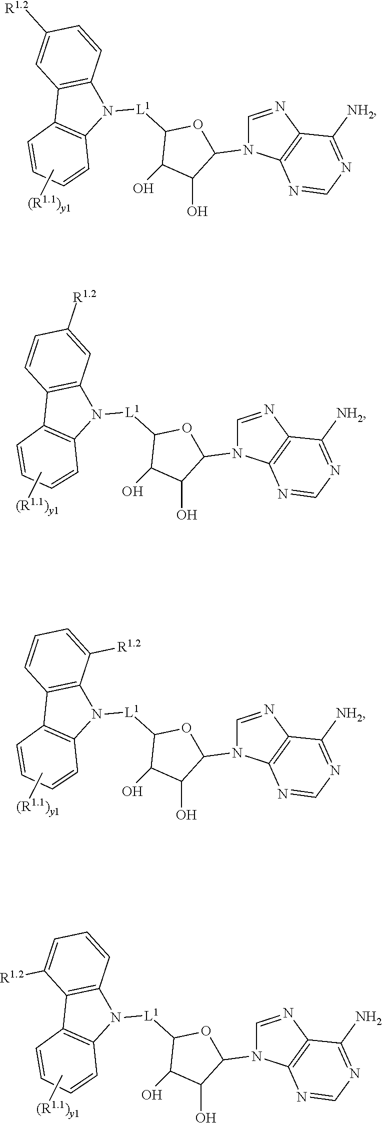

wherein R^{1.1}, y1, R^{1.2}, and L¹ are as described herein, including embodiments.

In embodiments, the compound has the formula:

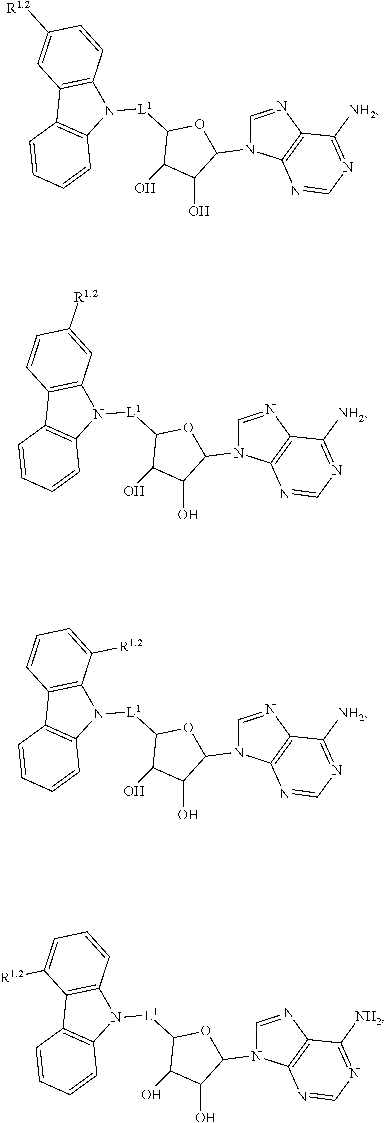

wherein R^{1.2} and L¹ are as described herein, including embodiments.

In embodiments, the compound has the formula:

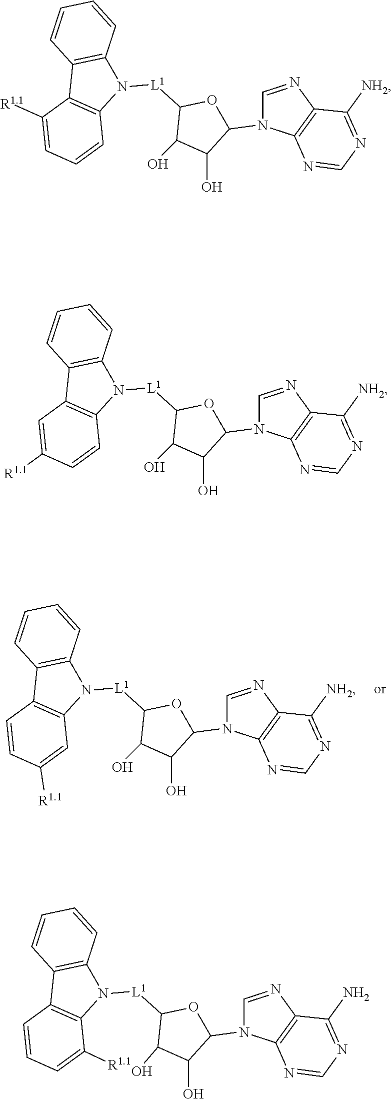

wherein R^{1.1} and L¹ are as described herein, including embodiments.

In embodiments, the compound has the formula:

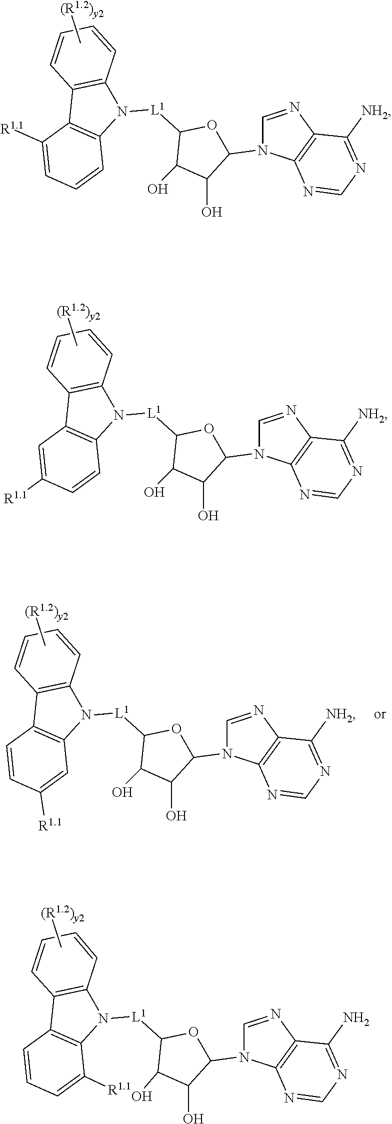

wherein R^{1.1}, y2, R^{1.2}, and L¹ are as described herein, including embodiments.

In embodiments, the compound has the formula:

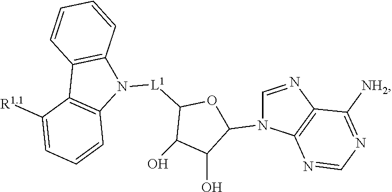

wherein L¹ and R^{1.1} are as described herein, including embodiments.

In embodiments, the compound has the formula:

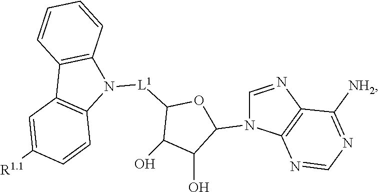

wherein L¹ and R^{1.1} are as described herein, including embodiments.

In embodiments, the compound has the formula:

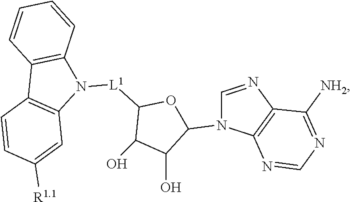

wherein L¹ and R^{1.1} are as described herein, including embodiments.

In embodiments, the compound has the formula:

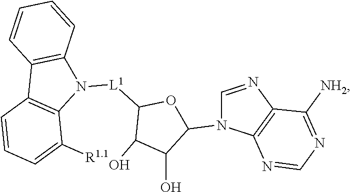

wherein L¹ and R^{1.1} are as described herein, including embodiments.

In embodiments, the compound has the formula:

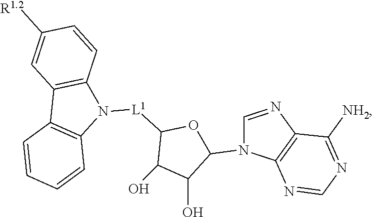

wherein L¹ and R^{1.2} are as described herein, including embodiments.

In embodiments, the compound has the formula:

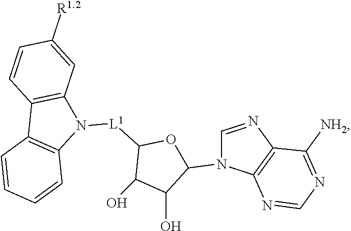

wherein L¹ and R^{1.2} are as described herein, including embodiments.

In embodiments, the compound has the formula:

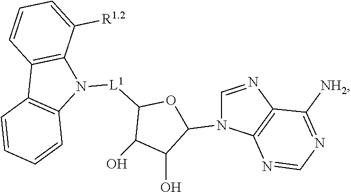

wherein L¹ and R^{1.2} are as described herein, including embodiments.

In embodiments, the compound has the formula:

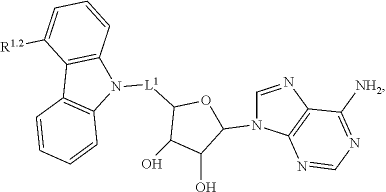

wherein L¹ and R^{1.2} are as described herein, including embodiments.

In embodiments, the compound has the formula:

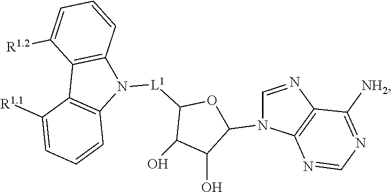

wherein L¹ R^{1.1}, and R^{1.2} are as described herein, including embodiments.

In embodiments, the compound has the formula:

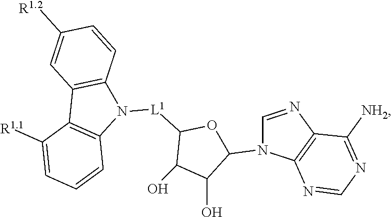

wherein L¹, R^{1.1}, and R^{1.2} are as described herein, including embodiments.

In embodiments, the compound has the formula:

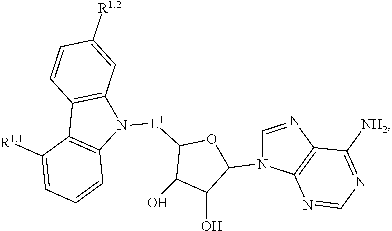

wherein L¹, R^{1.1}, and R^{1.2} are as described herein, including embodiments.

In embodiments, the compound has the formula:

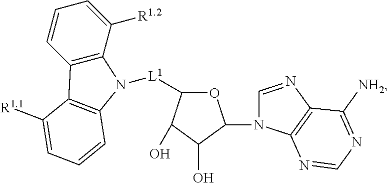

wherein L¹, R^{1.1}, and R^{1.2} are as described herein, including embodiments.

In embodiments, the compound has the formula:

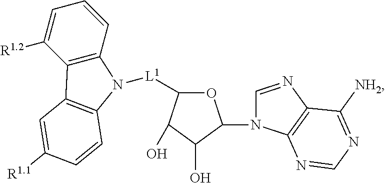

wherein L¹, R^{1.1}, and R^{1.2} are as described herein, including embodiments.

In embodiments, the compound has the formula:

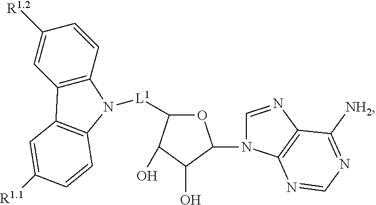

wherein L¹, R^{1.1}, and R^{1.2} are as described herein, including embodiments.

In embodiments, the compound has the formula:

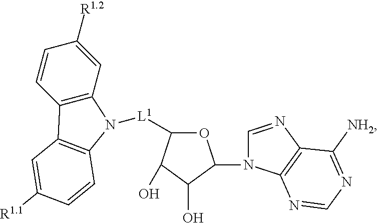

wherein L¹, R^{1.1}, and R^{1.2} are as described herein, including embodiments.

In embodiments, the compound has the formula:

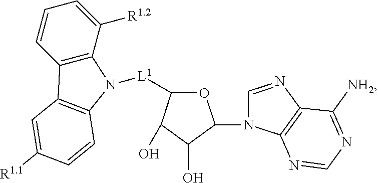

wherein L¹, R^{1.1}, and R^{1.2} are as described herein, including embodiments.

In embodiments, the compound has the formula:

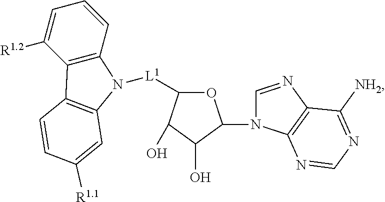

wherein L¹, R^{1.1}, and R^{1.2} are as described herein, including embodiments.

In embodiments, the compound has the formula:

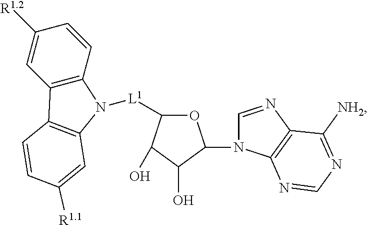

wherein L¹, R^{1.1}, and R^{1.2} are as described herein, including embodiments.

In embodiments, the compound has the formula:

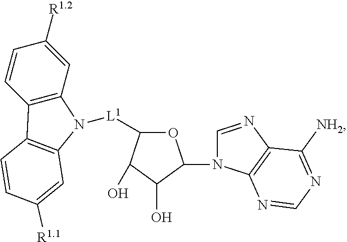

wherein L¹, R^{1.1}, and R^{1.2} are as described herein, including embodiments.

In embodiments, the compound has the formula:

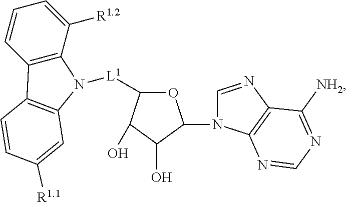

wherein L¹, R^{1.1}, and R^{1.2} are as described herein, including embodiments.

In embodiments, the compound has the formula:

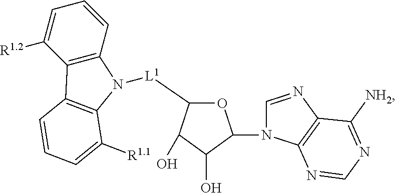

wherein L¹, R^{1.1}, and R^{1.2} are as described herein, including embodiments.

In embodiments, the compound has the formula:

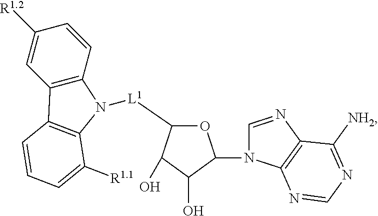

wherein L¹, R^{1.1}, and R^{1.2} are as described herein, including embodiments.

In embodiments, the compound has the formula:

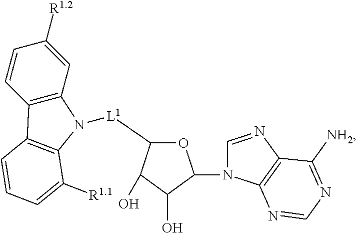

wherein L¹, R^{1.1}, and R^{1.2} are as described herein, including embodiments.

In embodiments, the compound has the formula:

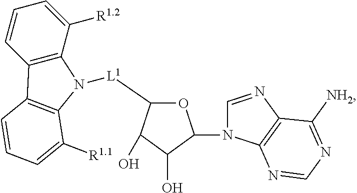

wherein L¹, R^{1.1}, and R^{1.2} are as described herein, including embodiments.

In an aspect is provided a vaccine formulation, wherein the vaccine composition includes an immunogenic agent, an adenosine N-6 methylation agonist or an adenosine N-6 demethylation antagonist, and an adjuvant (e.g., a vaccine adjuvant).

In embodiments, the compounds disclosed herein, including the compounds of Formula I, II, III, IV, or V and embodiments thereof, are capable of boosting a subject's immune response to an RNA virus infection (e.g., Zika infection), relative to the absence of the RNA 2′-O-methyl transferase inhibitors disclosed herein.

In embodiments, the compound has the formula:

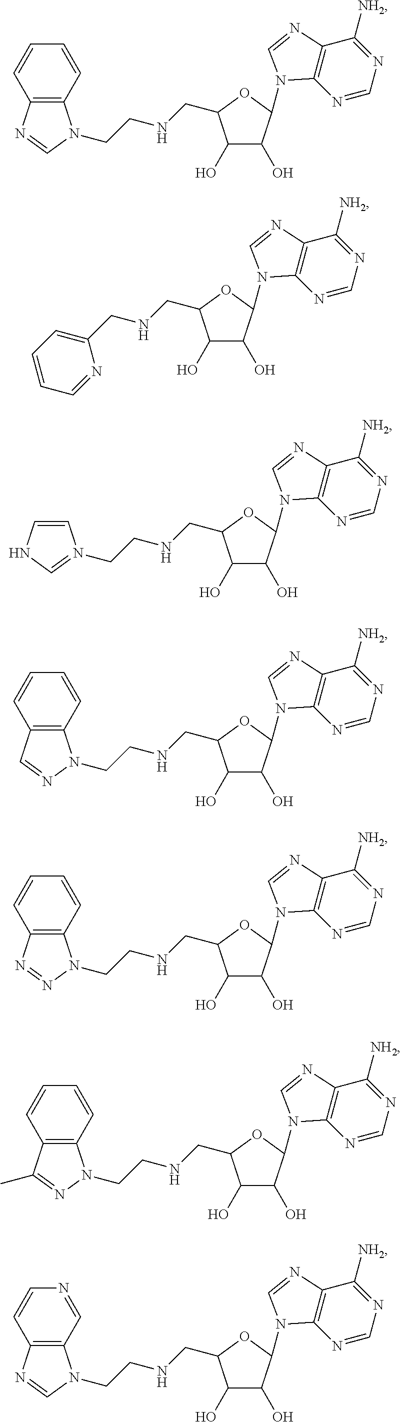

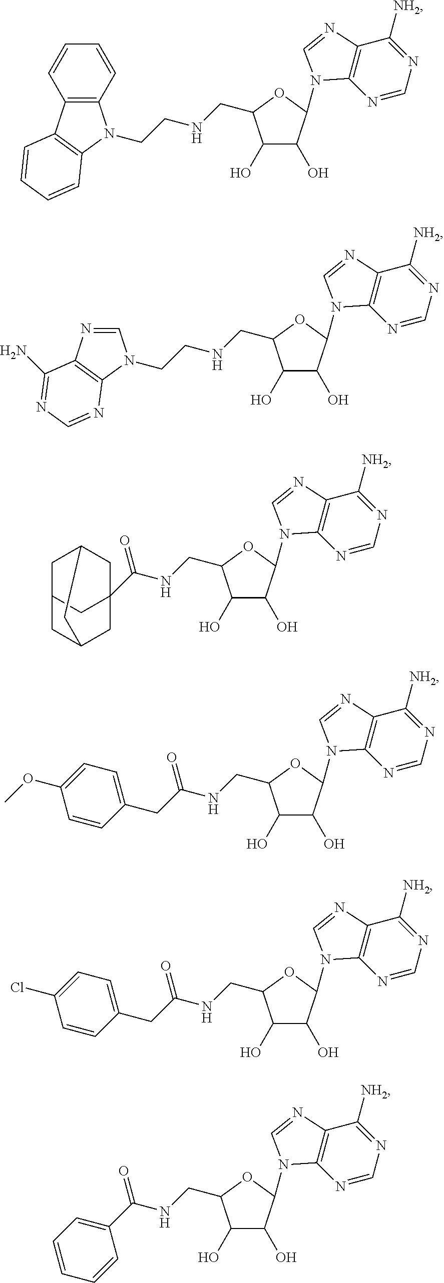 [SMILES: Nc1ncnc2c1ncn2C1OC(CNCCn2c3ccccc3c3ccccc32)C(O)C1O ; Nc1ncnc2c1ncn2CCNCC1OC(n2cnc3c(N)ncnc32)C(O)C1O ; Nc1ncnc2c1ncn2C1OC(CNC(=O)C23CC4CC(CC(C4)C2)C3)C(O)C1O ; COc1ccc(CC(=O)NCC2OC(n3cnc4c(N)ncnc43)C(O)C2O)cc1 ; Nc1ncnc2c1ncn2C1OC(CNC(=O)Cc2ccc(Cl)cc2)C(O)C1O ; Nc1ncnc2c1ncn2C1OC(CNC(=O)c2ccccc2)C(O)C1O]

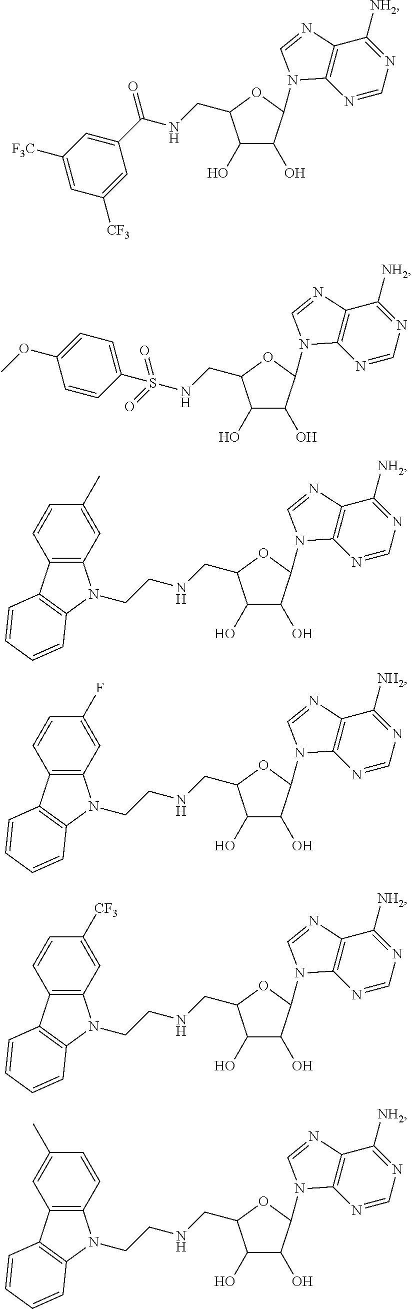

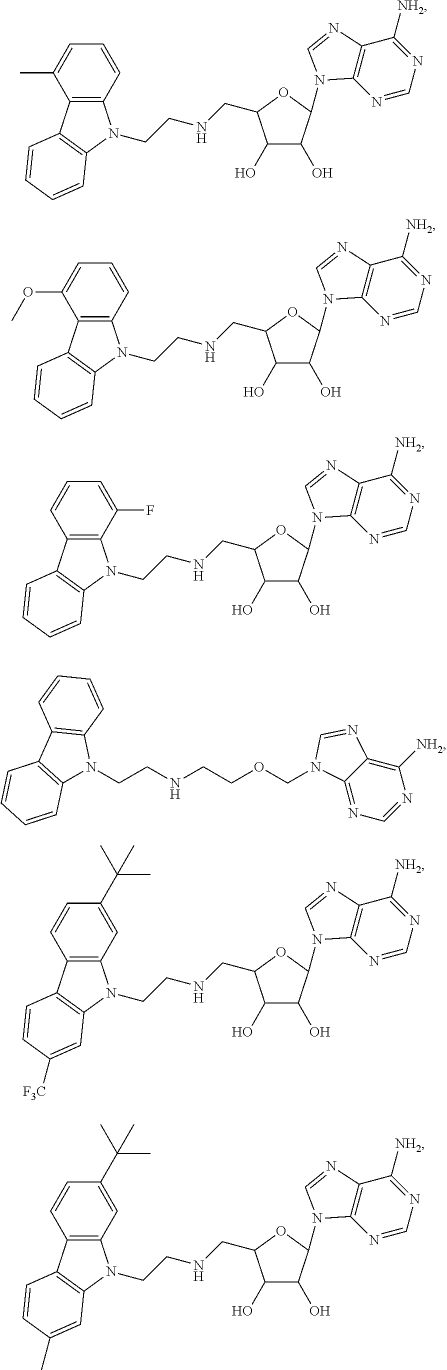

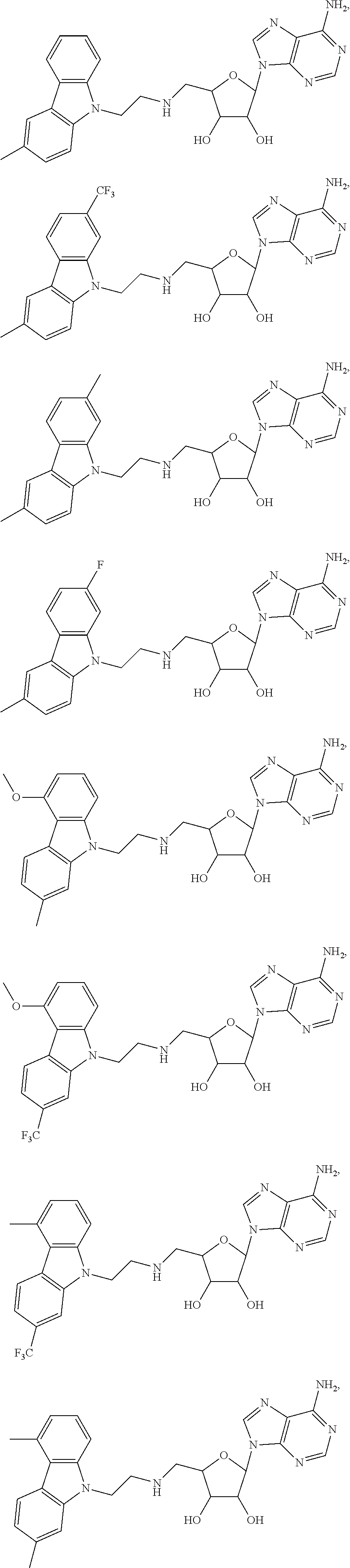

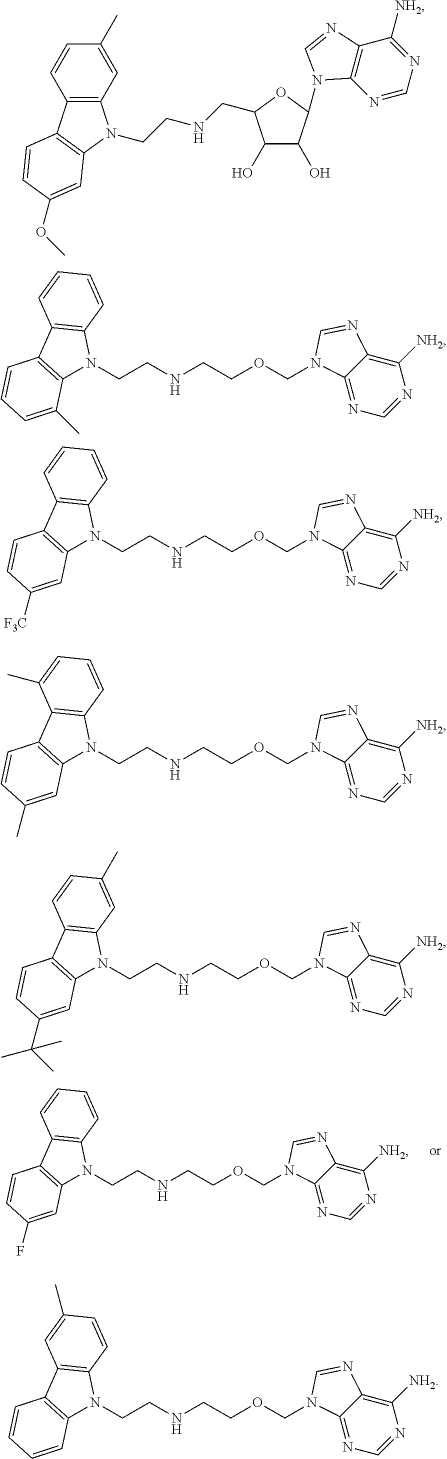

In embodiments, z1 and z2 are independently integers from 0 to 5. In embodiments, z1 and z2 are independently integers from 0 to 3. In embodiments, z1 and z2 are 1. In embodiments, z1 is an integer from 0 to 3, and z2 is 0. In embodiments, z1 is 1 and z2 is 0. In embodiments, z2 is an integer from 0 to 3, and z1 is 0. In embodiments, z1 is 0, and z2 is 1. In embodiments, y1 and y2 are 0. In embodiments, y1 and y2 are 1.

In embodiments, y1 is 0 and y2 is 1. In embodiments, y1 is 1 and y2 is 0. In embodiments, y1 and y2 are 1. In embodiments, y1 is 1, y2 is 0, and R^{1.1} is —CH₃ or —CH₂F. In embodiments, y1 is 0, y2 is 1, and R^{1.2} is —CH₂F, —CF₃, —CH₃, —OCH₃ or —C(CH₃)₃.

In embodiments y3 is 0. In embodiments y3 is 1. In embodiments y3 is 2. In embodiments y3 is 3. In embodiments y3 is 4. In embodiments y3 is 5. In embodiments y3 is 6. In embodiments y3 is 7. In embodiments y3 is 8.

In embodiments, the compound is not S-adenosylhomocysteien or sinefungin.

In some embodiments, the compound is any one of the compounds described herein (e.g., in an aspect, embodiment, claim, figure, table, or example). In embodiments, the compound is not a compound described herein (e.g., in an aspect, embodiment, claim, figure, table, or example).

### III. Pharmaceutical Compositions

In another aspect, there is provided a pharmaceutical composition including a agent (e.g., a compound described herein, nucleic acid, antibody) and a pharmaceutically acceptable excipient. In embodiments, there is provided a pharmaceutical composition including a compound (e.g., a compound described herein) and a pharmaceutically acceptable excipient.

In another aspect, there is provided a pharmaceutical composition including an RNA compound, the RNA compound encoding an RNA virus structural protein. In embodiments, the RNA compound is a nucleic acid. In embodiments, the RNA compound is a nucleic acid sequence disclosed herein (e.g., in Table 5). In embodiments, the RNA virus structural protein is a Zika virus structural protein. In embodiments, the RNA virus structural protein is a viral premembrane protein (prM), viral envelope protein (Env), a capsid protein (C) or a membrane protein (M). The term “Zika virus structural protein” or the like refer, in the usual and customary sense, to structural proteins encoded within the Zika virus genome. See e.g., Abbink, P., et al., Science 2016, 353:1129-1132, which is incorporated herein in its entirety for all purposes.

In embodiments, the RNA compound is modified on at least one 2′ position with a 2′O-methyl and/or modified on at least one adenosine at the N6 adenine position with a methyl functionality. It is understood that the terms “m6A,” “adenosine N-6 methylation” or the like refer to methylation at the N-6 position of adenosine.

### IV. Methods of Use

In an aspect is provided a method of treating or preventing a Zika viral infection in a subject in need thereof, the method including administering an effective amount of an adenosine N-6 methylation agonist or an adenosine N-6 demethylation antagonist. In embodiments, the method includes administering a combination (e.g., two or more) of adenosine N-6 methylation agonists (e.g., RNA methyltransferase complex agonist, a N6-adenosine-methyltransferase (METTL3) agonist, or a Methyltransferase-Like Protein 14 (METTL14) agonist, or a DNA sequence described herein or the RNA sequence corresponding to the DNA nucleic acid sequence described herein). In embodiments, the method includes administering a combination (e.g., two or more) of adenosine N-6 demethylation antagonists (e.g., adenosine N-6 demethylation antagonist is an alkB homolog 5 RNA demethylase (ALKBH5) antagonist or an alpha-ketoglutarate-dependent dioxygenase (FTO) antagonist, or a DNA sequence described herein or the RNA sequence corresponding to the DNA nucleic acid sequence described herein).

In embodiments, the adenosine N-6 methylation agonist is a RNA methyltransferase complex agonist, a N6-adenosine-methyltransferase (METTL3) agonist, or a Methyltransferase-Like Protein 14 (METTL14) agonist. In embodiments, the adenosine N-6 methylation agonist binds to METTL3 and modulates the activity of METTL3 or the signaling pathway of METTL3 (e.g., increases, relative to the absence of the agonist, the overall number of adenosine N-6 methylation events). In embodiments, the adenosine N-6 methylation agonist contacts METTL3 and modulates the activity of METTL3 (e.g., increases, relative to the absence of the agonist, the overall number of adenosine N-6 methylation events). In embodiments, the adenosine N-6 methylation agonist binds to METTL14 and modulates the activity of METTL14 or the signaling pathway of METTL14 (e.g., increases, relative to the absence of the agonist, the overall number of adenosine N-6 methylation events). In embodiments, the adenosine N-6 methylation agonist contacts METTL14 and modulates the activity of METTL14 (e.g., increases, relative to the absence of the agonist, the overall number of adenosine N-6 methylation events).

In embodiments, the adenosine N-6 methylation agonist is a RNA methyltransferase complex (e.g., both METTL3 and METT14) agonist. In embodiments, the adenosine N-6 methylation agonist is a N6-adenosine-methyltransferase (METTL3) agonist. In embodiments, the adenosine N-6 methylation agonist is a Methyltransferase-Like Protein 14 (METTL14) agonist. In embodiments, the adenosine N-6 methylation agonist is a METTL3 or a METTL14 agonist. In embodiments, the adenosine N-6 methylation agonist is an adenosine N-6 methylation antisense nucleic acid agonist. In embodiments, the N-6 methylation antisense nucleic acid agonist is an N-6 methylation RNAi agonist. In embodiments, the adenosine N-6 methylation agonist is an adenosine N-6 methylation aptamer agonist or an adenosine N-6 methylation antibody agonist. In embodiments, the adenosine N-6 methylation agonist is an adenosine N-6 methylation aptamer agonist or an adenosine N-6 methylation antibody agonist. In embodiments, the adenosine N-6 methylation agonist is an adenosine N-6 methylation aptamer agonist. In embodiments, the adenosine N-6 methylation agonist is an adenosine N-6 methylation antibody agonist. In embodiments, the adenosine N-6 methylation agonist is a sequence described herein.

In embodiments, the adenosine N-6 demethylation antagonist is an alkB homolog 5 RNA demethylase (ALKBH5) antagonist or an alpha-ketoglutarate-dependent dioxygenase (FTO) antagonist. In embodiments, the adenosine N-6 demethylation antagonist is an alkB homolog 5 RNA demethylase (ALKBH5) antagonist. In embodiments, the adenosine N-6 demethylation antagonist is an alpha-ketoglutarate-dependent dioxygenase (FTO) antagonist.

In embodiments, the adenosine N-6 demethylation antagonist binds to ALKBH5 and modulates the function or activity of ALKBH5 (e.g., decreases, relative to the absence of the antagonist, the overall number of adenosine N-6 demethylation events). In embodiments, the adenosine N-6 demethylation antagonist contacts ALKBH5 and modulates the function or activity of ALKBH5 (e.g., decreases, relative to the absence of the antagonist, the overall number of adenosine N-6 demethylation events). In embodiments, ALKBH5 is human ALKBH5. In embodiments, ALKBH5 is not viral ALKBH5. In embodiments, the adenosine N-6 demethylation antagonist is meclofenamic acid, cassic acid (i.e. Rhein), citrate, pyridine-2,4-dicarboxylate (PDCA), N-oxalylglycine, or succinate.

In embodiments, the adenosine N-6 demethylation antagonist binds to FTO and modulates the function or activity of FTO (e.g., decreases, relative to the absence of the antagonist, the overall number of adenosine N-6 demethylation events). In embodiments, the adenosine N-6 demethylation antagonist contacts FTO and modulates the function or activity of FTO (e.g., decreases, relative to the absence of the antagonist, the overall number of adenosine N-6 demethylation events). In embodiments, the FTO is human FTO. In embodiments, the FTO is not viral FTO. In embodiments, the adenosine N-6 demethylation antagonist is meclofenamic acid, N-oxalyglycine, cassic acid (i.e. Rhein), or 4-chloro-6-(6′-chloro-7′-hydroxy-2′,4′,4′-trimethyl-chroman-2′-yl)benzene-1,3-diol (CHTB).

In embodiments, the adenosine N-6 demethylation antagonist is an adenosine N-6 demethylation antisense nucleic acid antagonist. In embodiments, the N-6 demethylation antisense nucleic acid antagonist is an adenosine N-6 demethylation RNAi antagonist. In embodiments, the adenosine N-6 demethylation antagonist is an adenosine N-6 demethylation RNAi antagonist. In embodiments, the adenosine N-6 demethylation antagonist is an adenosine N-6 demethylation aptamer antagonist or an adenosine N-6 demethylation antibody antagonist. In embodiments, the adenosine N-6 demethylation antagonist is an adenosine N-6 demethylation aptamer antagonist. In embodiments, the adenosine N-6 demethylation antagonist is an adenosine N-6 demethylation antibody antagonist. In embodiments, the adenosine N-6 demethylation antagonist is an adenosine N-6 demethylation CRISPR antagonist (e.g., a nucleic acid sequence described herein capable of antagonizing adenosine N-6 demethylation). In embodiments, the adenosine N-6 demethylation antagonist decreases, relative to the absence of the antagonist, the number of demethylation events (i.e. removing a —CH₃ moiety to the 6-position of adenosine), or:

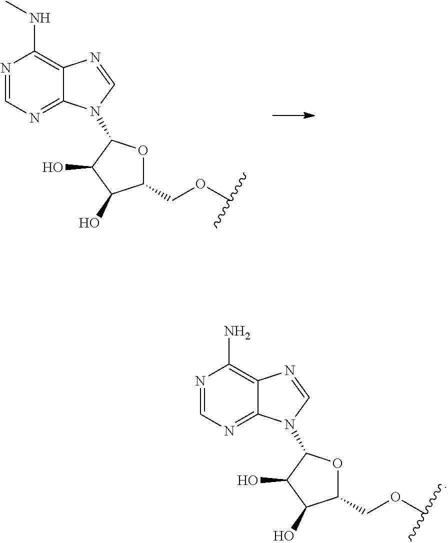 [SMILES: *OC[C@H]1O[C@@H](n2cnc3c(NC)ncnc32)[C@H](O)[C@@H]1O ; *OC[C@H]1O[C@@H](n2cnc3c(N)ncnc32)[C@H](O)[C@@H]1O]

In embodiments, the adenosine N-6 demethylation antagonist is an adenosine N-6 demethylation CRISPR antagonist (e.g., a nucleic acid sequence described herein capable of antagonizing adenosine N-6 demethylation). In embodiments, the adenosine N-6 demethylation antagonist is a sequence described herein.

In embodiments, the adenosine N-6 methylation agonist is an adenosine N-6 methylation antisense nucleic acid agonist and the adenosine N-6 demethylation antagonist is an adenosine N-6 demethylation antisense nucleic acid antagonist. In embodiments, the N-6 methylation antisense nucleic acid agonist is an N-6 methylation RNAi agonist and said adenosine N-6 demethylation antisense nucleic acid antagonist is an adenosine N-6 demethylation RNAi antagonist. In embodiments, the adenosine N-6 methylation agonist increases relative to the absence of the agonist the overall density of adenosine N-6 methylation events (i.e. adding a —CH₃ moiety to the 6-position of adenosine), or:

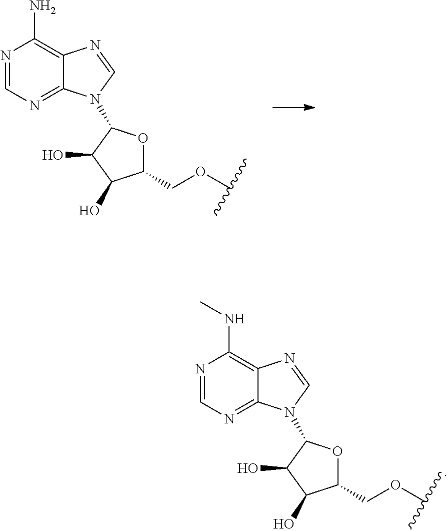 [SMILES: *OC[C@H]1O[C@@H](n2cnc3c(N)ncnc32)[C@H](O)[C@@H]1O ; *OC[C@H]1O[C@@H](n2cnc3c(NC)ncnc32)[C@H](O)[C@@H]1O]

In embodiments, the method further includes administering an immunogenic agent (e.g., a dsRNA virus).

In embodiments, the immunogenic agent and the adenosine N-6 methylation agonist or the adenosine N-6 demethylation antagonist together with a vaccine adjuvant form a vaccine formulation.

In embodiments, the adenosine N-6 methylation agonist or the adenosine N-6 demethylation antagonist are capable of activating the immune system of the subject (e.g., a subject in need thereof).

In an aspect is provided a method of treating or preventing a Zika viral infection in a subject in need thereof, the method including administering an effective amount of an RNA 2′-O-methyl transferase inhibitor (e.g., an inhibitor of viral 2′-O-methyl transferase). In embodiments the method is treating a Zika viral infection in a subject in need thereof. In embodiments, the method is preventing a Zika viral infection in a subject in need thereof. In embodiments, treating does not include preventing.

In embodiments, the RNA 2′-O-methyl transferase inhibitor is an RNA 2′-O-methyl transferase antisense inhibitor, an RNA 2′-O-methyl transferase CRISPR inhibitor (e.g., a nucleic acid sequence described herein capable of inhibiting RNA 2′-O-methyl transferase), an RNA 2′-O-methyl aptamer inhibitor, or an RNA 2′-O-methyl transferase antibody inhibitor. In embodiments, the RNA 2′-O-methyl transferase inhibitor is an RNA 2′-O-methyl transferase antisense inhibitor. In embodiments, the RNA 2′-O-methyl transferase inhibitor is an RNA 2′-O-methyl transferase CRISPR inhibitor (e.g., a nucleic acid sequence, or the RNA sequence corresponding to a DNA nucleic acid sequence described herein (i.e., wherein all instances of thymine are replaced with uracil), capable of inhibiting RNA 2′-O-methyl transferase). In embodiments, the RNA 2′-O-methyl transferase inhibitor is an RNA 2′-O-methyl aptamer inhibitor. In embodiments, the RNA 2′-O-methyl transferase inhibitor is an RNA 2′-O-methyl transferase antibody inhibitor (e.g., e.g., an antibody capable of inhibiting RNA 2′-O-methyl transferase. In embodiments, the RNA 2′-O-methyl transferase inhibitor is a nonstructural protein 5 (NS5) inhibitor (e.g., an NS5 inhibitor which is capable of inhibiting RNA 2′-O-methyl transferase or capable of inhibiting the methylation of a 2′—OH of one or more of an adenosine, guanosine, uracil or cytosine in viral RNA). In embodiments, the RNA 2′-O-methyl transferase inhibitor is S-adenosylmethionine, a chemical analogue of S-adenosylmethionine, or a small molecule functional analogue of S-adenosylmethionine. In embodiments, the RNA 2′-O-methyl transferase inhibitor is S-adenosylmethionine. In embodiments, the RNA 2′-O-methyl transferase inhibitor is a chemical analogue of S-adenosylmethionine. In embodiments, the RNA 2′-O-methyl transferase inhibitor is a small molecule (e.g., less than 900 Da) functional analogue of S-adenosylmethionine.

In embodiments, the RNA 2′-O-methyl transferase inhibitor is capable of inhibiting the methylation of a 2′—OH of one or more of an adenosine, guanosine, uracil or cytosine in viral RNA. In embodiments, the RNA 2′-O-methyl transferase inhibitor is capable of inhibiting the methylation of a 2′—OH of an adenosine, guanosine, uracil or cytosine in a viral RNA. In embodiments, the RNA 2′-O-methyl transferase inhibitor is capable of inhibiting the methylation of a 2′—OH of one or more of an adenosine, guanosine, uracil or cytosine in a human RNA. In embodiments, the RNA 2′-O-methyl transferase inhibitor is capable of inhibiting the methylation of a 2′—OH of an adenosine, guanosine, uracil or cytosine in a human RNA. In embodiments, the RNA 2′-O-methyl transferase inhibitor is capable of inhibiting the methylation of a 2′—OH the Cap nucleotide (e.g., 2′—OH methylation on the terminating nucleotide, for example:

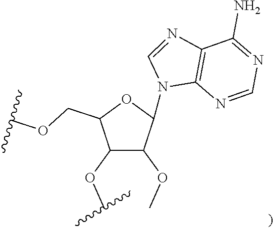 [SMILES: *OCC1OC(n2cnc3c(N)ncnc32)C(OC)C1O*]

of viral RNA. In embodiments, the RNA 2′-O-methyl transferase inhibitor (e.g., a compound described herein) binds to viral RNA 2′-O-methyl transferase and inhibits the activity of the RNA 2′-O-methyl transferase relative to the absence of the inhibitor, (e.g., decreases methylation of a 2′—OH of one or more of an adenosine, guanosine, uracil or cytosine in viral RNA). In embodiments, RNA 2′-O-methyl transferase inhibitor contacts the RNA 2′-O-methyl transferase (e.g., viral RNA 2′-O-methyl transferase) and modulates the activity of the RNA 2′-O-methyl transferase (e.g., decreases, relative to the absence of the inhibitor, the overall number of methylation events at the 2′—OH of one or more of an adenosine, guanosine, uracil or cytosine in viral RNA). In embodiments, the RNA 2′-O-methyl transferase (e.g., viral RNA 2′-O-methyl transferase) is inhibited or contacted by a compound described herein. In embodiments, the RNA 2′-O-methyl transferase is viral RNA 2′-O-methyl transferase. In embodiments, the RNA 2′-O-methyl transferase is not human RNA 2′-O-methyl transferase.

In embodiments, the RNA 2′-O-methyl transferase is a Flavivirus RNA 2′-O-methyl transferase. In embodiments, the RNA 2′-O-methyl transferase is Absettarov RNA 2′-O-methyl transferase. In embodiments, the RNA 2′-O-methyl transferase is Alkhurma RNA 2′-O-methyl transferase (ALKV). In embodiments, the RNA 2′-O-methyl transferase is Deer tick RNA 2′-O-methyl transferase (DT). In embodiments, the RNA 2′-O-methyl transferase is Gadgets Gully RNA 2′-O-methyl transferase (GGYV). In embodiments, the RNA 2′-O-methyl transferase is Kadam RNA 2′-O-methyl transferase (KADV). In embodiments, the RNA 2′-O-methyl transferase is Karshi RNA 2′-O-methyl transferase. In embodiments, the RNA 2′-O-methyl transferase is Kyasanur Forest disease RNA 2′-O-methyl transferase (KFDV). In embodiments, the RNA 2′-O-methyl transferase is Langat RNA 2′-O-methyl transferase (LGTV). In embodiments, the RNA 2′-O-methyl transferase is Louping ill RNA 2′-O-methyl transferase (LIV). In embodiments, the RNA 2′-O-methyl transferase is Mogiana tick RNA 2′-O-methyl transferase (MGTV). In embodiments, the RNA 2′-O-methyl transferase is Ngoye RNA 2′-O-methyl transferase (NGOV). In embodiments, the RNA 2′-O-methyl transferase is Omsk hemorrhagic fever RNA 2′-O-methyl transferase (OHFV). In embodiments, the RNA 2′-O-methyl transferase is Powassan RNA 2′-O-methyl transferase (POWV). In embodiments, the RNA 2′-O-methyl transferase is Royal Farm RNA 2′-O-methyl transferase (RFV). In embodiments, the RNA 2′-O-methyl transferase is Sokuluk RNA 2′-O-methyl transferase (SOKV). In embodiments, the RNA 2′-O-methyl transferase is Tick-borne encephalitis RNA 2′-O-methyl transferase (TBEV). In embodiments, the RNA 2′-O-methyl transferase is Turkish sheep encephalitis RNA 2′-O-methyl transferase (TSE). In embodiments, the RNA 2′-O-methyl transferase is Kama RNA 2′-O-methyl transferase (KAMV). In embodiments, the RNA 2′-O-methyl transferase is Meaban RNA 2′-O-methyl transferase (MEAV). In embodiments, the RNA 2′-O-methyl transferase is Saumarez Reef RNA 2′-O-methyl transferase (SREV). In embodiments, the RNA 2′-O-methyl transferase is Tyuleniy RNA 2′-O-methyl transferase (TYUV). In embodiments, the RNA 2′-O-methyl transferase is Aedes flaviRNA 2′-O-methyl transferase. In embodiments, the RNA 2′-O-methyl transferase is Barkedji RNA 2′-O-methyl transferase. In embodiments, the RNA 2′-O-methyl transferase is Calbertado RNA 2′-O-methyl transferase. In embodiments, the RNA 2′-O-methyl transferase is Cell fusing agent RNA 2′-O-methyl transferase. In embodiments, the RNA 2′-O-methyl transferase is Chaoyang RNA 2′-O-methyl transferase. In embodiments, the RNA 2′-O-methyl transferase is Culex flaviRNA 2′-O-methyl transferase. In embodiments, the RNA 2′-O-methyl transferase is Culex theileri flaviRNA 2′-O-methyl transferase. In embodiments, the RNA 2′-O-methyl transferase is Culiseta flaviRNA 2′-O-methyl transferase. In embodiments, the RNA 2′-O-methyl transferase is Donggang RNA 2′-O-methyl transferase. In embodiments, the RNA 2′-O-methyl transferase is Hanko RNA 2′-O-methyl transferase. In embodiments, the RNA 2′-O-methyl transferase is Ilomantsi RNA 2′-O-methyl transferase. In embodiments, the RNA 2′-O-methyl transferase is Kamiti River RNA 2′-O-methyl transferase. In embodiments, the RNA 2′-O-methyl transferase is Lammi RNA 2′-O-methyl transferase. In embodiments, the RNA 2′-O-methyl transferase is Marisma mosquito RNA 2′-O-methyl transferase. In embodiments, the RNA 2′-O-methyl transferase is Nakiwogo RNA 2′-O-methyl transferase. In embodiments, the RNA 2′-O-methyl transferase is Nounané RNA 2′-O-methyl transferase. In embodiments, the RNA 2′-O-methyl transferase is Nhumirim RNA 2′-O-methyl transferase. In embodiments, the RNA 2′-O-methyl transferase is Nienokoue RNA 2′-O-methyl transferase. In embodiments, the RNA 2′-O-methyl transferase is Palm Creek RNA 2′-O-methyl transferase (PCV). In embodiments, the RNA 2′-O-methyl transferase is Spanish Culex flaviRNA 2′-O-methyl transferase. In embodiments, the RNA 2′-O-methyl transferase is Spanish Ochlerotatus flaviRNA 2′-O-methyl transferase. In embodiments, the RNA 2′-O-methyl transferase is Quang Binh RNA 2′-O-methyl transferase. In embodiments, the RNA 2′-O-methyl transferase is Xishuangbanna flaviRNA 2′-O-methyl transferase. In embodiments, the RNA 2′-O-methyl transferase is Aroa RNA 2′-O-methyl transferase (AROAV). In embodiments, the RNA 2′-O-methyl transferase is Bussuquara RNA 2′-O-methyl transferase (BSQV). In embodiments, the RNA 2′-O-methyl transferase is Iguape RNA 2′-O-methyl transferase (IGUV). In embodiments, the RNA 2′-O-methyl transferase is Dengue RNA 2′-O-methyl transferase (DENV). In embodiments, the RNA 2′-O-methyl transferase is Kedougou RNA 2′-O-methyl transferase (KEDV). In embodiments, the RNA 2′-O-methyl transferase is Bussuquara RNA 2′-O-methyl transferase. In embodiments, the RNA 2′-O-methyl transferase is Cacipacore RNA 2′-O-methyl transferase (CPCV). In embodiments, the RNA 2′-O-methyl transferase is Koutango RNA 2′-O-methyl transferase (KOUV). In embodiments, the RNA 2′-O-methyl transferase is Ilheus RNA 2′-O-methyl transferase (ILHV). In embodiments, the RNA 2′-O-methyl transferase is Japanese encephalitis RNA 2′-O-methyl transferase (JEV). In embodiments, the RNA 2′-O-methyl transferase is Murray Valley encephalitis RNA 2′-O-methyl transferase (MVEV). In embodiments, the RNA 2′-O-methyl transferase is Alfuy RNA 2′-O-methyl transferase. In embodiments, the RNA 2′-O-methyl transferase is Rocio RNA 2′-O-methyl transferase (ROCV). In embodiments, the RNA 2′-O-methyl transferase is St. Louis encephalitis RNA 2′-O-methyl transferase (SLEV). In embodiments, the RNA 2′-O-methyl transferase is Usutu RNA 2′-O-methyl transferase (USUV). In embodiments, the RNA 2′-O-methyl transferase is West Nile RNA 2′-O-methyl transferase (WNV). In embodiments, the RNA 2′-O-methyl transferase is Yaounde RNA 2′-O-methyl transferase (YAOV). In embodiments, the RNA 2′-O-methyl transferase is Kokobera RNA 2′-O-methyl transferase (KOKV). In embodiments, the RNA 2′-O-methyl transferase is New Mapoon RNA 2′-O-methyl transferase (NMV). In embodiments, the RNA 2′-O-methyl transferase is Stratford RNA 2′-O-methyl transferase (STRV). In embodiments, the RNA 2′-O-methyl transferase is Bagaza RNA 2′-O-methyl transferase (BAGV). In embodiments, the RNA 2′-O-methyl transferase is Baiyangdian RNA 2′-O-methyl transferase (BYDV). In embodiments, the RNA 2′-O-methyl transferase is Duck egg drop syndrome RNA 2′-O-methyl transferase (BYDV). In embodiments, the RNA 2′-O-methyl transferase is Ilheus RNA 2′-O-methyl transferase (ILHV). In embodiments, the RNA 2′-O-methyl transferase is Jiangsu RNA 2′-O-methyl transferase (JSV). In embodiments, the RNA 2′-O-methyl transferase is Israel turkey meningoencephalomyelitis RNA 2′-O-methyl transferase (ITV). In embodiments, the RNA 2′-O-methyl transferase is Ntaya RNA 2′-O-methyl transferase (NTAV). In embodiments, the RNA 2′-O-methyl transferase is Tembusu RNA 2′-O-methyl transferase (TMUV). In embodiments, the RNA 2′-O-methyl transferase is Spondweni RNA 2′-O-methyl transferase (SPOV). In embodiments, the RNA 2′-O-methyl transferase is Zika RNA 2′-O-methyl transferase (ZIKV). In embodiments, the RNA 2′-O-methyl transferase is Banzi RNA 2′-O-methyl transferase (BANV). In embodiments, the RNA 2′-O-methyl transferase is Bamaga RNA 2′-O-methyl transferase (BGV). In embodiments, the RNA 2′-O-methyl transferase is Bouboui RNA 2′-O-methyl transferase (BOUV). In embodiments, the RNA 2′-O-methyl transferase is Edge Hill RNA 2′-O-methyl transferase (EHV). In embodiments, the RNA 2′-O-methyl transferase is Jugra RNA 2′-O-methyl transferase (JUGV). In embodiments, the RNA 2′-O-methyl transferase is Saboya RNA 2′-O-methyl transferase (SABV). In embodiments, the RNA 2′-O-methyl transferase is Sepik RNA 2′-O-methyl transferase (SEPV). In embodiments, the RNA 2′-O-methyl transferase is Uganda S RNA 2′-O-methyl transferase (UGSV). In embodiments, the RNA 2′-O-methyl transferase is Wesselsbron RNA 2′-O-methyl transferase (WESSV). In embodiments, the RNA 2′-O-methyl transferase is Yellow fever RNA 2′-O-methyl transferase (YFV). In embodiments, the RNA 2′-O-methyl transferase is Tamana bat RNA 2′-O-methyl transferase (TABV). In embodiments, the RNA 2′-O-methyl transferase is Entebbe bat RNA 2′-O-methyl transferase (ENTV). In embodiments, the RNA 2′-O-methyl transferase is Sokoluk RNA 2′-O-methyl transferase. In embodiments, the RNA 2′-O-methyl transferase is Yokose RNA 2′-O-methyl transferase (YOKV). In embodiments, the RNA 2′-O-methyl transferase is Apoi RNA 2′-O-methyl transferase (APOIV). In embodiments, the RNA 2′-O-methyl transferase is Cowbone Ridge RNA 2′-O-methyl transferase (CRV). In embodiments, the RNA 2′-O-methyl transferase is Jutiapa RNA 2′-O-methyl transferase (JUTV). In embodiments, the RNA 2′-O-methyl transferase is Modoc RNA 2′-O-methyl transferase (MODV). In embodiments, the RNA 2′-O-methyl transferase is Sal Vieja RNA 2′-O-methyl transferase (SVV). In embodiments, the RNA 2′-O-methyl transferase is San Perlita RNA 2′-O-methyl transferase (SPV). In embodiments, the RNA 2′-O-methyl transferase is Bukalasa bat RNA 2′-O-methyl transferase (BBV). In embodiments, the RNA 2′-O-methyl transferase is Carey Island RNA 2′-O-methyl transferase (CIV). In embodiments, the RNA 2′-O-methyl transferase is Dakar bat RNA 2′-O-methyl transferase (DBV). In embodiments, the RNA 2′-O-methyl transferase is Montana myotis leukoencephalitis RNA 2′-O-methyl transferase (MMLV). In embodiments, the RNA 2′-O-methyl transferase is Phnom Penh bat RNA 2′-O-methyl transferase (PPBV). In embodiments, the RNA 2′-O-methyl transferase is Rio Bravo RNA 2′-O-methyl transferase (RBV). In embodiments, the RNA 2′-O-methyl transferase is Bamaga RNA 2′-O-methyl transferase. In embodiments, the RNA 2′-O-methyl transferase is Hanko RNA 2′-O-methyl transferase. In embodiments, the RNA 2′-O-methyl transferase is Ochlerotatus caspius flaviRNA 2′-O-methyl transferase. In embodiments, the RNA 2′-O-methyl transferase is Palm Creek RNA 2′-O-methyl transferase. In embodiments, the RNA 2′-O-methyl transferase is Parramatta River RNA 2′-O-methyl transferase. In embodiments, the RNA 2′-O-methyl transferase is Soybean cyst nematode RNA 2′-O-methyl transferase 5. In embodiments, the RNA 2′-O-methyl transferase is Aedes flaviRNA 2′-O-methyl transferase. In embodiments, the RNA 2′-O-methyl transferase is Aedes cinereus flaviRNA 2′-O-methyl transferase. In embodiments, the RNA 2′-O-methyl transferase is Aedes vexans flaviRNA 2′-O-methyl transferase. In embodiments, the RNA 2′-O-methyl transferase is Culex theileri flaviRNA 2′-O-methyl transferase. In embodiments, the RNA 2′-O-methyl transferase is or hepatitis C RNA 2′-O-methyl transferase (HCV).

In embodiments, the RNA 2′-O-methyl transferase inhibitor is a compound provided herein, including all embodiments thereof. The compound may be of the formula:

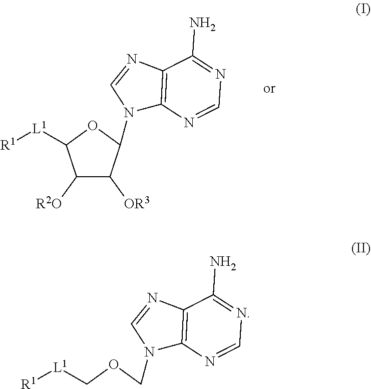

L¹ is a bond, —O—, —S—, —NH—, —C(O)NH—, —C(O)—, —S(O)₂NH—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene. R¹ is hydrogen, oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OR^{1A} (e.g., —OH), —NHR^{1A}(e.g., —NH₂), —COOR^{1A} (e.g., —COOH)—CONR^{1A} (e.g., —CONH₂), —NHC(O)R^{1A} (e.g., —NHC(O)H), —NO₂, —SR^{1A} (e.g., —SH), —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OC H₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl. R^{1A} is hydrogen, halogen, —CF₃, —CN, —OH, —NH₂, —N₃, —SH, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl. R² is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl. R³ is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

In embodiments, the RNA 2′-O-methyl transferase inhibitor is a compound provided herein, including all embodiments thereof. The compound may be of the formula: formula:

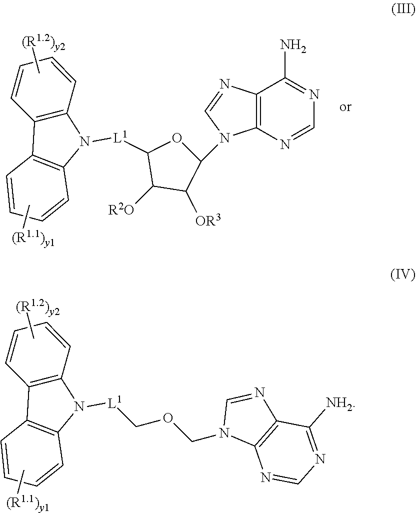

symbols y1 and y2 are independently an integer from 0 to 4. R^{1.1} and R^{1.2} independently are halogen, —CF₃, —CBr₃, —CCl₃, —CI₃, —CHF₂, —CHBr₂, —CHCl₂, —CHI₂, —CH₂F, —CH₂Br, —CH₂Cl, —CH₂ I, —OCF₃, —OCH₃, —OCBr₃, —OCCl₃, —OCI₃, —OCHF₂, —OCHBr₂, —OCHCl₂, —OCHI₂, —OCH₂F, —OC H₂Br, —OCH₂Cl, —OCH₂I, —CN, —OH, —NH₂, —COOH, —CONH₂, —NO₂, —SH, —SO₃H, —SO₄H, —SO₂—NH₂, —NHNH₂, —ONH₂, —NHC(O)NHNH₂, NHC(O)NH₂, —NHSO₂H, —NHC(O)H, —NHC(O)OH, —NHOH, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In an aspect is provided a method of treating or preventing an RNA virus infection in a subject in need thereof, the method including administering an effective amount of a compound provided herein, including all embodiments thereof. The compound may be of the formula:

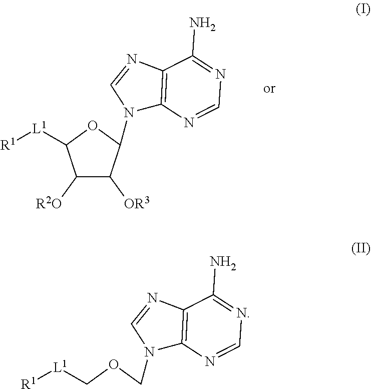

L¹ is a bond, —O—, —S—, —NH—, —C(O)NH—, —C(O)—, —S(O)₂NH—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene. R¹ is hydrogen, oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OR^{1A} (e.g., —OH), —NHR^{1A}(e.g., —NH₂), —COOR^{1A} (e.g., —COOH)—CONR^{1A} (e.g., —CONH₂), —NHC(O)R^{1A} (e.g., —NHC(O)H), —NO₂, —SR^{1A} (e.g., —SH), —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OC H₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl. R^{1A} is hydrogen, halogen, —CF₃, —CN, —OH, —NH₂, —N₃, —SH, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl. R² is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl. R³ is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

In embodiments, the RNA virus infection is an HIV viral infection, West Nile viral infection, Dengue viral infection, Japanese encephalitis or a Zika viral infection. In embodiments, the RNA virus infection is an HIV viral infection. In embodiments, the RNA virus infection is a West Nile viral infection. In embodiments, the RNA virus infection is a Dengue viral infection. In embodiments, the RNA virus infection is Japanese encephalitis. In embodiments, the RNA virus infection is a Zika viral infection.

Further to any method or composition disclosed herein, and embodiment thereof, in embodiments the RNA virus is a flavivirus. In embodiments, the flavivirus is Absettarov virus, Alkhurma virus (ALKV), Deer tick virus (DT), Gadgets Gully virus (GGYV), Kadam virus (KADV), Karshi virus, Kyasanur Forest disease virus (KFDV), Langat virus (LGTV), Louping ill virus (LIV), Mogiana tick virus (MGTV), Ngoye virus (NGOV), Omsk hemorrhagic fever virus (OHFV), Powassan virus (POWV), Royal Farm virus (RFV), Sokuluk virus (SOKV), Tick-borne encephalitis virus (TBEV), Turkish sheep encephalitis virus (TSE), Kama virus (KAMV), Meaban virus (MEAV), Saumarez Reef virus (SREV), Tyuleniy virus (TYUV), Aedes flavivirus, Barkedji virus, Calbertado virus, Cell fusing agent virus, Chaoyang virus, Culex flavivirus, Culex theileri flavivirus, Culiseta flavivirus, Donggang virus, Hanko virus, Ilomantsi virus, Kamiti River virus, Lammi virus, Marisma mosquito virus, Nakiwogo virus, Nounané virus, Nhumirim virus, Nienokoue virus, Palm Creek virus (PCV), Spanish Culex flavivirus, Spanish Ochlerotatus flavivirus, Quang Binh virus, Xishuangbanna flavivirus, Aroa virus (AROAV), Bussuquara virus (BSQV), Iguape virus (IGUV), Dengue virus (DENV), Kedougou virus (KEDV), Bussuquara virus, Cacipacore virus (CPCV), Koutango virus (KOUV), Ilheus virus (ILHV), Japanese encephalitis virus (JEV), Murray Valley encephalitis virus (MVEV), Alfuy virus, Rocio virus (ROCV), St. Louis encephalitis virus (SLEV), Usutu virus (USUV), West Nile virus (WNV), Yaounde virus (YAOV), Kokobera virus (KOKV), New Mapoon virus (NMV), Stratford virus (STRV), Bagaza virus (BAGV), Baiyangdian virus (BYDV), Duck egg drop syndrome virus (BYDV), Ilheus virus (ILHV), Jiangsu virus (JSV), Israel turkey meningoencephalomyelitis virus (ITV), Ntaya virus (NTAV), Tembusu virus (TMUV), Spondweni virus (SPOV), Zika virus (ZIKV), Banzi virus (BANV), Bamaga virus (BGV), Bouboui virus (BOUV), Edge Hill virus (EHV), Jugra virus (JUGV), Saboya virus (SABV), Sepik virus (SEPV), Uganda S virus (UGSV), Wesselsbron virus (WESSV), Yellow fever virus (YFV), Tamana bat virus (TABV), Entebbe bat virus (ENTV), Sokoluk virus, Yokose virus (YOKV), Apoi virus (APOIV), Cowbone Ridge virus (CRV), Jutiapa virus (JUTV), Modoc virus (MODV), Sal Vieja virus (SVV), San Perlita virus (SPV), Bukalasa bat virus (BBV), Carey Island virus (CIV), Dakar bat virus (DBV), Montana myotis leukoencephalitis virus (MMLV), Phnom Penh bat virus (PPBV), Rio Bravo virus (RBV), Bamaga virus, Hanko virus, Ochlerotatus caspius flavivirus, Palm Creek virus, Parramatta River virus, Soybean cyst nematode virus 5, Aedes flavivirus, Aedes cinereus flavivirus, Aedes vexans flavivirus, Culex theileri flavivirus, or hepatitis C virus (HCV).

In embodiments, the RNA virus is Absettarov virus, Alkhurma virus (ALKV), Deer tick virus (DT), Gadgets Gully virus (GGYV), Kadam virus (KADV), Karshi virus, Kyasanur Forest disease virus (KFDV), Langat virus (LGTV), Louping ill virus (LIV), Mogiana tick virus (MGTV), Ngoye virus (NGOV), Omsk hemorrhagic fever virus (OHFV), Powassan virus (POWV), Royal Farm virus (RFV), Sokuluk virus (SOKV), Tick-borne encephalitis virus (TBEV), Turkish sheep encephalitis virus (TSE), Kama virus (KAMV), Meaban virus (MEAV), Saumarez Reef virus (SREV), Tyuleniy virus (TYUV), Aedes flavivirus, Barkedji virus, Calbertado virus, Cell fusing agent virus, Chaoyang virus, Culex flavivirus, Culex theileri flavivirus, Culiseta flavivirus, Donggang virus, Hanko virus, Ilomantsi virus, Kamiti River virus, Lammi virus, Marisma mosquito virus, Nakiwogo virus, Nounané virus, Nhumirim virus, Nienokoue virus, Palm Creek virus (PCV), Spanish Culex flavivirus, Spanish Ochlerotatus flavivirus, Quang Binh virus, Xishuangbanna flavivirus, Aroa virus (AROAV), Bussuquara virus (BSQV), Iguape virus (IGUV), Dengue virus (DENV), Kedougou virus (KEDV), Bussuquara virus, Cacipacore virus (CPCV), Koutango virus (KOUV), Ilheus virus (ILHV), Japanese encephalitis virus (JEV), Murray Valley encephalitis virus (MVEV), Alfuy virus, Rocio virus (ROCV), St. Louis encephalitis virus (SLEV), Usutu virus (USUV), West Nile virus (WNV), Yaounde virus (YAOV), Kokobera virus (KOKV), New Mapoon virus (NMV), Stratford virus (STRV), Bagaza virus (BAGV), Baiyangdian virus (BYDV), Duck egg drop syndrome virus (BYDV), Ilheus virus (ILHV), Jiangsu virus (JSV), Israel turkey meningoencephalomyelitis virus (ITV), Ntaya virus (NTAV), Tembusu virus (TMUV), Spondweni virus (SPOV), Zika virus (ZIKV), Banzi virus (BANV), Bamaga virus (BGV), Bouboui virus (BOUV), Edge Hill virus (EHV), Jugra virus (JUGV), Saboya virus (SABV), Sepik virus (SEPV), Uganda S virus (UGSV), Wesselsbron virus (WESSV), Yellow fever virus (YFV), Tamana bat virus (TABV), Entebbe bat virus (ENTV), Sokoluk virus, Yokose virus (YOKV), Apoi virus (APOIV), Cowbone Ridge virus (CRV), Jutiapa virus (JUTV), Modoc virus (MODV), Sal Vieja virus (SVV), San Perlita virus (SPV), Bukalasa bat virus (BBV), Carey Island virus (CIV), Dakar bat virus (DBV), Montana myotis leukoencephalitis virus (MMLV), Phnom Penh bat virus (PPBV), Rio Bravo virus (RBV), Bamaga virus, Hanko virus, Ochlerotatus caspius flavivirus, Palm Creek virus, Parramatta River virus, Soybean cyst nematode virus 5, Aedes flavivirus, Aedes cinereus flavivirus, Aedes vexans flavivirus, Culex theileri flavivirus, or hepatitis C virus (HCV).

In embodiments, the RNA virus is Absettarov virus. In embodiments, the RNA virus is Alkhurma virus (ALKV). In embodiments, the RNA virus is Deer tick virus (DT). In embodiments, the RNA virus is Gadgets Gully virus (GGYV). In embodiments, the RNA virus is Kadam virus (KADV). In embodiments, the RNA virus is Karshi virus. In embodiments, the RNA virus is Kyasanur Forest disease virus (KFDV). In embodiments, the RNA virus is Langat virus (LGTV). In embodiments, the RNA virus is Louping ill virus (LIV). In embodiments, the RNA virus is Mogiana tick virus (MGTV). In embodiments, the RNA virus is Ngoye virus (NGOV). In embodiments, the RNA virus is Omsk hemorrhagic fever virus (OHFV). In embodiments, the RNA virus is Powassan virus (POWV). In embodiments, the RNA virus is Royal Farm virus (RFV). In embodiments, the RNA virus is Sokuluk virus (SOKV). In embodiments, the RNA virus is Tick-borne encephalitis virus (TBEV). In embodiments, the RNA virus is Turkish sheep encephalitis virus (TSE). In embodiments, the RNA virus is Kama virus (KAMV). In embodiments, the RNA virus is Meaban virus (MEAV). In embodiments, the RNA virus is Saumarez Reef virus (SREV). In embodiments, the RNA virus is Tyuleniy virus (TYUV). In embodiments, the RNA virus is Aedes flavivirus. In embodiments, the RNA virus is Barkedji virus. In embodiments, the RNA virus is Calbertado virus. In embodiments, the RNA virus is Cell fusing agent virus. In embodiments, the RNA virus is Chaoyang virus. In embodiments, the RNA virus is Culex flavivirus. In embodiments, the RNA virus is Culex theileri flavivirus. In embodiments, the RNA virus is Culiseta flavivirus. In embodiments, the RNA virus is Donggang virus. In embodiments, the RNA virus is Hanko virus. In embodiments, the RNA virus is Ilomantsi virus. In embodiments, the RNA virus is Kamiti River virus. In embodiments, the RNA virus is Lammi virus. In embodiments, the RNA virus is Marisma mosquito virus. In embodiments, the RNA virus is Nakiwogo virus. In embodiments, the RNA virus is Nounané virus. In embodiments, the RNA virus is Nhumirim virus. In embodiments, the RNA virus is Nienokoue virus. In embodiments, the RNA virus is Palm Creek virus (PCV). In embodiments, the RNA virus is Spanish Culex flavivirus. In embodiments, the RNA virus is Spanish Ochlerotatus flavivirus. In embodiments, the RNA virus is Quang Binh virus. In embodiments, the RNA virus is Xishuangbanna flavivirus. In embodiments, the RNA virus is Aroa virus (AROAV). In embodiments, the RNA virus is Bussuquara virus (BSQV). In embodiments, the RNA virus is Iguape virus (IGUV). In embodiments, the RNA virus is Dengue virus (DENV). In embodiments, the RNA virus is Kedougou virus (KEDV). In embodiments, the RNA virus is Bussuquara virus. In embodiments, the RNA virus is Cacipacore virus (CPCV). In embodiments, the RNA virus is Koutango virus (KOUV). In embodiments, the RNA virus is Ilheus virus (ILHV). In embodiments, the RNA virus is Japanese encephalitis virus (JEV). In embodiments, the RNA virus is Murray Valley encephalitis virus (MVEV). In embodiments, the RNA virus is Alfuy virus. In embodiments, the RNA virus is Rocio virus (ROCV). In embodiments, the RNA virus is St. Louis encephalitis virus (SLEV). In embodiments, the RNA virus is Usutu virus (USUV). In embodiments, the RNA virus is West Nile virus (WNV). In embodiments, the RNA virus is Yaounde virus (YAOV). In embodiments, the RNA virus is Kokobera virus (KOKV). In embodiments, the RNA virus is New Mapoon virus (NMV). In embodiments, the RNA virus is Stratford virus (STRV). In embodiments, the RNA virus is Bagaza virus (BAGV). In embodiments, the RNA virus is Baiyangdian virus (BYDV). In embodiments, the RNA virus is Duck egg drop syndrome virus (BYDV). In embodiments, the RNA virus is Ilheus virus (ILHV). In embodiments, the RNA virus is Jiangsu virus (JSV). In embodiments, the RNA virus is Israel turkey meningoencephalomyelitis virus (ITV). In embodiments, the RNA virus is Ntaya virus (NTAV). In embodiments, the RNA virus is Tembusu virus (TMUV). In embodiments, the RNA virus is Spondweni virus (SPOV). In embodiments, the RNA virus is Zika virus (ZIKV). In embodiments, the RNA virus is Banzi virus (BANV). In embodiments, the RNA virus is Bamaga virus (BGV). In embodiments, the RNA virus is Bouboui virus (BOUV). In embodiments, the RNA virus is Edge Hill virus (EHV). In embodiments, the RNA virus is Jugra virus (JUGV). In embodiments, the RNA virus is Saboya virus (SABV). In embodiments, the RNA virus is Sepik virus (SEPV). In embodiments, the RNA virus is Uganda S virus (UGSV). In embodiments, the RNA virus is Wesselsbron virus (WESSV). In embodiments, the RNA virus is Yellow fever virus (YFV). In embodiments, the RNA virus is Tamana bat virus (TABV). In embodiments, the RNA virus is Entebbe bat virus (ENTV). In embodiments, the RNA virus is Sokoluk virus. In embodiments, the RNA virus is Yokose virus (YOKV). In embodiments, the RNA virus is Apoi virus (APOIV). In embodiments, the RNA virus is Cowbone Ridge virus (CRV). In embodiments, the RNA virus is Jutiapa virus (JUTV). In embodiments, the RNA virus is Modoc virus (MODV). In embodiments, the RNA virus is Sal Vieja virus (SVV). In embodiments, the RNA virus is San Perlita virus (SPV). In embodiments, the RNA virus is Bukalasa bat virus (BBV). In embodiments, the RNA virus is Carey Island virus (CIV). In embodiments, the RNA virus is Dakar bat virus (DBV). In embodiments, the RNA virus is Montana myotis leukoencephalitis virus (MMLV). In embodiments, the RNA virus is Phnom Penh bat virus (PPBV). In embodiments, the RNA virus is Rio Bravo virus (RBV). In embodiments, the RNA virus is Bamaga virus. In embodiments, the RNA virus is Hanko virus. In embodiments, the RNA virus is Ochlerotatus caspius flavivirus. In embodiments, the RNA virus is Palm Creek virus. In embodiments, the RNA virus is Parramatta River virus. In embodiments, the RNA virus is Soybean cyst nematode virus 5. In embodiments, the RNA virus is Aedes flavivirus. In embodiments, the RNA virus is Aedes cinereus flavivirus. In embodiments, the RNA virus is Aedes vexans flavivirus. In embodiments, the RNA virus is Culex theileri flavivirus. In embodiments, the RNA virus is or hepatitis C virus (HCV).

In embodiments, the RNA virus is of the Order Nidovirales (e.g., Family Arteriviridae, Family Coronaviridae (e.g., Coronavirus or SARS), Family Mesoniviridae, Family Roniviridae). In embodiments, the RNA virus is of the Order Picornavirales, Family Dicistroviridae, Family Iflaviridae, Family Marnaviridae, Family Picornaviridae (e.g., Poliovirus, Rhinovirus (a common cold virus), Hepatitis A virus), Family Secoviridae includes subfamily Comovirinae, Genus Bacillariornavirus, Genus Dicipivirus, Genus Labymavirus, Genus Sequiviridae, or Species Kelp fly virus. In embodiments, the RNA virus is of the Order Tymovirales, Family Alphaflexiviridae, Family Betaflexiviridae, Family Gammaflexiviridae, or Family Tymoviridae.

In embodiments, the RNA virus is of the Family Alphatetraviridae, Family Alvernaviridae, Family Astroviridae, Family Barnaviridae, Family Benyviridae, Family Bromoviridae, Family Caliciviridae (e.g., Norwalk virus), Family Carmotetraviridae, Family Closteroviridae, Family Flaviviridae (e.g., Yellow fever virus, West Nile virus, Hepatitis C virus, Dengue fever virus, or Zika virus), Family Fusariviridae, Family Hepeviridae, Family Leviviridae, Family Luteoviridae (e.g., Barley yellow dwarf virus), Family Polycipiviridae, Family Narnaviridae, Family Nodaviridae, Family Permutotetraviridae, Family Potyviridae, Family Statovirus, Family Togaviridae (e.g., Rubella virus, Ross River virus, Sindbis virus, or Chikungunya virus), Family Tombusviridae, or Family Virgaviridae.

In embodiments, the RNA virus is of the Genus Blunervirus, Genus Cilevirus, Genus Higrevirus, Genus Idaeovirus, Genus Negevirus, Genus Ourmiavirus, Genus Polemovirus, Genus Sinaivirus, or Genus Sobemovirus.

In embodiments, the RNA virus is Acyrthosiphon pisum virus, Bastrovirus, Blackford virus, Blueberry necrotic ring blotch virus, Chara australis virus, Extra small virus, Jingmen tick virus, Le Blanc virus, Nesidiocoris tenuis virus 1, Nylanderia fulva virus 1, Orsay virus, Plasmopara halstedii virus, Rosellinia necatrix fusarivirus 1, Santeuil virus, or Solenopsis invicta virus 3.

In embodiments, the method of treatment (e.g., method of treating an RNA viral infection or Zika virus infection) includes administering a compound (e.g., a compound described herein, including embodiments) disclosed in herein (e.g., in a Table, Example, claim, or embodiment).

In embodiments, the adenosine N-6 methylation CRISPR agonist, adenosine N-6 demethylation CRISPR antagonist, or RNA 2′-O-methyl transferase CRISPR inhibitor is a sequence described herein, including embodiments.

In embodiments, the adenosine N-6 methylation CRISPR agonist, adenosine N-6 demethylation CRISPR antagonist, or RNA 2′-O-methyl transferase CRISPR inhibitor is SEQ ID NO:1, or the RNA sequence corresponding to SEQ ID NO:1 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:2, or the RNA sequence corresponding to SEQ ID NO:2 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:3, or the RNA sequence corresponding to SEQ ID NO:3 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:4, or the RNA sequence corresponding to SEQ ID NO:4 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:5, or the RNA sequence corresponding to SEQ ID NO:5 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:6, or the RNA sequence corresponding to SEQ ID NO:6 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:7, or the RNA sequence corresponding to SEQ ID NO:7 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:8, or the RNA sequence corresponding to SEQ ID NO:8 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:9, or the RNA sequence corresponding to SEQ ID NO:9 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO: 10, or the RNA sequence corresponding to SEQ ID NO: 10 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO: 11, or the RNA sequence corresponding to SEQ ID NO:11 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:12, or the RNA sequence corresponding to SEQ ID NO: 12 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO: 13, or the RNA sequence corresponding to SEQ ID NO: 13 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO: 14, or the RNA sequence corresponding to SEQ ID NO: 14 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO: 15, or the RNA sequence corresponding to SEQ ID NO: 15 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:16, or the RNA sequence corresponding to SEQ ID NO: 16 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO: 17, or the RNA sequence corresponding to SEQ ID NO: 17 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:18, or the RNA sequence corresponding to SEQ ID NO:18 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO: 19, or the RNA sequence corresponding to SEQ ID NO: 19 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:20, or the RNA sequence corresponding to SEQ ID NO:20 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:21, or the RNA sequence corresponding to SEQ ID NO:21 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:22, or the RNA sequence corresponding to SEQ ID NO:22 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:23, or the RNA sequence corresponding to SEQ ID NO:23 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:24, or the RNA sequence corresponding to SEQ ID NO:24 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:25, or the RNA sequence corresponding to SEQ ID NO:25 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:26, or the DNA sequence corresponding to SEQ ID NO:26 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:27, or the DNA sequence corresponding to SEQ ID NO:27 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:28, or the DNA sequence corresponding to SEQ ID NO:28 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:29, or the DNA sequence corresponding to SEQ ID NO:29 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:30, or the DNA sequence corresponding to SEQ ID NO:30 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:31, or the DNA sequence corresponding to SEQ ID NO:31 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:32, or the DNA sequence corresponding to SEQ ID NO:32 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:33, or the DNA sequence corresponding to SEQ ID NO:33 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:34, or the DNA sequence corresponding to SEQ ID NO:34 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:35, or the DNA sequence corresponding to SEQ ID NO:35 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:36, or the RNA sequence corresponding to SEQ ID NO:36 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:37, or the RNA sequence corresponding to SEQ ID NO:37 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:38, or the RNA sequence corresponding to SEQ ID NO:38 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:39, or the RNA sequence corresponding to SEQ ID NO:39 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:40, or the RNA sequence corresponding to SEQ ID NO:40 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:41, or the RNA sequence corresponding to SEQ ID NO:41 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:42, or the RNA sequence corresponding to SEQ ID NO:42 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:43, or the RNA sequence corresponding to SEQ ID NO:43 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:44, or the RNA sequence corresponding to SEQ ID NO:44 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:45, or the RNA sequence corresponding to SEQ ID NO:45 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:46, or the RNA sequence corresponding to SEQ ID NO:46 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:47, or the RNA sequence corresponding to SEQ ID NO:47 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:48, or the RNA sequence corresponding to SEQ ID NO:48 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:49, or the RNA sequence corresponding to SEQ ID NO:49 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:50, or the RNA sequence corresponding to SEQ ID NO:50 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:51, or the RNA sequence corresponding to SEQ ID NO:51 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:52, or the RNA sequence corresponding to SEQ ID NO:52 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:53, or the RNA sequence corresponding to SEQ ID NO:53 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:54, or the RNA sequence corresponding to SEQ ID NO:54 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:55, or the RNA sequence corresponding to SEQ ID NO:55 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:56, or the RNA sequence corresponding to SEQ ID NO:56 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:57, or the RNA sequence corresponding to SEQ ID NO:57 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:58, or the RNA sequence corresponding to SEQ ID NO:58 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:59, or the RNA sequence corresponding to SEQ ID NO:59 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:60, or the RNA sequence corresponding to SEQ ID NO:60 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:61, or the RNA sequence corresponding to SEQ ID NO:61 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:62, or the RNA sequence corresponding to SEQ ID NO:62 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:63, or the RNA sequence corresponding to SEQ ID NO:63 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:64, or the RNA sequence corresponding to SEQ ID NO:64 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:65, or the RNA sequence corresponding to SEQ ID NO:65 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:66, or the RNA sequence corresponding to SEQ ID NO:66 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:67, or the RNA sequence corresponding to SEQ ID NO:67 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:68, or the RNA sequence corresponding to SEQ ID NO:68 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:69, or the RNA sequence corresponding to SEQ ID NO:69 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:70, or the RNA sequence corresponding to SEQ ID NO:70 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:71, or the RNA sequence corresponding to SEQ ID NO:71 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:72, or the RNA sequence corresponding to SEQ ID NO:72 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:73, or the RNA sequence corresponding to SEQ ID NO:73 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:74, or the RNA sequence corresponding to SEQ ID NO:74 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:75, or the RNA sequence corresponding to SEQ ID NO:75 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:76, or the RNA sequence corresponding to SEQ ID NO:76 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:77, or the RNA sequence corresponding to SEQ ID NO:77 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:78, or the RNA sequence corresponding to SEQ ID NO:78 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:79, or the RNA sequence corresponding to SEQ ID NO:79 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:80, or the RNA sequence corresponding to SEQ ID NO:80 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:81, or the RNA sequence corresponding to SEQ ID NO:81 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:82, or the RNA sequence corresponding to SEQ ID NO:82 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO: 83, or the RNA sequence corresponding to SEQ ID NO:83 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:84, or the RNA sequence corresponding to SEQ ID NO:84 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:85, or the RNA sequence corresponding to SEQ ID NO:85 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:86, or the RNA sequence corresponding to SEQ ID NO:86 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO: 87, or the RNA sequence corresponding to SEQ ID NO:87 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:88, or the RNA sequence corresponding to SEQ ID NO:88 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:89, or the RNA sequence corresponding to SEQ ID NO:89 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:90, or the RNA sequence corresponding to SEQ ID NO:90 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:91, or the RNA sequence corresponding to SEQ ID NO:91 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:92, or the RNA sequence corresponding to SEQ ID NO:92 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:93, or the RNA sequence corresponding to SEQ ID NO:93 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:94, or the RNA sequence corresponding to SEQ ID NO:94 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:95, or the RNA sequence corresponding to SEQ ID NO:95 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:96, or the RNA sequence corresponding to SEQ ID NO:96 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:97, or the RNA sequence corresponding to SEQ ID NO:97 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:98, or the RNA sequence corresponding to SEQ ID NO:98 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:99, or the RNA sequence corresponding to SEQ ID NO:99 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:100, or the RNA sequence corresponding to SEQ ID NO: 100 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:101, or the RNA sequence corresponding to SEQ ID NO:101 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:102, or the RNA sequence corresponding to SEQ ID NO:102 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO: 103, or the RNA sequence corresponding to SEQ ID NO: 103 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:104, or the RNA sequence corresponding to SEQ ID NO: 104 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:105, or the RNA sequence corresponding to SEQ ID NO:105 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO: 106, or the RNA sequence corresponding to SEQ ID NO: 106 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:107, or the RNA sequence corresponding to SEQ ID NO: 107 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:108, or the RNA sequence corresponding to SEQ ID NO:108 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO: 109, or the RNA sequence corresponding to SEQ ID NO: 109 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO: 110, or the RNA sequence corresponding to SEQ ID NO:110 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:111, or the RNA sequence corresponding to SEQ ID NO:111 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO: 112, or the RNA sequence corresponding to SEQ ID NO: 112 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO: 113, or the RNA sequence corresponding to SEQ ID NO: 113 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:114, or the RNA sequence corresponding to SEQ ID NO:114 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO: 115, or the RNA sequence corresponding to SEQ ID NO:115 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO: 116, or the RNA sequence corresponding to SEQ ID NO: 116 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:117, or the RNA sequence corresponding to SEQ ID NO:117 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO: 118, or the RNA sequence corresponding to SEQ ID NO:118 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO: 119, or the RNA sequence corresponding to SEQ ID NO: 119 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:120, or the RNA sequence corresponding to SEQ ID NO:120 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO: 121, or the RNA sequence corresponding to SEQ ID NO:121 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:122, or the RNA sequence corresponding to SEQ ID NO: 122 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:123, or the RNA sequence corresponding to SEQ ID NO:123 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO: 124, or the RNA sequence corresponding to SEQ ID NO: 124 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:125, or the RNA sequence corresponding to SEQ ID NO: 125 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:126, or the RNA sequence corresponding to SEQ ID NO:126 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO: 127, or the RNA sequence corresponding to SEQ ID NO: 127 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:128, or the RNA sequence corresponding to SEQ ID NO: 128 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:129, or the RNA sequence corresponding to SEQ ID NO:129 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO: 130, or the RNA sequence corresponding to SEQ ID NO: 130 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:131, or the RNA sequence corresponding to SEQ ID NO:131 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:132, or the RNA sequence corresponding to SEQ ID NO:132 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO: 133, or the RNA sequence corresponding to SEQ ID NO:133 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:134, or the RNA sequence corresponding to SEQ ID NO: 134 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:135, or the RNA sequence corresponding to SEQ ID NO:135 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO: 136, or the RNA sequence corresponding to SEQ ID NO:136 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:137, or the RNA sequence corresponding to SEQ ID NO: 137 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:138, or the RNA sequence corresponding to SEQ ID NO:138 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO: 139, or the RNA sequence corresponding to SEQ ID NO: 139 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:140, or the RNA sequence corresponding to SEQ ID NO: 140 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:141, or the RNA sequence corresponding to SEQ ID NO:141 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO: 142, or the RNA sequence corresponding to SEQ ID NO: 142 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:143, or the RNA sequence corresponding to SEQ ID NO: 143 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:144, or the RNA sequence corresponding to SEQ ID NO:144 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO: 145, or the RNA sequence corresponding to SEQ ID NO: 145 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:146, or the RNA sequence corresponding to SEQ ID NO: 146 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:147, or the RNA sequence corresponding to SEQ ID NO:147 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO: 148, or the RNA sequence corresponding to SEQ ID NO: 148 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:149, or the RNA sequence corresponding to SEQ ID NO: 149 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:150, or the RNA sequence corresponding to SEQ ID NO:150 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO: 151, or the RNA sequence corresponding to SEQ ID NO:151 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:152, or the RNA sequence corresponding to SEQ ID NO: 152 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:153, or the RNA sequence corresponding to SEQ ID NO:153 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO: 154, or the RNA sequence corresponding to SEQ ID NO: 154 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:155, or the RNA sequence corresponding to SEQ ID NO:155 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:156, or the RNA sequence corresponding to SEQ ID NO:156 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO: 157, or the RNA sequence corresponding to SEQ ID NO: 157 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:158, or the RNA sequence corresponding to SEQ ID NO:158 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:159, or the RNA sequence corresponding to SEQ ID NO:159 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO: 160, or the RNA sequence corresponding to SEQ ID NO: 160 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:161, or the RNA sequence corresponding to SEQ ID NO: 161 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:162, or the RNA sequence corresponding to SEQ ID NO:162 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO: 163, or the RNA sequence corresponding to SEQ ID NO: 163 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:164, or the RNA sequence corresponding to SEQ ID NO: 164 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:165, or the RNA sequence corresponding to SEQ ID NO:165 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO: 166, or the RNA sequence corresponding to SEQ ID NO: 166 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:167, or the RNA sequence corresponding to SEQ ID NO: 167 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:168, or the RNA sequence corresponding to SEQ ID NO:168 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO: 169, or the RNA sequence corresponding to SEQ ID NO: 169 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:170, or the RNA sequence corresponding to SEQ ID NO: 170 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:171, or the RNA sequence corresponding to SEQ ID NO:171 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO: 172, or the RNA sequence corresponding to SEQ ID NO: 172 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:173, or the RNA sequence corresponding to SEQ ID NO: 173 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:174, or the RNA sequence corresponding to SEQ ID NO:174 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO: 175, or the RNA sequence corresponding to SEQ ID NO: 175 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:176, or the RNA sequence corresponding to SEQ ID NO: 176 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:177, or the RNA sequence corresponding to SEQ ID NO:177 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO: 178, or the RNA sequence corresponding to SEQ ID NO:178 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:179, or the RNA sequence corresponding to SEQ ID NO: 179 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:180, or the RNA sequence corresponding to SEQ ID NO:180 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO: 181, or the RNA sequence corresponding to SEQ ID NO:181 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:182, or the RNA sequence corresponding to SEQ ID NO: 182 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:183, or the RNA sequence corresponding to SEQ ID NO:183 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO: 184, or the RNA sequence corresponding to SEQ ID NO:184 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:185, or the RNA sequence corresponding to SEQ ID NO: 185 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:186, or the RNA sequence corresponding to SEQ ID NO:186 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO: 187, or the RNA sequence corresponding to SEQ ID NO:187 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:188, or the RNA sequence corresponding to SEQ ID NO:188 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:189, or the RNA sequence corresponding to SEQ ID NO:189 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO: 190, or the RNA sequence corresponding to SEQ ID NO: 190 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:191, or the RNA sequence corresponding to SEQ ID NO: 191 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:192, or the RNA sequence corresponding to SEQ ID NO:192 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO: 193, or the RNA sequence corresponding to SEQ ID NO: 193 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:194, or the RNA sequence corresponding to SEQ ID NO: 194 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:195, or the RNA sequence corresponding to SEQ ID NO:195 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO: 196, or the RNA sequence corresponding to SEQ ID NO: 196 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:197, or the RNA sequence corresponding to SEQ ID NO: 197 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:198, or the RNA sequence corresponding to SEQ ID NO:198 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO: 199, or the RNA sequence corresponding to SEQ ID NO: 199 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:200, or the RNA sequence corresponding to SEQ ID NO:200 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:201, or the RNA sequence corresponding to SEQ ID NO:201 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:202, or the RNA sequence corresponding to SEQ ID NO:202 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:203, or the RNA sequence corresponding to SEQ ID NO:203 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:204, or the RNA sequence corresponding to SEQ ID NO:204 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:205, or the RNA sequence corresponding to SEQ ID NO:205 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:206, or the RNA sequence corresponding to SEQ ID NO:206 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:207, or the RNA sequence corresponding to SEQ ID NO:207 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:208, or the RNA sequence corresponding to SEQ ID NO:208 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:209, or the RNA sequence corresponding to SEQ ID NO:209 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:210, or the RNA sequence corresponding to SEQ ID NO:210 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:211, or the RNA sequence corresponding to SEQ ID NO:211 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:212, or the RNA sequence corresponding to SEQ ID NO:212 (i.e., wherein all instances of thymine are replaced with uracil). In embodiments, the adenosine N-6 methylation CRISPR agonist, adenosine N-6 demethylation CRISPR antagonist, or RNA 2′-O-methyl transferase CRISPR inhibitor is SEQ ID NO:213, or the RNA sequence corresponding to SEQ ID NO:213 (i.e., wherein all instances of thymine are replaced with uracil).

In embodiments, the adenosine N-6 methylation CRISPR agonist, adenosine N-6 demethylation CRISPR antagonist, or RNA 2′-O-methyl transferase CRISPR inhibitor is a nucleic acid sequence, e.g., a DNA sequence described herein or the RNA sequence corresponding to the DNA sequence described herein (i.e., wherein all instances of thymine are replaced with uracil). In embodiments, the nucleic acid sequence has at least 80% (80% or more) sequence identity to a nucleic acid sequence described herein, e.g., a DNA sequence described herein or the RNA sequence corresponding to the DNA sequence described herein (i.e., wherein all instances of thymine are replaced with uracil). In embodiments, the nucleic acid sequence may have 80%, 85%, 90%, 91%, 92%, 93%, 94%, 95%, 96%, 97%, 98%, 99% or 100% sequence identity to a nucleic acid sequence described herein, e.g., a DNA sequence described herein or the RNA sequence corresponding to the DNA sequence described herein (i.e., wherein all instances of thymine are replaced with uracil), such as SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117, SEQ ID NO:118, SEQ ID NO:119, SEQ ID NO:120, SEQ ID NO:121, SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151, SEQ ID NO:152, SEQ ID NO:153, SEQ ID NO:154, SEQ ID NO:155, SEQ ID NO:156, SEQ ID NO:157, SEQ ID NO:158, SEQ ID NO:159, SEQ ID NO:160, SEQ ID NO:161, SEQ ID NO:162, SEQ ID NO:163, SEQ ID NO:164, SEQ ID NO:165, SEQ ID NO:166, SEQ ID NO:167, SEQ ID NO:168, SEQ ID NO:169, SEQ ID NO:170, SEQ ID NO:171, SEQ ID NO:172, SEQ ID NO:173, SEQ ID NO:174, SEQ ID NO:175, SEQ ID NO:176, SEQ ID NO:177, SEQ ID NO:178, SEQ ID NO:179, SEQ ID NO:180, SEQ ID NO:181, SEQ ID NO:182, SEQ ID NO:183, SEQ ID NO:184, SEQ ID NO:185, SEQ ID NO:186, SEQ ID NO:187, SEQ ID NO:188, SEQ ID NO:189, SEQ ID NO:190, SEQ ID NO:191, SEQ ID NO:192, SEQ ID NO:193, SEQ ID NO:194, SEQ ID NO:195, SEQ ID NO:196, SEQ ID NO:197, SEQ ID NO:198, SEQ ID NO:199, SEQ ID NO:200, SEQ ID NO:201, SEQ ID NO:202, SEQ ID NO:203, SEQ ID NO:204, SEQ ID NO:205, SEQ ID NO:206, SEQ ID NO:207, SEQ ID NO:208, SEQ ID NO:209, SEQ ID NO:210, SEQ ID NO:211, SEQ ID NO:212, or SEQ ID NO:213.

### V. EMBODIMENTS

In a first aspect, there is provided a method of treating or preventing a Zika viral infection in a subject in need thereof. The method includes administering an effective amount of an adenosine N-6 methylation agonist or an adenosine N-6 demethylation antagonist.

In another aspect, there is provided a method of treating or preventing a Zika viral infection in a subject in need thereof, the method including administering an effective amount of an RNA 2′-O-methyl transferase inhibitor.

In another aspect, there is provided a method of treating or preventing an RNA virus infection in a subject in need thereof. The method includes administering an effective amount of a compound of the formula:

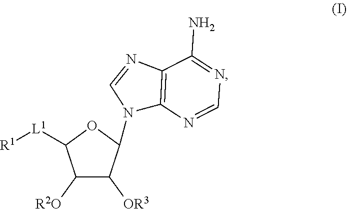

wherein, L¹ is a bond, —O—, —S—, —NH—, —C(O)NH—, —C(O)—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene; R¹ is hydrogen, halogen, —CF₃, —CN, —OH, —NH₂, —N₃, —SH, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl; R² is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl; and R³ is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

In another aspect, there is provided a compound of the formula:

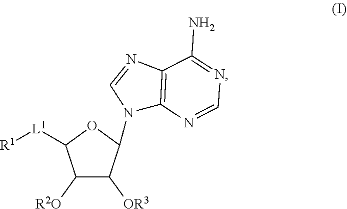

wherein, L¹ is a bond, —O—, —S—, —NH—, —C(O)NH—, —C(O)—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene; R¹ is hydrogen, halogen, —CF₃, —CN, —OH, —NH₂, —N₃, —SH, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl; R² is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl; and R³ is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

In another aspect, there is provided a pharmaceutical composition including a compound disclosed herein and a pharmaceutically acceptable excipient.

In another aspect, there is provided a vaccine composition including a compound disclosed herein, a vaccine adjuvant and a immunogenic agent.

In another aspect, there is provided a pharmaceutical composition comprising an RNA compound, said RNA compound encoding an RNA virus structural protein.

### Embodiment P1

A method of treating or preventing a Zika viral infection in a subject in need thereof, the method comprising administering an effective amount of an adenosine N-6 methylation agonist or an adenosine N-6 demethylation antagonist.

### Embodiment P2

The method of embodiment P1, wherein said adenosine N-6 methylation agonist is a RNA methyltransferase complex agonist, a METTL3 agonist or a METTL14 agonist.

### Embodiment P3

The method of embodiment P1, wherein said adenosine N-6 demethylation antagonist is a ALKBH5 antagonist or a FTO antagonist.

### Embodiment P4

The method of one of embodiments P1 to P3, wherein said adenosine N-6 methylation agonist is an adenosine N-6 methylation antisense nucleic acid agonist and said adenosine N-6 demethylation antagonist is an adenosine N-6 demethylation antisense nucleic acid antagonist.

### Embodiment P5

The method of embodiment P4, wherein said N-6 methylation antisense nucleic acid agonist is an N-6 methylation RNAi agonist and said adenosine N-6 demethylation antisense nucleic acid antagonist is an adenosine N-6 demethylation RNAi antagonist.

### Embodiment P6

The method of one of embodiments P1 to P3, wherein said adenosine N-6 methylation agonist is an adenosine N-6 methylation aptamer agonist or an adenosine N-6 methylation antibody agonist.

### Embodiment P7

The method of one of embodiments P1 to P3, wherein said adenosine N-6 demethylation antagonist is an adenosine N-6 demethylation aptamer antagonist or an adenosine N-6 demethylation antibody antagonist.

### Embodiment P8

The method of one of embodiments P1 to P7, further comprising administering an immunogenic agent.

### Embodiment P9

The method of one of embodiments P1 to P3, wherein said adenosine N-6 demethylation antagonist is an adenosine N-6 demethylation CRISPR antagonist or an adenosine N-6 demethylation CRISPR antagonist.

### Embodiment P10

The method of embodiment P9, wherein said immunogenic agent and said adenosine N-6 methylation agonist or said adenosine N-6 demethylation antagonist together with a vaccine adjuvant form a vaccine formulation.

### Embodiment P11

The method of one of embodiments P1 to P10, wherein said adenosine N-6 methylation agonist or said adenosine N-6 demethylation antagonist are capable of activating the immune system of said subject.

### Embodiment P12

A method of treating or preventing a Zika viral infection in a subject in need thereof, the method comprising administering an effective amount of an RNA 2′-O-methyl transferase inhibitor.

### Embodiment P13

The method of embodiment P12, wherein said RNA 2′-O-methyl transferase inhibitor is an RNA 2′-O-methyl transferase antisense inhibitor, an RNA 2′-O-methyl CRISPR inhibitor, an RNA 2′-O-methyl aptamer inhibitor or an RNA 2′-O-methyl transferase antibody inhibitor.

### Embodiment P14

The method of one of embodiments P12 or P13, wherein said RNA 2′-O-methyl transferase inhibitor is a nonstructural protein 5(NS5) inhibitor.

### Embodiment P15

The method of embodiment P12, wherein said RNA 2′-O-methyl transferase inhibitor is S-adenosylmethionine, a chemical analogue of S-adenosylmethionine or a small molecule functional analogue of S-adenosylmethionine.

### Embodiment P16

The method of embodiment P12, wherein said RNA 2′-O-methyl transferase inhibitor is:

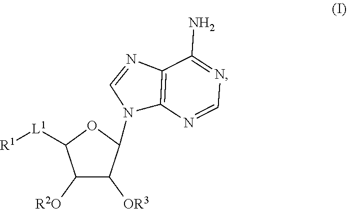

wherein, L¹ is a bond, —O—, —S—, —NH—, —C(O)NH—, —C(O)—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene; R¹ is hydrogen, halogen, —CF₃, —CN, —OH, —NH₂, —N₃, —SH, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl; R² is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl; and R³ is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

### Embodiment P17

The method of embodiment P16, wherein R² and R³ are hydrogen.

### Embodiment P18

The method of embodiment P16 or P17, wherein L¹ is —(CH₂)_{z1}—NH—(CH2)_{z2}—, —(CH2)_{z1}—S—(CH2)_{z2}—, —(CH2)_{z1}—O—(CH2)_{z2}—, —(CH₂)_{z1}—C(O)NH—(CH₂)_{z2}—, —(CH₂)_{z1}—C(O)O—(CH₂)_{z2}—, wherein z1 and z2 are independently integers from 0 to 10.

### Embodiment P19

The method of embodiment P18, wherein z1 and z2 are independently integers from 0 to 5.

### Embodiment P20

The method of embodiment P18, wherein z1 and z2 are independently integers from 0 to 3.

### Embodiment P21

The method of embodiment P18, wherein z1 and z2 are 1.

### Embodiment P22

The method of embodiment P18, wherein z1 is an integer from 0 to 3, and z2 is 0.

### Embodiment P23

The method of embodiment P18, wherein z1 is 1, and z2 is 0.

### Embodiment P24

The method of embodiment P18, wherein z2 is an integer from 0 to 3, and z1 is 0.

### Embodiment P25

The method of embodiment P18, wherein z1 is 0, and z2 is 1.

### Embodiment P26

The method of one of embodiments P16 to P25, wherein R¹ is substituted or unsubstituted cycloalkyl, substituted or unsubstituted heterocycloalkyl, substituted or unsubstituted aryl or substituted or unsubstituted heteroaryl.

### Embodiment P27

The method of embodiment P26, wherein R¹ is substituted or unsubstituted heterocycloalkyl, substituted or unsubstituted aryl or substituted or unsubstituted heteroaryl.

### Embodiment P28

The method of embodiment P26, wherein R¹ is substituted or unsubstituted purinyl, substituted or unsubstituted imidazopyridinyl, substituted or unsubstituted imidazopyridinyl, substituted or unsubstituted benzoimidazolyl, substituted or unsubstituted benzotriazolyl, substituted or unsubstituted indazolyl, substituted or unsubstituted dihydropurinonyl, substituted or unsubstituted pyrazolopyridinyl, substituted or unsubstituted dihydropurinedionyl, substituted or unsubstituted furanyl, substituted or unsubstituted dihydrofuroquiozalinyl, substituted or unsubstituted pyrimidoindolyl, substituted or unsubstituted carbazolyl, substituted or unsubstituted indenyl, substituted or unsubstituted dihydrocyclopenta-isoindolyl, substituted or unsubstituted pyridinyl, substituted or unsubstituted pyrimidinyl, substituted or unsubstituted pyridazinyl, substituted or unsubstituted triazinyl, substituted or unsubstituted quinolinyl, substituted or unsubstituted quinazolinyl, substituted or unsubstituted quinoxalinyl, substituted or unsubstituted pyrrolyl, substituted or unsubstituted imidazolyl, substituted or unsubstituted oxazolyl, substituted or unsubstituted isoxazolyl, substituted or unsubstituted thiazolyl, substituted or unsubstituted isothizolyl, substituted or unsubstituted triazolyl, substituted or unsubstituted oxadiazolyl, substituted or unsubstituted thiadiazolyl, substituted or unsubstituted indolyl, substituted or unsubstituted indalinyl, substituted or unsubstituted benzofuranyl, substituted or unsubstituted benzothiophenyl, substituted or unsubstituted acridinyl, or substituted or unsubstituted phenyl.

### Embodiment P29

The method of embodiment P28, wherein R¹ is substituted with -L¹-R^{1A}, wherein R^{1A} is halogen, —CF₃, —CN, —OH, —NH₂, —N₃, —SH, —COOH, —CONH₂, R^{1B}-substituted or unsubstituted alkyl, R^{1B}-substituted or unsubstituted heteroalkyl, R^{1B}-substituted or unsubstituted cycloalkyl, R^{1B}-substituted or unsubstituted heterocycloalkyl, R^{1B}-substituted or unsubstituted aryl or R^{1B}-substituted or unsubstituted heteroaryl; R^{1B} is halogen, —CF₃, —CN, —OH, —NH₂, —N₃, —SH, —COOH, —CONH₂, R^{1C}-substituted or unsubstituted alkyl, R^{1C}-substituted or unsubstituted heteroalkyl, R^{1C}-substituted or unsubstituted cycloalkyl, R^{1C}-substituted or unsubstituted heterocycloalkyl, R^{1C}-substituted or unsubstituted aryl or R^{1C}-substituted or unsubstituted heteroaryl; R^{1C} is independently halogen, —CF₃, —CN, —OH, —NH₂, —N₃, —SH, —COOH, —CONH₂, unsubstituted alkyl, unsubstituted heteroalkyl, unsubstituted cycloalkyl, unsubstituted heterocycloalkyl, unsubstituted aryl or unsubstituted heteroaryl; and L^{1A} is a bond, —O—, —S—, —NH—, —C(O)NH—, —C(O)—, R^{1D}-substituted or unsubstituted alkylene, R^{1D}-substituted or unsubstituted heteroalkylene, R^{1D}-substituted or unsubstituted cycloalkylene, R^{1D}-substituted or unsubstituted heterocycloalkylene, R^{1D}-substituted or unsubstituted arylene or R^{1D}-substituted or unsubstituted heteroarylene; R^{1D} is halogen, —CF₃, —CN, —OH, —NH₂, —N₃, —SH, —COOH, —CONH₂, R^{1E}-substituted or unsubstituted alkyl, R^{1E}-substituted or unsubstituted heteroalkyl, R^{1E}-substituted or unsubstituted cycloalkyl, R^{1E}-substituted or unsubstituted heterocycloalkyl, R^{1E}-substituted or unsubstituted aryl or R^{1E}-substituted or unsubstituted heteroaryl; and R^{1E} is halogen, —CF₃, —CN, —OH, —NH₂, —N₃, —SH, —COOH, —CONH₂, unsubstituted alkyl, unsubstituted heteroalkyl, unsubstituted cycloalkyl, unsubstituted heterocycloalkyl, unsubstituted aryl or unsubstituted heteroaryl.

### Embodiment P30

A method of treating or preventing an RNA virus infection in a subject in need thereof, the method comprising administering an effective amount of a compound of the formula:

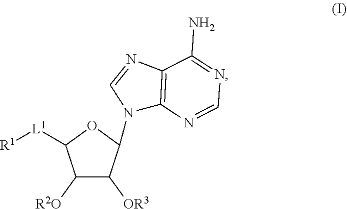

wherein,
L¹ is a bond, —O—, —S—, —NH—, —C(O)NH—, —C(O)—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene; R¹ is hydrogen, halogen, —CF₃, —CN, —OH, —NH₂, —N₃, —SH, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl; R² is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl; and R³ is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

### Embodiment P31

The method of embodiment P30, wherein R² and R³ are hydrogen.

### Embodiment P32

The method of embodiment P30 or P31, wherein L¹ is —(CH₂)_{z1}—NH—(CH₂)_{z2}—, —(CH₂)_{z1}—S—(CH₂)_{z2}—, —(CH₂)_{z1}—O—(CH₂)_{z2}—, —(CH₂)_{z1}—C(O)NH—(CH₂)_{z2}—, —(CH₂)_{z1}—C(O)O—(CH₂)_{z2}—, wherein z1 and z2 are independently integers from 0 to 10.

### Embodiment P33

The method of embodiment P32, wherein z1 and z2 are independently integers from 0 to 5.

### Embodiment P34

The method of embodiment P32, wherein z1 and z2 are independently integers from 0 to 3.

### Embodiment P35

The method of embodiment P32, wherein z1 and z2 are 1.

### Embodiment P36

The method of embodiment P32, wherein z1 is an integer from 0 to 3, and z2 is 0.

### Embodiment P37

The method of embodiment P32, wherein z1 is 1, and z2 is 0.

### Embodiment P38

The method of embodiment P32, wherein z2 is an integer from 0 to 3, and z1 is 0.

### Embodiment P39

The method of embodiment P32, wherein z1 is 0, and z2 is 1.

### Embodiment P40

The method of one of embodiments P30 to P39, wherein R¹ is substituted or unsubstituted cycloalkyl, substituted or unsubstituted heterocycloalkyl, substituted or unsubstituted aryl or substituted or unsubstituted heteroaryl.

### Embodiment P41

The method of embodiment P40, wherein R¹ is substituted or unsubstituted heterocycloalkyl, substituted or unsubstituted aryl or substituted or unsubstituted heteroaryl.

### Embodiment P42

The method of embodiment P40, wherein R¹ is substituted or unsubstituted purinyl, substituted or unsubstituted imidazopyridinyl, substituted or unsubstituted imidazopyridinyl, substituted or unsubstituted benzoimidazolyl, substituted or unsubstituted benzotriazolyl, substituted or unsubstituted indazolyl, substituted or unsubstituted dihydropurinonyl, substituted or unsubstituted pyrazolopyridinyl, substituted or unsubstituted dihydropurinedionyl, substituted or unsubstituted furanyl, substituted or unsubstituted dihydrofuroquiozalinyl, substituted or unsubstituted pyrimidoindolyl, substituted or unsubstituted carbazolyl, substituted or unsubstituted indenyl, substituted or unsubstituted dihydrocyclopenta-isoindolyl, substituted or unsubstituted pyridinyl, substituted or unsubstituted pyrimidinyl, substituted or unsubstituted pyridazinyl, substituted or unsubstituted triazinyl, substituted or unsubstituted quinolinyl, substituted or unsubstituted quinazolinyl, substituted or unsubstituted quinoxalinyl, substituted or unsubstituted pyrrolyl, substituted or unsubstituted imidazolyl, substituted or unsubstituted oxazolyl, substituted or unsubstituted isoxazolyl, substituted or unsubstituted thiazolyl, substituted or unsubstituted isothizolyl, substituted or unsubstituted triazolyl, substituted or unsubstituted oxadiazolyl, substituted or unsubstituted thiadiazolyl, substituted or unsubstituted indolyl, substituted or unsubstituted pyrazolopyridinyl, substituted or unsubstituted benzofuranyl, substituted or unsubstituted benzothiophenyl, substituted or unsubstituted acridinyl, or substituted or unsubstituted phenyl.

### Embodiment P43

The method of embodiment P42, wherein R¹ is substituted with -L¹-R^{1A}, wherein R^{1A} is halogen, —CF₃, —CN, —OH, —NH₂, —N₃, —SH, —COOH, —CONH₂, R^{1B}-substituted or unsubstituted alkyl, R^{1B}-substituted or unsubstituted heteroalkyl, R^{1B}-substituted or unsubstituted cycloalkyl, R^{1B}-substituted or unsubstituted heterocycloalkyl, R^{1B}-substituted or unsubstituted aryl or R^{1B}-substituted or unsubstituted heteroaryl; R^{1B} is halogen, —CF₃, —CN, —OH, —NH₂, —N₃, —SH, —COOH, —CONH₂, R^{1C}-substituted or unsubstituted alkyl, R^{1C}-substituted or unsubstituted heteroalkyl, R^{1C}-substituted or unsubstituted cycloalkyl, R^{1C}-substituted or unsubstituted heterocycloalkyl, R^{1C}-substituted or unsubstituted aryl or R^{1C}-substituted or unsubstituted heteroaryl; R^{1C} is halogen, —CF₃, —CN, —OH, —NH₂, —N₃, —SH, —COOH, —CONH₂, unsubstituted alkyl, unsubstituted heteroalkyl, unsubstituted cycloalkyl, unsubstituted heterocycloalkyl, unsubstituted aryl or unsubstituted heteroaryl; and L^{1A} is a bond, —O—, —S—, —NH—, —C(O)NH—, —C(O)—, R^{1D}-substituted or unsubstituted alkylene, R^{1D}-substituted or unsubstituted heteroalkylene, R^{1D}-substituted or unsubstituted cycloalkylene, R^{1D}-substituted or unsubstituted heterocycloalkylene, R^{1D}-substituted or unsubstituted arylene or R^{1D}-substituted or unsubstituted heteroarylene; R^{1D} is halogen, —CF₃, —CN, —OH, —NH₂, —N₃, —SH, —COOH, —CONH₂, R^{1E}-substituted or unsubstituted alkyl, R^{1E}-substituted or unsubstituted heteroalkyl, R^{1E} substituted or unsubstituted cycloalkyl, R^{1E}-substituted or unsubstituted heterocycloalkyl, R^{1E-}substituted or unsubstituted aryl or R^{1E}-substituted or unsubstituted heteroaryl; and R^{1E} is halogen, —CF₃, —CN, —OH, —NH₂, —N₃, —SH, —COOH, —CONH₂, unsubstituted alkyl, unsubstituted heteroalkyl, unsubstituted cycloalkyl, unsubstituted heterocycloalkyl, unsubstituted aryl or unsubstituted heteroaryl.

### Embodiment P44

The method of one of embodiments P30 to P43, wherein said RNA virus infection is an HIV infection, Dengue viral infection or a Zika viral infection.

### Embodiment P45

A compound of the formula:

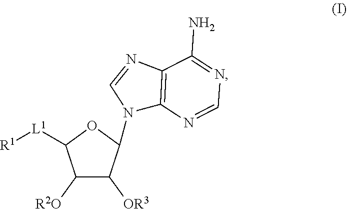

wherein,
L¹ is a bond, —O—, —S—, —NH—, —C(O)NH—, —C(O)—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene; R¹ is hydrogen, halogen, —CF₃, —CN, —OH, —NH₂, —N₃, —SH, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl; R² is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl; and R³ is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

### Embodiment P46

The compound of embodiment P45, wherein R² and R³ are hydrogen.

### Embodiment P47

The compound of embodiment P45 or P46, wherein L¹ is —(CH₂)_{z1}—NH—(CH₂)_{z2}—, —(CH₂)_{z1}—S—(CH₂)_{z2}—, —(CH₂)_{z1}—O—(CH₂)_{z2}—, —(CH₂)_{z1}—C(O)NH—(CH₂)_{z2}—, —(CH₂)_{z1}—C(O)O—(CH₂)_{z2}—, wherein z1 and z2 are independently integers from 0 to 10.

### Embodiment P48

The compound of embodiment P47, wherein z1 and z2 are independently integers from 0 to 5.

### Embodiment P49

The compound of embodiment P47, wherein z1 and z2 are independently integers from 0 to 3.

### Embodiment P50

The compound of embodiment P47, wherein z1 and z2 are 1.

### Embodiment P51

The compound of embodiment P47, wherein z1 is an integer from 0 to 3, and z2 is 0.

### Embodiment P52

The compound of embodiment P47, wherein z1 is 1, and z2 is 0.

### Embodiment P53

The compound of embodiment P47, wherein z2 is an integer from 0 to 3, and z1 is 0.

### Embodiment P54

The compound of embodiment P47, wherein z1 is 0, and z2 is 1.

### Embodiment P55

The compound of one of embodiments P45 to P54, wherein R¹ is substituted or unsubstituted cycloalkyl, substituted or unsubstituted heterocycloalkyl, substituted or unsubstituted aryl or substituted or unsubstituted heteroaryl.

### Embodiment P56

The compound of embodiment P55, wherein R¹ is substituted or unsubstituted heterocycloalkyl, substituted or unsubstituted aryl or substituted or unsubstituted heteroaryl.

### Embodiment P57

The compound of embodiment P55, wherein R¹ is substituted or unsubstituted purinyl, substituted or unsubstituted imidazopyridinyl, substituted or unsubstituted imidazopyridinyl, substituted or unsubstituted benzoimidazolyl, substituted or unsubstituted benzotriazolyl, substituted or unsubstituted indazolyl, substituted or unsubstituted dihydropurinonyl, substituted or unsubstituted pyrazolopyridinyl, substituted or unsubstituted dihydropurinedionyl, substituted or unsubstituted furanyl, substituted or unsubstituted dihydrofuroquiozalinyl, substituted or unsubstituted pyrimidoindolyl, substituted or unsubstituted carbazolyl, substituted or unsubstituted indenyl, substituted or unsubstituted dihydrocyclopenta-isoindolyl, substituted or unsubstituted pyridinyl, substituted or unsubstituted pyrimidinyl, substituted or unsubstituted pyridazinyl, substituted or unsubstituted triazinyl, substituted or unsubstituted quinolinyl, substituted or unsubstituted quinazolinyl, substituted or unsubstituted quinoxalinyl, substituted or unsubstituted pyrrolyl, substituted or unsubstituted imidazolyl, substituted or unsubstituted oxazolyl, substituted or unsubstituted isoxazolyl, substituted or unsubstituted thiazolyl, substituted or unsubstituted isothizolyl, substituted or unsubstituted triazolyl, substituted or unsubstituted oxadiazolyl, substituted or unsubstituted thiadiazolyl, substituted or unsubstituted indolyl, substituted or unsubstituted pyrazolopyridinyl, substituted or unsubstituted benzofuranyl, substituted or unsubstituted benzothiophenyl, substituted or unsubstituted acridinyl, or substituted or unsubstituted phenyl.

### Embodiment P58

The compound of embodiment P57, wherein R¹ is substituted with -L¹-R^{1A}, wherein R^{1A} is halogen, —CF₃, —CN, —OH, —NH₂, —N₃, —SH, —COOH, —CONH₂, R^{1B}-substituted or unsubstituted alkyl, R^{1B}-substituted or unsubstituted heteroalkyl, R^{1B}-substituted or unsubstituted cycloalkyl, R^{1B}-substituted or unsubstituted heterocycloalkyl, R^{1B}-substituted or unsubstituted aryl or R^{1B}-substituted or unsubstituted heteroaryl; R^{1B} is halogen, —CF₃, —CN, —OH, —NH₂, —N₃, —SH, —COOH, —CONH₂, R^{1C}-substituted or unsubstituted alkyl, R^{1C}-substituted or unsubstituted heteroalkyl, R^{1C}-substituted or unsubstituted cycloalkyl, R^{1C}-substituted or unsubstituted heterocycloalkyl, R^{1C}-substituted or unsubstituted aryl or R^{1C}-substituted or unsubstituted heteroaryl; R^{1C} is halogen, —CF₃, —CN, —OH, —NH₂, —N₃, —SH, —COOH, —CONH₂, unsubstituted alkyl, unsubstituted heteroalkyl, unsubstituted cycloalkyl, unsubstituted heterocycloalkyl, unsubstituted aryl or unsubstituted heteroaryl; and L^{1A} is a bond, —O—, —S—, —NH—, —C(O)NH—, —C(O)—, R^{1D}-substituted or unsubstituted alkylene, R^{1D}-substituted or unsubstituted heteroalkylene, R^{1D}-substituted or unsubstituted cycloalkylene, R^{1D}-substituted or unsubstituted heterocycloalkylene, R^{1D}-substituted or unsubstituted arylene or R^{1D}-substituted or unsubstituted heteroarylene; R^{1D} is halogen, —CF₃, —CN, —OH, —NH₂, —N₃, —SH, —COOH, —CONH₂, R^{1E}-substituted or unsubstituted alkyl, R^{1E}-substituted or unsubstituted heteroalkyl, R^{1E}-substituted or unsubstituted cycloalkyl, R^{1E}-substituted or unsubstituted heterocycloalkyl, R^{1E} substituted or unsubstituted aryl or R^{1E}-substituted or unsubstituted heteroaryl; and R^{1E} is halogen, —CF₃, —CN, —OH, —NH₂, —N₃, —SH, —COOH, —CONH₂, unsubstituted alkyl, unsubstituted heteroalkyl, unsubstituted cycloalkyl, unsubstituted heterocycloalkyl, unsubstituted aryl or unsubstituted heteroaryl.

### Embodiment P59

A pharmaceutical composition comprising the compound of one of embodiments 45 to 55 and a pharmaceutically acceptable excipient.

### Embodiment P60

A vaccine composition comprising the compound of one of embodiments 45 to 55, a vaccine adjuvant and a immunogenic agent.

### Embodiment P61

A pharmaceutical composition comprising an RNA compound, said RNA compound encoding an RNA virus structural protein.

### Embodiment P62

The pharmaceutical composition of embodiment P61 wherein said RNA virus structural protein is a Zika virus structural protein.

### Embodiment P63

The pharmaceutical composition of one of embodiment P61 or P62 wherein said RNA virus structural protein is a viral premembrane protein (prM), viral envelope protein (Env), a capsid protein (C) or a membrane protein (M).

### Embodiment P64

The pharmaceutical composition of one of embodiment P61 to P64, wherein said RNA compound is modified on at least one 2′ position with a 2′ O-methyl and/or modified on at least one adenosine at the N6 adenine position with a methyl.

### ADDITIONAL EMBODIMENTS

### Embodiment 1

A method of treating or preventing a Zika viral infection in a subject in need thereof, the method comprising administering an effective amount of an adenosine N-6 methylation agonist or an adenosine N-6 demethylation antagonist.

### Embodiment 2

The method of embodiment 1, wherein said adenosine N-6 methylation agonist is a RNA methyltransferase complex agonist, a N6-adenosine-methyltransferase (METTL3) agonist, or a Methyltransferase-Like Protein 14 (METTL14) agonist.

### Embodiment 3

The method of embodiment 1 or 2, wherein said adenosine N-6 methylation agonist is a METTL3 or a METTL14 agonist.

### Embodiment 4

The method of embodiment 1, wherein said adenosine N-6 demethylation antagonist is an alkB homolog 5 RNA demethylase (ALKBH5) antagonist or an alpha-ketoglutarate-dependent dioxygenase (FTO) antagonist.

### Embodiment 5

The method of any one of embodiments 1 to 3, wherein said adenosine N-6 methylation agonist is an adenosine N-6 methylation antisense nucleic acid agonist and said adenosine N-6 demethylation antagonist is an adenosine N-6 demethylation antisense nucleic acid antagonist.

### Embodiment 6

The method of embodiment 5, wherein said N-6 methylation antisense nucleic acid agonist is an N-6 methylation RNAi agonist and said adenosine N-6 demethylation antisense nucleic acid antagonist is an adenosine N-6 demethylation RNAi antagonist.

### Embodiment 7

The method of any one of embodiments 1 to 3, wherein said adenosine N-6 methylation agonist is an adenosine N-6 methylation aptamer agonist or an adenosine N-6 methylation antibody agonist.

### Embodiment 8

The method of any one of embodiments 1 to 3, wherein said adenosine N-6 demethylation antagonist is an adenosine N-6 demethylation aptamer antagonist or an adenosine N-6 demethylation antibody antagonist.

### Embodiment 9

The method of any one of embodiments 1 to 8, further comprising administering an immunogenic agent.

### Embodiment 10

The method of any one of embodiments 1 to 3, wherein said adenosine N-6 methylation agonist is an adenosine N-6 methylation CRISPR agonist or adenosine N-6 demethylation antagonist is an adenosine N-6 demethylation CRISPR antagonist.

### Embodiment 11

The method of embodiment 9, wherein said immunogenic agent and said adenosine N-6 methylation agonist or said adenosine N-6 demethylation antagonist together with a vaccine adjuvant form a vaccine formulation.

### Embodiment 12

The method of any one of embodiments 1 to 10, wherein said adenosine N-6 methylation agonist or said adenosine N-6 demethylation antagonist are capable of activating the immune system of said subject.

### Embodiment 13

A method of treating or preventing a Zika viral infection in a subject in need thereof, the method comprising administering an effective amount of an RNA 2′-O-methyl transferase inhibitor.

### Embodiment 14

The method of embodiment 13, wherein said RNA 2′-O-methyl transferase inhibitor is an RNA 2′-O-methyl transferase antisense inhibitor, an RNA 2′-O-methyl transferase CRISPR inhibitor, an RNA 2′-O-methyl aptamer inhibitor or an RNA 2′-O-methyl transferase antibody inhibitor.

### Embodiment 15

The method of any one of embodiments 13 or 14, wherein said RNA 2′-O-methyl transferase inhibitor is a nonstructural protein 5 (NS5) inhibitor.

### Embodiment 16

The method of embodiment 13, wherein said RNA 2′-O-methyl transferase inhibitor is S-adenosylmethionine, a chemical analogue of S-adenosylmethionine, or a small molecule functional analogue of S-adenosylmethionine.

### Embodiment 17

The method of embodiment 13, wherein said RNA 2′-O-methyl transferase inhibitor is a compound of the formula:

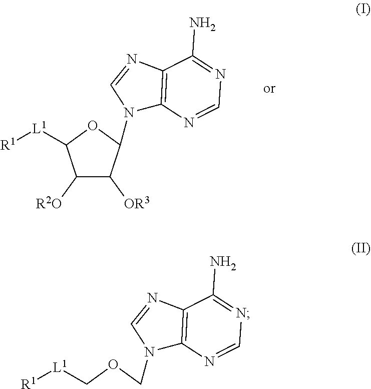

wherein,
L¹ is a bond, —O—, —S—, —NH—, —C(O)NH—, —C(O)—, —S(O)₂NH—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene; R¹ is hydrogen, halogen, —CF₃, —CN, —OR^{1A}, —NHR^{1A}, —N₃, —SR^{1A}, —COOR^{1A}, —CONHR^{1A}, —NHC(O)R^{1A}, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl; R^{1A} is hydrogen, halogen, —CF₃, —CN, —OH, —NH₂, —N₃, —SH, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl; R² is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl; and R³ is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

### Embodiment 18

The method of embodiment 17, wherein R² and R³ are hydrogen.

### Embodiment 19

The method of embodiments 17 or 18, wherein L¹ is —(CH₂)_{z1}—NH—(CH₂)_{z2}—, —(CH₂)_{z1}—S—(CH₂)_{z2}—, —(CH₂)_{z1}—O—(CH₂)_{z2}—, —(CH₂)_{z1}—C(O)NH—(CH₂)_{z2}—, —(CH₂)_{z1}—C(O)O—(CH₂)_{z2}—, or —(CH₂)_{z1}—S(O)₂NH(CH₂)_{z2}, wherein z1 and z2 are independently integers from 0 to 10.

### Embodiment 20

The method of embodiment 19, wherein z1 and z2 are independently integers from 0 to 5.

### Embodiment 21

The method of embodiment 19, wherein z1 and z2 are independently integers from 0 to 3.

### Embodiment 22

The method of embodiment 19, wherein z1 and z2 are 1.

### Embodiment 23

The method of embodiment 19, wherein z1 is an integer from 0 to 3, and z2 is 0.

### Embodiment 24

The method of embodiment 19, wherein z1 is 1, and z2 is 0.

### Embodiment 25

The method of embodiment 19, wherein z2 is an integer from 0 to 3, and z1 is 0.

### Embodiment 26

The method of embodiment 19, wherein z1 is 0, and z2 is 1.

### Embodiment 27

The method of any one of embodiments 17 to 26, wherein R¹ is substituted or unsubstituted cycloalkyl, substituted or unsubstituted heterocycloalkyl, substituted or unsubstituted aryl or substituted or unsubstituted heteroaryl.

### Embodiment 28

The method of embodiment 27, wherein R¹ is substituted or unsubstituted heterocycloalkyl, substituted or unsubstituted aryl or substituted or unsubstituted heteroaryl.

### Embodiment 29

The method of any one of embodiments 17 to 26, wherein R¹ is substituted or unsubstituted purinyl, substituted or unsubstituted imidazopyridinyl, substituted or unsubstituted imidazopyridinyl, substituted or unsubstituted benzoimidazolyl, substituted or unsubstituted benzotriazolyl, substituted or unsubstituted indazolyl, substituted or unsubstituted dihydropurinonyl, substituted or unsubstituted pyrazolopyridinyl, substituted or unsubstituted dihydropurinedionyl, substituted or unsubstituted furanyl, substituted or unsubstituted dihydrofuroquiozalinyl, substituted or unsubstituted pyrimidoindolyl, substituted or unsubstituted carbazolyl, substituted or unsubstituted indenyl, substituted or unsubstituted dihydrocyclopenta-isoindolyl, substituted or unsubstituted pyridinyl, substituted or unsubstituted pyrimidinyl, substituted or unsubstituted pyridazinyl, substituted or unsubstituted triazinyl, substituted or unsubstituted quinolinyl, substituted or unsubstituted quinazolinyl, substituted or unsubstituted quinoxalinyl, substituted or unsubstituted pyrrolyl, substituted or unsubstituted imidazolyl, substituted or unsubstituted oxazolyl, substituted or unsubstituted isoxazolyl, substituted or unsubstituted thiazolyl, substituted or unsubstituted isothizolyl, substituted or unsubstituted triazolyl, substituted or unsubstituted oxadiazolyl, substituted or unsubstituted thiadiazolyl, substituted or unsubstituted indolyl, substituted or unsubstituted pyrazolopyridinyl, substituted or unsubstituted benzofuranyl, substituted or unsubstituted benzothiophenyl, substituted or unsubstituted acridinyl, or substituted or unsubstituted phenyl.

### Embodiment 30

The method of embodiment any one of embodiments 17 to 29, wherein R^{1A} is hydrogen, halogen, —CF₃, —CN, —OH, —NH₂, —N₃, —SH, —COOH, —NHCOOH, —CONH₂, R^{1B}-substituted or unsubstituted alkyl, R^{1B}-substituted or unsubstituted heteroalkyl, R^{1B}-substituted or unsubstituted cycloalkyl, R^{1B}-substituted or unsubstituted heterocycloalkyl, R^{1B}-substituted or unsubstituted aryl or R^{1B}-substituted or unsubstituted heteroaryl; R^{1B} is independently halogen, —CF₃, —CN, —OH, —NH₂, —N₃, —NO₂, —SH, —COOH, —NHCOOH, —CONH₂, R^{1C}-substituted or unsubstituted alkyl, R^{1C}-substituted or unsubstituted heteroalkyl, R^{1C}-substituted or unsubstituted cycloalkyl, R^{1C}-substituted or unsubstituted heterocycloalkyl, R^{1C}-substituted or unsubstituted aryl or R^{1C}-substituted or unsubstituted heteroaryl; and R^{1C} is independently halogen, —CF₃, —CN, —OH, —NH₂, —N₃, —SH, —COOH, —NHCOOH, —CONH₂, unsubstituted alkyl, unsubstituted heteroalkyl, unsubstituted cycloalkyl, unsubstituted heterocycloalkyl, unsubstituted aryl or unsubstituted heteroaryl.

### Embodiment 31

The method of embodiment 29, wherein L¹ is a bond, —O—, —S—, —NH—, —C(O)NH—, —C(O)—, —S(O)₂NH—, R^{1D}-substituted or unsubstituted alkylene, R^{1D}-substituted or unsubstituted heteroalkylene, R^{1D}-substituted or unsubstituted cycloalkylene, R^{1D}-substituted or unsubstituted heterocycloalkylene, R^{1D}-substituted or unsubstituted arylene or R^{1D}-substituted or unsubstituted heteroarylene, wherein R^{1D} is halogen, —CF₃, —CN, —OH, —NH₂, —N₃, —SH, —COOH, —NHCOOH, —CONH₂, R^{1E}-substituted or unsubstituted alkyl, R^{1E}-substituted or unsubstituted heteroalkyl, R^{1E}-substituted or unsubstituted cycloalkyl, R^{1E}-substituted or unsubstituted heterocycloalkyl, R^{1E}-substituted or unsubstituted aryl or R^{1E}-substituted or unsubstituted heteroaryl; and wherein R^{1E} is halogen, —CF₃, —CN, —OH, —NH₂, —N₃, —SH, —COOH, —NHCOOH, —CONH₂, unsubstituted alkyl, unsubstituted heteroalkyl, unsubstituted cycloalkyl, unsubstituted heterocycloalkyl, unsubstituted aryl or unsubstituted heteroaryl.

### Embodiment 32

The method of embodiment 17, wherein the compound has the formula:

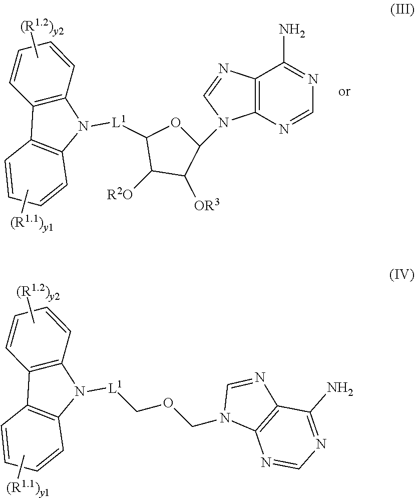

wherein,
y1 and y2 are independently an integer from 0 to 1,
R^{1′} and R^{1.2} independently are halogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCH₃, —OCBr₃, —OCCl₃, —OCI₃, —OCHF₂, —OCHBr₂, —OCHCl₂, —OCHI₂, —OCH₂F, —OCH₂Br, —OCH₂Cl, —OCH₂I, —CN, —O H, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

### Embodiment 33

The method of embodiment 32, wherein R² and R³ are hydrogen.

### Embodiment 34

The method of embodiment 32, wherein y1 and y2 are 0.

### Embodiment 35

The method of embodiment 32, wherein y1 and y2 are 1.

### Embodiment 36

The method of embodiment 32, wherein y1 is 0 and y2 is 1.

### Embodiment 37

The method of embodiment 32, wherein y1 is 1 and y2 is 0.

### Embodiment 38

The method of embodiment 32, wherein y1 is 1, y2 is 0, and R^{1.1} is —CH₃ or —CH₂F.

### Embodiment 39

The method of embodiment 32, wherein y1 is 0, y2 is 1, and R^{1.2} is —CH₂F, —CF₃, —CH₃, —OCH₃ or —C(CH₃)₃.

### Embodiment 40

The method of embodiment 32, wherein y1 and y2 are 1; and R^{1.2} is unsubstituted C₁-C₄ alkyl; and R^{1.1} is unsubstituted C₁-C₂ alkyl.

### Embodiment 41

The method of embodiment 32, wherein R^{1.1} is —CH₂F and R^{1.2} is —C(CH₃)₃; R^{1.1} is —CH₃ and R^{1.2} is —C(CH₃)₃; R^{1.1} is —CH₃ and R^{1.2} is —CF₃; R^{1.1} and R^{1.2} are each —CH₃; R^{1.1} is —CH₃ and R^{1.2} is —CH₂F; R^{1.1} is —CH₃ and R^{1.2} is —OCH₃; R^{1.1} is —CF₃ and R^{1.2} is —OCH₃; R^{1.1} is —CF₃ and R^{1.2} is —CH₃; R^{1.1} is —OCH₃ and R^{1.2} is —CH₃; or R^{1.1} is —OCH₃ and R^{1.2} is —CH₃.

### Embodiment 42

The method of any one of embodiments 32-41, wherein the compound has the formula:

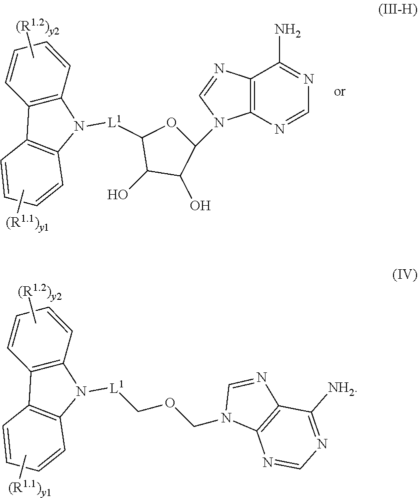

### Embodiment 43

The method of embodiment 42, wherein y1 is 1, y2 is 0, and R^{1.1} is —CH₃ or —CH₂F.

### Embodiment 44

The method of embodiment 42, wherein y1 is 0, y2 is 1, and R^{1.2} is —CH₂F, —CF₃, —CH₃, —OCH₃ or —C(CH₃)₃.

### Embodiment 45

The method of embodiment 42, wherein y1 and y2 are 1.

### Embodiment 46

The method of embodiment 45, wherein R^{1.1} is —CH₂F and R^{1.2} is —C(CH₃)₃; R^{1.1} is —CH₃ and R^{1.2} is —C(CH₃)₃; R^{1.1} is —CH₃ and R^{1.2} is —CF₃; R^{1.1} and R^{1.2} are each —CH₃; R^{1.1} is —CH₃ and R^{1.2} is —CH₂F; R^{1.1} is —CH₃ and R^{1.2} is —OCH₃; R^{1.1} is —CF₃ and R^{1.2} is —OCH₃; R^{1.1} is —CF₃ and R^{1.2} is —CH₃; R^{1.1} is —OCH₃ and R^{1.2} is —CH₃; or R^{1.1} is —OCH₃ and R^{1.2} is —CH₃.

### Embodiment 47

A method of treating or preventing an RNA virus infection in a subject in need thereof, the method comprising administering an effective amount of a compound of the formula:

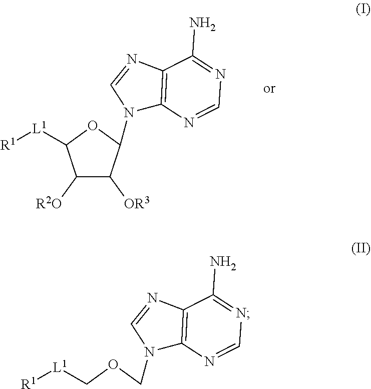

wherein,
L¹ is a bond, —O—, —S—, —NH—, —C(O)NH—, —C(O)—, —S(O)₂NH—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene;
R¹ is hydrogen, halogen, —CF₃, —CN, —OR^{1A}, —NHR^{1A}, —N₃, —SR^{1A}, —COOR^{1A}, —CONHR^{1A}, —NHC(O)R^{1A}, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl;
R^{1A} is hydrogen, halogen, —CF₃, —CN, —OH, —NH₂, —N₃, —SH, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl;
R² is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl; and
R³ is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

### Embodiment 48

The method of embodiment 47, wherein R² and R³ are hydrogen.

### Embodiment 49

The method of embodiment 47 or 48, wherein L¹ is —(CH₂)_{z1}—NH—(CH₂)_{z2}—, —(CH₂)_{z1}—S—(CH₂)_{z2}—, —(CH₂)_{z1}—O—(CH₂)_{z2}—, —(CH₂)_{z1}—C(O)NH—(CH₂)_{z2}—, —(CH₂)_{z1}—C(O)O—(CH₂)_{z2}—, or —(CH₂)_{z1}—S(O)₂NH(CH₂)_{z2}, wherein z1 and z2 are independently integers from 0 to 10.

### Embodiment 50

The method of embodiment 49, wherein z1 and z2 are independently integers from 0 to 5.

### Embodiment 51

The method of embodiment 49, wherein z1 and z2 are independently integers from 0 to 3.

### Embodiment 52

The method of embodiment 49, wherein z1 and z2 are 1.

### Embodiment 53

The method of embodiment 49, wherein z1 is an integer from 0 to 3, and z2 is 0.

### Embodiment 54

The method of embodiment 49, wherein z1 is 1, and z2 is 0.

### Embodiment 55

The method of embodiment 49, wherein z2 is an integer from 0 to 3, and z1 is 0.

### Embodiment 56

The method of embodiment 49, wherein z1 is 0, and z2 is 1.

### Embodiment 57

The method of any one of embodiments 47-56, wherein R¹ is substituted or unsubstituted cycloalkyl, substituted or unsubstituted heterocycloalkyl, substituted or unsubstituted aryl or substituted or unsubstituted heteroaryl.

### Embodiment 58

The method of embodiment 57, wherein R¹ is substituted or unsubstituted heterocycloalkyl, substituted or unsubstituted aryl or substituted or unsubstituted heteroaryl.

### Embodiment 59

The method of embodiment 47, wherein R¹ is substituted or unsubstituted purinyl, substituted or unsubstituted imidazopyridinyl, substituted or unsubstituted imidazopyridinyl, substituted or unsubstituted benzoimidazolyl, substituted or unsubstituted benzotriazolyl, substituted or unsubstituted indazolyl, substituted or unsubstituted dihydropurinonyl, substituted or unsubstituted pyrazolopyridinyl, substituted or unsubstituted dihydropurinedionyl, substituted or unsubstituted furanyl, substituted or unsubstituted dihydrofuroquiozalinyl, substituted or unsubstituted pyrimidoindolyl, substituted or unsubstituted carbazolyl, substituted or unsubstituted indenyl, substituted or unsubstituted dihydrocyclopenta-isoindolyl, substituted or unsubstituted pyridinyl, substituted or unsubstituted pyrimidinyl, substituted or unsubstituted pyridazinyl, substituted or unsubstituted triazinyl, substituted or unsubstituted quinolinyl, substituted or unsubstituted quinazolinyl, substituted or unsubstituted quinoxalinyl, substituted or unsubstituted pyrrolyl, substituted or unsubstituted imidazolyl, substituted or unsubstituted oxazolyl, substituted or unsubstituted isoxazolyl, substituted or unsubstituted thiazolyl, substituted or unsubstituted isothizolyl, substituted or unsubstituted triazolyl, substituted or unsubstituted oxadiazolyl, substituted or unsubstituted thiadiazolyl, substituted or unsubstituted indolyl, substituted or unsubstituted pyrazolopyridinyl, substituted or unsubstituted benzofuranyl, substituted or unsubstituted benzothiophenyl, substituted or unsubstituted acridinyl, or substituted or unsubstituted phenyl.

### Embodiment 60

The method of embodiment 47, wherein R^{1A} is hydrogen, halogen, —CF₃, —CN, —OH, —NH₂, —N₃, —SH, —COOH, —NHCOOH, —CONH₂, R^{1B}-substituted or unsubstituted alkyl, R^{1B}-substituted or unsubstituted heteroalkyl, R^{1B}-substituted or unsubstituted cycloalkyl, R^{1B}-substituted or unsubstituted heterocycloalkyl, R^{1B}-substituted or unsubstituted aryl or R^{1B}-substituted or unsubstituted heteroaryl; R^{1B} is independently halogen, —CF₃, —CN, —OH, —NH₂, —N₃, —NO₂, —SH, —COOH, —NHCOOH, —CONH₂, R^{1C}-substituted or unsubstituted alkyl, R^{1C}-substituted or unsubstituted heteroalkyl, R^{1C}-substituted or unsubstituted cycloalkyl, R^{1C}-substituted or unsubstituted heterocycloalkyl, R^{1C}-substituted or unsubstituted aryl or R^{1C}-substituted or unsubstituted heteroaryl; and R^{1C} is independently halogen, —CF₃, —CN, —OH, —NH₂, —N₃, —SH, —COOH, —NHCOOH, —CONH₂, unsubstituted alkyl, unsubstituted heteroalkyl, unsubstituted cycloalkyl, unsubstituted heterocycloalkyl, unsubstituted aryl or unsubstituted heteroaryl.

### Embodiment 61

The method of embodiment 60, wherein L¹ is a bond, —O—, —S—, —NH—, —C(O)NH—, —C(O)—, —S(O)₂NH—, R^{1D}-substituted or unsubstituted alkylene, R^{1D}-substituted or unsubstituted heteroalkylene, R^{1D}-substituted or unsubstituted cycloalkylene, R^{1D}-substituted or unsubstituted heterocycloalkylene, R^{1D}-substituted or unsubstituted arylene or R^{1D}-substituted or unsubstituted heteroarylene, wherein R^{1D} is halogen, —CF₃, —CN, —OH, —NH₂, —N₃, —SH, —COOH, —NHCOOH, —CONH₂, R^{1E}-substituted or unsubstituted alkyl, R^{1E}-substituted or unsubstituted heteroalkyl, R^{1E}-substituted or unsubstituted cycloalkyl, R^{1E}-substituted or unsubstituted heterocycloalkyl, R^{1E}-substituted or unsubstituted aryl or R^{1E}-substituted or unsubstituted heteroaryl; and wherein R^{1E} is halogen, —CF₃, —CN, —OH, —NH₂, —N₃, —SH, —COOH, —NHCOOH, —CONH₂, unsubstituted alkyl, unsubstituted heteroalkyl, unsubstituted cycloalkyl, unsubstituted heterocycloalkyl, unsubstituted aryl or unsubstituted heteroaryl.

### Embodiment 62

The method of any one of embodiments 47-61, wherein the compound has the formula:

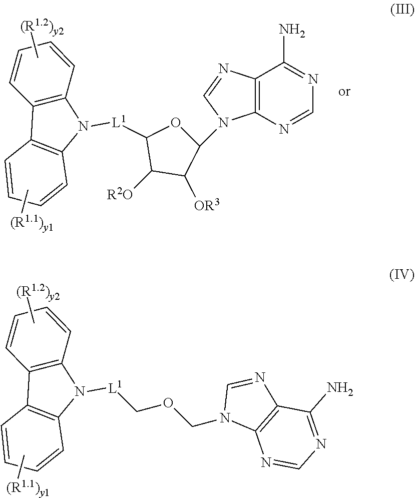

wherein,
y1 and y2 are independently an integer from 0 to 4,

R^{1.1} and R^{1.2} independently are halogen, —CF₃, —CBr₃, —CCl₃, —CI₃,

—CHF₂, —CHBr₂, —CHCl₂, —CHI₂, —CH₂F, —CH₂Br, —CH₂Cl, —CH₂I, —OCF₃, —OCH₃, —OCBr₃, —OCCl₃, —OCI₃, —OCHF₂, —OCHBr₂, —OCHCl₂, —OCHI₂, —OCH₂F, —OCH₂Br, —OCH₂Cl, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

### Embodiment 63

The method of embodiment 62, wherein R² and R³ are hydrogen.

### Embodiment 64

The method of embodiment 62, wherein y1 and y2 are 0.

### Embodiment 65

The method of embodiment 62, wherein y1 and y2 are 1.

### Embodiment 66

The method of embodiment 62, wherein y1 is 0 and y2 is 1.

### Embodiment 67

The method of embodiment 62, wherein y1 is 1 and y2 is 0.

### Embodiment 68

The method of embodiment 62, wherein y1 is 1, y2 is 0, and R^{1.1} is —CH₃ or —CH₂F.

### Embodiment 69

The method of embodiment 62, wherein y1 is 0, y2 is 1, and R^{1.2} is —CH₂F, —CF₃, —CH₃, —OCH₃ or —C(CH₃)₃.

### Embodiment 70

The method of embodiment 62, wherein y1 and y2 are 1.

### Embodiment 71

The method of embodiment 62, wherein R^{1.1} is —CH₂F and R^{1.2} is —C(CH₃)₃; R^{1.1} is —CH₃ and R^{1.2} is —C(CH₃)₃; R^{1.1} is —CH₃ and R^{1.2} is —CF₃; R^{1.1} and R^{1.2} are each —CH₃; R^{1.1} is —CH₃ and R^{1.2} is —CH₂F; R^{1.1} is —CH₃ and R^{1.2} is —OCH₃; R^{1.1} is —CF₃ and R^{1.2} is —OCH₃; R^{1.1} is —CF₃ and R^{1.2} is —CH₃; R^{1.1} is —OCH₃ and R^{1.2} is —CH₃; or R^{1.1} is —OCH₃ and R^{1.2} is —CH₃.

### Embodiment 72

The method of any one of embodiments 62-71, wherein the compound has the formula:

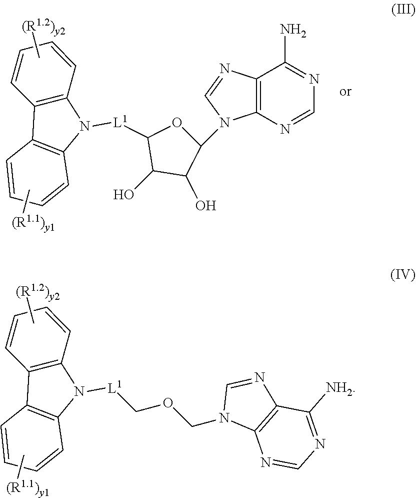

### Embodiment 73

The method of embodiment 72, wherein y1 is 1, y2 is 0, and R^{1.1} is —CH₃ or —CH₂F.

### Embodiment 74

The method of embodiments 72, wherein y1 is 0, y2 is 1, and R^{1.2} is —CH₂F, —CF₃, —CH₃, —OCH₃ or —C(CH₃)₃.

### Embodiment 75

The method of embodiment 72, wherein y1 and y2 are 1.

### Embodiment 76

The method of embodiment 72, wherein R^{1.1} is —CH₂F and R^{1.2} is —C(CH₃)₃; R^{1.1} is —CH₃ and R^{1.2} is —C(CH₃)₃; R^{1.1} is —CH₃ and R^{1.2} is —CF₃; R^{1.1} and R^{1.2} are each —CH₃; R^{1.1} is —CH₃ and R^{1.2} is —CH₂F; R^{1.1} is —CH₃ and R^{1.2} is —OCH₃; R^{1.1} is —CF₃ and R^{1.2} is —OCH₃; R^{1.1} is —CF₃ and R^{1.2} is —CH₃; R^{1.1} is —OCH₃ and R^{1.2} is —CH₃; or R^{1.1} is —OCH₃ and R^{1.2} is —CH₃.

### Embodiment 77

The method of any one of embodiments 47-76, wherein said RNA virus infection is an HIV viral infection, West Nile viral infection, Dengue viral infection, Japanese encephalitis or a Zika viral infection.

### Embodiment 78

A compound of the formula:

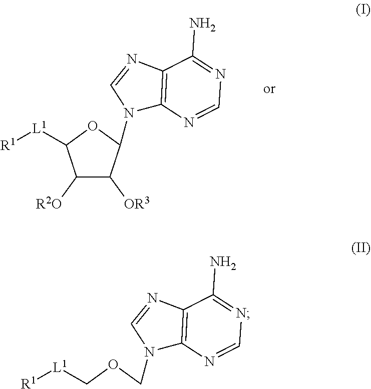

wherein,
L¹ is a bond, —O—, —S—, —NH—, —C(O)NH—, —C(O)—, —S(O)₂NH—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene;
R¹ is hydrogen, halogen, —CF₃, —CN, —OR^{1A}, —NHR^{1A}, —N₃, —SR^{1A}, —COOR^{1A}, —CONHR^{1A}, —NHC(O)R^{1A}, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl;
R^{1A} is hydrogen, halogen, —CF₃, —CN, —OH, —NH₂, —N₃, —SH, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl;
R² is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl; and
R³ is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

### Embodiment 79

The compound of embodiment 78, wherein R² and R³ are hydrogen.

### Embodiment 80

The compound of embodiments 78 or 79, wherein L¹ is —(CH₂)_{z1}—NH—(CH₂)_{z2}—, —(CH₂)_{z1}—S—(CH₂)_{z2}—, —(CH₂)_{z1}—O—(CH₂)_{z2}—, —(CH₂)_{z1}—C(O)NH—(CH₂)_{z2}—, —(CH₂)_{z1}—C(O)O—(CH₂)_{z2}—, or —(CH₂)_{z1}—S(O)₂NH(CH₂)_{z2}, wherein z1 and z2 are independently integers from 0 to 10.

### Embodiment 81

The compound of embodiment 80, wherein z1 and z2 are independently integers from 0 to 5.

### Embodiment 82

The compound of embodiment 80, wherein z1 and z2 are independently integers from 0 to 3.

### Embodiment 83

The compound of embodiment 80, wherein z1 and z2 are 1.

### Embodiment 84

The compound of embodiment 80, wherein z1 is an integer from 0 to 3, and z2 is 0.

### Embodiment 85

The compound of embodiment 80, wherein z1 is 1, and z2 is 0.

### Embodiment 86

The compound of embodiment 80, wherein z2 is an integer from 0 to 3, and z1 is 0.

### Embodiment 87

The compound of embodiment 80, wherein z1 is 0, and z2 is 1.

### Embodiment 88

The compound of any one of embodiments 78-87, wherein R¹ is substituted or unsubstituted cycloalkyl, substituted or unsubstituted heterocycloalkyl, substituted or unsubstituted aryl or substituted or unsubstituted heteroaryl.

### Embodiment 89

The compound of embodiment 88, wherein R¹ is substituted or unsubstituted heterocycloalkyl, substituted or unsubstituted aryl or substituted or unsubstituted heteroaryl.

### Embodiment 90

The compound of embodiment 78, wherein R¹ is substituted or unsubstituted purinyl, substituted or unsubstituted imidazopyridinyl, substituted or unsubstituted imidazopyridinyl, substituted or unsubstituted benzoimidazolyl, substituted or unsubstituted benzotriazolyl, substituted or unsubstituted indazolyl, substituted or unsubstituted dihydropurinonyl, substituted or unsubstituted pyrazolopyridinyl, substituted or unsubstituted dihydropurinedionyl, substituted or unsubstituted furanyl, substituted or unsubstituted dihydrofuroquiozalinyl, substituted or unsubstituted pyrimidoindolyl, substituted or unsubstituted carbazolyl, substituted or unsubstituted indenyl, substituted or unsubstituted dihydrocyclopenta-isoindolyl, substituted or unsubstituted pyridinyl, substituted or unsubstituted pyrimidinyl, substituted or unsubstituted pyridazinyl, substituted or unsubstituted triazinyl, substituted or unsubstituted quinolinyl, substituted or unsubstituted quinazolinyl, substituted or unsubstituted quinoxalinyl, substituted or unsubstituted pyrrolyl, substituted or unsubstituted imidazolyl, substituted or unsubstituted oxazolyl, substituted or unsubstituted isoxazolyl, substituted or unsubstituted thiazolyl, substituted or unsubstituted isothizolyl, substituted or unsubstituted triazolyl, substituted or unsubstituted oxadiazolyl, substituted or unsubstituted thiadiazolyl, substituted or unsubstituted indolyl, substituted or unsubstituted pyrazolopyridinyl, substituted or unsubstituted benzofuranyl, substituted or unsubstituted benzothiophenyl, substituted or unsubstituted acridinyl, or substituted or unsubstituted phenyl.

### Embodiment 91

The compound of one of embodiments 78 to 90, wherein R^{1A} is hydrogen, halogen, —CF₃, —CN, —OH, —NH₂, —N₃, —SH, —COOH, —NHCOOH, —CONH₂, R^{1B}-substituted or unsubstituted alkyl, R^{1B}-substituted or unsubstituted heteroalkyl, R^{1B}-substituted or unsubstituted cycloalkyl, R^{1B}-substituted or unsubstituted heterocycloalkyl, R^{1B}-substituted or unsubstituted aryl or R^{1B}-substituted or unsubstituted heteroaryl; R^{1B} is independently halogen, —CF₃, —CN, —OH, —NH₂, —N₃, —NO₂, —SH, —COOH, —NHCOOH, —CONH₂, R^{1C}-substituted or unsubstituted alkyl, R^{1C}-substituted or unsubstituted heteroalkyl, R^{1C}-substituted or unsubstituted cycloalkyl, R^{1C}-substituted or unsubstituted heterocycloalkyl, R^{1C}-substituted or unsubstituted aryl or R^{1C}-substituted or unsubstituted heteroaryl; and R^{1C} is independently halogen, —CF₃, —CN, —OH, —NH₂, —N₃, —SH, —COOH, —NHCOOH, —CONH₂, unsubstituted alkyl, unsubstituted heteroalkyl, unsubstituted cycloalkyl, unsubstituted heterocycloalkyl, unsubstituted aryl or unsubstituted heteroaryl.

### Embodiment 92

The compound of embodiment 91, wherein L¹ is a bond, —O—, —S—, —NH—, —C(O)NH—, —C(O)—, —S(O)₂NH—, R^{1D}-substituted or unsubstituted alkylene, R^{1D}-substituted or unsubstituted heteroalkylene, R^{1D}-substituted or unsubstituted cycloalkylene, R^{1D}-substituted or unsubstituted heterocycloalkylene, R^{1D}-substituted or unsubstituted arylene or R^{1D}-substituted or unsubstituted heteroarylene, wherein R^{1D} is halogen, —CF₃, —CN, —OH, —NH₂, —N₃, —SH, —COOH, —NHCOOH, —CONH₂, R^{1E}-substituted or unsubstituted alkyl, R^{1E}-substituted or unsubstituted heteroalkyl, R^{1E}-substituted or unsubstituted cycloalkyl, R^{1E}-substituted or unsubstituted heterocycloalkyl, R^{1E}-substituted or unsubstituted aryl or R^{1E}-substituted or unsubstituted heteroaryl; and wherein R^{1E} is halogen, —CF₃, —CN, —OH, —NH₂, —N₃, —SH, —COOH, —NHCOOH, —CONH₂, unsubstituted alkyl, unsubstituted heteroalkyl, unsubstituted cycloalkyl, unsubstituted heterocycloalkyl, unsubstituted aryl or unsubstituted heteroaryl.

### Embodiment 93

The compound of embodiment 78, wherein the compound has the formula:

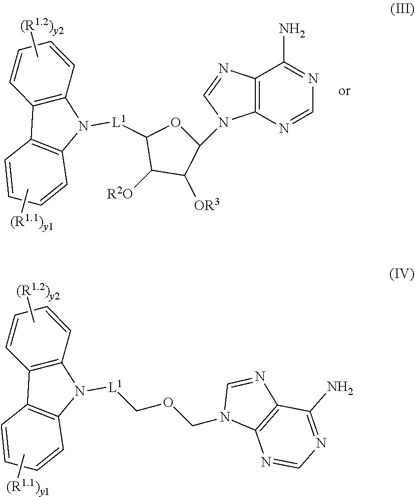

wherein,
y1 and y2 are independently an integer from 0 to 4,
R^{1.1} and R^{1.2} independently are halogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCH₃, —OCBr₃, —OCCl₃, —OCI₃, —OCHF₂, —OCHBr₂, —OCHCl₂, —OCHI₂, —OCH₂F, —OCH₂Br, —OCH₂Cl, —OCH₂I, —CN, —OH, —NH₂, —COO H, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

### Embodiment 94

The compound of embodiment 93, wherein R² and R³ are hydrogen.

### Embodiment 95

The compound of embodiment 93, wherein y1 and y2 are 0.

### Embodiment 96

The compound of embodiment 93, wherein y1 and y2 are 1.

### Embodiment 97

The compound of embodiment 93, wherein y1 is 0 and y2 is 1.

### Embodiment 98

The compound of embodiment 93, wherein y1 is 1 and y2 is 0.

### Embodiment 99

The compound of embodiment 93, wherein y1 is 1, y2 is 0, and R^{1.1} is —CH₃ or —CH₂F.

### Embodiment 100

The compound of embodiment 93, wherein y1 is 0, y2 is 1, and R^{1.2} is —CH₂F, —CF₃, —CH₃, —OCH₃ or —C(CH₃)₃.

### Embodiment 101

The compound of embodiment 93, wherein y1 and y2 are 1.

### Embodiment 102

The compound of embodiment 93, wherein R^{1.1} is —CH₂F and R^{1.2} is —C(CH₃)₃; R^{1.1} is —CH₃ and R^{1.2} is —C(CH₃)₃; R^{1.1} is —CH₃ and R^{1.2} is —CF₃; R^{1.1} and R^{1.2} are each —CH₃; R^{1.1} is —CH₃ and R^{1.2} is —CH₂F; R^{1.1} is —CH₃ and R^{1.2} is —OCH₃; R^{1.1} is —CF₃ and R^{1.2} is —OCH₃; R^{1.1} is —CF₃ and R^{1.2} is —CH₃; R^{1.1} is —OCH₃ and R^{1.2} is —CH₃; or R^{1.1} is —OCH₃ and R^{1.2} is —CH₃.

### Embodiment 103

The compound of embodiments 93-102, wherein the compound has the formula:

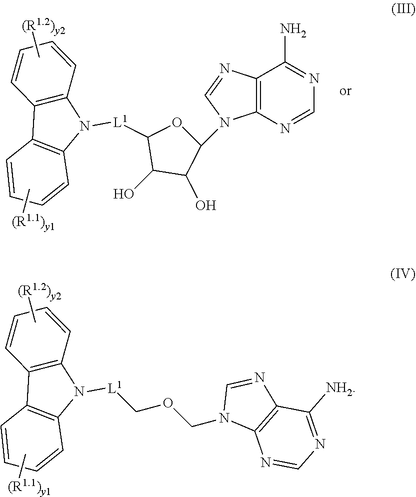

### Embodiment 104

The compound of embodiment 103, wherein y1 is 1, y2 is 0, and R^{1.1} is —CH₃ or —CH₂F.

### Embodiment 105

The compound of embodiment 103, wherein y1 is 0, y2 is 1, and R^{1.2} is —CH₂F, —CF₃, —CH₃, —OCH₃ or —C(CH₃)₃.

### Embodiment 106

The compound of embodiment 103, wherein y1 and y2 are 1.

### Embodiment 107

The compound of embodiment 103, wherein R^{1.1} is —CH₂F and R^{1.2} is —C(CH₃)₃; R^{1.1} is —CH₃ and R^{1.2} is —C(CH₃)₃; R^{1.1} is —CH₃ and R^{1.2} is —CF₃; R^{1.1} and R^{1.2} are each —CH₃; R^{1.1} is —CH₃ and R^{1.2} is —CH₂F; R^{1.1} is —CH₃ and R^{1.2} is —OCH₃; R^{1.1} is —CF₃ and R^{1.2} is —OCH₃; R^{1.1} is —CF₃ and R^{1.2} is —CH₃; R^{1.1} is —OCH₃ and R^{1.2} is —CH₃; or R^{1.1} is —OCH₃ and R^{1.2} is —CH₃.

### Embodiment 108

A pharmaceutical composition comprising the compound of any one of embodiments 78-107 and a pharmaceutically acceptable excipient.

### Embodiment 109

A vaccine composition comprising the compound of any one of embodiments 78-107, a vaccine adjuvant, and an immunogenic agent.

### Embodiment 110

A pharmaceutical composition comprising an RNA compound, said RNA compound encoding an RNA virus structural protein.

### Embodiment 111

The pharmaceutical composition of embodiment 110, wherein said RNA virus structural protein is a Zika virus structural protein.

### Embodiment 112

The pharmaceutical composition of embodiments 110 or 111, wherein said RNA virus structural protein is a viral premembrane protein (prM), viral envelope protein (Env), a capsid protein (C) or a membrane protein (M).

### Embodiment 113

The pharmaceutical composition of any one of embodiment 110-111, wherein said RNA compound is modified on at least one 2′ position with a 2′O-methyl and/or modified on at least one adenosine at the N6 adenine position with a methyl.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### EXAMPLES

### Example 1. Dynamics of Human and Viral RNA Methylation During ZIKA Virus Infection

Infection with the flavivirus ZIKA (ZIKV) causes neurological, immunological, and developmental defects through incompletely understood mechanisms. We report that ZIKV infection affects viral and human RNAs by altering the topology and function of N⁶-adenosine methylation (m⁶A), a modification affecting RNA structure and function. m⁶A nucleosides are abundant in ZIKV RNA, with 12 m⁶A peaks identified across full length ZIKV RNA. m⁶A in ZIKV RNA is controlled by host methyltransferases METTL3 and METTL14 and demethylases ALKBH5 and FTO, and knockdown of METTL3 or METTL14 increases, while silencing ALKBH5 and FTO decreases ZIKV production. YTHDF family proteins, which regulate the stability of m⁶A-modified RNA, bind to ZIKV RNA and their silencing increases ZIKV replication. Profiling of the m⁶A methylome of host mRNAs reveals that ZIKV infection alters m⁶A location in mRNAs, methylation motifs, and target genes modified by methyltransferases. Our results identify a mechanism by which ZIKV interacts with and alters host cell functions.

Introduction.

Human infection with ZIKA virus (ZIKV), a mosquito-borne flavivirus, has spread rapidly since the 2015 outbreak in Brazil, and the World Health Organization declared ZIKV infection an International Public Health Emergency in 2016(Fauci and Morens, 2016; Heymann et al., 2016; Petersen et al., 2016). ZIKV was discovered in 1947 (Driggers et al., 2016a) and, although it had previously caused only sporadic disease in Africa and Asia, more recent outbreaks occurred in Micronesia in 2007 and in French Polynesia in 2013(Broutet et al., 2016). ZIKV infection has been identified as the etiological agent of severe neurological defects, including microcephaly during fetal development (Driggers et al., 2016b) and neuronal injury associated with Guillain-Barré syndrome in adults (Dejnirattisai et al., 2016). New modes of viral transmission, including maternal-fetal (Brasil et al., 2016) and sexual transmission (Hills et al., 2016), have been reported. ZIKV can infect human skin explants, peripheral blood mononuclear cells, human neuroprogenitor cells, and human cerebral organoids (Dang et al., 2016a; Hamel et al., 2015; Tang et al., 2016). In mouse models, ZIKV may be neurotropic (Cugola et al., 2016; Lazear et al., 2016; Li et al., 2016; Mlakar et al., 2016; Sarno et al., 2016).

ZIKV and other members of the Flaviviridae family, such as dengue (DENV), West Nile (WNV), yellow fever (YFV), and Japanese encephalopathy (JEV), are positive (+) single-stranded RNA viruses. The ZIKV genome encodes a single polyprotein precursor that is cleaved by viral and host proteases to produce three structural and seven nonstructural proteins. Although our understanding of the molecular mechanisms involved in ZIKV infection of human cells has increased dramatically in the past few years, key determinants of ZIKV pathogenicity, such as cell-type specificity, mode of entry, and host factors essential for replication, are still largely unknown. In particular, there is a large gap in our understanding of the genetic and epigenetic regulatory mechanisms governing the viral life cycle and viral interactions with host cells.

As is the case with proteins and DNA, chemical modification of RNA affects its metabolism, function, and localization. More than 100 diverse chemical modifications of RNA nucleotides have been identified, most of which affect ribosomal and transfer RNAs. Modifications of mRNAs and long noncoding RNAs include the 5′-cap structure, N6-methylation of adenosine (m6A), and methylation of C₅ of cytosine (m5C) (Fu et al., 2014; Squires et al., 2012; Yi and Pan, 2011). m6A is the most prevalent internal modification of eukaryotic mRNA with unique distribution patterns (Dominissini et al., 2012; Meyer et al., 2012; Schwartz et al., 2014). While it is becoming increasingly clear that m6A plays an important regulatory role in physiological and pathological processes (Frayling et al., 2007; Jia et al., 2011; Zheng et al., 2013), little is known of the function of m⁶′A in the mammalian immune system or its influence on host-pathogen interactions.

Adenosine methylation is catalyzed by a large RNA methyltransferase complex (MTase), composed of two catalytic subunits (METTL3 and METTL14), a splicing factor (WTAP), a protein (KIAA1429), and other subunits not yet identified (Bokar et al., 1997; Liu et al., 2014; Ping et al., 2014; Schwartz et al., 2014), while removal of methyl groups is catalyzed by two RNA demethylases, FTO and ALKBH5 (Jia et al., 2011; Zheng et al., 2013). m6A is most abundant in translation start sites, stop codons, and 3′-UTRs (Dominissini et al., 2012; Meyer et al., 2012; Schwartz et al., 2014), suggesting that it plays important roles in mRNA biology. Indeed, m6A has been shown to contribute to mRNA stability (Geula et al., 2015; Wang et al., 2014a; Xu et al., 2014); RNA structure, with subsequent effects on RNA-protein interactions (Liu et al., 2015); translation (Meyer et al., 2015; Wang et al., 2015); mRNA nuclear export (Zheng et al., 2013); exon splicing (Zhao et al., 2014) by promoting binding of splicing factor SRSF2 (Zhao et al., 2014); circadian gene expression upon METTL3 depletion (Fustin et al., 2013); and embryonic stem cell pluripotency upon modulation of either METTL3 (Batista et al., 2014; Geula et al., 2015) or METTL14 (Wang et al., 2014b) expression. These findings substantiate the notion that RNA methylation is an epitranscriptomic mechanism that regulates gene expression. The precise sites and abundance of m6A are highly regulated under normal conditions, and it might be expected that non-physiological conditions such as stress, inflammation, and infection would perturb m6A homeostasis.

Although the m6A modification was identified in viral RNA several decades ago (e.g., Rous sarcoma virus, (Kane and Beemon, 1985) influenza virus (Krug et al., 1976) SV40 virus, (Finkel and Groner, 1983), its function and relevance to viral replication had remained unclear. Similarly, whether and how viral infections influence the dynamics of the host and viral RNA methylomes was relatively unknown until the recent publication of three reports describing m6A modification of HIV-1 RNA and the mechanisms by which it affects viral gene expression (Kennedy et al., 2016; Lichinchi et al., 2016; Tirumuru et al., 2016). These studies highlight the significance of this post-transcriptional RNA modification in virology.

The epitranscriptomic landscape of flaviviruses, including ZIKV, remains largely unexplored. N-7 and 2′-O ribose methylations (2′-O-Me) in the cap structure by the viral NS5 protein are required for the efficient translation of viral proteins and for evasion from host antiviral responses. NS5 mutation and loss of N7-methylation are lethal for WNV (Kroschewski et al., 2008; Zhang et al., 2008), and defects in 2′-O-Me dramatically decrease WNV fitness due to enhanced restriction by the host factor IFIT (Daffis et al., 2010). 2′-O-Me of internal adenosines have been detected in DENV and WNV RNA, suggesting a further layer of regulation (Dong et al., 2012), but to date, there have been no reports of internal m6A modifications in flaviviral RNA. Here, we investigated the topology and function of the m6A modification of viral and host RNA during ZIKV infection.

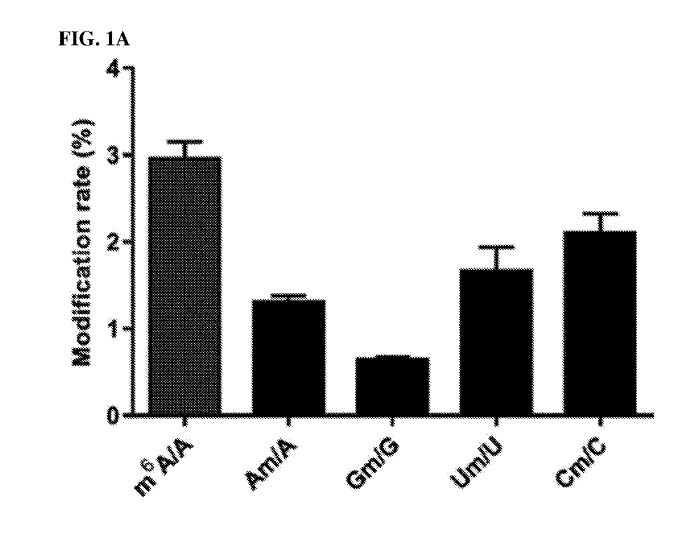

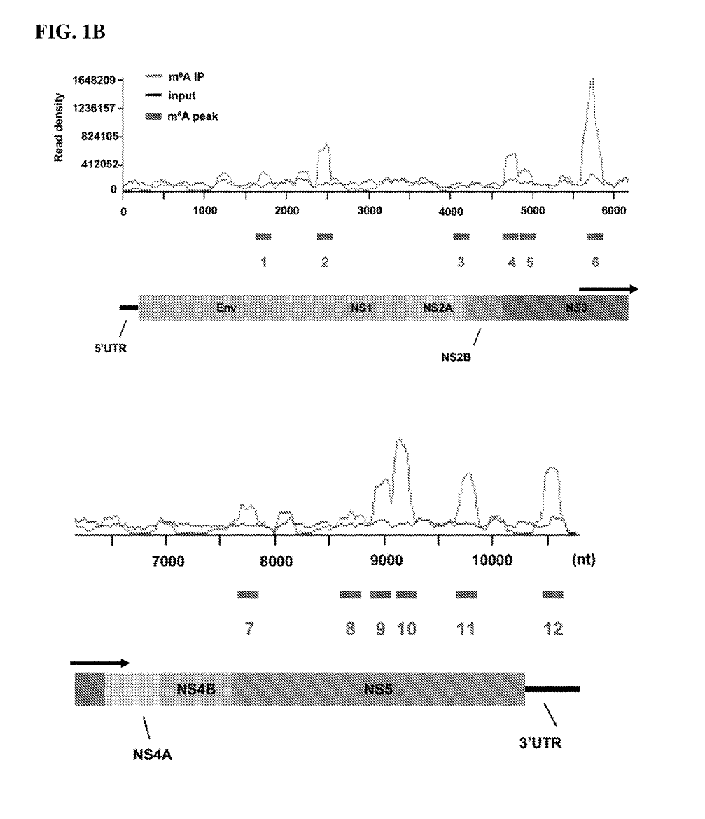

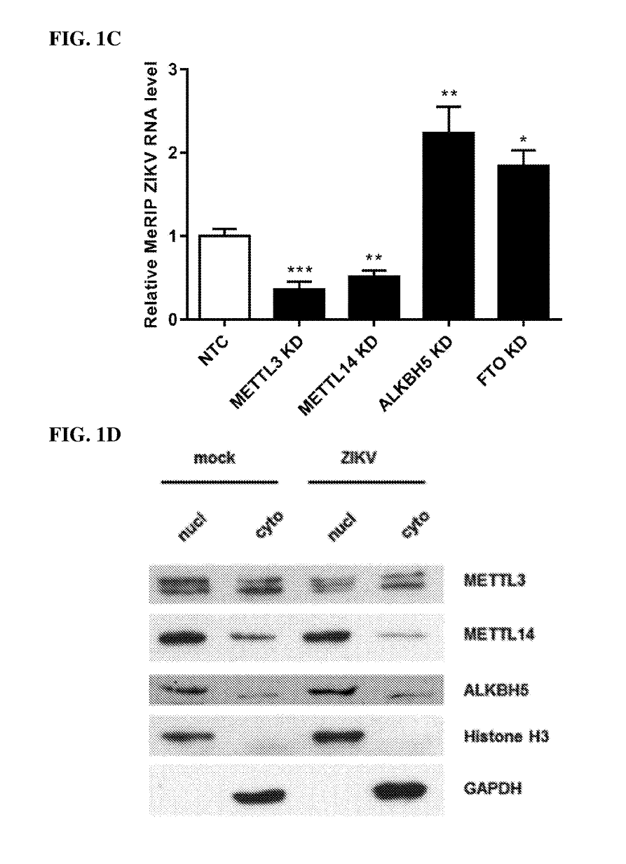

To investigate whether ZIKV viral RNA is methylated, human HEK293T cells were infected with MR766 ZIKV for 48 hours and viral RNA was purified from virions released into the supernatant. The presence of internal m⁶A and 2′-O-Me nucleosides (m⁶A, Am, Cm, Um, and Gm) in ZIKV RNA was quantified by LC-MS/MS according to published methods (Jia et al., 2011). Interestingly, ZIKV RNA contained a high level of methylation analyzed (FIG. 1A). All four nucleotides contained 2′-O-Me groups, with U and C showing the most extensive modification, followed by A and G (FIG. 1A). In particular, the abundance of m6A (about 3% of total adenosine) was strikingly high compared with mammalian mRNAs, where m6A accounts for only ˜0.4-0.5% of adenosines (Liu et al., 2014).

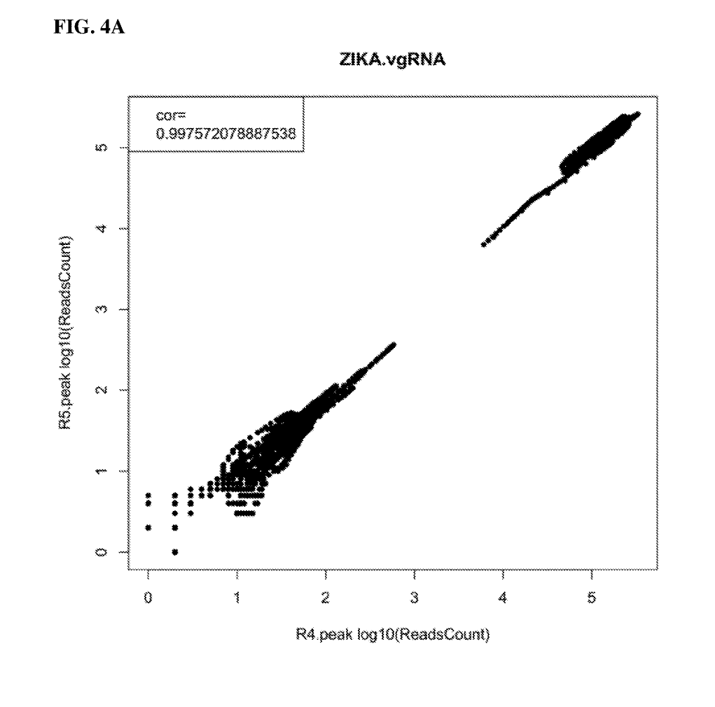

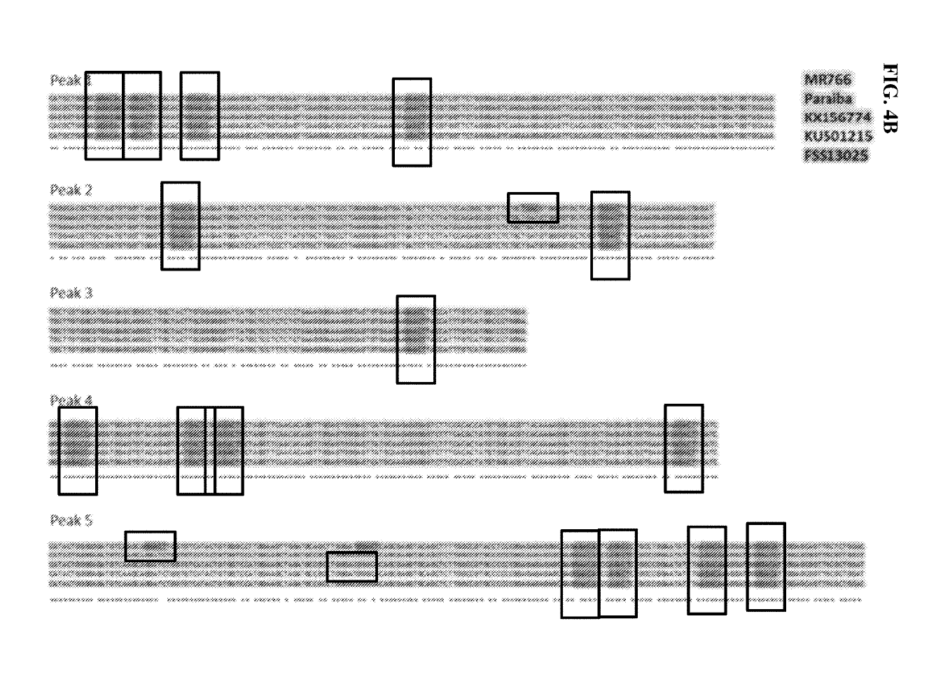

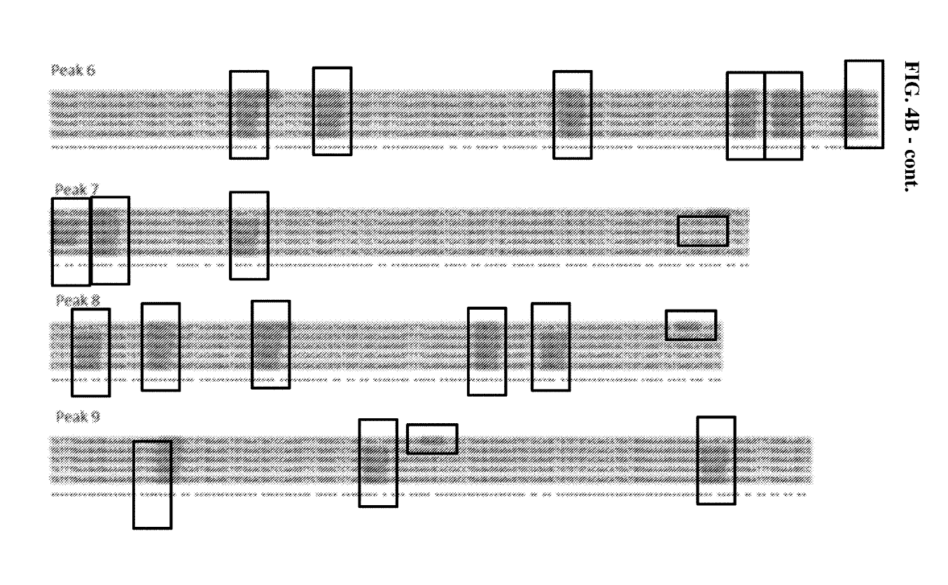

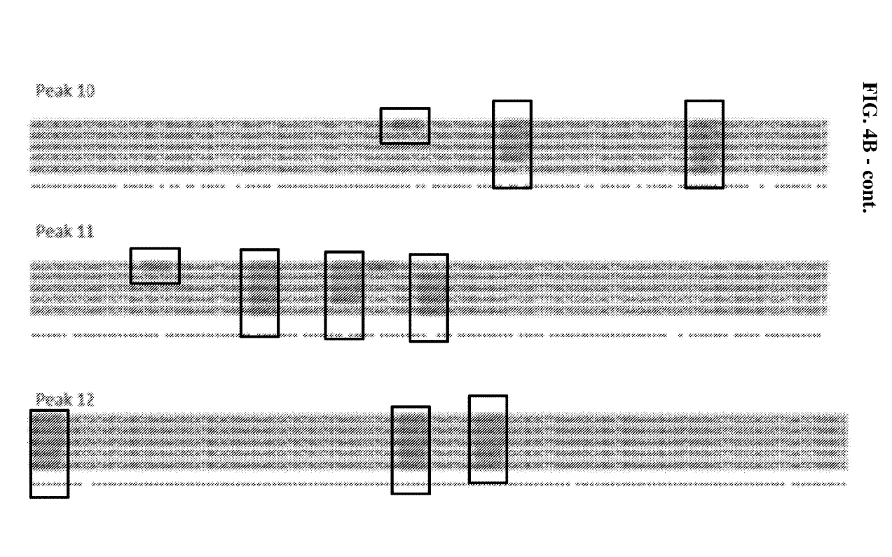

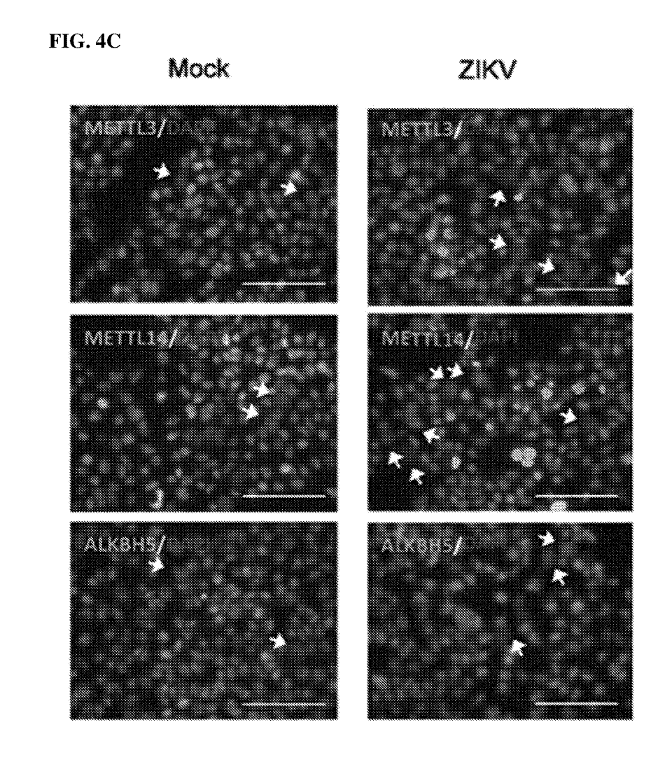

We next examined the topology of the m⁶A RNA ZIKV methylome by performing methylated RNA immunoprecipitation-sequencing (MeRIP-seq) experiments. For this, ZIKV RNA was purified from Vero cell supernatants, fragmented into 60-200 nucleotide lengths and immunoprecipitated with an m⁶A-specific antibody (see experimental procedures described further herein). The associated RNA was then sequenced and reads were mapped to identify the regions of the ZIKV genome enriched in m⁶A. We identified 12 discrete m⁶A peaks spanning the full length of ZIKV RNA (FIG. 1B, Table 1, and FIGS. 4A and 4B). We used a stringent peak calling method (FDR<0.01) and sequenced the methylome of ZIKV with very high depth leading to the identification of statistically significant peaks. The p values for all identified peaks were <1E-30, which includes peak 3 shown in FIG. 1B. Additionally, correlation test between two biological replicates of vgRNA showed a high correlation (0.998), further indicating a high replicability of our sequencing results (FIG. 4A). Of particular note, a cluster of m⁶A peaks was observed in the NS5 coding region and one peak was present in the 3′-UTR region.

TABLE 1
Nucleotide locations and log₂ (enrichment) of the 12 m⁶A peaks identified
in ZIKV RNA by m⁶A-seq (see FIG. 1B).

| m⁶A peak number | start | end | log₂ (enrichment) |
|---|---|---|---|
| 1 | 1652 | 1824 | 1.39 |
| 2 | 2379 | 2531 | 2.52 |
| 3 | 4041 | 4150 | 1.01 |
| 4 | 4658 | 4811 | 1.70 |
| 5 | 4812 | 4999 | 1.11 |
| 6 | 5625 | 5810 | 2.69 |
| 7 | 7673 | 7828 | 1.50 |
| 8 | 8651 | 8800 | 0.98 |
| 9 | 8904 | 9073 | 1.97 |
| 10 | 9080 | 9234 | 3.08 |
| 11 | 9696 | 9849 | 2.32 |
| 12 | 10465 | 10623 | 2.18 |

To determine if m⁶A sites were conserved among recent strains of ZIKV, we performed m⁶A consensus motif search within the 12 m⁶A peaks identified in the MR766 strain and compared these results to four recent ZIKV strains including a Brazilian strain from Paraiba, KX156774 from Panama, KU501215 from Puerto Rico, and FSS13025 from Cambodia. Three common m⁶A motifs, DRACH, MGACK, and UGAC, were analyzed in this analysis (FIG. 4B). Notably, with a few minor exceptions, the majority of the consensus m⁶A sequences found in MR766 share identity among the other viral strains, suggesting that the m⁶A landscape of ZIKV RNA is potentially conserved and not dependent on the particular strain under our investigation (FIG. 4B).

To date, there have been no reports of flaviviral enzymes with internal m⁶A MTase activity, and it is possible that ZIKV RNA adenosines are modified by host MTases and demethylases. To investigate this, we transduced 293T cells with shRNAs to knock down the MTases METTL3 METTL14 and the demethylases ALKBH5 and FTO and then examined m⁶A abundance in ZIKV RNA by MeRIP followed by RT-qPCR. The density of m⁶A on viral RNA was decreased by silencing of METTL3 and METTL14 and increased by depletion of ALKBH5 and FTO, compared with the abundance in cells expressing the control shRNA (FIG. 1C). These results provide evidence that deposition and removal of m⁶A on ZIKV RNA is mediated by host enzymes.

Since the replication of positive single-stranded flaviviruses takes place in the cytoplasm (Hamel et al., 2015), we examined the subcellular localization of METTL3, METTL14, and ALKBH5 proteins in mock- and ZIKV-infected 293T cells. Although METTL14 and ALKBH5 were more abundant in the nucleus, all three enzymes were readily detectable in both the nuclear and cytoplasmic fractions (FIG. 1D). Furthermore, there was no apparent redistribution of the enzymes upon ZIKV infection (FIGS. 1D and 4A-4C). The presence of METTL3, METTL14, and ALKBH5 in the cytoplasm was confirmed by immunofluorescence staining of uninfected and ZIKV-infected cells (FIG. 4C). Together, these results show that methylation and demethylation of viral RNA adenosine occurs in the cytoplasm of the host cell.

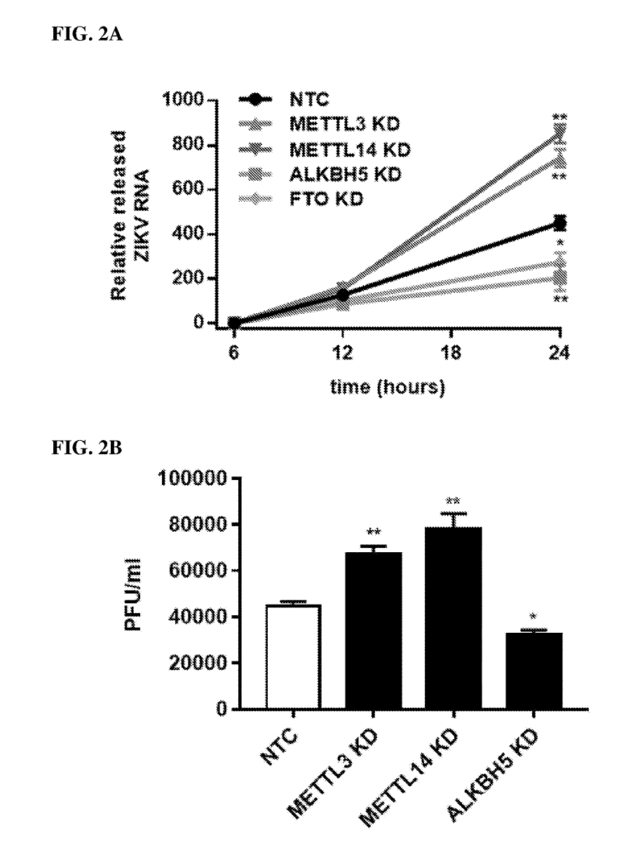

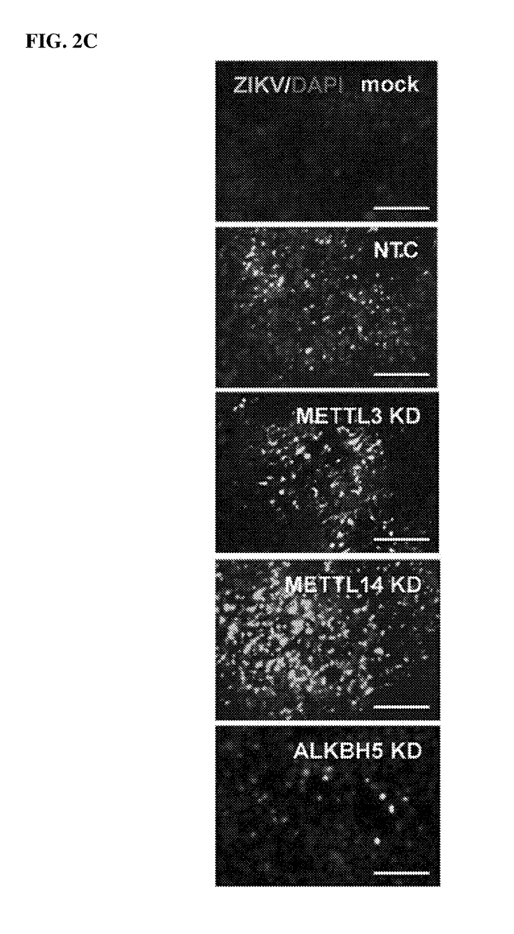

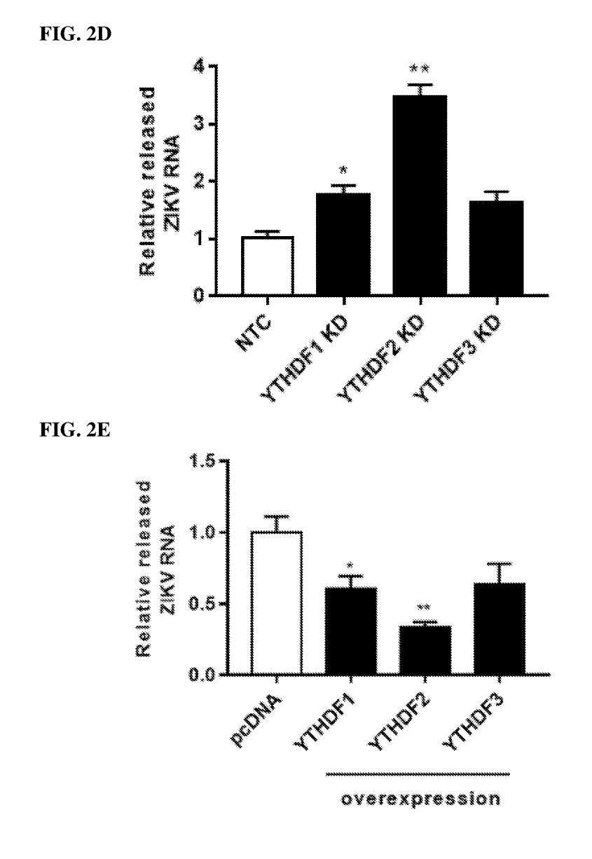

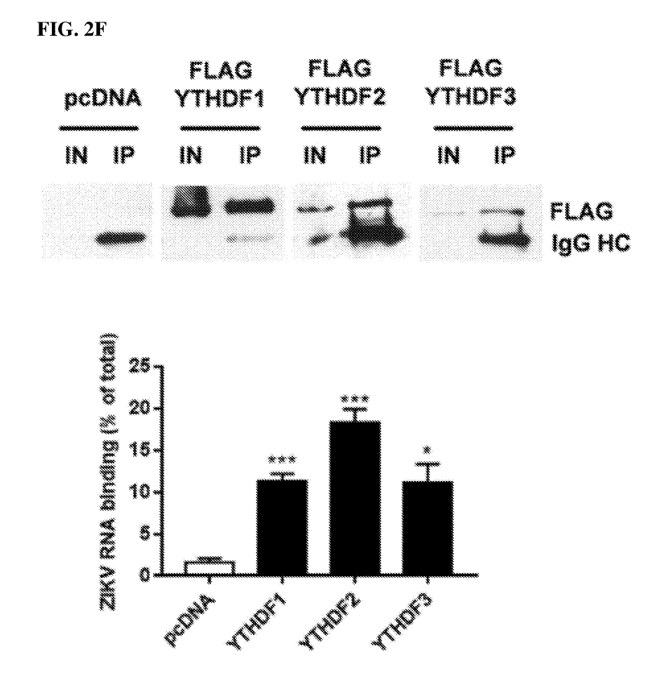

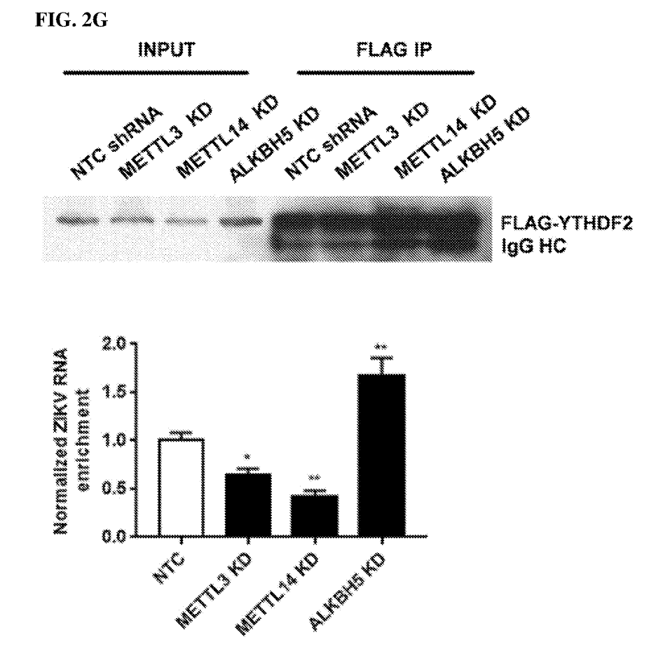

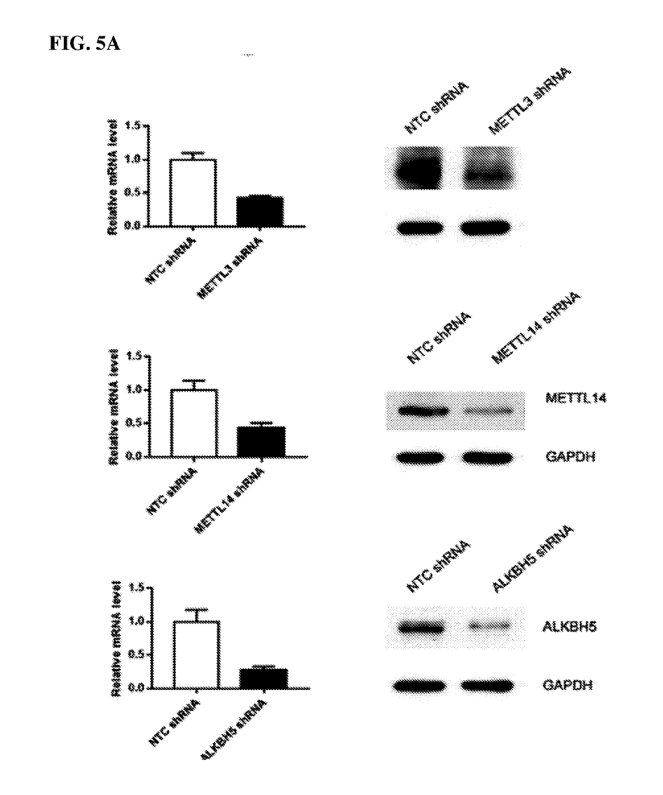

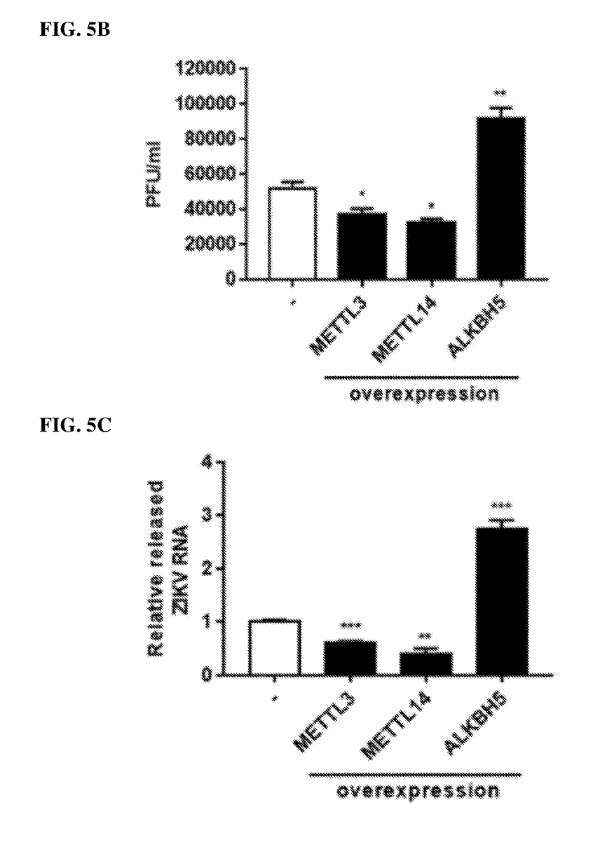

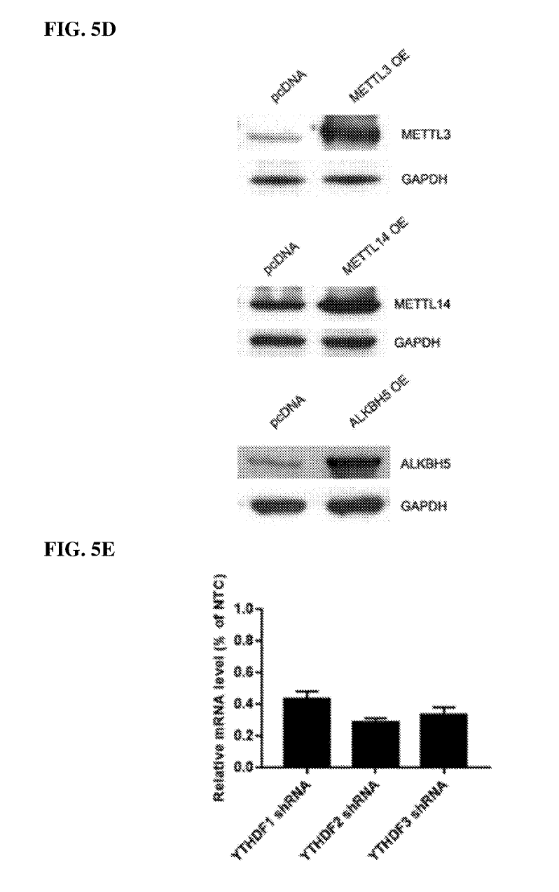

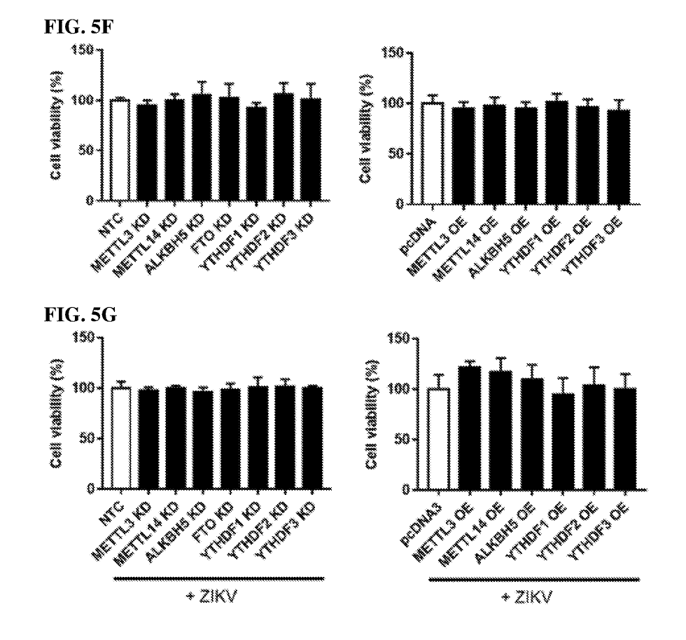

To evaluate whether perturbation of ZIKV m⁶A directly or indirectly affects ZIKV replication efficiency, we transduced 293T cells with METTL3, METTL14, ALKBH5, FTO, or control shRNAs. We found that the viral titer, ZIKV RNA levels in cell supernatants, and expression of ZIKV envelope protein were significantly increased by METTL3 and METTL14 knockdown and decreased by ALKBH5 knockdown, respectively (FIGS. 2A, 2B, and 2C). We confirmed the effects of METTL3, METTL14, and ALKBH5 on the ZIKV life cycle by overexpressing each protein and examining viral replication. Consistent with the results of the knockdown experiments, we found that the viral titer was decreased by METTL3 and METTL14 overexpression and increased by ALKBH5 overexpression without affecting the cell viability (FIGS. 5B-5D, 5F-5G). Thus, modulation of the m⁶A RNA landscape by host enzymes profoundly influences viral replication.

Members of the YTH domain protein family (YTHDF) bind to methylated RNA and regulate the stability (Wang et al., 2014a) and translation (Meyer et al., 2015; Wang et al., 2015; Zhou et al., 2015) of cellular m6A-modified transcripts. Recent studies have also shown that YTHDF proteins bind to HIV-1 RNA and regulate gene expression (Kennedy et al., 2016; Tirumuru et al., 2016). Therefore, we analyzed the effect of YTHDF1-3 silencing by RNAi on ZIKV replication. We observed an increase in ZIKV replication when these proteins were depleted (FIG. 2D and FIG. 5E). Interestingly, YTHDF2 knockdown resulted in the largest increase in ZIKV replication as compared to YTHDF1 and 3. Next, we asked whether YTHDF1, 2, and 3 proteins can bind to ZIKV RNA and, if so, whether they affect RNA metabolism and viral replication. For this, we overexpressed FLAG-tagged YTHDF1-3 proteins in 293T cells and then examined viral RNA abundance and YTHDF protein binding to methylated RNA. Overexpression of all three YTHDF proteins decreased ZIKV RNA expression, with YTHDF2 having the greatest effect (FIG. 2E). Furthermore, immunoprecipitation of the YTHDF proteins with anti-FLAG antibodies followed by RT-qPCR revealed that YTHDF1-3 proteins did indeed bind to ZIKV RNA, with YTHDF2 immunoprecipitates containing the greatest amount of viral RNA (FIG. 2F).

The results of the YTHDF knockdown and overexpression experiments (FIGS. 2D and 2E) suggest that host cell YTHDF2 may bind to and destabilize viral RNA (Wang et al., 2014a), possibly as a mechanism to regulate ZIKV infection. To determine whether modulation of m⁶A abundance on ZIKV RNA affects its binding to YTHDF2, we examined cells expressing control, METTL3, METTL14, or ALKBH5 shRNA. Notably, the association of ZIKV RNA with YTHDF2 was significantly reduced by METTL3 and METTL14 silencing, and conversely, significantly increased by ALKBH5 knockdown (FIG. 2G). Thus, taking all these results together, YTHDF2 modulates ZIKV RNA levels by directly binding to m⁶A-modified viral RNA.

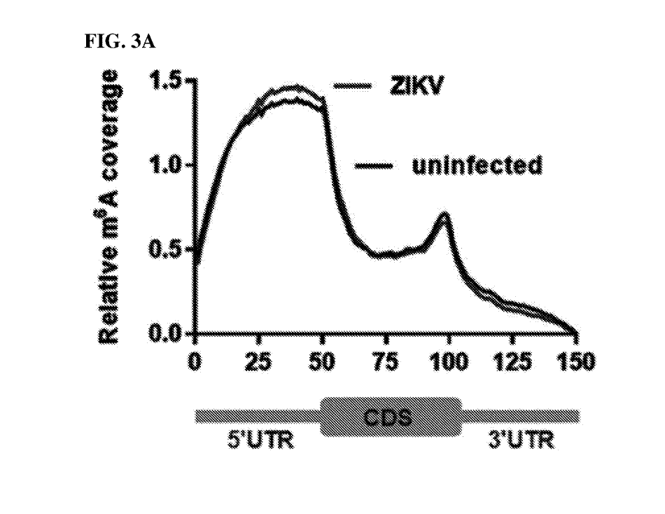

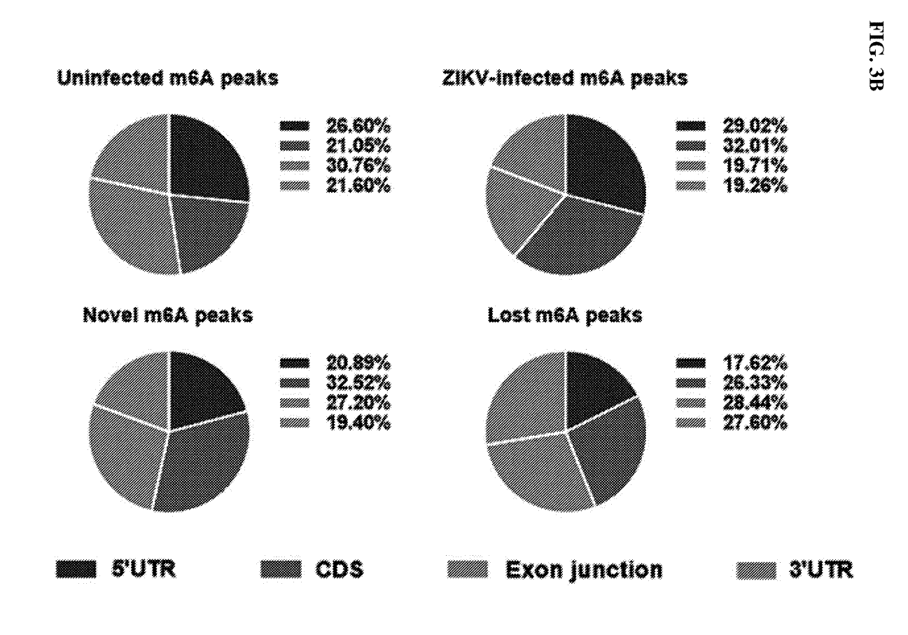

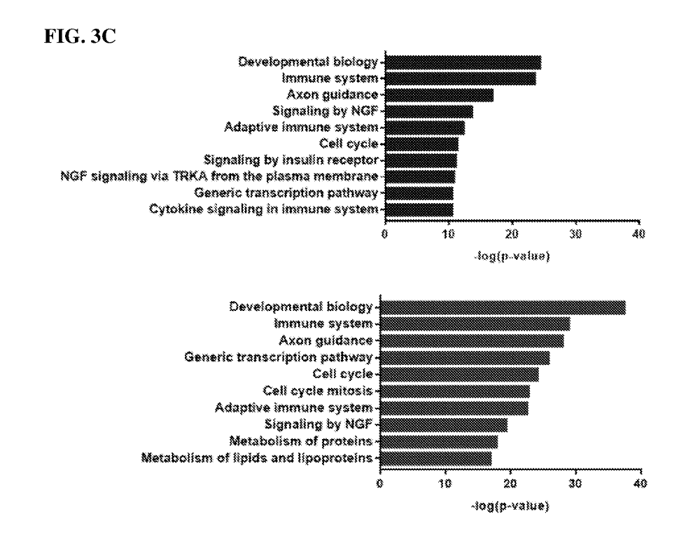

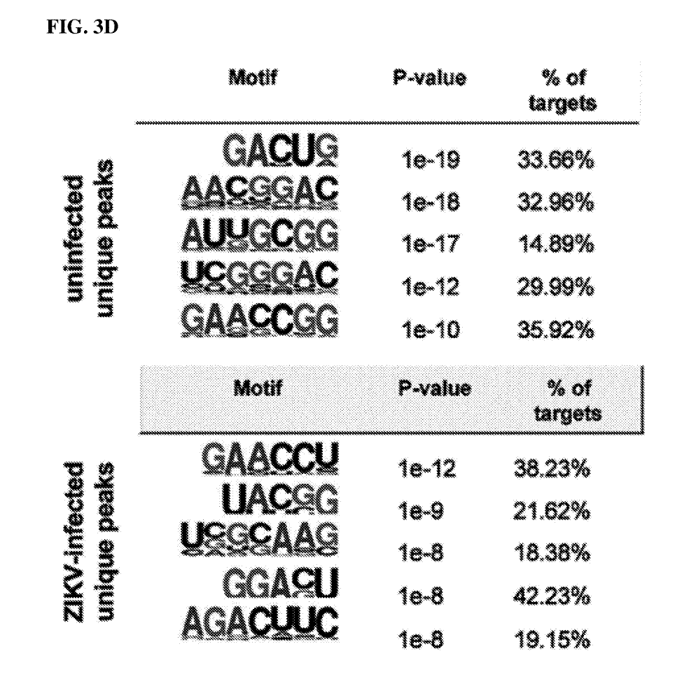

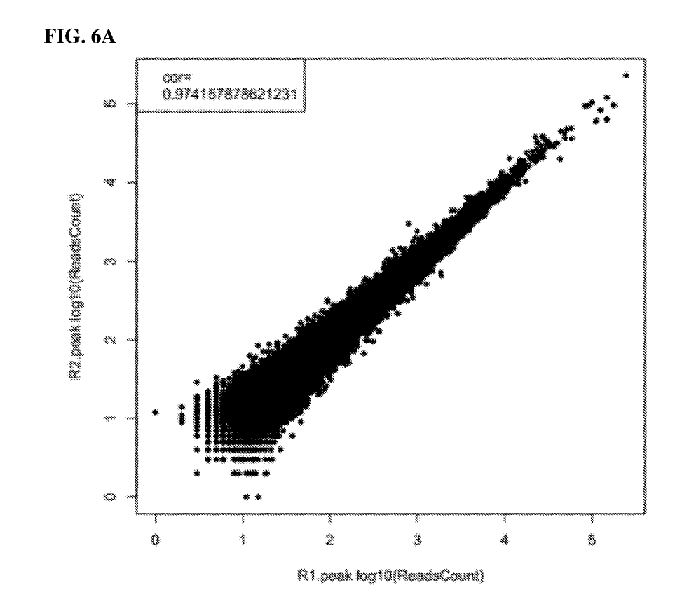

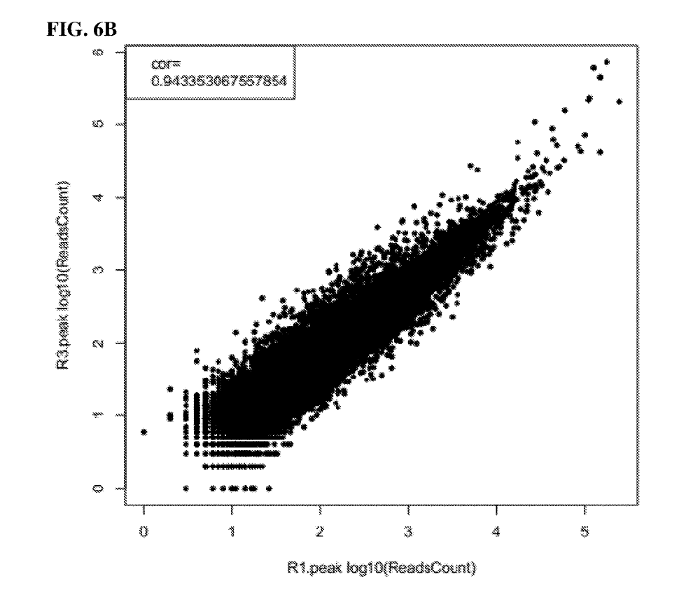

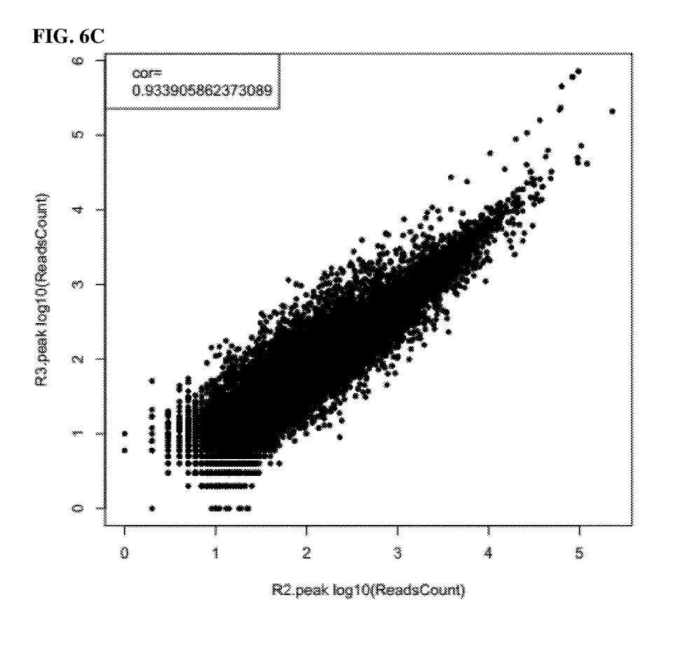

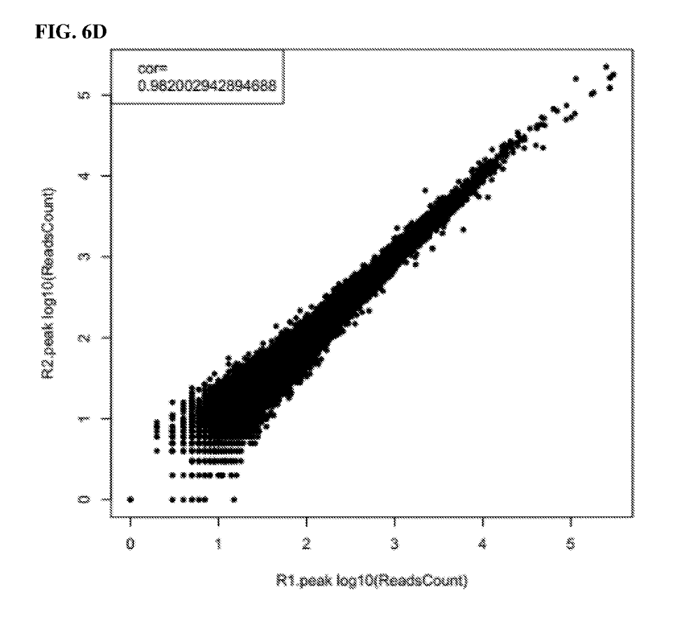

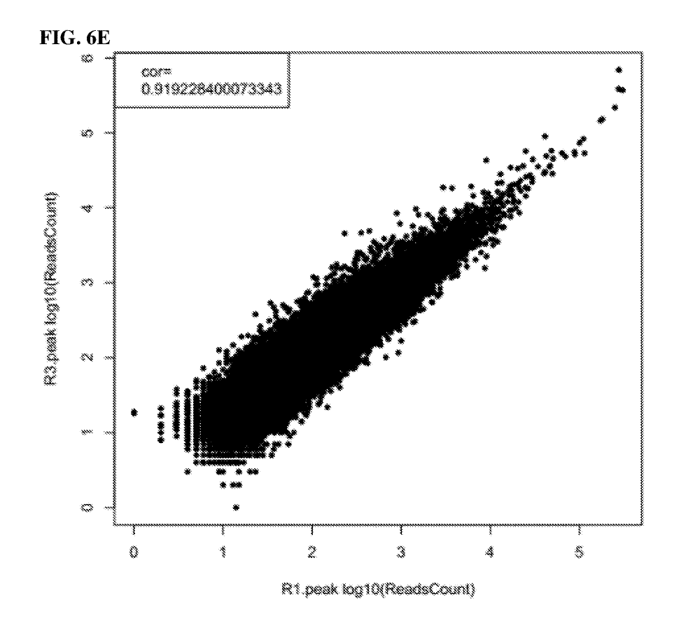

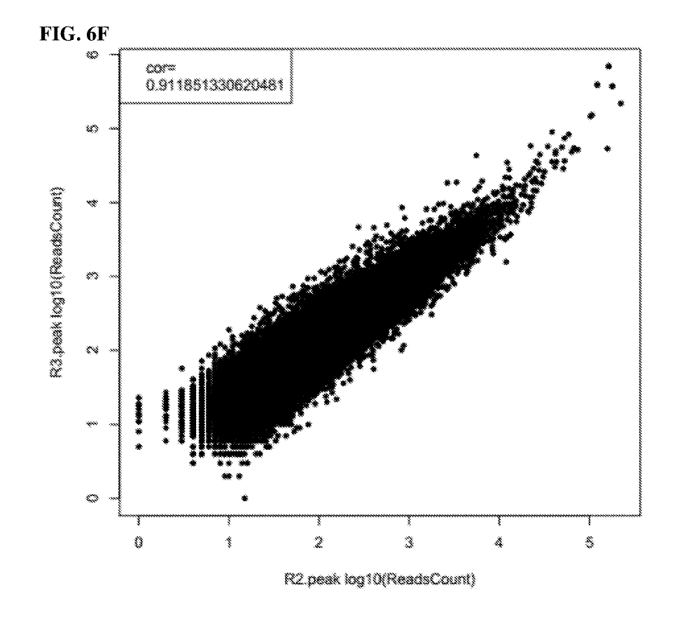

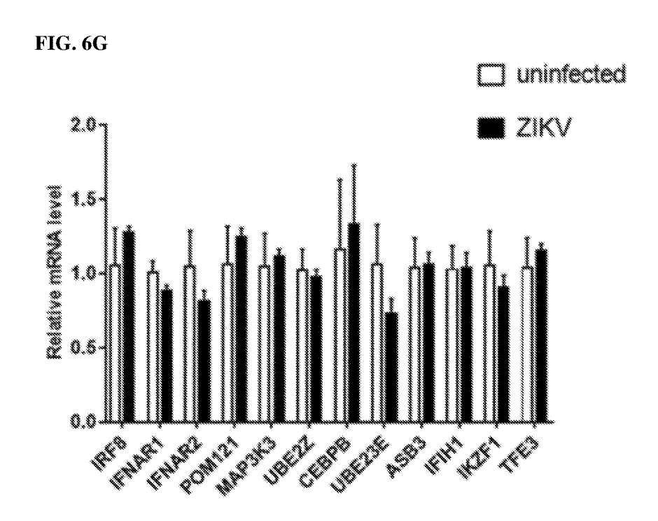

Since viral infection has a profound effect on gene and protein expression in the host cell, we examined the abundance and distribution of m⁶A on cellular transcripts by performing m⁶A-seq experiments on mRNA isolated from uninfected and ZIKV-infected 293T cells (FIGS. 6A-6B). Metagene analysis showed that ZIKV infection increased m⁶A levels in the 5′-UTR regions of the transcriptome and correspondingly decreased the levels in the 3′-UTRs (FIG. 3A). This change may represent a cellular response to the stress of viral infection, similar to the previously described change in m⁶A deposition in response to heat shock (Zhou et al., 2015). To examine the distribution of ZIKV-induced changes in more detail, we compared mRNA m⁶A peaks that were either newly gained or lost after ZIKV infection. We observed that the newly emerged m⁶A modifications were preferentially deposited in 5′-UTR and CDS and depleted in exon junctions and 3′-UTR regions, compared to the lost m⁶A peaks (FIG. 3B), suggesting that ZIKV infection might affect gene translation, alternative splicing, and mRNA stability as a consequence of differential deposition of m6A. Gene ontology (GO) analysis of the genes with changed m⁶A peaks identified a number of immune-related categories among the most enriched pathways in both the newly gained and lost m⁶A modifications (FIG. 3C). Furthermore, quantitative RNA expression analysis of a dozen representative genes showed that the newly methylated genes emerged upon infection had expression levels similar to uninfected conditions suggesting that the selective m⁶A deposition was not due to an RNA upregulation but rather a consequence of viral infection events (FIG. 6G). Thus, ZIKV infection appears to be sensed by the host MTase and demethylase machineries, which then initiate a directed reprogramming of the post-transcriptional landscape of cellular mRNAs (Table 2).

TABLE 2
ZIKV-enriched m2A genes - Immunity related

| | New m6A peak |
|---|---|
| Gene | location |
| CREBBP | CDS |
| NCAM1 | CDS |
| CLTC | CDS |
| KPNA2 | CDS |
| IRF2 | CDS |
| PML | CDS |
| IRF8 | 3′_UTR_junction |
| IFNAR2 | 3′_UTR |
| IFNAR1 | 3′_UTR |
| POM121 | 3′_UTR |
| SH2B1 | CDS |
| IRS1 | CDS |
| MAP3K3 | 3′_UTR |
| KIF3C | CDS |
| HUWE1 | CDS |
| UBE2E3 | 5′_UTR_junction |
| UBE2Z | 3′_UTR |
| TRIM9 | 5′_UTR |
| UBR2 | CDS |
| ASB3 | 5′_UTR |
| DZIP3 | CDS |
| TAP2 | CDS |
| AP1M1 | CDS |
| DYNC1LI2 | CDS |
| RAP1GAP2 | CDS |
| IFIH1 | 5′_UTR |
| APP | CDS |
| C4A | CDS |
| CEBPB | 3′_UTR_junction |
| IKZF1 | 5′_UTR |
| TFE3 | 5′_UTR_junction |
| CACTIN | CDS |
| WNT5A | CDS |
| CSF1 | CDS |
| ZBTB46 | CDS |
| BMP4 | CDS |
| PARP1 | CDS |
| C4B | CDS |
| SUSD4 | CDS |

We have performed a motif analysis of the newly emerged novel and lost peaks specifically to determine if there was any change in the top motifs between these two groups. The motif usage changes seem to occur on the overall level, with newly emerged novel ones preferring “AAC” while lost ones preferring “GAC” (FIG. 3D). The results showed clear differences in the consensus sequences of m⁶A, highlighting the possibility that the substrate specificity of m⁶A-related enzymes may change upon viral infection (FIG. 3D).

Epitranscriptomics has recently been identified as a new layer of regulation of RNA metabolism and function. Several aspects of RNA biology have been shown to be controlled by m⁶A deposition by METTL3/METTL14 MTases and removal by ALKBH5 and FTO demethylases. Here, we quantified and mapped the internal m⁶A modifications in ZIKV RNA and showed that the m⁶A abundance is controlled by host METTL3, METTL14, ALKBH5, and FTO. Subsequently, we investigated the role of m⁶A during ZIKV infection of human cells. We found that the depletion or over-expression of the central RNA methylation enzymes impact viral replication, demonstrating that the host RNA methyltransferase machinery acts as a negative post-transcriptional regulator of ZIKV virus. Moreover, ZIKV RNA binding to YTHDF proteins indicates another regulatory layer represented by the m⁶A binding proteins. We also explored the m⁶A landscape of cellular mRNAs in response to ZIKV infection, and identified two classes of transcripts which undergo specific m⁶A deposition or loss of the modification. This evidence suggests that RNA methylation machineries (“writers”, “erasers” and finally “readers”) are able to sense and respond to viral infection and modulate gene expression at the post-transcriptional level. The overall effects of m⁶A on viral replication may be due to a combination of direct events regulating viral RNA metabolism (i.e. YTHDF proteins' binding to ZIKV RNA) and indirect post-transcriptional regulation of host RNAs, which may act as pro- or anti-viral factors. For example, viral infection triggers RNA modification in host genes to suppress immune surveillance or to perturb host genetic networks for successful replication. In order to delineate the precise contribution of the viral and cell-mediated RNA modification events, further studies will be needed.

### References (Example 1)

Batista, P. J., Molinie, B., Wang, J., Qu, K., Zhang, J., Li, L., Bouley, D. M., Lujan, E., Haddad, B., Daneshvar, K., et al. (2014). m(6)A RNA modification controls cell fate transition in mammalian embryonic stem cells. Cell Stem Cell 15, 707-719; Bokar, J. A., Shambaugh, M. E., Polayes, D., Matera, A. G., and Rottman, F. M. (1997). Purification and cDNA cloning of the AdoMet-binding subunit of the human mRNA (N6-adenosine)-methyltransferase. RNA 3, 1233-1247; Brasil, P., Pereira, J. P., Jr., Raja Gabaglia, C., Damasceno, L., Wakimoto, M., Ribeiro Nogueira, R. M., Carvalho de Sequeira, P., Machado Siqueira, A., Abreu de Carvalho, L. M., Cotrim da Cunha, D., et al. (2016). Zika Virus Infection in Pregnant Women in Rio de Janeiro—Preliminary Report. The New England journal of medicine, 10.1056/NEJMoa1602412; Broutet, N., Krauer, F., Riesen, M., Khalakdina, A., Almiron, M., Aldighieri, S., Espinal, M., Low, N., and Dye, C. (2016). Zika Virus as a Cause of Neurologic Disorders. The New England journal of medicine 374, 1506-1509; Cugola, F. R., Fernandes, I. R., Russo, F. B., Freitas, B. C., Dias, J. L. M., Guimaries, K. P., Benazzato, C., Almeida, N., Pignatari, G. C., Romero, S., et al. (2016). The Brazilian Zika virus strain causes birth defects in experimental models. Nature, 10.1038/nature18296; Daffis, S., Szretter, K. J., Schriewer, J., Li, J., Youn, S., Errett, J., Lin, T. Y., Schneller, S., Zust, R., Dong, H., et al. (2010). 2′-O methylation of the viral mRNA cap evades host restriction by IFIT family members. Nature 468, 452-456; Dang, J., Tiwari, S. K., Lichinchi, G., Qin, Y., Patil, V. S., Eroshkin, A. M., and Rana, T. M. (2016b). Zika Virus Depletes Neural Progenitors in Human Cerebral Organoids through Activation of the Innate Immune Receptor TLR3. Cell Stem Cell 19, 258-265; Dejnirattisai, W., Supasa, P., Wongwiwat, W., Rouvinski, A., Barba-Spaeth, G., Duangchinda, T., Sakuntabhai, A., Cao-Lormeau, V. M., Malasit, P., Rey, F. A., et al. (2016). Dengue virus sero-cross-reactivity drives antibody-dependent enhancement of infection with zika virus. Nat Immunol; Dominissini, D., Moshitch-Moshkovitz, S., Schwartz, S., Salmon-Divon, M., Ungar, L., Osenberg, S., Cesarkas, K., Jacob-Hirsch, J., Amariglio, N., Kupiec, M., et al. (2012). Topology of the human and mouse m6A RNA methylomes revealed by m6A-seq. Nature 485, 201-206; Dong, H., Chang, D. C., Hua, M. H., Lim, S. P., Chionh, Y. H., Hia, F., Lee, Y. H., Kukkaro, P., Lok, S. M., Dedon, P. C., et al. (2012). 2′-0 methylation of internal adenosine by flavivirus NS5 methyltransferase. PLoS pathogens 8, e1002642; Driggers, R. W., Ho, C. Y., Korhonen, E. M., Kuivanen, S., Jaaskelainen, A. J., Smura, T., Rosenberg, A., Hill, D. A., DeBiasi, R. L., Vezina, G., et al. (2016a). Zika Virus Infection with Prolonged Maternal Viremia and Fetal Brain Abnormalities. The New England journal of medicine, 10; Driggers, R. W., Ho, C. Y., Korhonen, E. M., Kuivanen, S., Jaaskelainen, A. J., Smura, T., Rosenberg, A., Hill, D. A., DeBiasi, R. L., Vezina, G., et al. (2016b). Zika Virus Infection with Prolonged Maternal Viremia and Fetal Brain Abnormalities. The New England journal of medicine 374, 2142-2151; Fauci, A. S., and Morens, D. M. (2016). Zika Virus in the Americas—Yet Another Arbovirus Threat. The New England journal of medicine 374, 601-604; Finkel, D., and Groner, Y. (1983). Methylations of adenosine residues (m6A) in pre-mRNA are important for formation of late simian virus 40 mRNAs. Virology 131, 409-425; Frayling, T. M., Timpson, N. J., Weedon, M. N., Zeggini, E., Freathy, R. M., Lindgren, C. M., Perry, J. R., Elliott, K. S., Lango, H., Rayner, N. W., et al. (2007). A common variant in the FTO gene is associated with body mass index and predisposes to childhood and adult obesity. Science 316, 889-894; Fu, Y., Dominissini, D., Rechavi, G., and He, C. (2014). Gene expression regulation mediated through reversible m(6)A RNA methylation. Nat Rev Genet 15, 293-306; Fustin, J. M., Doi, M., Yamaguchi, Y., Hida, H., Nishimura, S., Yoshida, M., Isagawa, T., Morioka, M. S., Kakeya, H., Manabe, I., et al. (2013). RNA-methylation-dependent RNA processing controls the speed of the circadian clock. Cell 155, 793-806; Geula, S., Moshitch-Moshkovitz, S., Dominissini, D., Mansour, A. A., Kol, N., Salmon-Divon, M., Hershkovitz, V., Peer, E., Mor, N., Manor, Y. S., et al. (2015). Stem cells. m6A mRNA methylation facilitates resolution of naive pluripotency toward differentiation. Science 347, 1002-1006; Hamel, R., Dejarnac, O., Wichit, S., Ekchariyawat, P., Neyret, A., Luplertlop, N., Perera-Lecoin, M., Surasombatpattana, P., Talignani, L., Thomas, F., et al. (2015). Biology of Zika Virus Infection in Human Skin Cells. J Virol 89, 8880-8896; Heymann, D. L., Hodgson, A., Sall, A. A., Freedman, D. O., Staples, J. E., Althabe, F., Baruah, K., Mahmud, G., Kandun, N., Vasconcelos, P. F., et al. (2016). Zika virus and microcephaly: why is this situation a PHEIC? Lancet 387, 719-721; Hills, S. L., Russell, K., Hennessey, M., Williams, C., Oster, A. M., Fischer, M., and Mead, P. (2016). Transmission of Zika Virus Through Sexual Contact with Travelers to Areas of Ongoing Transmission—Continental United States, 2016. MMWR Morb Mortal Wkly Rep 65, 215-216; Jia, G., Fu, Y., Zhao, X., Dai, Q., Zheng, G., Yang, Y., Yi, C., Lindahl, T., Pan, T., Yang, Y. G., et al. (2011). N6-methyladenosine in nuclear RNA is a major substrate of the obesity-associated FTO. Nature chemical biology 7, 885-887; Kane, S. E., and Beemon, K. (1985). Precise localization of m6A in Rous sarcoma virus RNA reveals clustering of methylation sites: implications for RNA processing. Molecular and cellular biology 5, 2298-2306; Kennedy, E. M., Bogerd, H. P., Kornepati, A. V., Kang, D., Ghoshal, D., Marshall, J. B., Poling, B. C., Tsai, K., Gokhale, N. S., Horner, S. M., et al. (2016). Posttranscriptional m(6)A Editing of HIV-1 mRNAs Enhances Viral Gene Expression. Cell Host Microbe 19, 675-685; Kroschewski, H., Lim, S. P., Butcher, R. E., Yap, T. L., Lescar, J., Wright, P. J., Vasudevan, S. G., and Davidson, A. D. (2008). Mutagenesis of the dengue virus type 2 NS5 methyltransferase domain. The Journal of biological chemistry 283, 19410-19421; Krug, R. M., Morgan, M. A., and Shatkin, A. J. (1976). Influenza viral mRNA contains internal N6-methyladenosine and 5′-terminal 7-methylguanosine in cap structures. Journal of virology 20, 45-53; Lazear, H. M., Govero, J., Smith, A. M., Platt, D. J., Fernandez, E., Miner, J. J., and Diamond, M. S. (2016). A Mouse Model of Zika Virus Pathogenesis. Cell Host Microbe, 10.1016/j.chom.2016.1003.1010; Li, C., Xu, D., Ye, Q., Hong, S., Jiang, Y., Liu, X., Zhang, N., Shi, L., Qin, C. F., and Xu, Z. (2016). Zika Virus Disrupts Neural Progenitor Development and Leads to Microcephaly in Mice. Cell Stem Cell, 10.1016/j.stem.2016.1004.1017; Lichinchi, G., Gao, S., Saletore, Y., Gonzalez, G. M., Bansal, V., Wang, Y., Mason, C. E., and Rana, T. M. (2016). Dynamics of the human and viral m6A RNA methylomes during HIV-1 infection of T cells. Nature Microbiology 1, doi:10.1038/nmicrobiol.2016.1011; Liu, J., Yue, Y., Han, D., Wang, X., Fu, Y., Zhang, L., Jia, G., Yu, M., Lu, Z., Deng, X., et al. (2014). A METTL3-METTL14 complex mediates mammalian nuclear RNA N6-adenosine methylation. Nature chemical biology 10, 93-95; Liu, N., Dai, Q., Zheng, G., He, C., Parisien, M., and Pan, T. (2015). N(6)-methyladenosine-dependent RNA structural switches regulate RNA-protein interactions. Nature 518, 560-564; Meyer, K. D., Patil, D. P., Zhou, J., Zinoviev, A., Skabkin, M. A., Elemento, O., Pestova, T. V., Qian, S. B., and Jaffrey, S. R. (2015). 5′ UTR m(6)A Promotes Cap-Independent Translation. Cell 163, 999-1010; Meyer, K. D., Saletore, Y., Zumbo, P., Elemento, O., Mason, C. E., and Jaffrey, S. R. (2012). Comprehensive analysis of mRNA methylation reveals enrichment in 3′ UTRs and near stop codons. Cell 149, 1635-1646; Mlakar, J., Korva, M., Tul, N., Popovic, M., Poljsak-Prijatelj, M., Mraz, J., Kolenc, M., Resman Rus, K., Vesnaver Vipotnik, T., Fabjan Vodusek, V., et al. (2016). Zika Virus Associated with Microcephaly. N Engl J Med 374, 951-958; Petersen, L. R., Jamieson, D. J., Powers, A. M., and Honein, M. A. (2016). Zika Virus. The New England journal of medicine 374, 1552-1563; Ping, X. L., Sun, B. F., Wang, L., Xiao, W., Yang, X., Wang, W. J., Adhikari, S., Shi, Y., Lv, Y., Chen, Y. S., et al. (2014). Mammalian WTAP is a regulatory subunit of the RNA N6-methyladenosine methyltransferase. Cell research 24, 177-189; Sarno, M., Sacramento, G. A., Khouri, R., do Rosario, M. S., Costa, F., Archanjo, G., Santos, L. A., Nery, N., Jr., Vasilakis, N., Ko, A. I., et al. (2016). Zika Virus Infection and Stillbirths: A Case of Hydrops Fetalis, Hydranencephaly and Fetal Demise. PLoS Negl Trop Dis 10, e0004517; Schwartz, S., Mumbach, M. R., Jovanovic, M., Wang, T., Maciag, K., Bushkin, G. G., Mertins, P., Ter-Ovanesyan, D., Habib, N., Cacchiarelli, D., et al. (2014). Perturbation of m6A writers reveals two distinct classes of mRNA methylation at internal and 5′ sites. Cell Rep 8, 284-296; Squires, J. E., Patel, H. R., Nousch, M., Sibbritt, T., Humphreys, D. T., Parker, B. J., Suter, C. M., and Preiss, T. (2012). Widespread occurrence of 5-methylcytosine in human coding and non-coding RNA. Nucleic acids research 40, 5023-5033; Tang, H., Hammack, C., Ogden, S. C., Wen, Z., Qian, X., Li, Y., Yao, B., Shin, J., Zhang, F., Lee, E. M., et al. (2016). Zika Virus Infects Human Cortical Neural Progenitors and Attenuates Their Growth. Cell Stem Cell 18, 587-590; Tirumuru, N., Zhao, B. S., Lu, W., Lu, Z., He, C., and Wu, L. (2016). N(6)-methyladenosine of HIV-1 RNA regulates viral infection and HIV-1 Gag protein expression. eLife 5; Wang, X., Lu, Z., Gomez, A., Hon, G. C., Yue, Y., Han, D., Fu, Y., Parisien, M., Dai, Q., Jia, G., et al. (2014a). N6-methyladenosine-dependent regulation of messenger RNA stability. Nature 505, 117-120; Wang, X., Zhao, B. S., Roundtree, I. A., Lu, Z., Han, D., Ma, H., Weng, X., Chen, K., Shi, H., and He, C. (2015). N(6)-methyladenosine Modulates Messenger RNA Translation Efficiency. Cell 161, 1388-1399; Wang, Y., Li, Y., Toth, J. I., Petroski, M. D., Zhang, Z., and Zhao, J. C. (2014b). N6-methyladenosine modification destabilizes developmental regulators in embryonic stem cells. Nat Cell Biol 16, 191-198; Xu, C., Wang, X., Liu, K., Roundtree, I. A., Tempel, W., Li, Y., Lu, Z., He, C., and Min, J. (2014). Structural basis for selective binding of m6A RNA by the YTHDC1 YTH domain. Nature chemical biology 10, 927-929; Yi, C., and Pan, T. (2011). Cellular dynamics of RNA modification. Acc Chem Res 44, 1380-1388; Zhang, B., Dong, H., Zhou, Y., and Shi, P. Y. (2008). Genetic interactions among the West Nile virus methyltransferase, the RNA-dependent RNA polymerase, and the 5′ stem-loop of genomic RNA. Journal of virology 82, 7047-7058; Zhao, X., Yang, Y., Sun, B. F., Shi, Y., Yang, X., Xiao, W., Hao, Y. J., Ping, X. L., Chen, Y. S., Wang, W. J., et al. (2014). FTO-dependent demethylation of N6-methyladenosine regulates mRNA splicing and is required for adipogenesis. Cell research 24, 1403-1419; Zheng, G., Dahl, J. A., Niu, Y., Fedorcsak, P., Huang, C. M., Li, C. J., Vagbo, C. B., Shi, Y., Wang, W. L., Song, S. H., et al. (2013). ALKBH5 is a mammalian RNA demethylase that impacts RNA metabolism and mouse fertility. Molecular cell 49, 18-29; Zhou, J., Wan, J., Gao, X., Zhang, X., Jaffrey, S. R., and Qian, S. B. (2015). Dynamic m(6)A mRNA methylation directs translational control of heat shock response. Nature 526, 591-594.

### Experimental and Methods Description

ZIKV Production and Cell Infection.

All studies were conducted in accordance with approved IRB protocols by the University of California, San Diego. All animal work was approved by the Institutional Review Board at the University of California, San Diego and was performed in accordance with Institutional Animal Care and Use Committee guidelines. ZIKV MR766 virus was expanded by inoculation of Vero cells at an MOI of 5 as previously described (Dang et al., 2016b).

Immunofluorescence Staining of Flavivirus Envelope Protein.

293T cells previously transduced with NTC, METTL3, METTL14, FTO, or ALKBHS shRNAs were seeded in 24-well plates pretreated with poly-L-lysine (Sigma) one day before infection, and then infected as described above. At 24 h post-infection, cells were washed three times with PBS and fixed in 4% paraformaldehyde in PBS for 20 min at room temperature. Cells were permeabilized in PBS containing 0.2% Triton X-100 for 10 min at room temperature and then blocked with 3% BSA in PBS for 2 h. Cells were stained with primary antibody (1:1000) overnight at 4° C., washed three times with PBS, and then stained with Alexa 488-conjugated secondary antibody (1:1000) for 1 h at room temperature. The nuclei were stained with DAPI, and cells were analyzed using a Leica microscope.

m⁶A-seq.

High-throughput sequencing of the ZIKV methylome was carried out by m⁶A-seq following the previously published protocol (Dominissini et al., 2012). In brief, total cellular RNA (containing ZIKV RNA) was extracted and purified by the RiboMinus Eukaryote System v2 (Thermo Fisher) to remove contaminating rRNA. Purified RNA was sonicated to ˜200 nt lengths, mixed with 2.5 mg of affinity-purified anti-m⁶A polyclonal antibody (202003; Synaptic Systems, Goettingen, Germany) in IP buffer (150 mM NaCl, 0.1% NP-40, 10 mM Tris-HCl, pH 7.4), and incubated for 2 h at 4° C. The antibody was collected by incubation with protein A beads (Invitrogen) at 4° C. for 2 h, and the beads were washed three times and eluted with m⁶A-free nucleotide solution. RNA in the eluate was purified using RNA Clean & Concentrator kit (Zymo Research) and used for library generation with a Stranded mRNA Library Prep kit (Illumina). Sequencing was performed using an Illumina HiSeq 2000 according to the manufacturer's instructions.

Accession Numbers.

The GEO accession number for the RNA-seq data reported in this paper is GEO number GSE87516.

Lentiviral Preparation, Cell Transduction, and Viral RNA Quantification.

293T cells (10⁶) were transfected with pLKO shRNA (1 μg), psPAX.2 (0.75 μg), and pMD2.G (0.25 ag) vectors using Lipofectamine 2000 and Opti-MEM (Invitrogen) according to the manufacturer's instructions. The medium was replaced with complete DMEM at 4 h post-transfection, and 48 h later, the supernatant was collected, filtered through a 0.22 μm membrane, and incubated with 293T cells in the presence of 1 μg/ml polybrene. After 12 h, the medium was removed and replaced with complete DMEM. At 3 days post-transduction, 293T cells were infected with MR766 virus at an MOI of 5. After 12 h, the medium was changed. Viral supernatants were collected and total cellular RNA was prepared at 48 h after infection. Viral titer was assessed as described above. Total RNA was extracted using TRIzol reagent (Invitrogen). mRNA expression was measured using iScript Reverse Transcription Supermix for RT-qPCR and iTaq Universal SYBR Green Supermix (Bio-Rad) for qPCR. Viral RNA released into the supernatant was measured using an iScript One Step RT-PCR kit.

Plaque-Forming Unit Assay. Confluent Vero cells were inoculated with four serial 10-fold dilutions of supernatants from infected cells. After 2 h, the cells were washed, overlaid with plaque medium containing 0.4% agarose, and cultured at 37° C. for 4 days. Cells were then fixed with 3.7% formaldehyde, stained with 0.1% crystal violet in 20% ethanol, and plaques counted.

METTL3, METTL14, and ALKBH5 Overexpression.

METTL3, METTL14, and ALKBH5 overexpression plasmids were constructed by cloning the corresponding cDNAs into the mammalian expression vector pcDNA3 (Invitrogen). 293T cells were transfected using Lipofectamine 2000 and Opti-MEM. The medium was replaced with complete DMEM after 4 h, and 48 h later, the cells were infected with MR766 virus at an MOI of 5. Viral supernatants were collected and total cellular RNA was prepared at 24 h post-infection. Viral titer and RNA were quantified as described above.

FLAG-YTHDF1-3 Overexpression and Immunoprecipitation.

293T cells (2×10⁶) were transfected with 2 μg FLAG-YTHDF or control pcDNA plasmids using Lipofectamine 2000 and Opti-MEM. After 4 h, the medium was replaced with complete DMEM, and 20 h later, the cells were infected with ZIKV at an MOI of 5. At 24 h post-infection, cells were lysed with Pierce IP lysis buffer supplemented with protease inhibitors and RNaseOUT. An aliquot of cleared lysate (5%) was saved as input. The remaining lysate was incubated overnight at 4° C. with 2 μg of anti-FLAG antibody (clone M2, Sigma) and 50 μl of Protein G Dynabeads (Invitrogen). Beads were washed three times with lysis buffer and divided in half for RNA extraction and RT-qPCR analysis or for western blot analysis.

Liquid Chromatography-Tandem Mass Spectrometry. Genomic RNA from MR766 ZIKV was isolated from Vero cells as described previously (Dang et al., 2016), and modified nucleoside levels were quantified using LC-MS/MS as described (Jia et al., 2011). Briefly, 50 ng of viral genomic RNA was digested by nuclease P1 (2 U, Wako) in 25 μl of buffer containing 25 mM NaCl and 2.5 mM ZnCl₂ at 42° C. for 2 h, and then NH₄HCO₃ (1 M, 3 al) and alkaline phosphatase (0.5 U, Sigma) were added and the samples were incubated at 37° C. for an additional 2 h. The samples were diluted to 50 μl with nuclease-free water, filtered (0.22 μm pore size, 4 mm diameter, Millipore), and 10 μl aliquots were taken for analysis. Nucleosides were separated on a reverse phase ultra-performance liquid chromatography C18 column with in-line mass spectrometry detection using an Agilent 6410 QQQ triple-quadrupole LC mass spectrometer in positive electrospray ionization mode. Each modified nucleoside was quantified by the ratio of modified to unmodified nucleoside levels.

Data Analysis.

For data analysis, after removing the adapter, the reads were mapped to human genome (hg38) and ZIKA genome (NC_012532) by using Tophat2 (Kim et al., 2013). The peak calling method was modified from the published method (Dominissini et al., 2012). To call m⁶A peaks, the longest isoform of each Human RefSeq gene (and the whole genome of ZIKV) was scanned using a 100 bp sliding window with 10 bp steps. To reduce bias from potential inaccurate gene structure annotation and the arbitrary use of the longest isoform, windows with read counts less than 1/20 of the top window in both m⁶A immunoprecipitate and input samples were excluded. For each gene, the read count in each window was normalized by the median count of all windows of that gene. A negative binomial model was used to identify the differential windows between immunoprecipitate and input samples by using the edgeR package (Robinson et al., 2010), for each and eventually combining information from all three replicates in the two groups. The window was called as positive if the FDR was <1% and the log 2 (enrichment score) was ≥1. Overlapping positive windows were merged. The following were calculated to obtain the enrichment score of each peak (or window): (a) read counts of the immunoprecipitated sample in the current peak/window, (b) median read counts of the immunoprecipitated sample in all 100 bp windows on the current mRNA, (c) read counts of the input sample in the current peak/window, and (d) median read counts of the input sample in all 100 bp windows on the current mRNA. The enrichment score of each window was calculated as ([a×d])/([b×c]).

Primer List.

Primers useful in the experimental procedures disclosed herein are tabulated following.

| Primer | Sequence |
|---|---|
| GAPDH Fwd | TGGCGGGGAAGTCAG (SEQ ID NO: 1) |
| GAPDH Rev | CGGAGGAGAAATCGGGC (SEQ ID NO: 2) |
| ZIKV Fwd | TTGGTCATGATACTGCTGATTGC (SEQ ID NO: 3) |
| ZIKV Rev | CCCTCCACGAAGTCTCTATTGC (SEQ ID NO: 4) |
| METTL3 Fwd | GACACGTGGAGCTCTATCCA (SEQ ID NO: 5) |
| METTL3 Rev | GGAAGGTTGGAGACAATGCT (SEQ ID NO: 6) |
| METTL14 Fwd | TCCCAAATCTAAATCTGACCG (SEQ ID NO: 7) |
| METTL14 Rev | CTCTAAAGCCACCTCTTTCTC (SEQ ID NO: 8) |
| ALKBH5 Fwd | AGGGACCCTGCTCTGAAAC (SEQ ID NO: 9) |
| ALKBH5 Rev | TCCTTGTCCATCTCCAGGAT (SEQ ID NO: 10) |

Shrna Sequences.

shRNA sequences useful in the experimental procedures disclosed herein are tabulated following.

| shRNA | Sequence |
|---|---|
| NTC | CCGCAGGTATGCACGCGT (SEQ ID NO: 11) |
| METTL3-1 | CCGGGCAAGTATGTTCACTATGAAACTCGAGTTTCATAGTG |
| | AACATACTTGCTTTTTG (SEQ ID NO: 12) |
| METTL3-2 | CCGGGCCAAGGAACAATCCATTGTTCTCGAGAACAATGGAT |
| | TGTTCCTTGGCTTTTTG (SEQ ID NO: 13) |
| METTL14-1 | CCGGCCATGTACTTACAAGCCGATACTCGAGTATCGGCTTG |
| | TAAGTACATGGTTTTT (SEQ ID NO: 14) |
| METTL14-2 | CCGGGCCGTGGACGAGAAAGAAATACTCGAGTATTTCTTTC |
| | TCGTCCACGGCTTTTT (SEQ ID NO: 15) |
| ALKBH5-1 | CCGGGAAAGGCTGTTGGCATCAATACTCGAGTATTGATGCC |
| | AACAGCCTTTCTTTTTG (SEQ ID NO: 16) |
| ALKBH5-2 | CCGGCCACCCAGCTATGCTTCAGATCTCGAGATCTGAAGCA |
| | TAGCTGGGTGGTTTTTG (SEQ ID NO: 17) |
| FTO-1 | CCGGCGGTTCACAACCTCGGTTTAGCTCGAGCTAAACCGAG |
| | GTTGTGAACCGTTTTTG (SEQ ID NO: 18) |
| FTO-2 | CCGGTCACCAAGGAGACTGCTATTTCTCGAGAAATAGCAGT |
| | CTCCTTGGTGATTTTTG (SEQ ID NO: 19) |
| YTHDF1-1 | CCGGCCCTACCTGTCCAGCTATTACCTCGAGGTAATAGCTG |
| | GACAGGTAGGGTTTTTG (SEQ ID NO: 20) |
| YTHDF1-2 | CCGGCCCGAAAGAGTTTGAGTGGAACTCGAGTTCCACTCAA |
| | ACTCTTTCGGGTTTTTG (SEQ ID NO: 21) |
| YTHDF2-1 | CCGGGCTACTCTGAGGACGATATTCCTCGAGGAATATCGTC |
| | CTCAGAGTAGCTTTTTG (SEQ ID NO: 22) |
| YTHDF2-2 | CCGGCGGTCCATTAATAACTATAACCTCGAGGTTATAGTTAT |
| | TAATGGACCGTTTTTG (SEQ ID NO: 23) |
| YTHDF3-1 | CCGGTAAGTCAAAGAAGACGTATTACTCGAGTAATACGTCT |
| | TCTTTGACTTATTTTTG (SEQ ID NO: 24) |
| YTHDF3-2 | CCGGGAAGTCTGTTGTGGACTATAACTCGAGTTATAGTCCA |
| | CAACAGACTTCTTTTTG (SEQ ID NO: 25) |

Dang, J., Tiwari, S. K., Lichinchi, G., Qin, Y., Patil, V. S., Eroshkin, A. M., and Rana, T. M. (2016). Zika Virus Depletes Neural Progenitors in Human Cerebral Organoids through Activation of the Innate Immune Receptor TLR3. Cell Stem Cell 19, 258-265; Dominissini, D., Moshitch-Moshkovitz, S., Schwartz, S., Salmon-Divon, M., Ungar, L., Osenberg, S., Cesarkas, K., Jacob-Hirsch, J., Amariglio, N., Kupiec, M., et al. (2012). Topology of the human and mouse m6A RNA methylomes revealed by m6A-seq. Nature 485, 201-206; Jia, G., Fu, Y., Zhao, X., Dai, Q., Zheng, G., Yang, Y., Yi, C., Lindahl, T., Pan, T., Yang, Y. G., et al. (2011). N6-methyladenosine in nuclear RNA is a major substrate of the obesity-associated FTO. Nature chemical biology 7, 885-887; Kim, D., Pertea, G., Trapnell, C., Pimentel, H., Kelley, R., and Salzberg, S. L. (2013). TopHat2: accurate alignment of transcriptomes in the presence of insertions, deletions and gene fusions. Genome Biol 14, R36; Robinson, M. D., McCarthy, D. J., and Smyth, G. K. (2010). edgeR: a Bioconductor package for differential expression analysis of digital gene expression data. Bioinformatics 26, 139-140.

### Example 2—Design, Synthesis and Structure of ZIKV Inhibitors

The chemical structures of S-adenosylmethionine (SAM), S-adenosylhomocystein (an analog of SAM), and sinefungin are as follows:

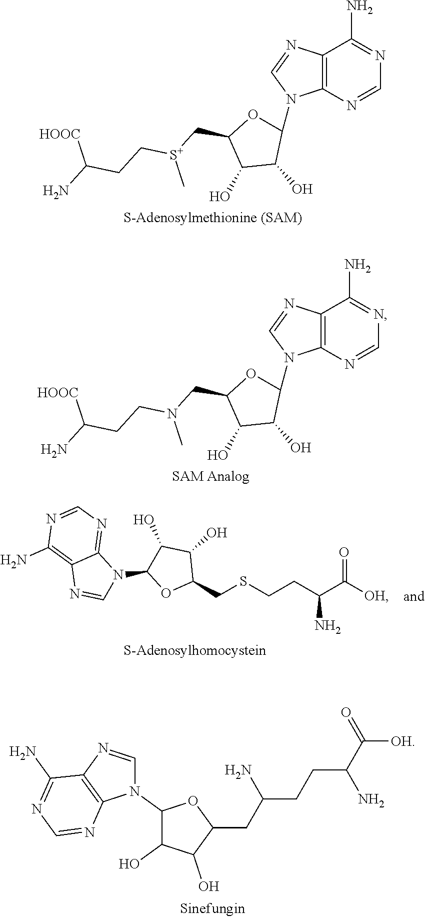 [SMILES: C[S+](CCC(N)C(=O)O)C[C@H]1OC(n2cnc3c(N)ncnc32)[C@H](O)[C@@H]1O ; CN(CCC(N)C(=O)O)C[C@H]1OC(n2cnc3c(N)ncnc32)[C@H](O)[C@@H]1O ; Nc1ncnc2c1ncn2[C@@H]1O[C@H](CSCC[C@H](N)C(=O)O)[C@@H](O)[C@H]1O ; "and" ; Nc1ncnc2c1ncn2C1OC(CC(N)CCC(N)C(=O)O)C(O)C1O]

A chemical synthesis scheme useful for the compounds disclosed herein is shown in Scheme 1.

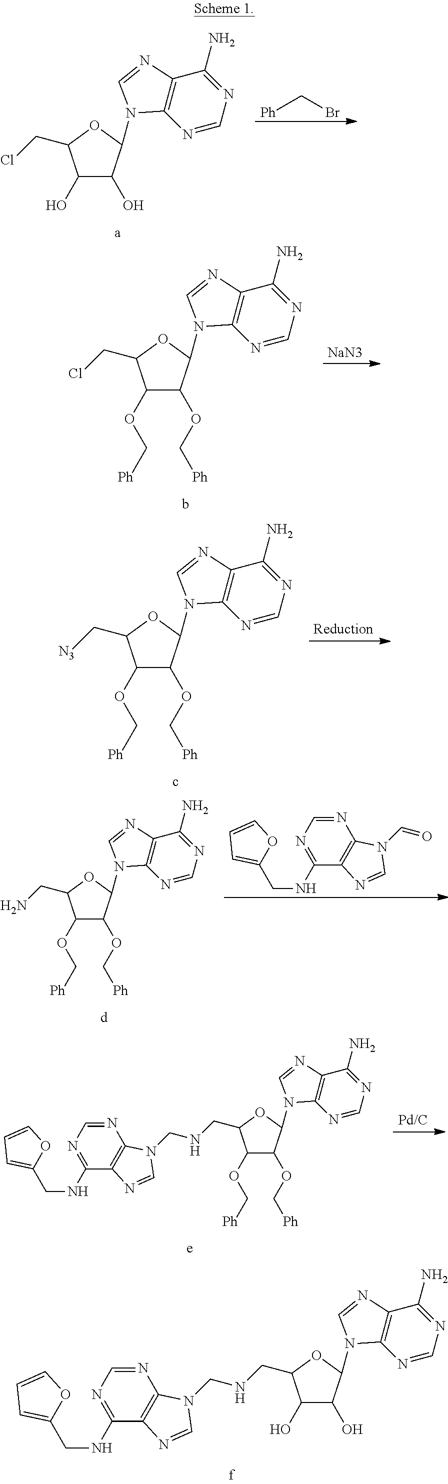

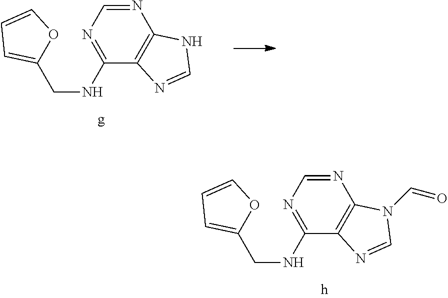 [SMILES: c1coc(CNc2ncnc3[nH]cnc23)c1 ; O=Cn1cnc2c(NCc3ccco3)ncnc21]

A further chemical synthesis scheme useful for the compounds disclosed herein is shown in Scheme 2.

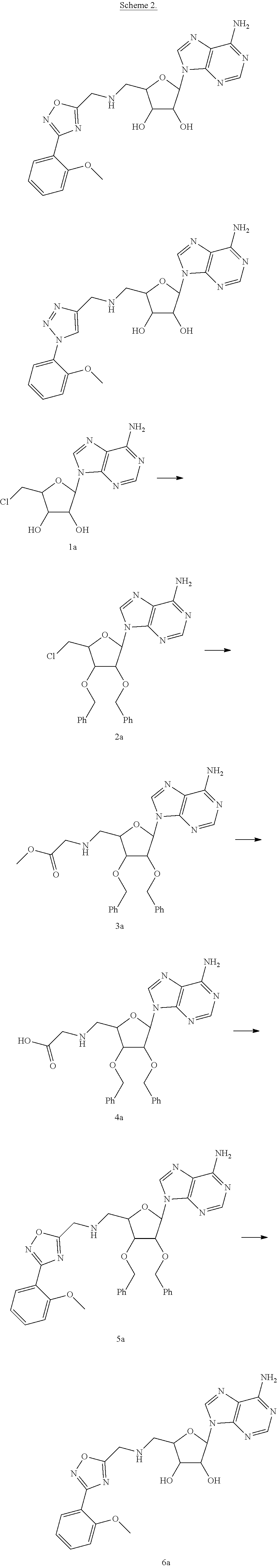

A further chemical synthesis scheme useful for the compounds disclosed herein is shown in Scheme 3.

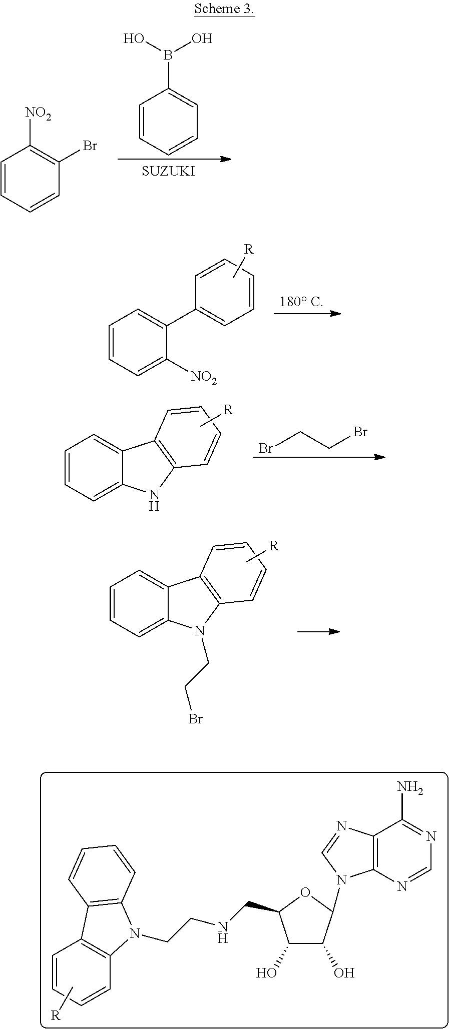

A further chemical synthesis scheme useful for the compounds disclosed herein is shown in Scheme 4.

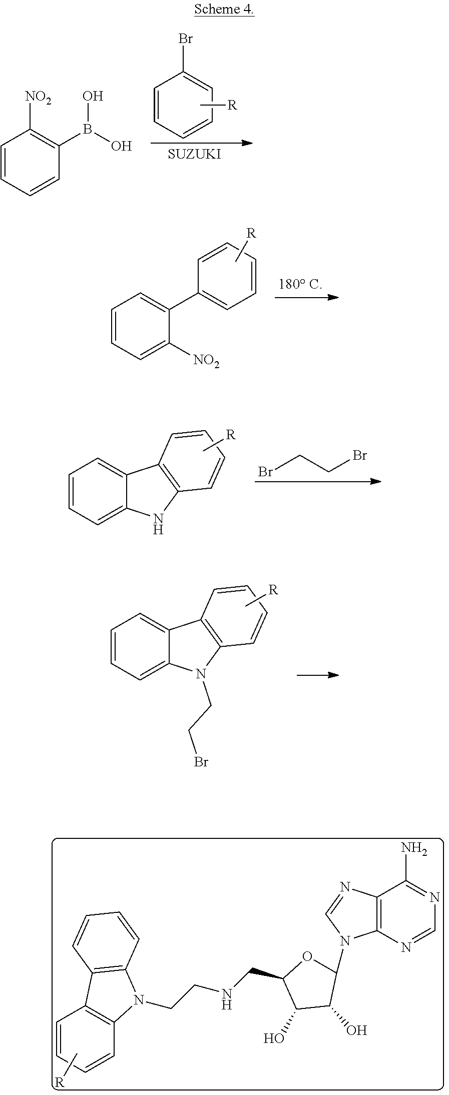

A further chemical synthesis scheme useful for the compounds disclosed herein is shown in Scheme 5.

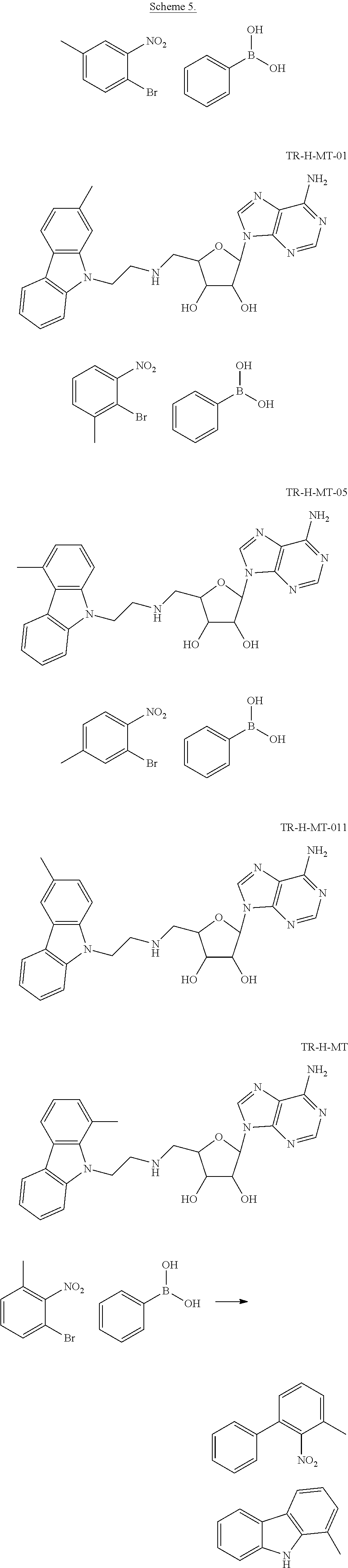

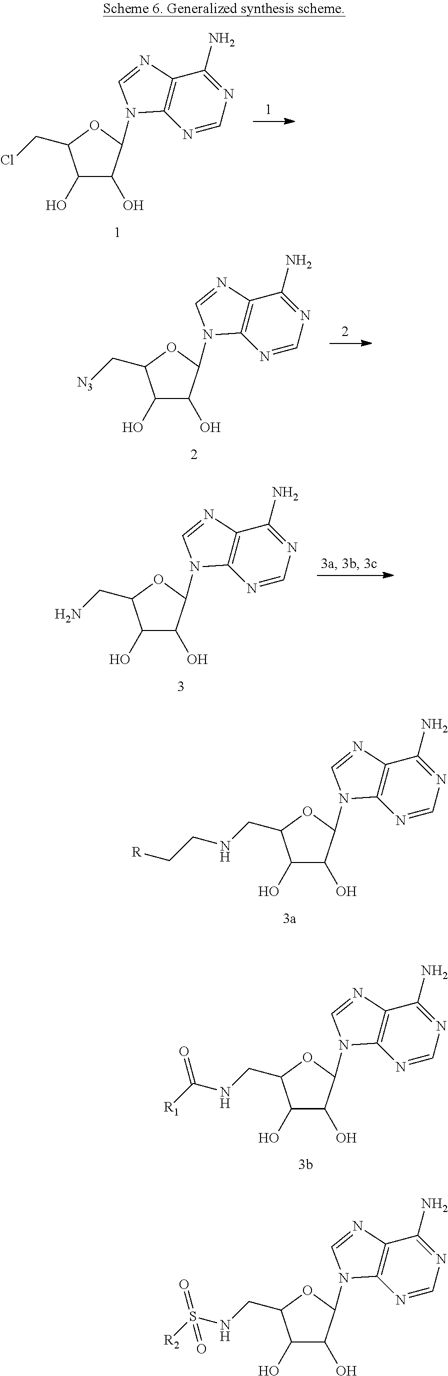

1. NaN₃, DMF, 1000 C, 3 h 2. Pd/C, MeOH, RT, 12 h. 3a. R—CH₂CH₂Br, Neat Reaction, sealed tube, 12 h. 3b. RCOCl, DMF, TEA, RT, 2 h. 3c. R—SO₂Cl, DMF, RT, 12 h. R (according to Scheme 6)=carbazole, benzimidazole, benzotriazole, pyridine, imidazole, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene), R¹ (according to Scheme 6)=substituted phenyl acidchlorides; R² (according to Scheme 6)=substituted phenyl sulfonylchlorides.

Step 1:

A suspension of compound 2 (200 mg, 0.685 mmol) and NaN3 (50 mg, 0.77 mmol) in dry DMF (1.0 mL) was heated at 150° C. under Ar for 3 h. After cooling to ambient temperature the excess of DMF was removed under rotavapour. The crude reaction mixture was added directly to a flash chromatography column packed with 20% methanol/DCM yielded compound as a white solid. 1H NMR (CD3OD): δ=3.58 (dd, J=4.4, 12.7 Hz, 1H), 3.84 (dd, J=7.0, 12.7 Hz, 1H), 4.33 (dd, J=4.0, 6.1 Hz, 1H,), 4.53 (ddd, J=4.4, 6.1, 7.0 Hz, 1H), 4.88 (dd, J=4.0, 4.0 Hz, 1H), 5.99 (d, J=4.0 Hz, 1H), 8.22 (s, 1H), 8.27 (s, 1H) ppm.

Step 2:

To a stirred solution of SM (1 eq) in MeOH and followed by the addition of 10% Pd/C under H2 balloon. The reaction mixture was stirred for 24 h at room temperature. After completion of reaction the reaction mixture was filtered through celite bed. The solvent as evaporated under vaccum and the crude product was used for next step without any further purification.

Step 3a:

To a solution of SM (2.0 mmol) in dry MeOH were added DIPEA (5.0 mmol) and alkyl halide (2.0 mmol) under nitrogen, and the mixture was stirred at 130° C. for 12 h in sealed tube. The reaction mixture concentrated under reduced pressure. The residue was purified by column chromatography (DCM:MeOH, 9:1→18:2) to give the 3a compound as a solid.

Step 3b:

To a stirred solution of SM (2 mmol) in DMF at 0° C. was added acid chloride (2 mmol) and the solution was stirred for 2 hours at room temperature. The reaction mixture concentrated under reduced pressure and crude material was purified by column chromatography (DCM:MeOH, 9:1→18:2) yielded compound 3b as a solid.

Step 3c:

To a stirred solution of SM (2 mmol) in DMF at 0° C. was added sulfonyl chloride (2 mmol) and the solution was stirred for 12 hours at room temperature. The reaction mixture concentrated under reduced pressure and crude material was purified by column chromatography (DCM:MeOH, 9:1→18:2) yielded compound 3c as a solid.

Found TR-H-08 was an active compound:

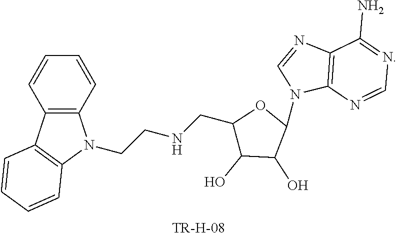 [SMILES: Nc1ncnc2c1ncn2C1OC(CNCCn2c3ccccc3c3ccccc32)C(O)C1O]

TR-H-01: 1H NMR (CD3OD): δ=8.22 (m, 2H), 8.06 (s, 1H), 7.88 (m, 1H,), 7.66 (m, 1H), 7.53 (m, 2H), 5.98 (d, J=6 Hz, 1H), 4.79 (t, J=10.8 Hz, 1H), 4.56 (t, J=12 Hz, 2H), 4.20 (m, 2H), 3.25 (m, 3H) ppm.

TR-H-02: 1H NMR (CD3OD): δ=8.50 (d, J=8.4 Hz, 1H), 8.29 (s, 1H), 8.13 (s, 1H), 7.76 (m, 1H,), 7.66 (d, J=8.4 Hz, 1H), 7.53 (t, J=10.4 Hz, 1H), 5.91 (d, J=6 Hz, 1H), 4.68 (t, J=10.8 Hz, 1H), 4.56 (t, J=12 Hz, 2H), 4.22 (m, 2H), 3.35 (s, 1H), 3.09 (m, 2H) ppm.

TR-H-03: 1H NMR (CD3OD): δ=8.29 (s, 1H), 8.22 (s, 1H), 7.96 (s, 1H,), 7.66 (s, 1H), 6.01 (d, J=6 Hz, 1H), 4.68 (t, J=10.8 Hz, 1H), 4.56 (t, J=12 Hz, 2H), 3.25 (m, 2H), 3.09 (m, 4H) ppm.

TR-H-04: 1H NMR (CD3OD): δ=8.19 (s, 1H), 8.03 (s, 1H), 7.86 (s, 1H,), 7.66 (d, J=8.4 Hz, 1H), 7.53 (d, J=8.4 Hz, 1H), 7.33 (t, J=15.6 Hz, 1H), 7.09 (t, J=15 Hz, 1H), 5.91 (d, J=6 Hz, 1H), 4.78 (t, J=10.8 Hz, 1H), 4.56 (t, J=12 Hz, 2H), 4.22 (m, 2H), 3.25 (m, 2H), 3.09 (m, 2H) ppm.

TR-H-05: 1H NMR (CD3OD): δ=8.22 (s, 1H), 8.03 (s, 1H), 7.92 (d, J=8.4 Hz, 1H), 7.79 (d, J=8.4 Hz, 1H), 7.53 (t, J=15 Hz, 1H), 7.40 (t, J=15 Hz, 1H), 5.95 (d, J=6 Hz, 1H), 4.99 (t, J=12 Hz, 2H), 4.76 (t, J=10.8 Hz, 1H), 4.31 (m, 2H), 3.60 (m, 2H), 3.40 (m, 2H) ppm.

TR-H-06: 1H NMR (CD3OD): δ=8.22 (m, 2H), 8.06 (d, J=7.8 Hz, 1H), 7.76 (s, 1H,), 7.04 (m, 1H), 6.01 (d, J=6.6 Hz, 1H), 4.84 (m, 1H), 4.67 (m, 1H), 4.48 (m, 1H), 4.36 (m, 1H), 3.83 (m, 2H), 3.61 (m, 2H), 2.35 (s, 3H) ppm.

TR-H-07: 1H NMR (CD3OD): δ=8.20 (m, 2H), 8.04 (d, J=7.8 Hz, 1H), 7.82 (s, 1H,), 7.04 (m, 1H), 5.89 (d, J=6.6 Hz, 1H), 4.74 (m, 1H), 4.77 (m, 1H), 4.40 (m, 1H), 4.36 (m, 1H), 3.63 (m, 2H), 3.41 (m, 2H) ppm.

TR-H-08: 1H NMR (CD3OD): δ=8.10 (s, 1H), 8.02 (d, J=7.2 Hz, 2H), 7.96 (s, 1H,), 7.47 (d, J=7.8 Hz, 2H), 7.37 (t, J=15, H), 7.15 (t, J=15 Hz, 2H), 5.88 (d, J=5.4 Hz, 1H), 4.37 (t, J=10.2 Hz, 1H), 4.52 (t, J=13.2 Hz, 2H), 4.22 (m, 2H), 3.23-3.05 (m, 5H) ppm.

TR-H-09: 1H NMR (DMSO): δ=8.32 (s, 1H), 8.12 (d, J=10.8 Hz, 2H), 8.07 (s, 1H), 7.30 (s, 2H), 7.15 (s, 2H), 5.82 (d, J=6 Hz, 1H), 5.40 (d, J=6 Hz, 1H), 5.16 (d, J=6 Hz, 1H), 4.67 (m, 1H), 4.37 (t, J=12 Hz, 2H), 4.06 (m, 1H), 3.94 (m, 1H), 3.15 (d, J=4.8 Hz, 1H), 2.97 (m, 4H) ppm.

TR-H-10: 1H NMR (CD3OD): δ=8.28 (s, 1H), 8.23 (s, 1H), 5.93 (d, J=5.4 Hz, 1H), 4.79 (t, J=10.2 Hz, 1H), 4.18 (m, 2H), 3.71 (m, 2H), 3.52 (m, 1H), 3.22 (m, 1H), 2.0 (m, 3H), 1.85 (m, 6H), 1.73 (m, 7H), 1.37 (m, 11H) ppm.

TR-H-12: 1H NMR (CD3OD): δ=8.44 (s, 1H), 8.34 (s, 1H), 7.17 (d, J=8.4 Hz, 2H,), 6.79 (d, J=8.4 Hz, 2H), 6.02 (d, J=6 Hz, 1H), 4.67 (t, J=10.8 Hz, 1H), 4.18 (m, 2H), 3.74 (s, 3H), 3.59 (m, 2H), 3.45 (m, 2H) ppm.

TR-H-13: 1H NMR (CD3OD): δ=8.23 (s, 1H), 8.22 (s, 1H), 7.26 (m, 4H,), 5.93 (d, J=6 Hz, 1H), 4.76 (t, J=10.8 Hz, 1H), 4.16 (m, 2H), 3.73 (m, 1H), 3.57 (m, 1H), 3.50 (m, 2H) ppm.

TR-H-14: 1H NMR (CD3OD): δ=8.26 (s, 1H), 7.89 (s, 1H), 7.81 (d, J=7.8 Hz, 2H,), 7.55 (m, 1H), 7.46 (m, 2H), 5.95 (d, J=6 Hz, 1H), 4.85 (t, J=11.4 Hz, 1H), 4.37 (t, J=9 Hz, 1H), 4.29 (m, 1H), 3.96 (m, 1H), 3.65 (m, 1H) ppm.

TR-H-15: 1H NMR (CD3OD): δ=8.40 (s, 2H), 8.28 (s, 1H), 8.17 (s, 1H,), 7.95 (s, 1H), 5.98 (d, J=4.8 Hz, 1H), 4.84 (t, J=10.8 Hz, 1H), 4.43 (t, J=9.6 Hz, 1H), 4.28 (m, 1H), 3.91 (m, 1H), 3.79 (m, 1H) ppm.

TR-H-16: 1H NMR (CD3OD): δ=8.29 (s, 1H), 8.22 (s, 1H), 7.05 (d, J=8.4 Hz, 2H,), 6.96 (d, J=8.4 Hz, 2H), 5.86 (d, J=6 Hz, 1H), 4.85 (m, 1H), 4.20 (m, 2H), 3.85 (m, 3H), 3.81 (s, 3H) ppm.

Analogs synthesized according to Scheme 6 include:

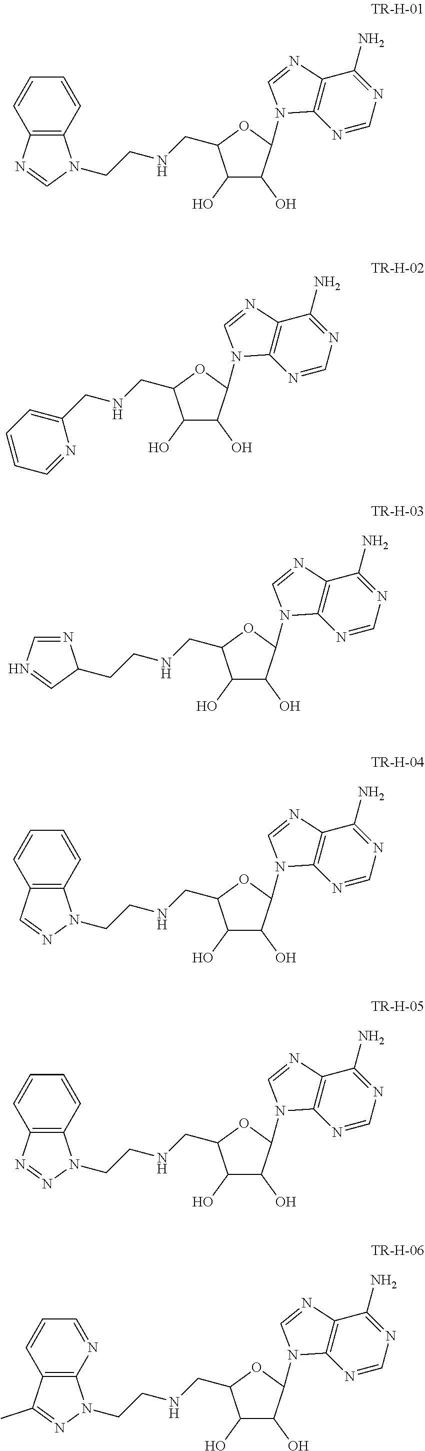 [SMILES: Nc1ncnc2c1ncn2C1OC(CNCCn2cnc3ccccc32)C(O)C1O ; Nc1ncnc2c1ncn2C1OC(CNCc2ccccn2)C(O)C1O ; Nc1ncnc2c1ncn2C1OC(CNCCC2C=NC=N2)C(O)C1O ; Nc1ncnc2c1ncn2C1OC(CNCCn2ncc3ccccc32)C(O)C1O ; Nc1ncnc2c1ncn2C1OC(CNCCn2nnc3ccccc32)C(O)C1O ; Cc1nn(CCNCC2OC(n3cnc4c(N)ncnc43)C(O)C2O)c2ncccc12]

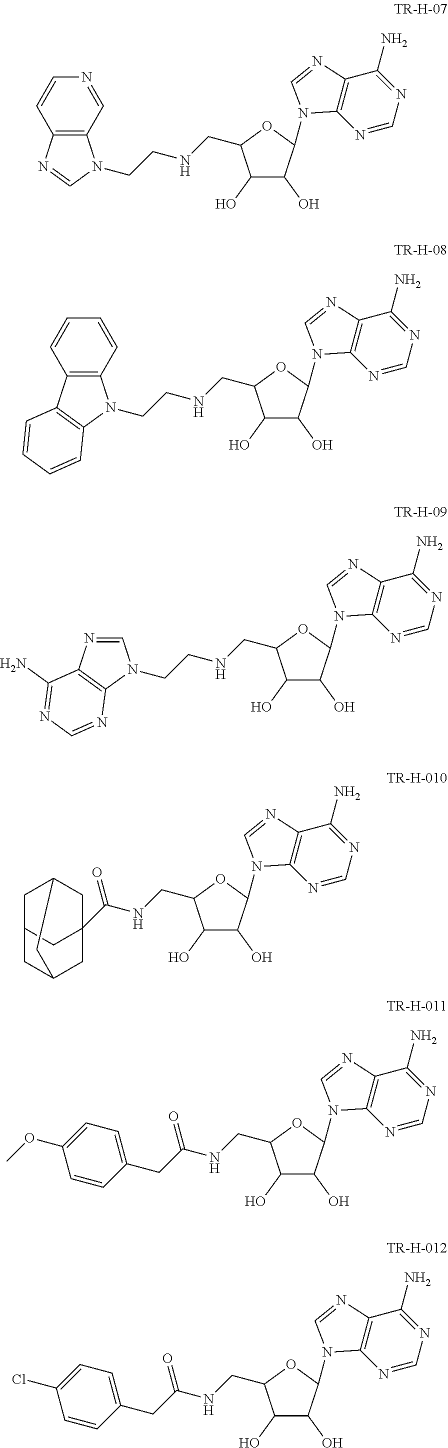 [SMILES: Nc1ncnc2c1ncn2C1OC(CNCCn2cnc3ccncc32)C(O)C1O ; Nc1ncnc2c1ncn2C1OC(CNCCn2c3ccccc3c3ccccc32)C(O)C1O ; Nc1ncnc2c1ncn2CCNCC1OC(n2cnc3c(N)ncnc32)C(O)C1O ; Nc1ncnc2c1ncn2C1OC(CNC(=O)C23CC4CC(CC(C4)C2)C3)C(O)C1O ; COc1ccc(CC(=O)NCC2OC(n3cnc4c(N)ncnc43)C(O)C2O)cc1 ; Nc1ncnc2c1ncn2C1OC(CNC(=O)Cc2ccc(Cl)cc2)C(O)C1O]

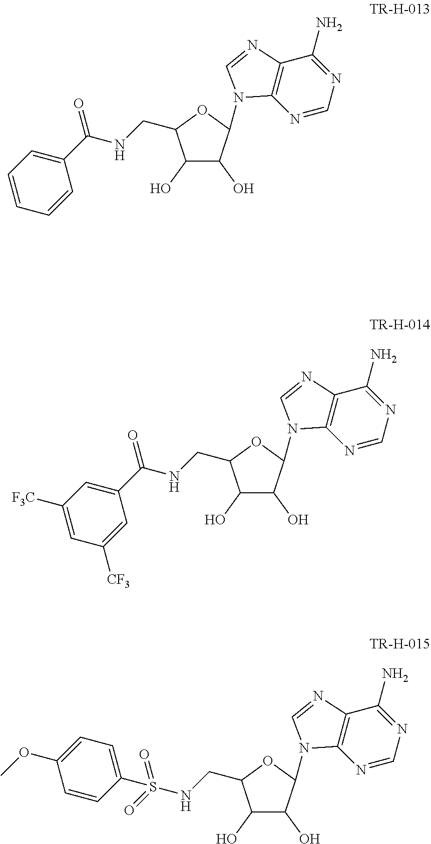 [SMILES: Nc1ncnc2c1ncn2C1OC(CNC(=O)c2ccccc2)C(O)C1O ; Nc1ncnc2c1ncn2C1OC(CNC(=O)c2cc(C(F)(F)F)cc(C(F)(F)F)c2)C(O)C1O ; COc1ccc(S(=O)(=O)NCC2OC(n3cnc4c(N)ncnc43)C(O)C2O)cc1]

Scheme 7. Step 1. NaN3, DMF, 1000 C, 3 h 2. Pd/C, MeOH, RT, 12 h. 3a. RCH2CH2Br, Neat Reaction, sealedtube, 12 h. 4. Pd(OAc)₂, TPP, K₂CO₃, 1,2dichlorobenzene. 5. 1,2dibromoethane, 30% NaOH, TBAB. R (according to Scheme 7)=—CH₃, —OMe, —F, —CF₃, or a substituent group described herein.

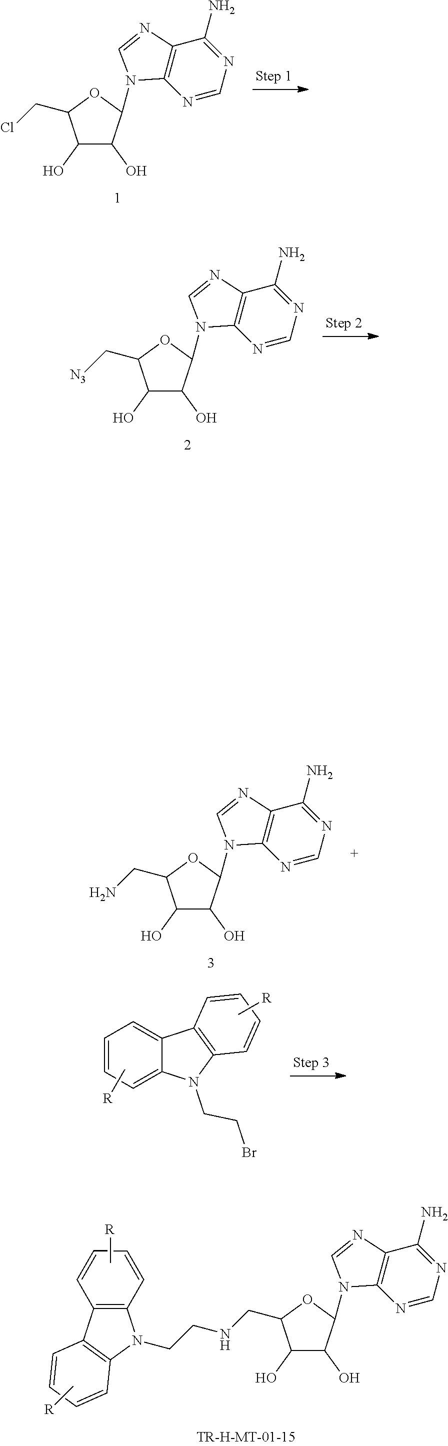

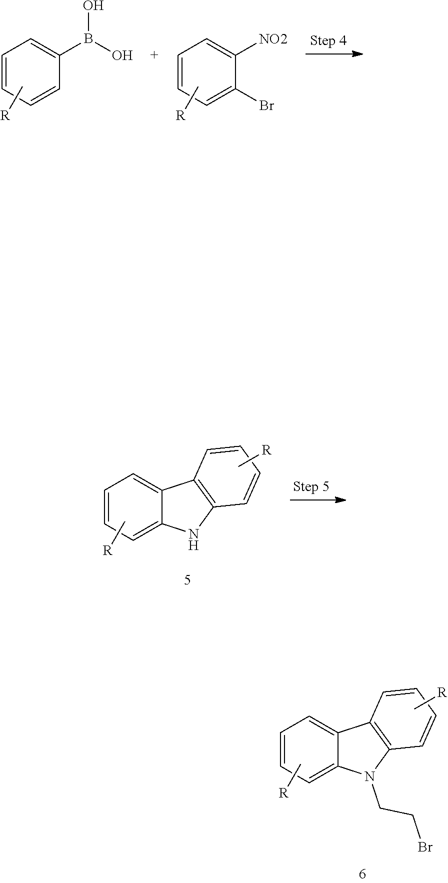

Step 1:

A suspension of compound 2 (200 mg, 0.685 mmol) and NaN3 (50 mg, 0.77 mmol) in dry DMF (1.0 mL) was heated at 150° C. under Ar for 3 h. After cooling to ambient temperature the excess of DMF was removed under rotavapour. The crude reaction mixture was added directly to a flash chromatography column packed with 20% methanol/DCM yielded compound as a white solid. 1H NMR (CD3OD): δ=3.58 (dd, J=4.4, 12.7 Hz, 1H, H-5′b), 3.84 (dd, J=7.0, 12.7 Hz, 1H, H-5′a), 4.33 (dd, J=4.0, 6.1 Hz, 1H, H-3′), 4.53 (ddd, J=4.4, 6.1, 7.0 Hz, 1H, H-4′), 4.88 (dd, J=4.0, 4.0 Hz, 1H, H-2′), 5.99 (d, J=4.0 Hz, 1H, H-1′), 8.22 (s, 1H, H-2), 8.27 (s, 1H, H-8) ppm.

Step 2:

To a stirred solution of SM (1 eq) in MeOH and followed by the addition of 10% Pd/C under H2 balloon. The reaction mixture was stirred for 24 h at room temperature. After completion of reaction the reaction mixture was filtered through celite bed. The solvent as evaporated under vaccum and the crude product was used for next step without any further purification.

Step 3:

To a solution of SM (2.0 mmol) in dry MeOH were added DIPEA (5.0 mmol) and alkyl halide (2.0 mmol) under nitrogen, and the mixture was stirred at 130° C. for 12 h in sealed tube. The reaction mixture concentrated under reduced pressure. The residue was purified by column chromatography (DCM:MeOH, 9:1-18:2) to give the 3a compound as a solid.

Step 4:

To a re-sealable pressure tube equipped with magnetic stir bar were added obromonitrobenzene 1 (0.5 mmol), aryl boronic acid 2 (0.65 mmol), Pd(OAc) (0.01 mmol, 2 mol %), PPh3 (1.25 mmol, 250 mol %), K2CO3 (1 mmol, 200 mol %) and o-DCB (1 mL). The mixture was heated at 180° C. (oil bath temperature) for 24-48 hr, at which point the reaction mixture was allowed to cool to ambient temperature. The reaction mixture was filtered through a pad of celite and the resulting liquor was concentrated in vacuo and purified by flash column chromatography (SiO2).

Step 5:

A suspension of compound 5 (1 eq) in dry 1.2dibromoethane (10 mL) and followed by the addition of 30% NaOH solution and TBAB (2 eq) was heated at 90° C. for 3 h. After cooling to ambient temperature the excess of 1,2DBE was removed under rotavapour. The crude reaction mixture was added directly to a flash chromatography column packed with 10% EA/Hexane yielded compound as a solid.

TR-H-MT-01: 1H NMR (CD3OD): δ=8.09 (s, 1H), 7.96 (d, J=7.8 Hz, 1H), 7.92 (s, 1H,), 7.87 (d, J=7.8 Hz, 1H), 7.44 (d, J=7.8 Hz, 1H), 7.32 (m, 1H), 7.12 (t, J=15 Hz, 1H), 6.98 (t, J=15 Hz, 1H), 5.87 (d, J=5.4 Hz, 1H), 4.72 (t, J=10.8 Hz, 1H), 4.49 (m, 2H), 4.19 (m, 2H), 3.18 (m, 2H), 3.06 (m, 2H), 2.48 (s, 3H) ppm.

TR-H-MT-02: 1H NMR (CD3OD): δ=8.10 (s, 1H), 7.96 (d, J=7.2 Hz, 2H), 7.93 (s, 1H,), 7.46 (d, J=8.4 Hz, 1H), 7.33 (t, J=14.4 Hz, 1H), 7.23 (d, J=12.6 Hz, 1H), 7.14 (t, J=15 Hz, 1H), 5.85 (d, J=5.4 Hz, 1H), 4.73 (t, J=10.8 Hz, 1H), 4.45 (m, 2H), 4.18 (m, 2H), 3.13 (m, 2H), 3.00 (m, 2H), 2.48 (s, 3H) ppm.

TR-H-MT-03: 1H NMR (CD3OD): δ=8.35 (d, J=7.8 Hz, 1H), 8.31 (s, 1H), 8.25 (d, J=7.2 Hz, 2H), 8.10 (s, 1H,), 8.04 (s, 1H), 7.68 (d, J=8.4 Hz, 1H), 7.68 (d, J=8.4 Hz, 1H), 7.48 (m, 2H), 7.31 (s, 2H), 7.25 (t, J=15 Hz, 1H), 5.84 (m, 1H), 4.62 (m, 2H), 4.56 (m, 1H), 4.10 (m, 1H), 3.95 (m, 1H), 3.16 (m, 1H), 2.97 (s, 3H) ppm.

TR-H-MT-04: 1H NMR (CD3OD): δ=8.09 (s, 1H), 7.86 (d, J=7.2 Hz, 2H), 7.93 (s, 1H,), 7.36 (d, J=8.4 Hz, 1H), 7.33 (t, J=14.4 Hz, 1H), 7.13 (d, J=12.6 Hz, 1H), 7.14 (t, J=15 Hz, 1H), 5.85 (d, J=5.4 Hz, 1H), 4.73 (t, J=10.8 Hz, 1H), 4.35 (m, 2H), 4.18 (m, 2H), 3.13 (m, 2H), 3.00 (m, 2H), 2.48 (s, 3H) ppm.

TR-H-MT-05: 1H NMR (CD3OD): δ=8.09 (m, 2H), 7.89 (s, 1H), 7.49 (d, J=8.4 Hz, 1H), 7.34 (3, 2H), 7.24 (t, J=15 Hz, 1H), 7.15 (t, J=14.4 Hz, 1H), 6.92 (d, J=7.2 Hz, 1H), 4.71 (t, J=10.8 Hz, 1H), 4.50 (m, 2H), 4.18 (m, 2H), 3.15 (m, 2H), 3.02 (m, 2H), 2.81 (s, 3H) ppm.

TR-H-MT-06: 1H NMR (CD3OD): δ=8.18 (m, 1H), 8.10 (s, 1H), 7.97 (s, 1H,), 7.43 (d, J=8.4 Hz, 1H), 7.21 (m, 2H), 7.12 (t, J=15 Hz, 1H), 7.06 (d, J=7.8 Hz, 1H), 6.67 (d, J=7.8 Hz, 1H), 5.86 (d, J=5.4 Hz, 1H), 4.71 (t, J=11.4 Hz, 1H), 4.45 (m, 2H), 4.17 (m, 2H), 3.16-3.05 (m, 2H), 2.98 (m, 2H) ppm.

TR-H-MT-07: 1H NMR (CD3OD): δ=8.11 (s, 1H), 8.01 (d, J=7.8 Hz, 2H), 7.97 (s, 1H,), 7.82 (d, J=7.2 Hz, 1H), 7.50 (d, J=8.4 Hz, 1H), 7.40 (t, J=15 Hz, 1H), 7.13 (m, 3H), 5.86 (d, J=6 Hz, 1H), 4.37 (t, J=10.2 Hz, 1H), 4.65 (m, 2H), 4.20 (m, 2H), 3.20 (m, 2H), 3.08 (m, 2H) ppm.

TR-H-MT-09: 1H NMR (CD3OD): δ=8.09 (s, 1H), 7.97 (d, J=7.2 Hz, 2H), 7.83 (s, 1H,), 7.36 (s, 1H), 7.33 (t, J=14.4 Hz, 1H), 7.23 (d, J=12.6 Hz, 1H), 7.14 (t, J=15 Hz, 1H), 5.95 (d, J=5.4 Hz, 1H), 4.83 (t, J=10.8 Hz, 1H), 4.55 (m, 2H), 4.08 (m, 2H), 3.23 (m, 2H), 3.10 (m, 2H), 2.38 (s, 3H) ppm.

TR-H-MT-010: 1H NMR (CD3OD): δ=8.07 (s, 1H), 7.84 (m, 3H), 7.43 (s, 1H,), 7.26 (s, 1H), 7.19 (d, J=7.8 Hz, 1H), 6.95 (d, J=7.2 Hz, 1H), 5.85 (d, J=5.4 Hz, 1H), 4.72 (t, J=9.6 Hz, 1H), 4.48 (m, 2H), 4.18 (m, 2H), 3.15 (m, 2H), 3.03 (m, 2H), 2.47 (s, 3H), 1.37 (s, 9H) ppm.

TR-H-MT-011: 1H NMR (CD3OD): δ=8.10 (s, 1H), 7.95 (d, J=7.8 Hz, 1H), 7.91 (s, 1H,), 7.87 (d, J=7.8 Hz, 1H), 7.43 (d, J=7.8 Hz, 1H), 7.32 (m, 1H), 7.12 (t, J=15 Hz, 1H), 6.98 (t, J=15 Hz, 1H), 5.87 (d, J=5.4 Hz, 1H), 4.73 (t, J=10.8 Hz, 1H), 4.39 (m, 2H), 4.19 (m, 2H), 3.18 (m, 2H), 3.06 (m, 2H), 2.48 (s, 3H) ppm.

TR-H-MT-012: 1H NMR (CD3OD): δ=8.09 (s, 1H), 7.96 (d, J=7.8 Hz, 1H), 7.92 (s, 1H,), 7.87 (d, J=7.8 Hz, 1H), 7.44 (d, J=7.8 Hz, 1H), 7.32 (m, 1H), 7.12 (t, J=15 Hz, 1H), 6.98 (t, J=15 Hz, 1H), 5.87 (d, J=5.4 Hz, 1H), 4.72 (t, J=10.8 Hz, 1H), 4.49 (m, 2H), 4.19 (m, 2H), 3.18 (m, 2H), 3.06 (m, 2H), 2.48 (s, 3H) ppm.

TR-H-MT-013: 1H NMR (CD3OD): δ=8.08 (s, 1H), 7.89 (s, 1H), 7.80 (d, J=7.8 Hz, 2H), 7.23 (s, 2H,), 6.94 (d, J=7.2 Hz, 2H), 5.86 (d, J=4.8 Hz, 1H), 4.71 (m, 1H), 4.43 (m, 2H), 4.19 (m, 2H), 3.35 (m, 1H), 3.14 (m, 2H), 3.04 (m, 2H), 2.45 (s, 6H) ppm.

TR-H-MT-014: 1H NMR (CD3OD): δ=8.10 (s, 1H), 7.96 (d, J=7.8 Hz, 1H), 7.92 (s, 1H,), 7.87 (d, J=7.8 Hz, 1H), 7.44 (d, J=7.8 Hz, 1H), 7.32 (m, 1H), 7.12 (t, J=15 Hz, 1H), 6.98 (t, J=15 Hz, 1H), 5.87 (d, J=5.4 Hz, 1H), 4.72 (t, J=10.8 Hz, 1H), 4.49 (m, 2H), 4.19 (m, 2H), 3.18 (m, 2H), 3.06 (m, 2H), 2.48 (s, 3H) ppm.

TR-H-MT-015: 1H NMR (CD3OD): δ=8.25 (s, 1H), 8.16 (s, 1H), 8.11 (d, J=7.8 Hz, 1H), 7.37 (m, 2H,), 7.15 (d, J=7.8 Hz, 1H), 7.06 (d, J=7.2 Hz, 1H), 6.76 (d, J=7.2 Hz, 1 H), 5.97 (d, J=5.4 Hz, 1H), 4.76 (m, 1H), 4.50 (m, 2H), 4.26 (m, 1H), 4.19 (m, 1H), 3.35 (m, 1H), 3.16 (m, 4H), 2.53 (s, 3H) ppm.

TR-H-MT-016: 1H NMR (CD3OD): δ=8.29 (d, J=8.4 Hz, 1H), 8.18 (s, 1H), 8.10 (s, 1H), 7.80 (m, 2H,), 7.39 (m, 2H), 7.14 (d, J=8.4 Hz, 1H), 6.75 (d, J=7.8 Hz, 1H), 5.84 (d, J=5.4 Hz, 1H), 4.69 (m, 1H), 4.58 (m, 2H), 4.21 (m, 2H), 4.09 (s, 3H), 3.73 (m, 1H), 3.60 (m, 1H), 3.24 (m, 2H), 3.13 (m, 2H) ppm.

TR-H-MT-017: 1H NMR (CD3OD): δ=8.20 (m, 1H), 8.08 (s, 1H), 7.85 (s, 1H), 7.67 (s, 1H,), 7.42 (m, 3H), 7.02 (d, J=7.2 Hz, 1H), 5.83 (d, J=5.4 Hz, 1H), 4.67 (m, 3H), 4.25 (m, 2H), 3.19 (m, 3H), 2.84 (s, 3H) ppm.

TR-H-MT-018: 1H NMR (CD3OD): δ=8.18 (m, 1H), 8.09 (s, 1H), 7.94 (s, d=7.8 Hz, 1H), 7.82 (s, 1H,), 7.30 (m, 2H), 7.21 (m, 1H), 6.98 (d, J=7.8 Hz, 1H), 6.91 (d, J=7.2 Hz, 1H), 5.85 (d, J=5.4 Hz, 1H), 4.70 (m, 1H), 4.50 (m, 2H), 4.21 (m, 2H), 3.22 (m, 2H), 3.11 (m, 2H), 2.78 (s, 3H), 2.48 (m, 2H) ppm.

TR-H-MT-019: 1H NMR (CD3OD): δ=8.10 (m, 1H), 7.89 (s, 1H), 7.80 (s, d=8.4 Hz, 1H), 7.76 (d, J=7.8 Hz, 1H,), 7.23 (m, 1H), 6.96 (m, 2H), 6.72 (m, 1H), 5.87 (d, J=6 Hz, 1H), 4.72 (m, 1H), 4.44 (m, 2H), 4.21 (m, 2H), 3.85 (s, 3H), 3.72 (m, 1H), 3.22 (m, 2H), 3.09 (m, 2H), 2.45 (s, 2H) ppm.

Analogs synthesized according to Scheme 7:

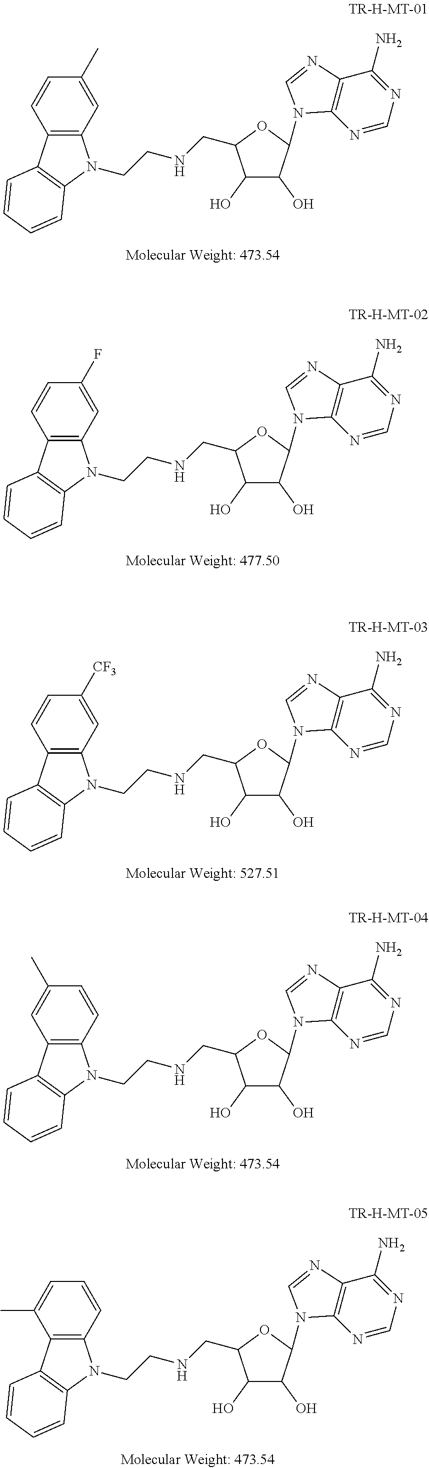 [SMILES: Cc1ccc2c3ccccc3n(CCNCC3OC(n4cnc5c(N)ncnc54)C(O)C3O)c2c1 ; Nc1ncnc2c1ncn2C1OC(CNCCn2c3ccccc3c3ccc(F)cc32)C(O)C1O ; Nc1ncnc2c1ncn2C1OC(CNCCn2c3ccccc3c3ccc(C(F)(F)F)cc32)C(O)C1O ; Cc1ccc2c(c1)c1ccccc1n2CCNCC1OC(n2cnc3c(N)ncnc32)C(O)C1O ; Cc1cccc2c1c1ccccc1n2CCNCC1OC(n2cnc3c(N)ncnc32)C(O)C1O]

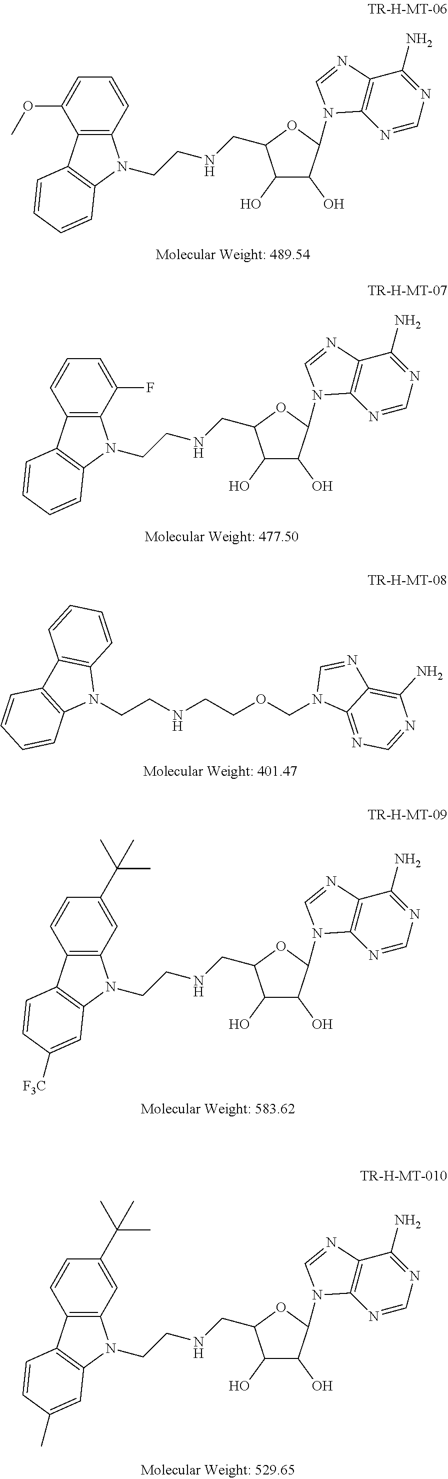 [SMILES: COc1cccc2c1c1ccccc1n2CCNCC1OC(n2cnc3c(N)ncnc32)C(O)C1O ; Nc1ncnc2c1ncn2C1OC(CNCCn2c3ccccc3c3cccc(F)c32)C(O)C1O ; Nc1ncnc2c1ncn2COCCNCCn1c2ccccc2c2ccccc21 ; CC(C)(C)c1ccc2c3ccc(C(F)(F)F)cc3n(CCNCC3OC(n4cnc5c(N)ncnc54)C(O)C3O)c2c1 ; Cc1ccc2c3ccc(C(C)(C)C)cc3n(CCNCC3OC(n4cnc5c(N)ncnc54)C(O)C3O)c2c1]

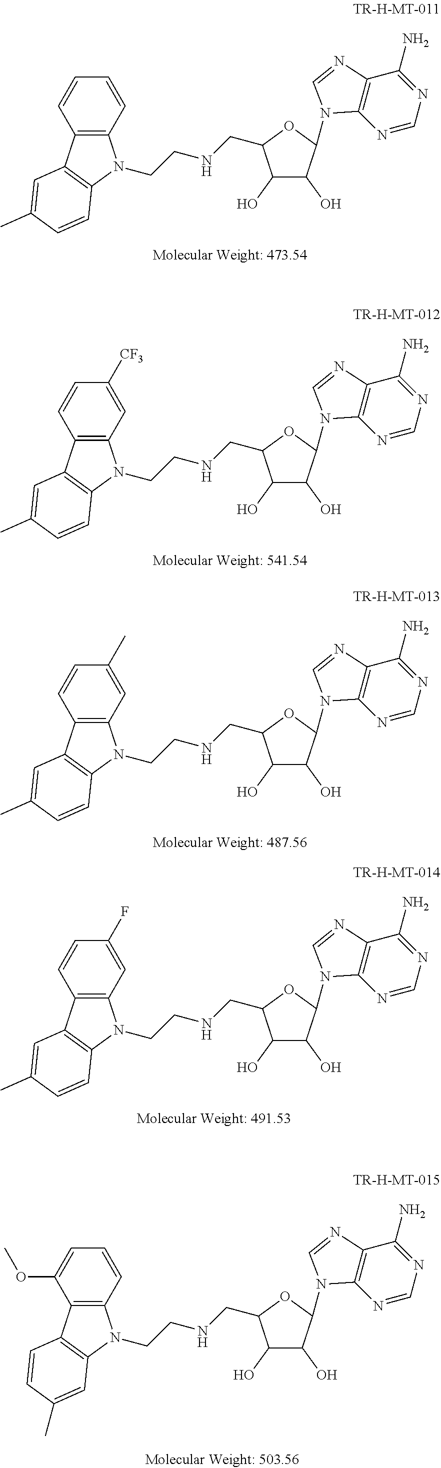 [SMILES: Cc1ccc2c(c1)c1ccccc1n2CCNCC1OC(n2cnc3c(N)ncnc32)C(O)C1O ; Cc1ccc2c(c1)c1ccc(C(F)(F)F)cc1n2CCNCC1OC(n2cnc3c(N)ncnc32)C(O)C1O ; Cc1ccc2c(c1)c1ccc(C)cc1n2CCNCC1OC(n2cnc3c(N)ncnc32)C(O)C1O ; Cc1ccc2c(c1)c1ccc(F)cc1n2CCNCC1OC(n2cnc3c(N)ncnc32)C(O)C1O ; COc1cccc2c1c1ccc(C)cc1n2CCNCC1OC(n2cnc3c(N)ncnc32)C(O)C1O]

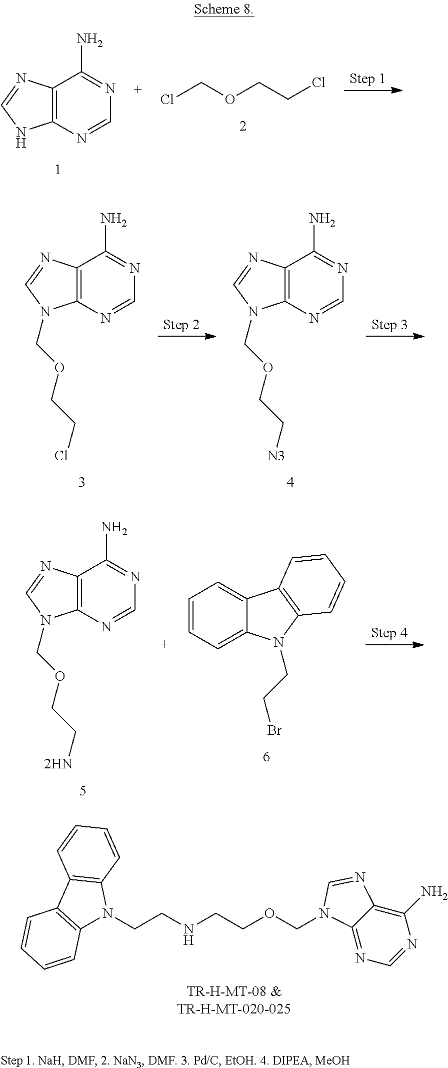

Step 1:

The SM (1) (7.26 mmol) was dissolved in DMF (20 mL) and cooled by stirring in an ice bath under N2. Compound (2) (7.26 mmol) was added in a single portion followed by 60% NaH/oil (9.2 mmol), added carefully in small portions over 10 min. The reaction mixture was stirred at room temperature for 3 h. Cold Ethylacetate (150 mL) and ice-water (50 mL) were added and the organic phase was separated. The aqueous phase was extracted with more Ethylacetate (100 mL) and the combined extracts were washed with water then brine, and dried over MgSO4. The solvents were evaporated and the residue was purified by column chromatography on silica gel (DCM:MeOH, 9:1) yielded compound as a white solid.

Step 2:

To stirred solution of SM(3) (1 equiv) in DMF and followed by the addition of NaN3 (1.5 equiv). The reaction mixture was stirred for 5 h at 120° C. After completion of reaction to the reaction mass add excess of water and ethyl acetate. The organic layer was dried overMgSO4 and concentrated. The crude mixture was purified by column chromatography with a gradient of (DCM:MeOH, 9:1) to afford title compound as a white solid.

Step 3:

To a stirred solution of SM(4) (1 eq) in EtOH and followed by the addition of 10% Pd/C under H2 balloon. The reaction mixture was stirred for 24 h at room temperature. After completion of reaction the reaction mixture was filtered through celite bed. The solvent as evaporated under vacuum and the crude product was used for next step without any further purification.

Step 4:

To a solution of SM (2.0 mmol) in dry MeOH were added DIPEA (5.0 mmol) and compound 6 (2.0 mmol) under nitrogen, and the mixture was stirred at 130° C. for 12 h in sealed tube. The reaction mixture concentrated under reduced pressure. The residue was purified by column chromatography (DCM:MeOH, 9:1) to give the 7 compound as a off white solid.

TR-H-M-08: 1H NMR (CD3OD): δ=8.18 (s, 1H), 8.11 (s, 1H), 8.07 (d, J=7.8 Hz, 2H), 7.49 (d, J=7.8 Hz, 2H,), 7.42 (t, J=15 Hz, 2H), 7.42 (t, J=14.4 Hz, 2H), 5.52 (s, 1H), 4.47 (t, J=13.2 Hz, 2H), 3.59 (m, 2H), 3.03 (m, 2H), 2.78 (m, 2H) ppm.

TR-H-MT-020: 1H NMR (CD3OD): δ=8.17 (m, 3H), 7.50 (m, 1H), 7.43 (m, 1H), 7.34 (m, 2H,), 7.22 (m, 1H), 6.99 (m, 1H), 5.54 (s, 2H), 4.51 (m, 2H), 3.64 (m, 2H), 3.11 (m, 2H), 2.88 (m, 2H), 2.84 (s, 3H) ppm.

TR-H-MT-021: 1H NMR (CD3OD): δ=8.24 (m, 1H), 8.16 (m, 3H), 7.88 (s, 1H), 7.52 (m, 3H,), 7.27 (m, 1H), 5.55 (s, 2H), 4.53 (m, 2H), 3.61 (m, 2H), 3.04 (m, 2H), 2.72 (m, 2H) ppm.

TR-H-MT-022: 1H NMR (CD3OD): δ=8.17 (m, 2H), 8.02 (m, 1H), 7.30 (m, 3H), 7.05 (m, 1H,), 6.96 (m, 1H), 5.54 (s, 2H), 4.48 (m, 2H), 3.64 (m, 2H), 3.10 (m, 2H), 2.88 (m, 2H), 2.80 (s, 3H), 2.54 (s, 3H) ppm.

TR-H-MT-023: 1H NMR (CD3OD): δ=8.26 (m, 1H), 8.06 (m, 3H), 7.78 (s, 1H), 7.33 (m, 3H,), 7.18 (m, 1H), 5.54 (s, 2H), 4.43 (m, 2H), 3.60 (m, 2H), 3.14 (m, 2H), 2.72 (m, 2H) ppm.

TR-H-MT-024: 1H NMR (CD3OD): δ=8.18 (m, 3H), 7.52 (m, 1H), 7.23 (m, 1H), 7.12 (m, 2H,), 7.02 (m, 1H), 6.99 (m, 1H), 5.55 (s, 2H), 4.49 (m, 2H), 3.60 (m, 2H), 3.10 (m, 2H), 2.78 (m, 2H), 2.74 (s, 3H), 0.99 (s, 9H) ppm.

TR-H-MT-025: 1H NMR (CD3OD): δ=8.19 (m, 2H), 7.42 (m, 1H), 7.32 (m, 2H), 7.22 (m, 2H,), 7.02 (m, 1H), 6.92 (m, 1H), 5.54 (s, 2H), 4.50 (m, 2H), 3.63 (m, 2H), 3.12 (m, 2H), 2.88 (m, 2H), 2.74 (s, 3H) ppm.

Analogs synthesized according to Scheme 8:

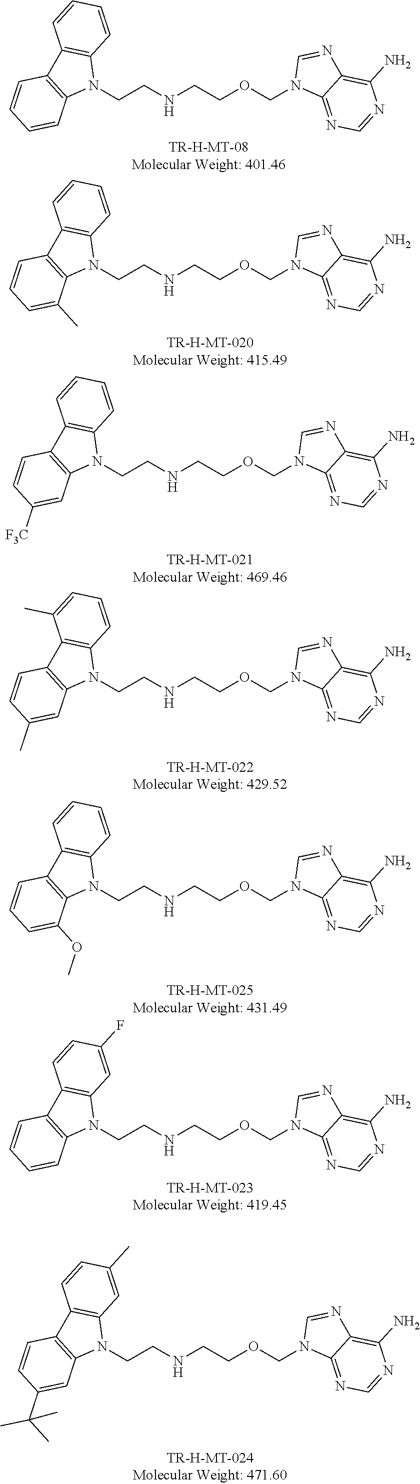

Representative scaffolds disclosed in Schemes 1-5 and in Tables 2-5 include, but are not intended to be limited to, the following:

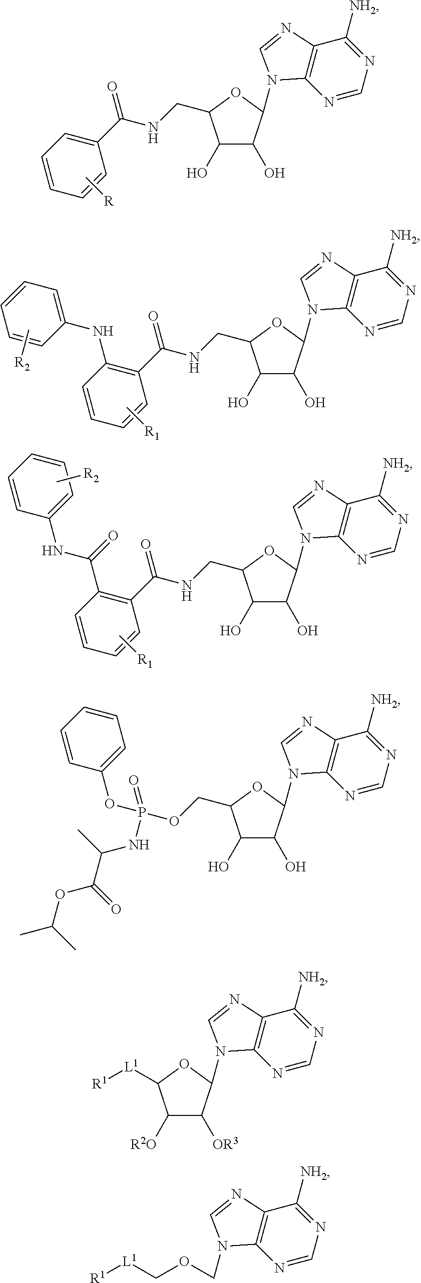

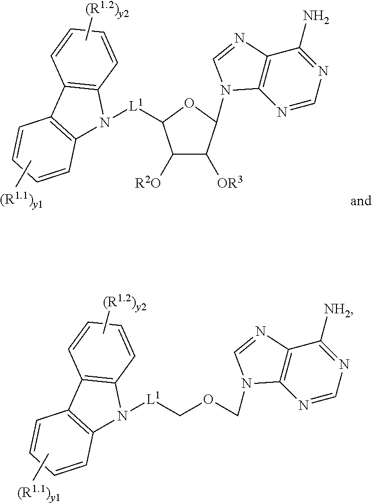

wherein substituents R, R¹, R², R³, R^{1.1}, R^{1.2} and L¹ include the substituents set forth in Schemes 1-5, Table 3, and as described herein (e.g., substituent group, size-limited substituent group, or lower substituent group).

TABLE 3
Compounds synthesized are set forth in the table.

| | |
|---|---|
| 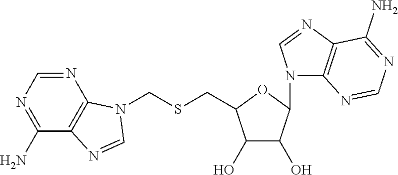 | 1 |
| 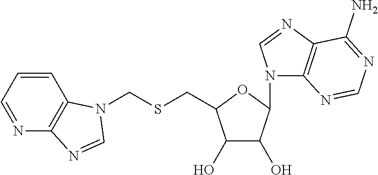 | 2 |
| 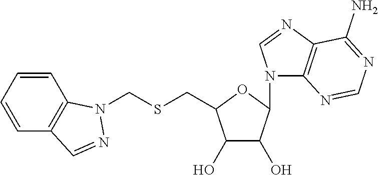 | 3 |
| 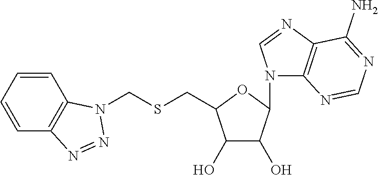 | 4 |
| 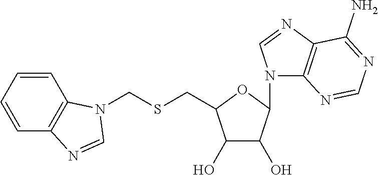 | 5 |
| 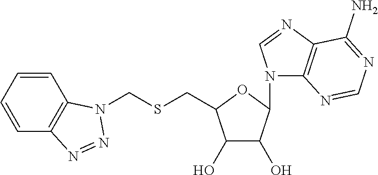 | 6 |
| 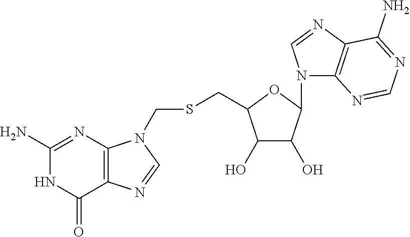 | 7 |
| 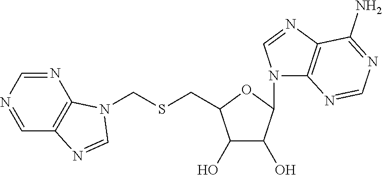 | 8 |
| 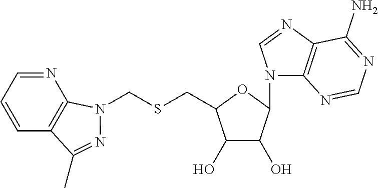 | 9 |
| 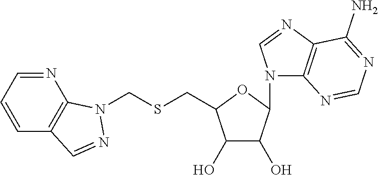 | 10 |
| 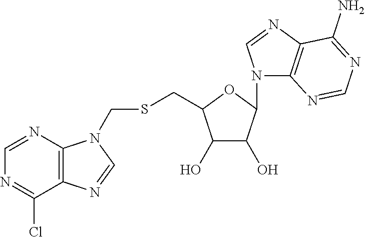 | 11 |
| 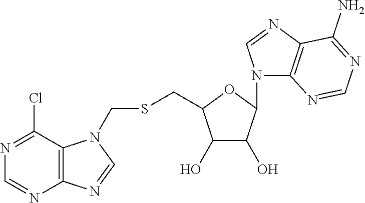 | 12 |
| 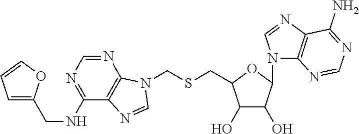 | 13 |
| 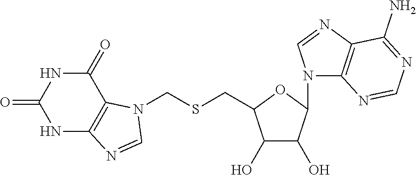 | 14 |
| 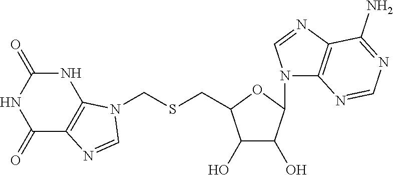 | 15 |
| 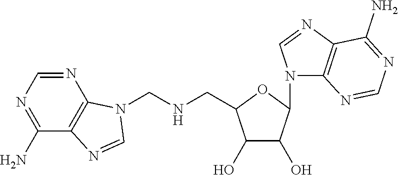 | 16 |
| 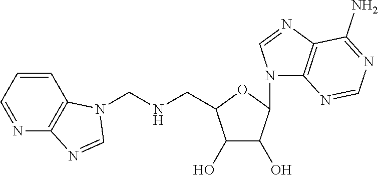 | 17 |
| 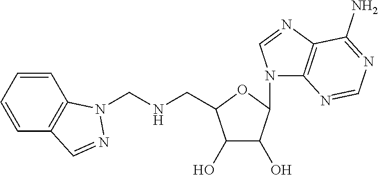 | 18 |
| 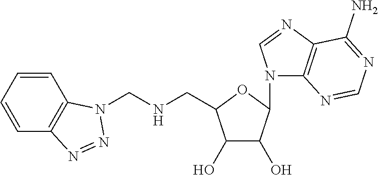 | 19 |
| 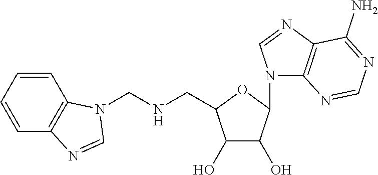 | 20 |
| 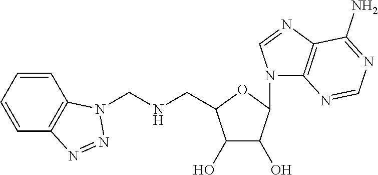 | 21 |
| 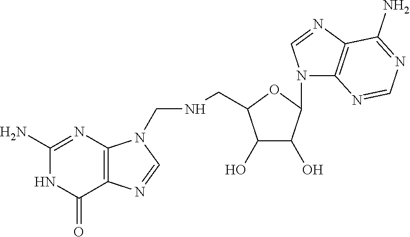 | 22 |
| 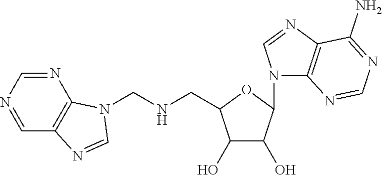 | 23 |
| 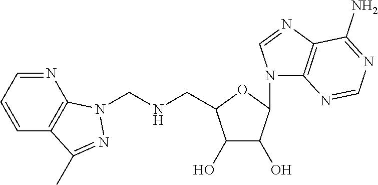 | 24 |
| 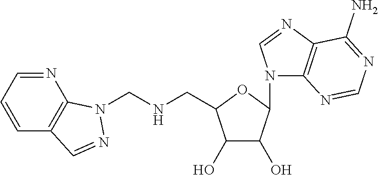 | 25 |
| 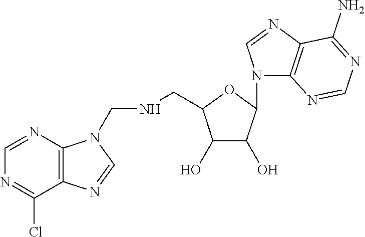 | 26 |
| 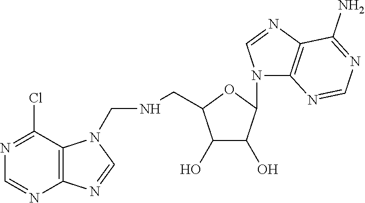 | 27 |
| 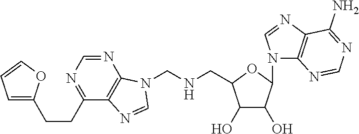 | 28 |
| 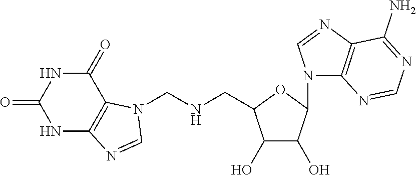 | 29 |
| 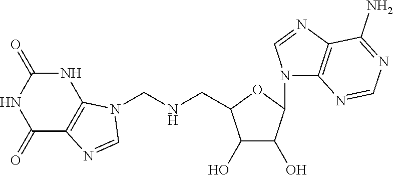 | 30 |
| 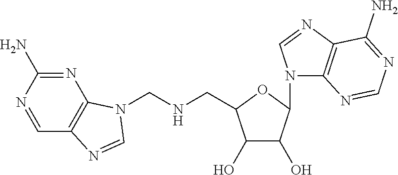 | 31 |
| 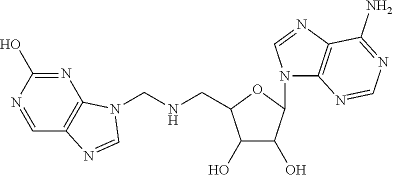 | 32 |
| 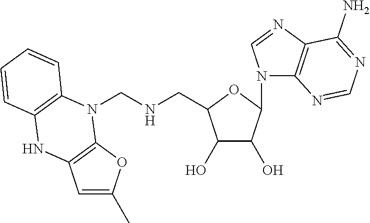 | 33 |
| 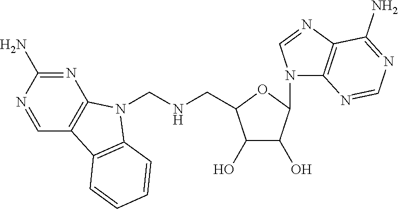 | 34 |
| 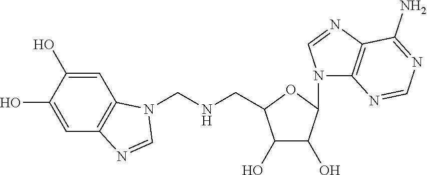 | 35 |
| 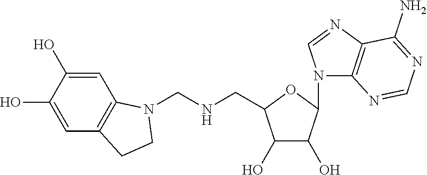 | 36 |
| 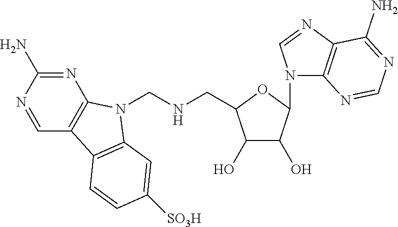 | 37 |
| 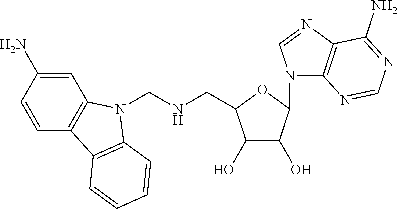 | 38 |
| 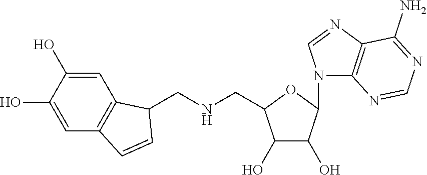 | 39 |
| 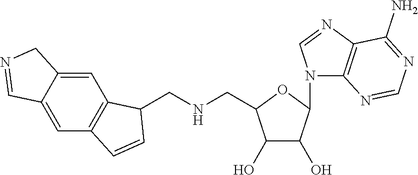 | 40 |
| 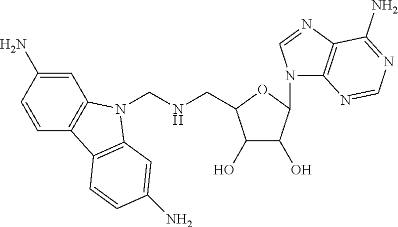 | 41 |
| 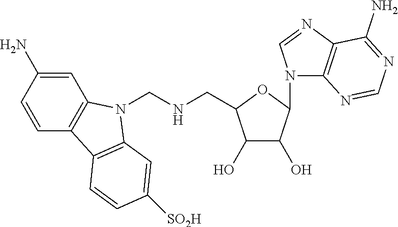 | 42 |
| 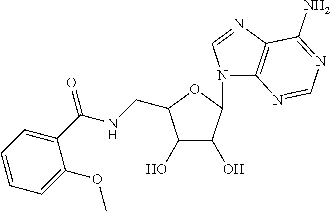 | 43 |
| 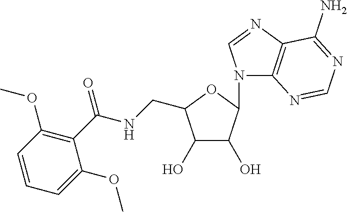 | 44 |
| 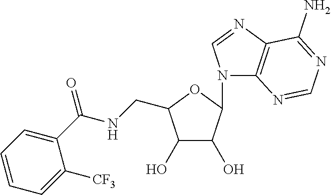 | 45 |
| 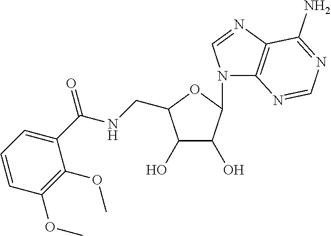 | 46 |
| 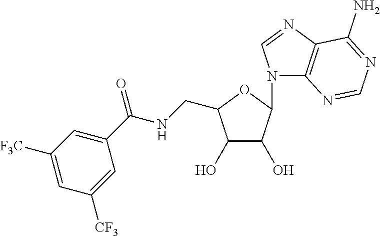 | 47 |
| 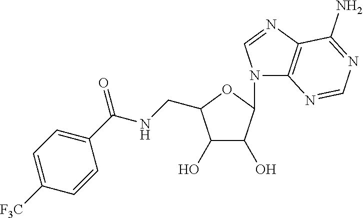 | 48 |
| 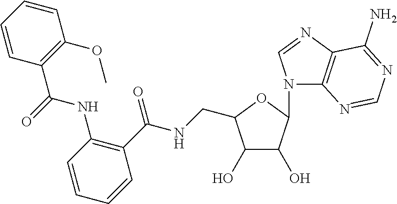 | 49 |
| 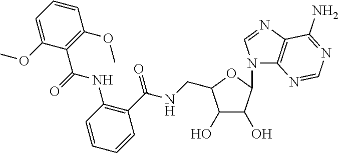 | 50 |
| 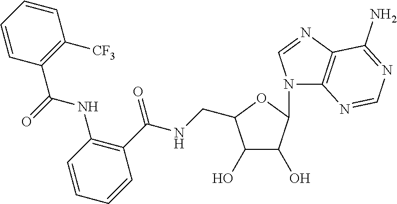 | 51 |
| 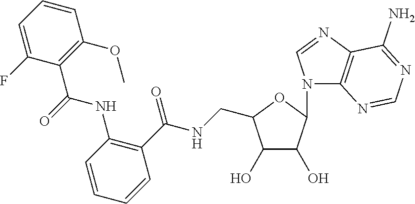 | 52 |
| 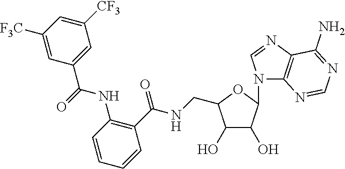 | 53 |
| 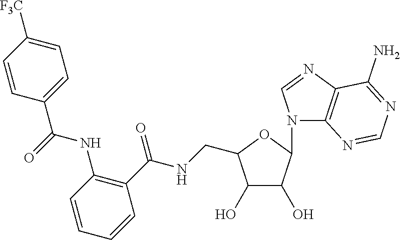 | 54 |
| 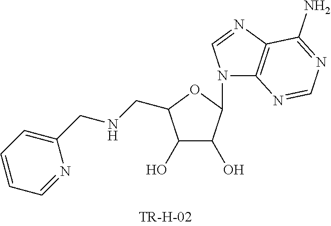 | |
| 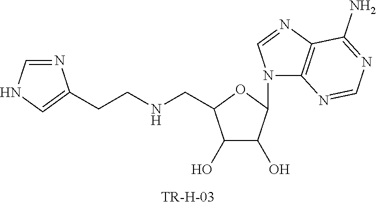 | |
| 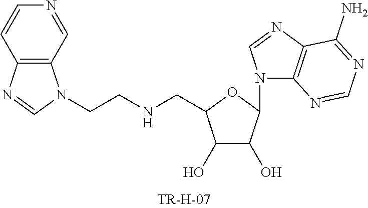 | |
| 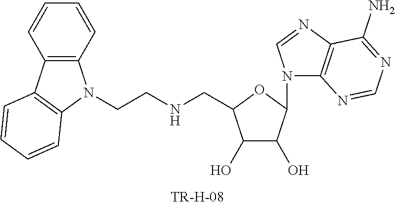 | |
| 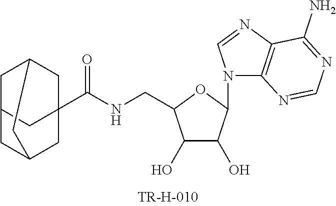 | |
| 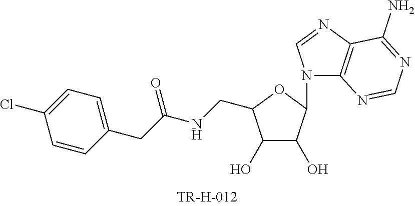 | |
| 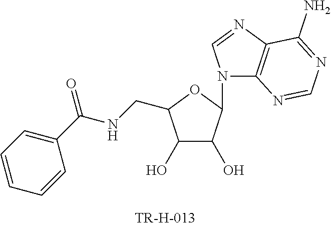 | |
| 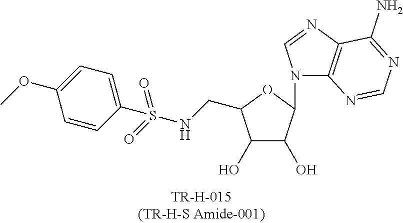 | |
| 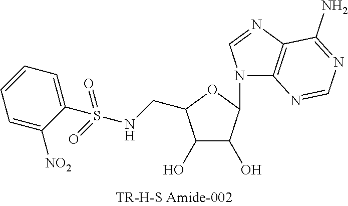 | |
| 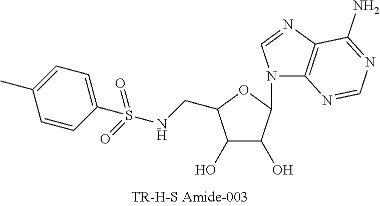 | |
| 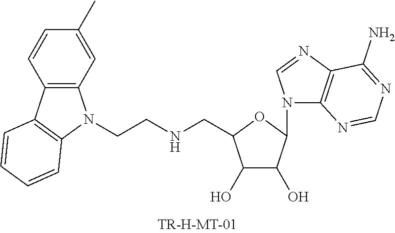 | |
| 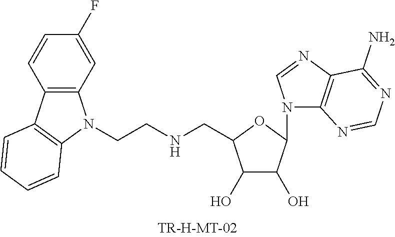 | |
| 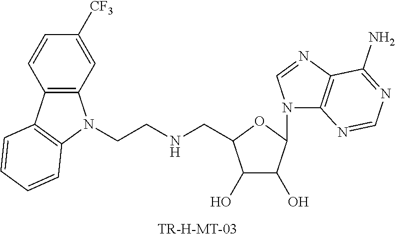 | |
| 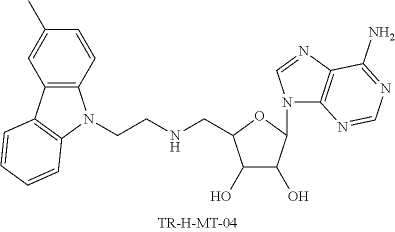 | |
| 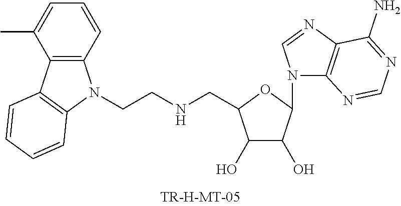 | |
| 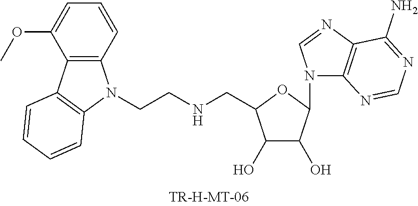 | |
| 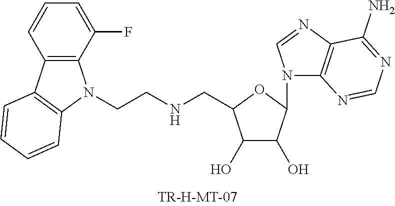 | |
| 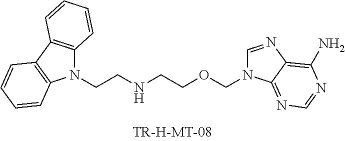 | |
| 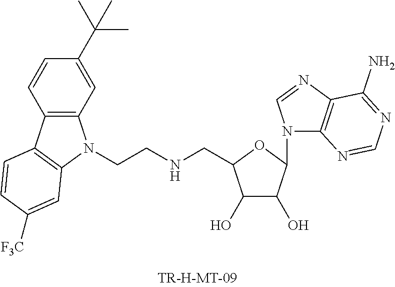 | |
| 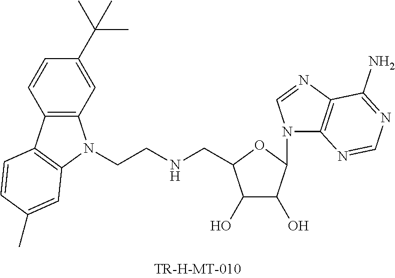 | |
| 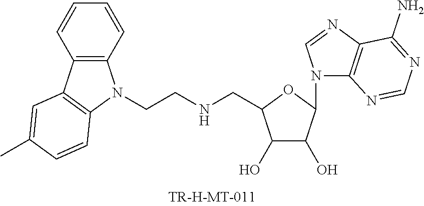 | |
| 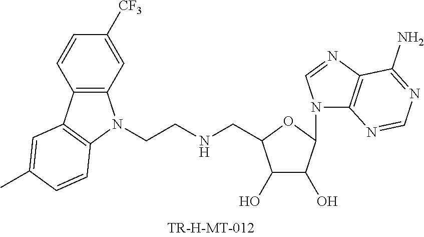 | |
| 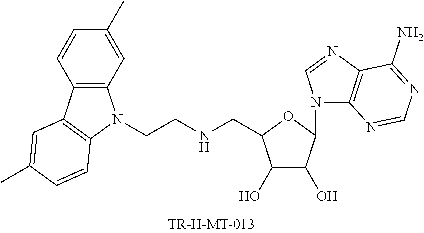 | |
| 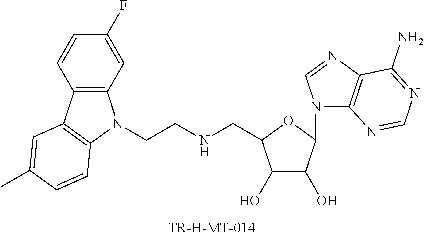 | |
| 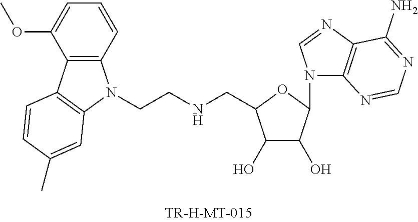 | |
| 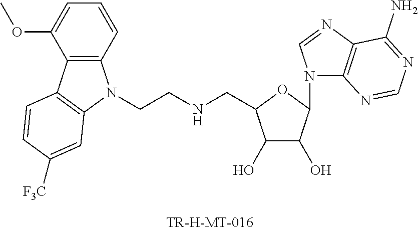 | |
| 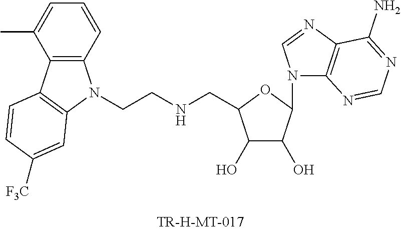 | |
| 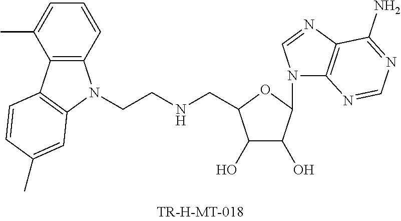 | |
| 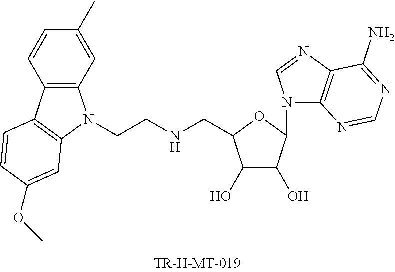 | |
| 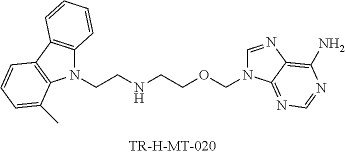 | |
| 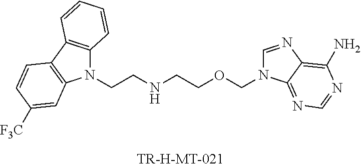 | |
| 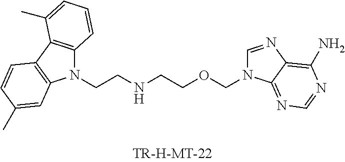 | |
| 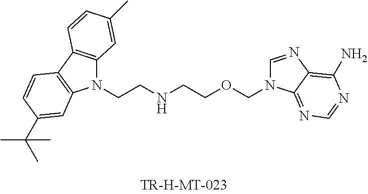 | |
| 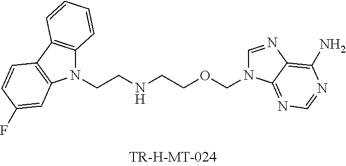 | |
| 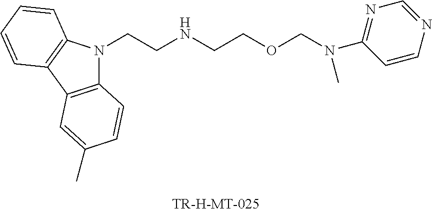 | |

### Example 3—Inhibitors of RNA 2′-O-Methyl Transferase

Inhibitors of RNA 2-‘O’methyl transferase, useful in the compositions and methods disclosed herein, include compounds set forth in Table 4 following.

TABLE 4

| Description | Chemical Structure |
|---|---|
| Ribavirin and its analogs by SAR | 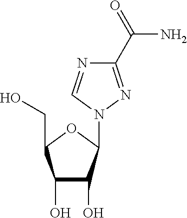 |
| Ara-C and its analogs by SAR | 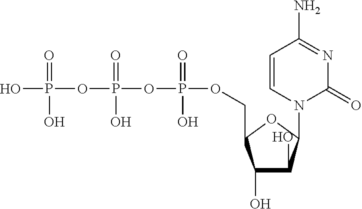 |
| Sofosbuvirand its analogs by SAR | 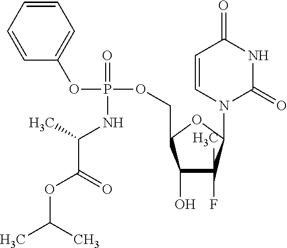 |
| Acyclovir and its analogs by SAR | 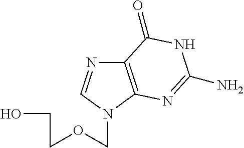 [SMILES: Nc1nc2c(ncn2COCCO)c(=O)[nH]1] |
| Gancyclovir and its analogs by SAR | 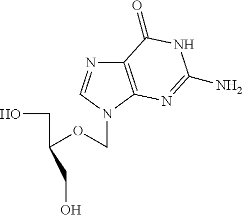 |

### Example 4—Inhibitor Activity Analysis

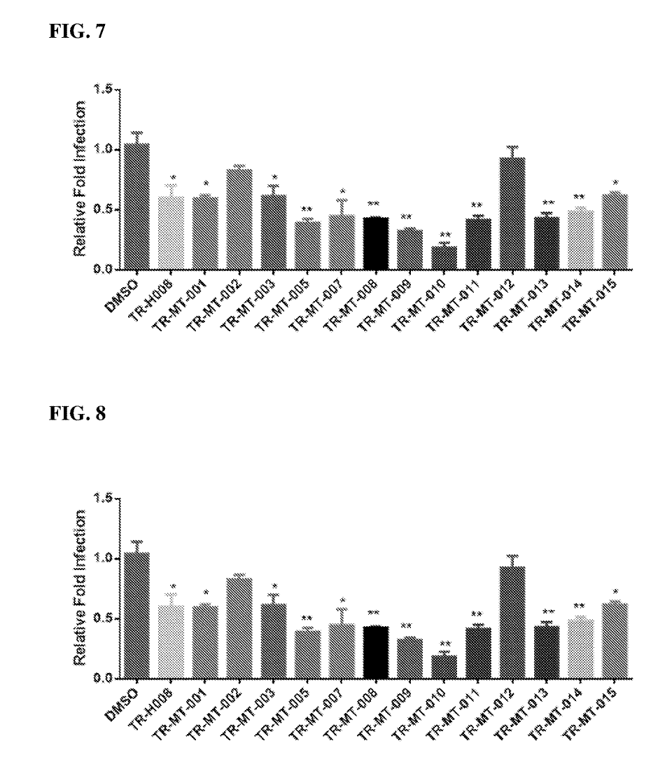

Effects of methyltransferase inhibitors on ZIKV replication was monitored using previously published methods (Dang, J., Tiwari, S. K., Lichinchi, G., Qin, Y., Patil, V. S., Eroshkin, A. M., and Rana, T. M. (2016); Zika Virus Depletes Neural Progenitors in Human Cerebral Organoids through Activation of the Innate Immune Receptor TLR3. Cell Stem Cell 19, 258-265; Lichinchi, G., Zhao, B. S., Wu, Y., Lu, Z., Qin, Y., He, C., and Rana, T. M. (2016). Dynamics of Human and Viral RNA Methylation during Zika Virus Infection. Cell Host Microbe 20, 666-673; Tiwari, S. K., Dang, J., Qin, Y., Lichinchi, G., Bansal, V., and Rana, T. M. (2017), which are incorporated herein in their entirety for all purposes). Zika virus infection reprograms global transcription of host cells to allow sustained infection. Emerg Microbes Infect 6, e24 for ZIKV infections of microglia cells. Microglial cell-line was plated 1×10⁶ cells per well in 24 well plate and cultured for 24 h. Cell were then treated in triplicate with series of methyltransferase inhibitors (10 μM) for one hour followed by infection with ZIKV (MR766, MOI:1) and changed the fresh medium after 2 hrs of ZIKV infection and re-added the drugs. After 24 h (FIG. 7) and 48 h (FIG. 8) of the infection collected the supernatant and did one-step qRT-PCR. Fold inhibition of ZIKV infection was normalized from DMSO control.

### Example 5—CRISPR sgRNA Sequences

TABLE 5
CRISPR sgRNA sequences, listing SEQ ID NO: 36-133. The leading cloning
nucleotides, ″CACCG″ and ″AAAC,″ are for cloning purposes. One of
ordinary skill in the art would recognize alternative cloning
nucleotides may be used. The sequences described herein may be used as
an adenosine N-6 methylation CRISPR agonist, adenosine N-6 demethylation
CRISPR antagonist, or RNA 2′-O-methyl transferase CRISPR inhibitor.

| ID | sgRNA name | Nucleotide sequence | SEQ ID |
|---|---|---|---|
| A1 | Mett13-sg1-UP | CACCGTAGGCACGGGACTATCACTA | SEQ ID NO: 36 |
| A2 | Mett13-sg2-UP | CACCGTCAGGTGATTACCGTAGAGA | SEQ ID NO: 37 |
| A3 | Mett13-sg3-UP | CACCGAGGTAGCAGGGACCATCGCA | SEQ ID NO: 38 |
| A4 | Mett13-sg4-UP | CACCGCTGAAGTGCAGCTTGCGACA | SEQ ID NO: 39 |
| A5 | Mett13-sg5-UP | CACCGACGCCGTTTCTGCCCTGCGA | SEQ ID NO: 40 |
| A6 | Mett13-sg6-UP | CACCGCTTGTCGCAAGCTGCACTTC | SEQ ID NO: 41 |
| A7 | Mett13-sg7-UP | CACCGGTTGAAAAGTTTCGCTCTCG | SEQ ID NO: 42 |
| A8 | Mett13-sg8-UP | CACCGGTTGGGGACAGTGCCGCTTC | SEQ ID NO: 43 |
| A9 | Mett13-sg9-UP | CACCGGCAGAAACGGCGTGCAGAAC | SEQ ID NO: 44 |
| B1 | Mett13-sg1-DN | AAACTAGTGATAGTCCCGTGCCTAC | SEQ ID NO: 45 |
| B2 | Mett13-sg2-DN | AAACTCTCTACGGTAATCACCTGAC | SEQ ID NO: 46 |
| B3 | Mett13-sg3-DN | AAACTGCGATGGTCCCTGCTACCTC | SEQ ID NO: 47 |
| B4 | Mett13-sg4-DN | AAACTGTCGCAAGCTGCACTTCAGC | SEQ ID NO: 48 |
| B5 | Mett13-sg5-DN | AAACTCGCAGGGCAGAAACGGCGTC | SEQ ID NO: 49 |
| B6 | Mett13-sg6-DN | AAACGAAGTGCAGCTTGCGACAAGC | SEQ ID NO: 50 |
| B7 | Mett13-sg7-DN | AAACCGAGAGCGAAACTTTTCAACC | SEQ ID NO: 51 |
| B8 | Mett13-sg8-DN | AAACGAAGCGGCACTGTCCCCAACC | SEQ ID NO: 52 |
| B9 | Mett13-sg9-DN | AAACGTTCTGCACGCCGTTTCTGCC | SEQ ID NO: 53 |
| C1 | Mett114-sg1-UP | CACCGGTCCAGTGTCTACAAAATGT | SEQ ID NO: 54 |
| C2 | Mett114-sg2-UP | CACCGCACTGAACTACTTACATGGG | SEQ ID NO: 55 |
| C3 | Mett114-sg3-UP | CACCGATCAACTTACTACTCTCCCA | SEQ ID NO: 56 |
| C4 | Mett114-sg4-UP | CACCGGCTGGACCTGGGATGATGTA | SEQ ID NO: 57 |
| C5 | Mett114-sg5-UP | CACCGCGCATACCTGCAGGTTTCTC | SEQ ID NO: 58 |
| C6 | Mett114-sg6-UP | CACCGACAGACCTCAGAATTTCATC | SEQ ID NO: 59 |
| C7 | Mett114-sg7-UP | CACCGAGATTCCAGTACCTTTCTTA | SEQ ID NO: 60 |
| C8 | Mett114-sg8-UP | CACCGCATCTGCTCCAAACTCAAAA | SEQ ID NO: 61 |
| C9 | Mett114-sg9-UP | CACCGACATCCCTGATGAAATTCTG | SEQ ID NO: 62 |
| C10 | Mett114-sg10-UP | CACCGCTAGTTGGGAGCTGAGAGTG | SEQ ID NO: 63 |
| D1 | Mett114-sg1-DN | AAACACATTTTGTAGACACTGGACC | SEQ ID NO: 64 |
| D2 | Mett114-sg2-DN | AAACCCCATGTAAGTAGTTCAGTGC | SEQ ID NO: 65 |
| D3 | Mett114-sg3-DN | AAACTGGGAGAGTAGTAAGTTGATC | SEQ ID NO: 66 |
| D4 | Mett114-sg4-DN | AAACTACATCATCCCAGGTCCAGCC | SEQ ID NO: 67 |
| D5 | Mett114-sg5-DN | AAACGAGAAACCTGCAGGTATGCGC | SEQ ID NO: 68 |
| D6 | Mett114-sg6-DN | AAACGATGAAATTCTGAGGTCTGTC | SEQ ID NO: 69 |
| D7 | Mett114-sg7-DN | AAACTAAGAAAGGTACTGGAATCTC | SEQ ID NO: 70 |
| D8 | Mett114-sg8-DN | AAACTTTTGAGTTTGGAGCAGATGC | SEQ ID NO: 71 |
| D9 | Mett114-sg9-DN | AAACCAGAATTTCATCAGGGATGTC | SEQ ID NO: 72 |
| D10 | Mett114-sg10-DN | AAACCACTCTCAGCTCCCAACTAGC | SEQ ID NO: 73 |
| E1 | Alkbh5-sg1-UP | CACCGCCCCGAGCGCAGCGACTACG | SEQ ID NO: 74 |
| E2 | Alkbh5-sg2-UP | CACCGAGAAAGACACGGACACGATG | SEQ ID NO: 75 |
| E3 | Alkbh5-sg3-UP | CACCGCCTGTACAACGAGCACACGG | SEQ ID NO: 76 |
| E4 | Alkbh5-sg4-UP | CACCGGTCACGCTCCCCCTGCGCAC | SEQ ID NO: 77 |
| E5 | Alkbh5-sg5-UP | CACCGGGTGGTGTCCCGCGCCGAGA | SEQ ID NO: 78 |
| E6 | Alkbh5-sg6-UP | CACCGATCTCGTCGACGTCGCCCGG | SEQ ID NO: 79 |
| E7 | Alkbh5-sg7-UP | CACCGAAGATTAGATGCACCGCGAT | SEQ ID NO: 80 |
| E8 | Alkbh5-sg8-UP | CACCGTATGCTTCAGATCGCCTGTC | SEQ ID NO: 81 |
| E9 | Alkbh5-sg9-UP | CACCGCTCAGGACATTAAGGAACGC | SEQ ID NO: 82 |
| E10 | Alkbh5-sg10-UP | CACCGGCAATCTTCCGAGGACTCAT | SEQ ID NO: 83 |
| F1 | Alkbh5-sg1-DN | AAACCGTAGTCGCTGCGCTCGGGGC | SEQ ID NO: 84 |
| F2 | Alkbh5-sg2-DN | AAACCATCGTGTCCGTGTCTTTCTC | SEQ ID NO: 85 |
| F3 | Alkbh5-sg3-DN | AAACCCGTGTGCTCGTTGTACAGGC | SEQ ID NO: 86 |
| F4 | Alkbh5-sg4-DN | AAACGTGCGCAGGGGGAGCGTGACC | SEQ ID NO: 87 |
| F5 | Alkbh5-sg5-DN | AAACTCTCGGCGCGGGACACCACCC | SEQ ID NO: 88 |
| F6 | Alkbh5-sg6-DN | AAACCCGGGCGACGTCGACGAGATC | SEQ ID NO: 89 |
| F7 | Alkbh5-sg7-DN | AAACATCGCGGTGCATCTAATCTTC | SEQ ID NO: 90 |
| F8 | Alkbh5-sg8-DN | AAACGACAGGCGATCTGAAGCATAC | SEQ ID NO: 91 |
| F9 | Alkbh5-sg9-DN | AAACGCGTTCCTTAATGTCCTGAGC | SEQ ID NO: 92 |
| F10 | Alkbh5-sg10-DN | AAACATGAGTCCTCGGAAGATTGCC | SEQ ID NO: 93 |
| G1 | Fto-sg1-UP | CACCGCGGTGAGTGGAACTAAACCG | SEQ ID NO: 94 |
| G2 | Fto-sg2-UP | CACCGGCAGTGTGAGAAAGGCCTCG | SEQ ID NO: 95 |
| G3 | Fto-sg3-UP | CACCGAGGAGCCCTATTTCGGCATG | SEQ ID NO: 96 |
| G4 | Fto-sg4-UP | CACCGAGTGTCTCGCATCCTCATCG | SEQ ID NO: 97 |
| G5 | Fto-sg5-UP | CACCGGCTTGTTTCGGGACGTGGTG | SEQ ID NO: 98 |
| G6 | Fto-sg6-UP | CACCGGTGTACAGCTATAGCTGCGA | SEQ ID NO: 99 |
| G7 | Fto-sg7-UP | CACCGGTTTAGTTCCACTCACCGTG | SEQ ID NO: 100 |
| G8 | Fto-sg8-UP | CACCGTGAAGAGGGATTGTTAATCC | SEQ ID NO: 101 |
| G9 | Fto-sg9-UP | CACCGAGATGATGAGTTCTATCAGC | SEQ ID NO: 102 |
| G10 | Fto-sg10-UP | CACCGATTAACAATCCCTCTTCACC | SEQ ID NO: 103 |
| H1 | Fto-sg1-DN | AAACCGGTTTAGTTCCACTCACCGC | SEQ ID NO: 104 |
| H2 | Fto-sg2-DN | AAACCGAGGCCTTTCTCACACTGCC | SEQ ID NO: 105 |
| H3 | Fto-sg3-DN | AAACCATGCCGAAATAGGGCTCCTC | SEQ ID NO: 106 |
| H4 | Fto-sg4-DN | AAACCGATGAGGATGCGAGACACTC | SEQ ID NO: 107 |
| H5 | Fto-sg5-DN | AAACCACCACGTCCCGAAACAAGCC | SEQ ID NO: 108 |
| H6 | Fto-sg6-DN | AAACTCGCAGCTATAGCTGTACACC | SEQ ID NO: 109 |
| H7 | Fto-sg7-DN | AAACCACGGTGAGTGGAACTAAACC | SEQ ID NO: 110 |
| H8 | Fto-sg8-DN | AAACGGATTAACAATCCCTCTTCAC | SEQ ID NO: 111 |
| H9 | Fto-sg9-DN | AAACGCTGATAGAACTCATCATCTC | SEQ ID NO: 112 |
| H10 | Fto-sg10-DN | AAACGGTGAAGAGGGATTGTTAATC | SEQ ID NO: 113 |
| K1 | NTC-sg1-UP | CACCGGCGAGGTATTCGGCTCCGCG | SEQ ID NO: 114 |
| K2 | NTC-sg2-UP | CACCGGCTTTCACGGAGGTTCGACG | SEQ ID NO: 115 |
| K3 | NTC-sg3-UP | CACCGATGTTGCAGTTCGGCTCGAT | SEQ ID NO: 116 |
| K4 | NTC-sg4-UP | CACCGACGTGTAAGGCGAACGCCTT | SEQ ID NO: 117 |
| K5 | NTC-sg5-UP | CACCGGACTCCGGGTACTAAATGTC | SEQ ID NO: 118 |
| K6 | NTC-sg6-UP | CACCGCCGCGCCGTTAGGGAACGAG | SEQ ID NO: 119 |
| K7 | NTC-sg7-UP | CACCGATTGTTCGACCGTCTACGGG | SEQ ID NO: 120 |
| K8 | NTC-sg8-UP | CACCGACCCATCGGGTGCGATATGG | SEQ ID NO: 121 |
| K9 | NTC-sg9-UP | CACCGCGGGCGTCACCTGCTAGTAA | SEQ ID NO: 122 |
| K10 | NTC-sg10-UP | CACCGGCTTCTACTCGCAACGTATT | SEQ ID NO: 123 |
| L1 | NTC-sg1-DN | AAACCGCGGAGCCGAATACCTCGCC | SEQ ID NO: 124 |
| L2 | NTC-sg2-DN | AAACCGTCGAACCTCCGTGAAAGCC | SEQ ID NO: 125 |
| L3 | NTC-sg3-DN | AAACATCGAGCCGAACTGCAACATC | SEQ ID NO: 126 |
| L4 | NTC-sg4-DN | AAACAAGGCGTTCGCCTTACACGTC | SEQ ID NO: 127 |
| L5 | NTC-sg5-DN | AAACGACATTTAGTACCCGGAGTCC | SEQ ID NO: 128 |
| L6 | NTC-sg6-DN | AAACCTCGTTCCCTAACGGCGCGGC | SEQ ID NO: 129 |
| L7 | NTC-sg7-DN | AAACCCCGTAGACGGTCGAACAATC | SEQ ID NO: 130 |
| L8 | NTC-sg8-DN | AAACCCATATCGCACCCGATGGGTC | SEQ ID NO: 131 |
| L9 | NTC-sg9-DN | AAACTTACTAGCAGGTGACGCCCGC | SEQ ID NO: 132 |
| L10 | NTC-sg10-DN | AAACAATACGTTGCGAGTAGAAGCC | SEQ ID NO: 133 |

TABLE 6
Additional CRISPR sgRNA sequences, listing SEQ ID NO: 134-231.

| ID | sgRNA name | Nucleotide sequence | SEQ ID |
|---|---|---|---|
| A1a | Mett13-sg1-UP | TAGGCACGGGACTATCACTA | SEQ ID NO: 134 |
| A2a | Mett13-sg2-UP | TCAGGTGATTACCGTAGAGA | SEQ ID NO: 135 |
| A3a | Mett13-sg3-UP | AGGTAGCAGGGACCATCGCA | SEQ ID NO: 136 |
| A4a | Mett13-sg4-UP | CTGAAGTGCAGCTTGCGACA | SEQ ID NO: 137 |
| A5a | Mett13-sg5-UP | ACGCCGTTTCTGCCCTGCGA | SEQ ID NO: 138 |
| A6a | Mett13-sg6-UP | CTTGTCGCAAGCTGCACTTC | SEQ ID NO: 139 |
| A7a | Mett13-sg7-UP | GTTGAAAAGTTTCGCTCTCG | SEQ ID NO: 140 |
| A8a | Mett13-sg8-UP | GTTGGGGACAGTGCCGCTTC | SEQ ID NO: 141 |
| A9a | Mett13-sg9-UP | GCAGAAACGGCGTGCAGAAC | SEQ ID NO: 142 |
| B1a | Mett13-sg1-DN | TAGTGATAGTCCCGTGCCTAC | SEQ ID NO: 143 |
| B2a | Mett13-sg2-DN | TCTCTACGGTAATCACCTGAC | SEQ ID NO: 144 |
| B3a | Mett13-sg3-DN | TGCGATGGTCCCTGCTACCTC | SEQ ID NO: 145 |
| B4a | Mett13-sg4-DN | TGTCGCAAGCTGCACTTCAGC | SEQ ID NO: 146 |
| B5a | Mett13-sg5-DN | TCGCAGGGCAGAAACGGCGTC | SEQ ID NO: 147 |
| B6a | Mett13-sg6-DN | GAAGTGCAGCTTGCGACAAGC | SEQ ID NO: 148 |
| B7a | Mett13-sg7-DN | CGAGAGCGAAACTTTTCAACC | SEQ ID NO: 149 |
| B8a | Mett13-sg8-DN | GAAGCGGCACTGTCCCCAACC | SEQ ID NO: 150 |
| B9a | Mett13-sg9-DN | GTTCTGCACGCCGTTTCTGCC | SEQ ID NO: 151 |
| C1a | Mett114-sg1-UP | GTCCAGTGTCTACAAAATGT | SEQ ID NO: 152 |
| C2a | Mett114-sg2-UP | CACTGAACTACTTACATGGG | SEQ ID NO: 153 |
| C3a | Mett114-sg3-UP | ATCAACTTACTACTCTCCCA | SEQ ID NO: 154 |
| C4a | Mett114-sg4-UP | GCTGGACCTGGGATGATGTA | SEQ ID NO: 155 |
| C5a | Mett114-sg5-UP | CGCATACCTGCAGGTTTCTC | SEQ ID NO: 156 |
| C6a | Mett114-sg6-UP | ACAGACCTCAGAATTTCATC | SEQ ID NO: 157 |
| C7a | Mett114-sg7-UP | AGATTCCAGTACCTTTCTTA | SEQ ID NO: 158 |
| C8a | Mett114-sg8-UP | CATCTGCTCCAAACTCAAAA | SEQ ID NO: 159 |
| C9a | Mett114-sg9-UP | ACATCCCTGATGAAATTCTG | SEQ ID NO: 160 |
| C10a | Mett114-sg10-UP | CTAGTTGGGAGCTGAGAGTG | SEQ ID NO: 161 |
| D1a | Mett114-sg1-DN | ACATTTTGTAGACACTGGACC | SEQ ID NO: 162 |
| D2a | Mett114-sg2-DN | CCCATGTAAGTAGTTCAGTGC | SEQ ID NO: 163 |
| D3a | Mett114-sg3-DN | TGGGAGAGTAGTAAGTTGATC | SEQ ID NO: 164 |
| D4a | Mett114-sg4-DN | TACATCATCCCAGGTCCAGCC | SEQ ID NO: 165 |
| D5a | Mett114-sg5-DN | GAGAAACCTGCAGGTATGCGC | SEQ ID NO: 166 |
| D6a | Mett114-sg6-DN | GATGAAATTCTGAGGTCTGTC | SEQ ID NO: 167 |
| D7a | Mett114-sg7-DN | TAAGAAAGGTACTGGAATCTC | SEQ ID NO: 168 |
| D8a | Mett114-sg8-DN | TTTTGAGTTTGGAGCAGATGC | SEQ ID NO: 169 |
| D9a | Mett114-sg9-DN | CAGAATTTCATCAGGGATGTC | SEQ ID NO: 170 |
| D10a | Mett114-sg10-DN | CACTCTCAGCTCCCAACTAGC | SEQ ID NO: 171 |
| E1a | Alkbh5-sg1-UP | CCCCGAGCGCAGCGACTACG | SEQ ID NO: 172 |
| E2a | Alkbh5-sg2-UP | AGAAAGACACGGACACGATG | SEQ ID NO: 173 |
| E3a | Alkbh5-sg3-UP | CCTGTACAACGAGCACACGG | SEQ ID NO: 174 |
| E4a | Alkbh5-sg4-UP | GTCACGCTCCCCCTGCGCAC | SEQ ID NO: 175 |
| E5a | Alkbh5-sg5-UP | GGTGGTGTCCCGCGCCGAGA | SEQ ID NO: 176 |
| E6a | Alkbh5-sg6-UP | ATCTCGTCGACGTCGCCCGG | SEQ ID NO: 177 |
| E7a | Alkbh5-sg7-UP | AAGATTAGATGCACCGCGAT | SEQ ID NO: 178 |
| E8a | Alkbh5-sg8-UP | TATGCTTCAGATCGCCTGTC | SEQ ID NO: 179 |
| E9a | Alkbh5-sg9-UP | CTCAGGACATTAAGGAACGC | SEQ ID NO: 180 |
| E10a | Alkbh5-sg10-UP | GCAATCTTCCGAGGACTCAT | SEQ ID NO: 181 |
| F1a | Alkbh5-sg1-DN | CGTAGTCGCTGCGCTCGGGGC | SEQ ID NO: 182 |
| F2a | Alkbh5-sg2-DN | CATCGTGTCCGTGTCTTTCTC | SEQ ID NO: 183 |
| F3a | Alkbh5-sg3-DN | CCGTGTGCTCGTTGTACAGGC | SEQ ID NO: 184 |
| F4a | Alkbh5-sg4-DN | GTGCGCAGGGGGAGCGTGACC | SEQ ID NO: 185 |
| F5a | Alkbh5-sg5-DN | TCTCGGCGCGGGACACCACCC | SEQ ID NO: 186 |
| F6a | Alkbh5-sg6-DN | CCGGGCGACGTCGACGAGATC | SEQ ID NO: 187 |
| F7a | Alkbh5-sg7-DN | ATCGCGGTGCATCTAATCTTC | SEQ ID NO: 188 |
| F8a | Alkbh5-sg8-DN | GACAGGCGATCTGAAGCATAC | SEQ ID NO: 189 |
| F9a | Alkbh5-sg9-DN | GCGTTCCTTAATGTCCTGAGC | SEQ ID NO: 190 |
| F10a | Alkbh5-sg10-DN | ATGAGTCCTCGGAAGATTGCC | SEQ ID NO: 191 |
| G1a | Fto-sg1-UP | CGGTGAGTGGAACTAAACCG | SEQ ID NO: 192 |
| G2a | Fto-sg2-UP | GCAGTGTGAGAAAGGCCTCG | SEQ ID NO: 193 |
| G3a | Fto-sg3-UP | AGGAGCCCTATTTCGGCATG | SEQ ID NO: 194 |
| G4a | Fto-sg4-UP | AGTGTCTCGCATCCTCATCG | SEQ ID NO: 195 |
| G5a | Fto-sg5-UP | GCTTGTTTCGGGACGTGGTG | SEQ ID NO: 196 |
| G6a | Fto-sg6-UP | GTGTACAGCTATAGCTGCGA | SEQ ID NO: 197 |
| G7a | Fto-sg7-UP | GTTTAGTTCCACTCACCGTG | SEQ ID NO: 198 |
| G8a | Fto-sg8-UP | TGAAGAGGGATTGTTAATCC | SEQ ID NO: 199 |
| G9a | Fto-sg9-UP | AGATGATGAGTTCTATCAGC | SEQ ID NO: 200 |
| G10a | Fto-sg10-UP | ATTAACAATCCCTCTTCACC | SEQ ID NO: 201 |
| H1a | Fto-sg1-DN | CGGTTTAGTTCCACTCACCGC | SEQ ID NO: 202 |
| H2a | Fto-sg2-DN | CGAGGCCTTTCTCACACTGCC | SEQ ID NO: 203 |
| H3a | Fto-sg3-DN | CATGCCGAAATAGGGCTCCTC | SEQ ID NO: 204 |
| H4a | Fto-sg4-DN | CGATGAGGATGCGAGACACTC | SEQ ID NO: 205 |
| H5a | Fto-sg5-DN | CACCACGTCCCGAAACAAGCC | SEQ ID NO: 206 |
| H6a | Fto-sg6-DN | TCGCAGCTATAGCTGTACACC | SEQ ID NO: 207 |
| H7a | Fto-sg7-DN | CACGGTGAGTGGAACTAAACC | SEQ ID NO: 208 |
| H8a | Fto-sg8-DN | GGATTAACAATCCCTCTTCAC | SEQ ID NO: 209 |
| H9a | Fto-sg9-DN | GCTGATAGAACTCATCATCTC | SEQ ID NO: 210 |
| H10a | Fto-sg10-DN | GGTGAAGAGGGATTGTTAATC | SEQ ID NO: 211 |
| K1a | NTC-sg1-UP | GCGAGGTATTCGGCTCCGCG | SEQ ID NO: 212 |
| K2a | NTC-sg2-UP | GCTTTCACGGAGGTTCGACG | SEQ ID NO: 213 |
| K3a | NTC-sg3-UP | ATGTTGCAGTTCGGCTCGAT | SEQ ID NO: 214 |
| K4a | NTC-sg4-UP | ACGTGTAAGGCGAACGCCTT | SEQ ID NO: 215 |
| K5a | NTC-sg5-UP | GACTCCGGGTACTAAATGTC | SEQ ID NO: 216 |
| K6a | NTC-sg6-UP | CCGCGCCGTTAGGGAACGAG | SEQ ID NO: 217 |
| K7a | NTC-sg7-UP | ATTGTTCGACCGTCTACGGG | SEQ ID NO: 218 |
| K8a | NTC-sg8-UP | ACCCATCGGGTGCGATATGG | SEQ ID NO: 219 |
| K9a | NTC-sg9-UP | CGGGCGTCACCTGCTAGTAA | SEQ ID NO: 220 |
| K10a | NTC-sg10-UP | GCTTCTACTCGCAACGTATT | SEQ ID NO: 221 |
| L1a | NTC-sg1-DN | CGCGGAGCCGAATACCTCGCC | SEQ ID NO: 222 |
| L2a | NTC-sg2-DN | CGTCGAACCTCCGTGAAAGCC | SEQ ID NO: 223 |
| L3a | NTC-sg3-DN | ATCGAGCCGAACTGCAACATC | SEQ ID NO: 224 |
| L4a | NTC-sg4-DN | AAGGCGTTCGCCTTACACGTC | SEQ ID NO: 225 |
| L5a | NTC-sg5-DN | GACATTTAGTACCCGGAGTCC | SEQ ID NO: 226 |
| L6a | NTC-sg6-DN | CTCGTTCCCTAACGGCGCGGC | SEQ ID NO: 227 |
| L7a | NTC-sg7-DN | CCCGTAGACGGTCGAACAATC | SEQ ID NO: 228 |
| L8a | NTC-sg8-DN | CCATATCGCACCCGATGGGTC | SEQ ID NO: 229 |
| L9a | NTC-sg9-DN | TTACTAGCAGGTGACGCCCGC | SEQ ID NO: 230 |
| L10a | NTC-sg10-DN | AATACGTTGCGAGTAGAAGCC | SEQ ID NO: 231 |

What is claimed is:

1. A method of treating or preventing a Zika viral infection in a subject in need thereof, the method comprising administering an effective amount of an adenosine N-6 methylation agonist or an adenosine N-6 demethylation antagonist.

2. The method of claim 1, wherein said adenosine N-6 methylation agonist is a RNA methyltransferase complex agonist, a N6-adenosine-methyltransferase (METTL3) agonist, or a Methyltransferase-Like Protein 14 (METTL14) agonist.

3. The method of claim 1, wherein said adenosine N-6 methylation agonist is a METTL3 or a METTL14 agonist.

4. The method of claim 1, wherein said adenosine N-6 demethylation antagonist is an alkB homolog 5 RNA demethylase (ALKBH5) antagonist or an alpha-ketoglutarate-dependent dioxygenase (FTO) antagonist.

5. The method of claim 1, wherein said adenosine N-6 methylation agonist is an adenosine N-6 methylation antisense nucleic acid agonist and said adenosine N-6 demethylation antagonist is an adenosine N-6 demethylation antisense nucleic acid antagonist.

6. The method of claim 5, wherein said N-6 methylation antisense nucleic acid agonist is an N-6 methylation RNAi agonist and said adenosine N-6 demethylation antisense nucleic acid antagonist is an adenosine N-6 demethylation RNAi antagonist.

7. The method of claim 1, wherein said adenosine N-6 methylation agonist is an adenosine N-6 methylation aptamer agonist or an adenosine N-6 methylation antibody agonist.

8. The method of claim 1, wherein said adenosine N-6 demethylation antagonist is an adenosine N-6 demethylation aptamer antagonist or an adenosine N-6 demethylation antibody antagonist.

9. The method of claim 1, further comprising administering an immunogenic agent.

10. The method of claim 1, wherein said adenosine N-6 methylation agonist is an adenosine N-6 methylation CRISPR agonist or adenosine N-6 demethylation antagonist is an adenosine N-6 demethylation CRISPR antagonist.

11. The method of claim 9, wherein said immunogenic agent and said adenosine N-6 methylation agonist or said adenosine N-6 demethylation antagonist together with a vaccine adjuvant form a vaccine formulation.

12. The method of claim 1, wherein said adenosine N-6 methylation agonist or said adenosine N-6 demethylation antagonist are capable of activating the immune system of said subject.

13. A method of treating or preventing a Zika viral infection in a subject in need thereof, the method comprising administering an effective amount of an RNA 2′-O-methyl transferase inhibitor.

14. The method of claim 13, wherein said RNA 2′-O-methyl transferase inhibitor is an RNA 2′-O-methyl transferase antisense inhibitor, an RNA 2′-O-methyl transferase CRISPR inhibitor, an RNA 2′-O-methyl aptamer inhibitor or an RNA 2′-O-methyl transferase antibody inhibitor.

15. The method of claim 13, wherein said RNA 2′-O-methyl transferase inhibitor is a nonstructural protein 5 (NS5) inhibitor.

16. The method of claim 13, wherein said RNA 2′-O-methyl transferase inhibitor is S-adenosylmethionine, a chemical analogue of S-adenosylmethionine, or a small molecule functional analogue of S-adenosylmethionine.

17. The method of claim 13, wherein said RNA 2′-O-methyl transferase inhibitor is a compound of the formula: 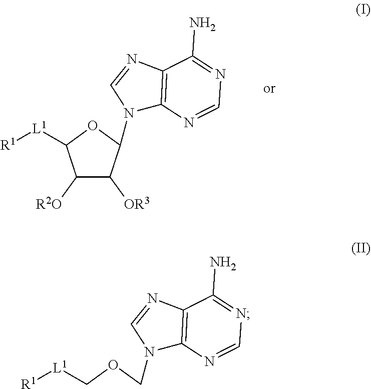
wherein,
L¹ is a bond, —O—, —S—, —NH—, —C(O)NH—, —C(O)—, —S(O)₂NH—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene;
R¹ is hydrogen, halogen, —CF₃, —CN, —OR^{1A}, —NHR^{1A}, —N₃, —SR^{1A}, —COOR^{1A}, —CONHR^{1A}, —NHC(O)R^{1A}, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl;
R^{1A} is hydrogen, halogen, —CF₃, —CN, —OH, —NH₂, —N₃, —SH, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl;
R² is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl; and
R³ is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

18. The method of claim 17, wherein R² and R³ are hydrogen.

19. The method of claim 17, wherein L¹ is —(CH₂)_{z1}—NH—(CH₂)_{z2}—, —(CH₂)_{z1}—S—(CH₂)_{z2}—, —(CH₂)_{z1}—O—(CH₂)_{z2}—, —(CH₂)_{z1}—C(O)NH—(CH₂)_{z2}—, —(CH₂)_{z1}—C(O)O—(CH₂)_{z2}—, or —(CH₂)_{z1}—S(O)₂NH(CH₂)_{z2}, wherein z1 and z2 are independently integers from 0 to 10.

20. The method of claim 19, wherein z1 and z2 are independently integers from 0 to 5.

21. The method of claim 19, wherein z1 and z2 are independently integers from 0 to 3.

22. The method of claim 19, wherein z1 and z2 are 1.

23. The method of claim 19, wherein z1 is an integer from 0 to 3, and z2 is 0.

24. The method of claim 19, wherein z1 is 1, and z2 is 0.

25. The method of claim 19, wherein z2 is an integer from 0 to 3, and z1 is 0.

26. The method of claim 19, wherein z1 is 0, and z2 is 1.

27. The method of claim 17, wherein R¹ is substituted or unsubstituted cycloalkyl, substituted or unsubstituted heterocycloalkyl, substituted or unsubstituted aryl or substituted or unsubstituted heteroaryl.

28. The method of claim 17, wherein R¹ is substituted or unsubstituted heterocycloalkyl, substituted or unsubstituted aryl or substituted or unsubstituted heteroaryl.

29. The method of claim 17, wherein R¹ is substituted or unsubstituted purinyl, substituted or unsubstituted imidazopyridinyl, substituted or unsubstituted imidazopyridinyl, substituted or unsubstituted benzoimidazolyl, substituted or unsubstituted benzotriazolyl, substituted or unsubstituted indazolyl, substituted or unsubstituted dihydropurinonyl, substituted or unsubstituted pyrazolopyridinyl, substituted or unsubstituted dihydropurinedionyl, substituted or unsubstituted furanyl, substituted or unsubstituted dihydrofuroquiozalinyl, substituted or unsubstituted pyrimidoindolyl, substituted or unsubstituted carbazolyl, substituted or unsubstituted indenyl, substituted or unsubstituted dihydrocyclopenta-isoindolyl, substituted or unsubstituted pyridinyl, substituted or unsubstituted pyrimidinyl, substituted or unsubstituted pyridazinyl, substituted or unsubstituted triazinyl, substituted or unsubstituted quinolinyl, substituted or unsubstituted quinazolinyl, substituted or unsubstituted quinoxalinyl, substituted or unsubstituted pyrrolyl, substituted or unsubstituted imidazolyl, substituted or unsubstituted oxazolyl, substituted or unsubstituted isoxazolyl, substituted or unsubstituted thiazolyl, substituted or unsubstituted isothizolyl, substituted or unsubstituted triazolyl, substituted or unsubstituted oxadiazolyl, substituted or unsubstituted thiadiazolyl, substituted or unsubstituted indolyl, substituted or unsubstituted pyrazolopyridinyl, substituted or unsubstituted benzofuranyl, substituted or unsubstituted benzothiophenyl, substituted or unsubstituted acridinyl, or substituted or unsubstituted phenyl.

30. The method of claim 17, wherein R^{1A} is hydrogen, halogen, —CF₃, —CN, —OH, —NH₂, —N₃, —SH, —COOH, —NHCOOH, —CONH₂, R^{1B}-substituted or unsubstituted alkyl, R^{1B}-substituted or unsubstituted heteroalkyl, R^{1B}-substituted or unsubstituted cycloalkyl, R^{1B}-substituted or unsubstituted heterocycloalkyl, R^{1B}-substituted or unsubstituted aryl or R^{1B}-substituted or unsubstituted heteroaryl;
R^{1B} is independently halogen, —CF₃, —CN, —OH, —NH₂, —N₃, —NO₂, —SH, —COOH, —NHCOOH, —CONH₂, R^{1C}-substituted or unsubstituted alkyl, R^{1C}-substituted or unsubstituted heteroalkyl, R^{1C}-substituted or unsubstituted cycloalkyl, R^{1C}-substituted or unsubstituted heterocycloalkyl, R^{1C}-substituted or unsubstituted aryl or R^{1C}-substituted or unsubstituted heteroaryl; and
R^{1C} is independently halogen, —CF₃, —CN, —OH, —NH₂, —N₃, —SH, —COOH, —NHCOOH, —CONH₂, unsubstituted alkyl, unsubstituted heteroalkyl, unsubstituted cycloalkyl, unsubstituted heterocycloalkyl, unsubstituted aryl or unsubstituted heteroaryl.

31. The method of claim 29, wherein L¹ is a bond, —O—, —S—, —NH—, —C(O)NH—, —C(O)—, —S(O)₂NH—, R^{1D}-substituted or unsubstituted alkylene, R^{1D}-substituted or unsubstituted heteroalkylene, R^{1D}-substituted or unsubstituted cycloalkylene, R^{1D}-substituted or unsubstituted heterocycloalkylene, R^{1D}-substituted or unsubstituted arylene or R^{1D}-substituted or unsubstituted heteroarylene, wherein
R^{1D} is halogen, —CF₃, —CN, —OH, —NH₂, —N₃, —SH, —COOH, —NHCOOH, —CONH₂, R^{1E}-substituted or unsubstituted alkyl, R^{1E}-substituted or unsubstituted heteroalkyl, R^{1E-}substituted or unsubstituted cycloalkyl, R^{1E}-substituted or unsubstituted heterocycloalkyl, R^{1E-}substituted or unsubstituted aryl or R^{1E}-substituted or unsubstituted heteroaryl; and wherein
R^{1E} is halogen, —CF₃, —CN, —OH, —NH₂, —N₃, —SH, —COOH, —NHCOOH, —CONH₂, unsubstituted alkyl, unsubstituted heteroalkyl, unsubstituted cycloalkyl, unsubstituted heterocycloalkyl, unsubstituted aryl or unsubstituted heteroaryl.

32. The method of claim 17, wherein the compound has the formula: 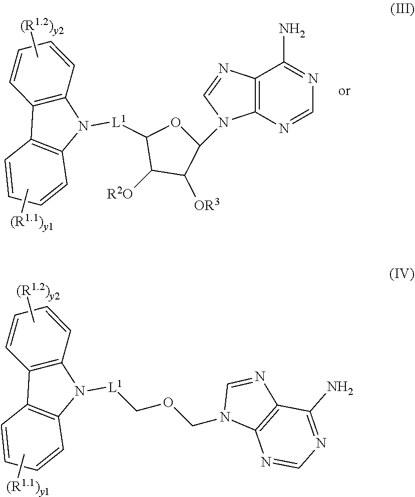
wherein,
y1 and y2 are independently an integer from 0 to 1,
R^{1.1} and R^{1.2} independently are halogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCH₃, —OCBr₃, —OCCl₃, —OCI₃, —OCHF₂, —OCHBr₂, —OCHCl₂, —OCHI₂, —OCH₂F, —OCH₂Br, —OCH₂Cl, —OCH₂I, —CN, —O H, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

33. The method of claim 32, wherein R² and R³ are hydrogen.

34. The method of claim 32, wherein y1 and y2 are 0.

35. The method of claim 32, wherein y1 and y2 are 1.

36. The method of claim 32, wherein y1 is 0 and y2 is 1.

37. The method of claim 32, wherein y1 is 1 and y2 is 0.

38. The method of claim 32, wherein y1 is 1, y2 is 0, and R^{1.1} is —CH₃ or —CH₂F.

39. The method of claim 32, wherein y1 is 0, y2 is 1, and R^{1.2} is —CH₂F, —CF₃, —CH₃, —OCH₃ or —C(CH₃)₃.

40. The method of claim 32, wherein y1 and y2 are 1; and R^{1.2} is unsubstituted 2 C₁-C₄ alkyl; and R^{1.1} is unsubstituted C₁-C₂ alkyl.

41. The method of claim 32, wherein
R^{1.1} is —CH₂F and R^{1.2} is —C(CH₃)₃;
R^{1.1} is —CH₃ and R^{1.2} is —C(CH₃)₃;
R^{1.1} is —CH₃ and R^{1.2} is —CF₃;
R^{1.1} and R^{1.2} are each —CH₃;
R^{1.1} is —CH₃ and R^{1.2} is —CH₂F;
R^{1.1} is —CH₃ and R^{1.2} is —OCH₃;
R^{1.1} is —CF₃ and R^{1.2} is —OCH₃;
R^{1.1} is —CF₃ and R^{1.2} is —CH₃;
R^{1.1} is —OCH₃ and R^{1.2} is —CH₃; or
R^{1.1} is —OCH₃ and R^{1.2} is —CH₃.

42. The method of claim 32, wherein the compound has the formula:

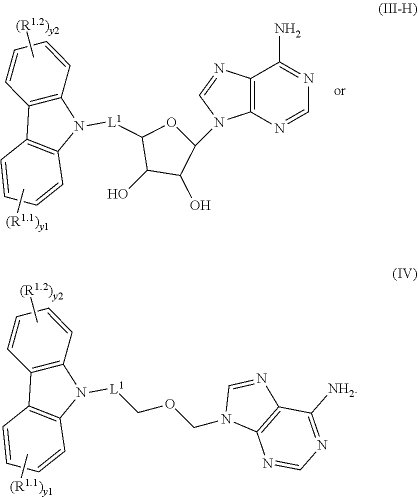

43. The method of claim 42, wherein y1 is 1, y2 is 0, and R^{1.1} is —CH₃ or —CH₂F.

44. The method of claim 42, wherein y1 is 0, y2 is 1, and R^{1.2} is —CH₂F, —CF₃, —CH₃, —OCH₃ or —C(CH₃)₃.

45. The method of claim 42, wherein y1 and y2 are 1.

46. The method of claim 45, wherein
R^{1.1} is —CH₂F and R^{1.2} is —C(CH₃)₃;
R^{1.1} is —CH₃ and R^{1.2} is —C(CH₃)₃;
R^{1.1} is —CH₃ and R^{1.2} is —CF₃;
R^{1.1} and R^{1.2} are each —CH₃;
R^{1.1} is —CH₃ and R^{1.2} is —CH₂F;
R^{1.1} is —CH₃ and R^{1.2} is —OCH₃;
R^{1.1} is —CF₃ and R^{1.2} is —OCH₃;
R^{1.1} is —CF₃ and R^{1.2} is —CH₃;
R^{1.1} is —OCH₃ and R^{1.2} is —CH₃; or
R^{1.1} is —OCH₃ and R^{1.2} is —CH₃.

47. A method of treating or preventing an RNA virus infection in a subject in need thereof, the method comprising administering an effective amount of a compound of the formula: 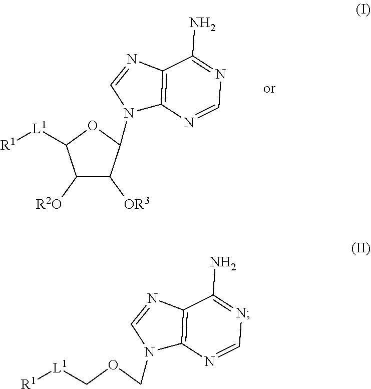
wherein,
L¹ is a bond, —O—, —S—, —NH—, —C(O)NH—, —C(O)—, —S(O)₂NH—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene;
R¹ is hydrogen, halogen, —CF₃, —CN, —OR^{1A}, —NHR^{1A}, —N₃, —SR^{1A}, —COOR^{1A}, —CONHR^{1A}, —NHC(O)R^{1A}, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl;
R^{1A} is hydrogen, halogen, —CF₃, —CN, —OH, —NH₂, —N₃, —SH, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl;
R² is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl; and
R³ is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

48. The method of claim 47, wherein R² and R³ are hydrogen.

49. The method of claim 47, wherein L¹ is —(CH₂)_{z1}—NH—(CH₂)_{z2}—, —(CH₂)_{z1}—S—(CH₂)_{z2}—, —(CH₂)_{z1}—O—(CH₂)_{z2}—, —(CH₂)_{z1}—C(O)NH—(CH₂)_{z2}—, —(CH₂)_{z1}—C(O)O—(CH₂)_{z2}—, or —(CH₂)_{z1}—S(O)₂NH(CH₂)_{z2}, wherein z1 and z2 are independently integers from 0 to 10.

50. The method of claim 49, wherein z1 and z2 are independently integers from 0 to 5.

51. The method of claim 49, wherein z1 and z2 are independently integers from 0 to 3.

52. The method of claim 49, wherein z1 and z2 are 1.

53. The method of claim 49, wherein z1 is an integer from 0 to 3, and z2 is 0.

54. The method of claim 49, wherein z1 is 1, and z2 is 0.

55. The method of claim 49, wherein z2 is an integer from 0 to 3, and z1 is 0.

56. The method of claim 49, wherein z1 is 0, and z2 is 1.

57. The method of claim 47, wherein R¹ is substituted or unsubstituted cycloalkyl, substituted or unsubstituted heterocycloalkyl, substituted or unsubstituted aryl or substituted or unsubstituted heteroaryl.

58. The method of claim 47, wherein R¹ is substituted or unsubstituted heterocycloalkyl, substituted or unsubstituted aryl or substituted or unsubstituted heteroaryl.

59. The method of claim 47, wherein R¹ is substituted or unsubstituted purinyl, substituted or unsubstituted imidazopyridinyl, substituted or unsubstituted imidazopyridinyl, substituted or unsubstituted benzoimidazolyl, substituted or unsubstituted benzotriazolyl, substituted or unsubstituted indazolyl, substituted or unsubstituted dihydropurinonyl, substituted or unsubstituted pyrazolopyridinyl, substituted or unsubstituted dihydropurinedionyl, substituted or unsubstituted furanyl, substituted or unsubstituted dihydrofuroquiozalinyl, substituted or unsubstituted pyrimidoindolyl, substituted or unsubstituted carbazolyl, substituted or unsubstituted indenyl, substituted or unsubstituted dihydrocyclopenta-isoindolyl, substituted or unsubstituted pyridinyl, substituted or unsubstituted pyrimidinyl, substituted or unsubstituted pyridazinyl, substituted or unsubstituted triazinyl, substituted or unsubstituted quinolinyl, substituted or unsubstituted quinazolinyl, substituted or unsubstituted quinoxalinyl, substituted or unsubstituted pyrrolyl, substituted or unsubstituted imidazolyl, substituted or unsubstituted oxazolyl, substituted or unsubstituted isoxazolyl, substituted or unsubstituted thiazolyl, substituted or unsubstituted isothizolyl, substituted or unsubstituted triazolyl, substituted or unsubstituted oxadiazolyl, substituted or unsubstituted thiadiazolyl, substituted or unsubstituted indolyl, substituted or unsubstituted pyrazolopyridinyl, substituted or unsubstituted benzofuranyl, substituted or unsubstituted benzothiophenyl, substituted or unsubstituted acridinyl, or substituted or unsubstituted phenyl.

60. The method of claim 47, wherein R^{1A} is hydrogen, halogen, —CF₃, —CN, —OH, —NH₂, —N₃, —SH, —COOH, —NHCOOH, —CONH₂, R^{1B}-substituted or unsubstituted alkyl, R^{1B}-substituted or unsubstituted heteroalkyl, R^{1B}-substituted or unsubstituted cycloalkyl, R^{1B}-substituted or unsubstituted heterocycloalkyl, R^{1B}-substituted or unsubstituted aryl or R^{1B}-substituted or unsubstituted heteroaryl;
R^{1B} is independently halogen, —CF₃, —CN, —OH, —NH₂, —N₃, —NO₂, —SH, —COOH, —NHCOOH, —CONH₂, R^{1C}-substituted or unsubstituted alkyl, R^{1C}-substituted or unsubstituted heteroalkyl, R^{1C}-substituted or unsubstituted cycloalkyl, R^{1C}-substituted or unsubstituted heterocycloalkyl, R^{1C}-substituted or unsubstituted aryl or R^{1C}-substituted or unsubstituted heteroaryl; and
R^{1C} is independently halogen, —CF₃, —CN, —OH, —NH₂, —N₃, —SH, —COOH, —NHCOOH, —CONH₂, unsubstituted alkyl, unsubstituted heteroalkyl, unsubstituted cycloalkyl, unsubstituted heterocycloalkyl, unsubstituted aryl or unsubstituted heteroaryl.

61. The method of claim 60, wherein L¹ is a bond, —O—, —S—, —NH—, —C(O)NH—, —C(O)—, —S(O)₂NH—, R^{1D}-substituted or unsubstituted alkylene, R^{1D}-substituted or unsubstituted heteroalkylene, R^{1D}-substituted or unsubstituted cycloalkylene, R^{1D}-substituted or unsubstituted heterocycloalkylene, R^{1D}-substituted or unsubstituted arylene or R^{1D}-substituted or unsubstituted heteroarylene, wherein
R^{1D} is halogen, —CF₃, —CN, —OH, —NH₂, —N₃, —SH, —COOH, —NHCOOH, —CONH₂, R^{1E}-substituted or unsubstituted alkyl, R^{1E}-substituted or unsubstituted heteroalkyl, R^{1E-}substituted or unsubstituted cycloalkyl, R^{1E}-substituted or unsubstituted heterocycloalkyl, R^{1E-}substituted or unsubstituted aryl or R^{1E}-substituted or unsubstituted heteroaryl; and wherein
R^{1E} is halogen, —CF₃, —CN, —OH, —NH₂, —N₃, —SH, —COOH, —NHCOOH, —CONH₂, unsubstituted alkyl, unsubstituted heteroalkyl, unsubstituted cycloalkyl, unsubstituted heterocycloalkyl, unsubstituted aryl or unsubstituted heteroaryl.

62. The method of claim 47, wherein the compound has the formula: 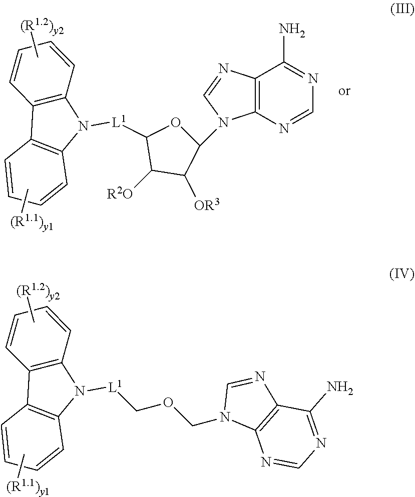
wherein,
y1 and y2 are independently an integer from 0 to 4,
R^{1.1} and R^{1.2} independently are halogen, —CF₃, —CBr₃, —CCl₃, —CI₃,
—CHF₂, —CHBr₂, —CHCl₂, —CHI₂, —CH₂F, —CH₂Br, —CH₂Cl, —CH₂I, —OCF₃, —OCH₃, —OCBr₃, —OCCl₃, —OCI₃, —OCHF₂, —OCHBr₂, —OCHCl₂, —OCHI₂, —OCH₂F, —OCH₂Br, —OCH₂Cl, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

63. The method of claim 62, wherein R² and R³ are hydrogen.

64. The method of claim 62, wherein y1 and y2 are 0.

65. The method of claim 62, wherein y1 and y2 are 1.

66. The method of claim 62, wherein y1 is 0 and y2 is 1.

67. The method of claim 62, wherein y1 is 1 and y2 is 0.

68. The method of claim 62, wherein y1 is 1, y2 is 0, and R^{1.1} is —CH₃ or —CH₂F.

69. The method of claim 62, wherein y1 is 0, y2 is 1, and R^{1.2} is —CH₂F, —CF₃, —CH₃, —OCH₃ or —C(CH₃)₃.

70. The method of claim 62, wherein y1 and y2 are 1.

71. The method of claim 62, wherein
R^{1.1} is —CH₂F and R^{1.2} is —C(CH₃)₃;
R^{1.1} is —CH₃ and R^{1.2} is —C(CH₃)₃;
R^{1.1} is —CH₃ and R^{1.2} is —CF₃;
R^{1.1} and R^{1.2} are each —CH₃;
R^{1.1} is —CH₃ and R^{1.2} is —CH₂F;
R^{1.1} is —CH₃ and R^{1.2} is —OCH₃;
R^{1.1} is —CF₃ and R^{1.2} is —OCH₃;
R^{1.1} is —CF₃ and R^{1.2} is —CH₃;
R^{1.1} is —OCH₃ and R^{1.2} is —CH₃; or
R^{1.1} is —OCH₃ and R^{1.2} is —CH₃.

72. The method of claim 62, wherein the compound has the formula:

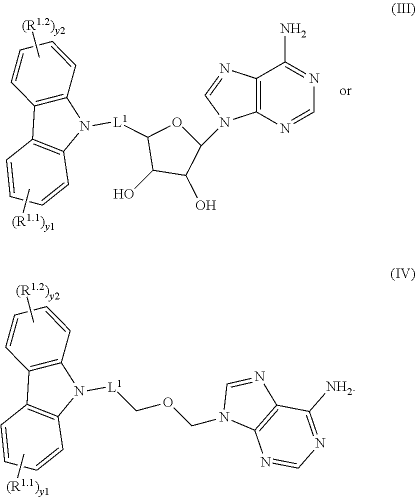

73. The method of claim 72, wherein y1 is 1, y2 is 0, and R^{1.1} is —CH₃ or —CH₂F.

74. The method of claim 72, wherein y1 is 0, y2 is 1, and R^{1.2} is —CH₂F, —CF₃, —CH₃, —OCH₃ or —C(CH₃)₃.

75. The method of claim 72, wherein y1 and y2 are 1.

76. The method of claim 72, wherein
R^{1.1} is —CH₂F and R^{1.2} is —C(CH₃)₃;
R^{1.1} is —CH₃ and R^{1.2} is —C(CH₃)₃;
R^{1.1} is —CH₃ and R^{1.2} is —CF₃;
R^{1.1} and R^{1.2} are each —CH₃;
R^{1.1} is —CH₃ and R^{1.2} is —CH₂F;
R^{1.1} is —CH₃ and R^{1.2} is —OCH₃;
R^{1.1} is —CF₃ and R^{1.2} is —OCH₃;
R^{1.1} is —CF₃ and R^{1.2} is —CH₃;
R^{1.1} is —OCH₃ and R^{1.2} is —CH₃; or
R^{1.1} is —OCH₃ and R^{1.2} is —CH₃.

77. The method of claim 47, wherein said RNA virus infection is an HIV viral infection, West Nile viral infection, Dengue viral infection, Japanese encephalitis or a Zika viral infection.

78. A compound of the formula: 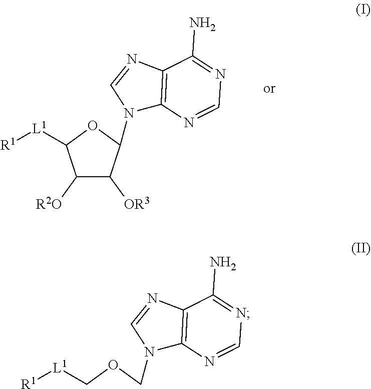
wherein,
L¹ is a bond, —O—, —S—, —NH—, —C(O)NH—, —C(O)—, —S(O)₂NH—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene;
R¹ is hydrogen, halogen, —CF₃, —CN, —OR^{1A}, —NHR^{1A}, —N₃, —SR^{1A}, —COOR^{1A}, —CONHR^{1A}, —NHC(O)R^{1A}, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl;
R^{1A} is hydrogen, halogen, —CF₃, —CN, —OH, —NH₂, —N₃, —SH, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl;
R² is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl; and
R³ is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

79. The compound of claim 78, wherein R² and R³ are hydrogen.

80. The compound of claim 78, wherein L¹ is —(CH₂)_{z1}—NH—(CH₂)_{z2}—, —(CH₂)_{z1}—S—(CH₂)_{z2}—, —(CH₂)_{z1}—O—(CH₂)_{z2}—, —(CH₂)_{z1}—C(O)NH—(CH₂)_{z2}—, —(CH₂)_{z1}—C(O)O—(CH₂)_{z2}—, or —(CH₂)_{z1}—S(O)₂NH(CH₂)_{z2}, wherein z1 and z2 are independently integers from 0 to 10.

81. The compound of claim 80, wherein z1 and z2 are independently integers from 0 to 5.

82. The compound of claim 80, wherein z1 and z2 are independently integers from 0 to 3.

83. The compound of claim 80, wherein z1 and z2 are 1.

84. The compound of claim 80, wherein z1 is an integer from 0 to 3, and z2 is 0.

85. The compound of claim 80, wherein z1 is 1, and z2 is 0.

86. The compound of claim 80, wherein z2 is an integer from 0 to 3, and z1 is 0.

87. The compound of claim 80, wherein z1 is 0, and z2 is 1.

88. The compound of claim 78, wherein R¹ is substituted or unsubstituted cycloalkyl, substituted or unsubstituted heterocycloalkyl, substituted or unsubstituted aryl or substituted or unsubstituted heteroaryl.

89. The compound of claim 78, wherein R¹ is substituted or unsubstituted heterocycloalkyl, substituted or unsubstituted aryl or substituted or unsubstituted heteroaryl.

90. The compound of claim 78, wherein R¹ is substituted or unsubstituted purinyl, substituted or unsubstituted imidazopyridinyl, substituted or unsubstituted imidazopyridinyl, substituted or unsubstituted benzoimidazolyl, substituted or unsubstituted benzotriazolyl, substituted or unsubstituted indazolyl, substituted or unsubstituted dihydropurinonyl, substituted or unsubstituted pyrazolopyridinyl, substituted or unsubstituted dihydropurinedionyl, substituted or unsubstituted furanyl, substituted or unsubstituted dihydrofuroquiozalinyl, substituted or unsubstituted pyrimidoindolyl, substituted or unsubstituted carbazolyl, substituted or unsubstituted indenyl, substituted or unsubstituted dihydrocyclopenta-isoindolyl, substituted or unsubstituted pyridinyl, substituted or unsubstituted pyrimidinyl, substituted or unsubstituted pyridazinyl, substituted or unsubstituted triazinyl, substituted or unsubstituted quinolinyl, substituted or unsubstituted quinazolinyl, substituted or unsubstituted quinoxalinyl, substituted or unsubstituted pyrrolyl, substituted or unsubstituted imidazolyl, substituted or unsubstituted oxazolyl, substituted or unsubstituted isoxazolyl, substituted or unsubstituted thiazolyl, substituted or unsubstituted isothizolyl, substituted or unsubstituted triazolyl, substituted or unsubstituted oxadiazolyl, substituted or unsubstituted thiadiazolyl, substituted or unsubstituted indolyl, substituted or unsubstituted pyrazolopyridinyl, substituted or unsubstituted benzofuranyl, substituted or unsubstituted benzothiophenyl, substituted or unsubstituted acridinyl, or substituted or unsubstituted phenyl.

91. The compound of claim 78, wherein R^{1A} is hydrogen, halogen, —CF₃, —CN, —OH, —NH₂, —N₃, —SH, —COOH, —NHCOOH, —CONH₂, R^{1B}-substituted or unsubstituted alkyl, R^{1B}-substituted or unsubstituted heteroalkyl, R^{1B}-substituted or unsubstituted cycloalkyl, R^{1B}-substituted or unsubstituted heterocycloalkyl, R^{1B}-substituted or unsubstituted aryl or R^{1B}-substituted or unsubstituted heteroaryl;
R^{1B} is independently halogen, —CF₃, —CN, —OH, —NH₂, —N₃, —NO₂, —SH, —COOH, —NHCOOH, —CONH₂, R^{1C}-substituted or unsubstituted alkyl, R^{1C}-substituted or unsubstituted heteroalkyl, R^{1C}-substituted or unsubstituted cycloalkyl, R^{1C}-substituted or unsubstituted heterocycloalkyl, R^{1C}-substituted or unsubstituted aryl or R^{1C}-substituted or unsubstituted heteroaryl; and
R^{1C} is independently halogen, —CF₃, —CN, —OH, —NH₂, —N₃, —SH, —COOH, —NHCOOH, —CONH₂, unsubstituted alkyl, unsubstituted heteroalkyl, unsubstituted cycloalkyl, unsubstituted heterocycloalkyl, unsubstituted aryl or unsubstituted heteroaryl.

92. The compound of claim 91, wherein L¹ is a bond, —O—, —S—, —NH—, —C(O)NH—, —C(O)—, —S(O)₂NH—, R^{1D}-substituted or unsubstituted alkylene, R^{1D}-substituted or unsubstituted heteroalkylene, R^{1D}-substituted or unsubstituted cycloalkylene, R^{1D}-substituted or unsubstituted heterocycloalkylene, R^{1D}-substituted or unsubstituted arylene or R^{1D}-substituted or unsubstituted heteroarylene, wherein
R^{1D} is halogen, —CF₃, —CN, —OH, —NH₂, —N₃, —SH, —COOH, —NHCOOH, —CONH₂, R^{1E}-substituted or unsubstituted alkyl, R^{1E}-substituted or unsubstituted heteroalkyl, R^{1E-}substituted or unsubstituted cycloalkyl, R^{1E}-substituted or unsubstituted heterocycloalkyl, R^{1E-}substituted or unsubstituted aryl or R^{1E}-substituted or unsubstituted heteroaryl; and wherein
R^{1E} is halogen, —CF₃, —CN, —OH, —NH₂, —N₃, —SH, —COOH, —NHCOOH, —CONH₂, unsubstituted alkyl, unsubstituted heteroalkyl, unsubstituted cycloalkyl, unsubstituted heterocycloalkyl, unsubstituted aryl or unsubstituted heteroaryl.

93. The compound of claim 78, wherein the compound has the formula: 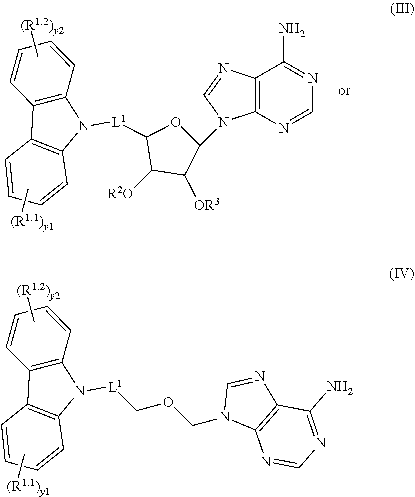
wherein,
y1 and y2 are independently an integer from 0 to 4,
R^{1.1} and R^{1.2} independently are halogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCH₃, —OCBr3, —OCCl₃, —OCI₃, —OCHF₂, —OCHBr₂, —OCHCl₂, —OCHI₂, —OCH₂F, —OCH₂Br, —OCH₂Cl, —OCH₂I, —CN, —OH, —NH₂, —COO H, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

94. The compound of claim 93, wherein R² and R³ are hydrogen.

95. The compound of claim 93, wherein y1 and y2 are 0.

96. The compound of claim 93, wherein y1 and y2 are 1.

97. The compound of claim 93, wherein y1 is 0 and y2 is 1.

98. The compound of claim 93, wherein y1 is 1 and y2 is 0.

99. The compound of claim 93, wherein y1 is 1, y2 is 0, and R^{1.1} is —CH₃ or —CH₂F.

100. The compound of claim 93, wherein y1 is 0, y2 is 1, and R^{1.2} is —CH₂F, —CF₃, —CH₃, —OCH₃ or —C(CH₃)₃.

101. The compound of claim 93, wherein y1 and y2 are 1.

102. The compound of claim 93, wherein
R^{1.1} is —CH₂F and R^{1.2} is —C(CH₃)₃;
R^{1.1} is —CH₃ and R^{1.2} is —C(CH₃)₃;
R^{1.1} is —CH₃ and R^{1.2} is —CF₃;
R^{1.1} and R^{1.2} are each —CH₃;
R^{1.1} is —CH₃ and R^{1.2} is —CH₂F;
R^{1.1} is —CH₃ and R^{1.2} is —OCH₃;
R^{1.1} is —CF₃ and R^{1.2} is —OCH₃;
R^{1.1} is —CF₃ and R^{1.2} is —CH₃;
R^{1.1} is —OCH₃ and R^{1.2} is —CH₃; or
R^{1.1} is —OCH₃ and R^{1.2} is —CH₃.

103. The compound of claim 93, wherein the compound has the formula:

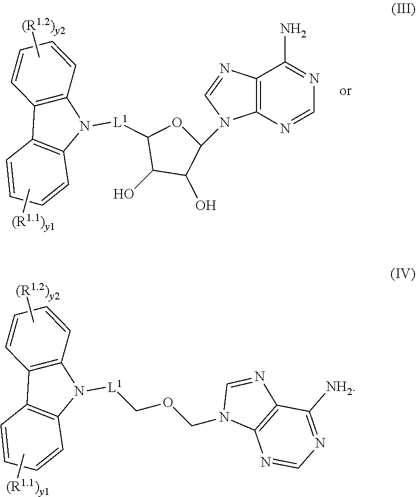

104. The compound of claim 103, wherein y1 is 1, y2 is 0, and R^{1.1} is —CH₃ or —CH₂F.

105. The compound of claim 103, wherein y1 is 0, y2 is 1, and R^{1.2} is —CH₂F, —CF₃, —CH₃, —OCH₃ or —C(CH₃)₃.

106. The compound of claim 103, wherein y1 and y2 are 1.

107. The compound of claim 103, wherein
R^{1.1} is —CH₂F and R^{1.2} is —C(CH₃)₃;
R^{1.1} is —CH₃ and R^{1.2} is —C(CH₃)₃;
R^{1.1} is —CH₃ and R^{1.2} is —CF₃;
R^{1.1} and R^{1.2} are each —CH₃;
R^{1.1} is —CH₃ and R^{1.2} is —CH₂F;
R^{1.1} is —CH₃ and R^{1.2} is —OCH₃;
R^{1.1} is —CF₃ and R^{1.2} is —OCH₃;
R^{1.1} is —CF₃ and R^{1.2} is —CH₃;
R^{1.1} is —OCH₃ and R^{1.2} is —CH₃; or
R^{1.1} is —OCH₃ and R^{1.2} is —CH₃.

108. A pharmaceutical composition comprising the compound of any one of claims 78-107 and a pharmaceutically acceptable excipient.

109. A vaccine composition comprising the compound of any one of claims 78-107, a vaccine adjuvant, and an immunogenic agent.

110. A pharmaceutical composition comprising an RNA compound, said RNA compound encoding an RNA virus structural protein.

111. The pharmaceutical composition of claim 110, wherein said RNA virus structural protein is a Zika virus structural protein.

112. The pharmaceutical composition of claim 110, wherein said RNA virus structural protein is a viral premembrane protein (prM), viral envelope protein (Env), a capsid protein (C) or a membrane protein (M).

113. The pharmaceutical composition of claim 110, wherein said RNA compound is modified on at least one 2′ position with a 2′O-methyl and/or modified on at least one adenosine at the N6 adenine position with a methyl.
